SEQUENCE LISTING (130 sequences)

SEQ ID NO: 1 - DNA, 954 bp, Synthetic construct
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 2 - DNA, 954 bp, synthetic construct
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtacagcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 3 - DNA, 954 bp, synthetic construct
  source 1..954: mol_type = other DNA
atgaaggtgctgtgggctgccctgctggtgactttcctggctggctgccaggctaaggtggagcaggctgtggagactgagcctgagcctgagctgaggcagcagactgagtggcagtctgggcagaggtgggagctggccctgggcaggttctgggactatctgaggtgggtgcagaccctgtctgagcaggtgcaggaggagctgctgagctctcaggtgactcaggagctgagggccctgatggatgagaccatgaaggagctgaaggcttacaagtctgagctggaggagcagctgacccctgtggctgaggagaccagagccaggctgagcaaggagctgcaggctgctcaggctaggctgggggctgacatggaggatgtgtgtggcaggctggtgcagtactctggggaggtgcaggccatgctgggccagagcactgaggagctgagggtgaggctggcttctcacctgaggaagctgagaaagaggctgctgagggatgctgatgatctgcagaagaggctggctgtgtaccaggctggggccagggaaggggctgagaggggcctgtctgccattagggagaggctggggcccctggtggagcaggggagggtgagggctgccactgtgggctctctggctggccagcccctgcaggagagagcccaggcttggggggagaggctgagggccaggatggaggagatgggctctaggaccagggataggctggatgaggtgaaggagcaggtggctgaggtcagggctaagctggaggagcaggcccagcagatcaggctgcaggctgaggccttccaggccaggctgaagagctggtttgagcccctggtggaggacatgcagaggcagtgggctgggctggtggagaaggtgcaggctgctgtggggacctctgctgcccctgtgccctctgacaatcactga

SEQ ID NO: 4 - DNA, 954 bp, synthetic construct
  source 1..954: mol_type = other DNA
atgaaggtgctgtgggctgccctgctggtgaccttcctggctggctgccaggccaaggtggagcaggctgtggagactgagcctgagcctgagctgaggcagcagactgagtggcagtctggccagaggtgggagctggccctgggcagattctgggactacctgaggtgggtgcagaccctgtctgagcaggtgcaggaggagctgctgagcagccaggtgacccaggagctgagggccctgatggatgagaccatgaaggagctgaaggcctataagtctgagctggaggagcagctgacccctgtggctgaggagaccagggctaggctgagcaaggagctgcaggctgcccaggctaggctgggggctgacatggaggatgtgtgtgggaggctggtgcagtactctggggaggtgcaggccatgctgggccagagcactgaggagctgagggtgaggctggccagccacctgaggaagctgagaaagaggctgctgagggatgctgatgatctgcagaagagactggctgtgtaccaggctggggccagggagggggctgaaaggggcctgtctgctatcagagagaggctgggccccctggtggagcagggcagggtgagggctgccactgtgggcagcctggctgggcagcccctgcaggaaagagcccaggcttggggggagagactgagggccaggatggaggagatgggcagcaggactagggacaggctggatgaggtgaaggagcaggtggctgaggtgagagccaagctggaggagcaggcccagcagatcaggctgcaggctgaggccttccaggccaggctgaagagctggtttgagcccctggtggaggatatgcagagacagtgggctggcctggtggagaaggtgcaggctgctgtgggcacctctgctgcccctgtgccttctgacaaccattga

SEQ ID NO: 5 - DNA, 954 bp, synthetic construct
  source 1..954: mol_type = other DNA
atgaaggtgctgtgggctgccctgctggtgacctttctggctgggtgccaggccaaggtggagcaggctgtggagactgagcctgagcctgagctgaggcagcagactgagtggcagtctgggcagaggtgggagctggccctggggaggttctgggactacctgaggtgggtgcagactctgtctgagcaggtgcaggaggagctgctgtctagccaggtgacccaggagctgagggccctgatggatgagaccatgaaggagctgaaggcctacaagtctgagctggaagagcagctgacccctgtggctgaggagaccagggctaggctgagcaaggaactgcaggctgctcaggccaggctgggggctgacatggaagatgtgtgtgggaggctggtgcagtactctggggaggtgcaggccatgctgggccagagcactgaggagctgagggtgaggctggccagccacctgaggaagctgaggaagaggctgctgagggatgctgatgacctgcagaagaggctggctgtgtaccaggctggggccagggagggggctgagaggggcctgtctgctatcagggagaggctgggccccctggtggagcagggcagggtgagggctgccactgtgggcagcctggctgggcagcctctgcaggagagggcccaggcctggggggagaggctgagagccaggatggaggagatgggctctaggactagggacaggctggatgaggtgaaggagcaggtggctgaggtgagggccaagctggaggagcaggctcagcagatcaggctgcaggctgaggctttccaggccaggctgaagtcttggtttgagcccctggtggaggatatgcagaggcagtgggctggcctggtggagaaggtgcaggctgctgtgggcacctctgctgcccctgtgccctctgacaaccattga

SEQ ID NO: 6 - DNA, 954 bp, synthetic construct
  source 1..954: mol_type = other DNA
atgaaggtgctgtgggctgccctgctggtgaccttcctggctggctgtcaggccaaggtggagcaggctgtggagactgagcctgagccagagctgaggcagcagactgagtggcagtctgggcagaggtgggagctggctctggggaggttctgggactacctgagatgggtgcagaccctgtctgaacaggtgcaggaggagctgctgagcagccaggtgacccaggagctgagagccctgatggatgagaccatgaaggagctgaaggcctacaagtctgagctggaggagcagctgactcctgtggctgaggagactagggccaggctgtctaaggagctgcaggctgcccaggccaggctgggggctgacatggaggatgtgtgtggcaggctggtgcagtactctggggaggtccaggccatgctgggccagagcactgaggagctgagggtgaggctggccagccacctgaggaagctgaggaagaggctgctgagggatgctgatgacctgcagaaaaggctggctgtgtaccaggctggggccagggagggggctgagagagggctgtctgccattagagagaggctgggccccctggtggagcagggcagggtgagggctgctactgtgggcagcctggctgggcagcctctgcaggagagggcccaggcctggggggagaggctgagggctaggatggaggagatgggctctaggaccagggacaggctggatgaggtgaaggagcaggtggctgaggtgagggccaagctggaggagcaggcccagcagatcaggctgcaggctgaggccttccaggctaggctgaagagctggtttgagcccctggtggaggacatgcagaggcagtgggctgggctggtggagaaggtgcaggctgctgtggggacctctgctgctcctgtgccctctgacaaccactga

SEQ ID NO: 7 - DNA, 954 bp, synthetic construct
  source 1..954: mol_type = other DNA
atgaaggtgctgtgggctgccctgctggtgaccttcctggctgggtgccaggctaaggtggagcaggctgtggagactgagcctgaacctgaactgaggcagcagactgagtggcagtctggccagaggtgggagctggctctggggaggttctgggactacctgaggtgggtgcagactctgtctgagcaggtgcaggaggagctgctgagcagccaggtgacccaggagctgagggccctgatggatgagaccatgaaggagctgaaggcctacaagtctgagctggaggagcagctgacccctgtggctgaggaaaccagggccaggctgtctaaggagctgcaggctgctcaggctaggctgggggctgacatggaggatgtgtgtgggagactggtgcagtattctggggaggtgcaggccatgctgggccagagcactgaggagctgagggtgaggctggctagccacctgagaaagctgaggaagaggctgctgagggatgctgatgatctgcagaagaggctggctgtgtatcaggctggggccagggagggggctgagaggggcctgtctgccatcagagagaggctggggcctctggtggagcagggcagggtgagggctgctactgtgggcagcctggctggccagcccctgcaggagagggcccaggcctggggggagaggctgagggctaggatggaggagatgggcagcagaaccagggacaggctggatgaggtgaaggagcaggtggctgaggtgagagccaagctggaggagcaggctcagcagatcaggctgcaggctgaggctttccaggctaggctgaagtcttggtttgagcccctggtggaagatatgcagaggcagtgggctggcctggtggagaaggtgcaggctgctgtgggcacctctgctgcccctgtgccctctgacaatcattga

SEQ ID NO: 8 - DNA, 954 bp, synthetic construct
  source 1..954: mol_type = other DNA
atgaaggtgctgtgggctgctctgctggtgaccttcctggctgggtgccaggccaaggtggagcaggctgtggagactgagccagagcctgagctgaggcagcagactgagtggcagtctggccagaggtgggagctggccctgggcaggttctgggactacctgaggtgggtgcagaccctgtctgagcaggtgcaggaggagctgctgagctctcaggtgactcaggagctgagggccctgatggatgagaccatgaaggagctgaaggcctacaagtctgagctggaggagcagctgacccctgtggctgaggagaccagggccaggctgagcaaggagctgcaggctgcccaggccaggctgggggctgacatggaggatgtgtgtggcaggctggtgcagtactctggggaggtgcaggccatgctgggccagtctactgaggagctgagggtgaggctggccagccatctgaggaagctgaggaagaggctgctgagggatgctgatgatctgcagaagaggctggctgtgtaccaggctggggccagggagggggctgagaggggcctgtctgccattagggagaggctggggcccctggtggagcagggcagggtgagggctgccactgtggggtctctggctggccagcccctgcaggagagggctcaggcctggggggagaggctgagggccagaatggaggaaatgggctctaggaccagggacaggctggatgaggtgaaggagcaggtggctgaggtgagggctaagctggaggagcaggctcagcagatcaggctgcaggctgaggccttccaggccaggctgaagagctggtttgagcccctggtggaagacatgcagaggcagtgggctggcctggtggagaaggtgcaggctgctgtgggcacctctgctgcccctgtgccctctgataaccactga

SEQ ID NO: 9 - DNA, 954 bp, synthetic construct
  source 1..954: mol_type = other DNA
atgaaggtgctgtgggctgccctgctggtgactttcctggctggctgtcaggccaaggtggagcaggctgtggagactgagcctgagcctgagctgaggcagcagactgagtggcagtctggccagaggtgggagctggccctgggcaggttctgggattacctgaggtgggtgcagaccctgtctgagcaggtgcaggaggagctgctgagctctcaggtgacccaggagctgagggccctgatggatgagaccatgaaggagctgaaggcctacaagtctgagctggaggagcagctgacccctgtggctgaggagaccagagctaggctgagcaaggagctgcaggctgctcaggccaggctgggggctgacatggaggatgtgtgtggcagactggtgcagtactctggggaggtgcaggctatgctggggcagagcactgaggagctgagagtgaggctggcctctcacctgaggaagctgaggaagaggctgctgagggatgctgatgacctgcagaagaggctggctgtctaccaggctggggccagggagggggctgagaggggcctgtctgccatcagggagaggctgggccccctggtggagcagggcagggtgagagctgccactgtggggagcctggctggccagcccctgcaggagagggcccaggcctggggggagaggctgagggctaggatggaggagatgggcagcaggaccagggacaggctggatgaggtgaaggagcaggtggctgaggtgagggccaagctggaggagcaggcccagcagattaggctgcaggctgaggccttccaggccaggctgaagagctggtttgagcctctggtggaggacatgcagagacagtgggctggcctggtggagaaggtgcaggctgctgtggggacctctgctgctccagtgccctctgataatcactga

SEQ ID NO: 10 - DNA, 954 bp, synthetic construct
  source 1..954: mol_type = other DNA
atgaaggtgctgtgggctgccctgctggtgaccttcctggctgggtgccaggctaaggtggagcaggctgtggagactgagcctgagcctgagctgagacagcagactgagtggcagtctggccagaggtgggagctggccctggggaggttctgggactacctgaggtgggtgcagaccctgtctgagcaggtgcaggaggagctgctgtcttctcaggtgacccaggagctgagggccctgatggatgagaccatgaaggagctgaaggcctacaagtctgagctggaggagcagctgactcctgtggctgaggagaccagggccaggctgtctaaggagctgcaggctgctcaggccaggctgggggctgacatggaggatgtgtgtggcaggctggtgcagtactctggggaggtgcaggccatgctggggcagagcactgaggagctgagggtgaggctggccagccatctgaggaagctgaggaagaggctgctgagggatgctgatgacctgcagaagaggctggctgtgtatcaggctggggccagggagggggctgagaggggcctgtctgccattagggagaggctgggccccctggtggagcaggggagggtgagggctgccactgtggggagcctggctgggcagcccctgcaggagagggcccaggcttggggggagaggctgagggccaggatggaggagatggggagcaggaccagggataggctggatgaggtcaaggagcaggtggctgaggtgagggctaagctggaggaacaggcccagcagatcaggctgcaggctgaggctttccaggccagactgaagagctggtttgagcccctggtggaggacatgcagaggcagtgggctggcctggtggagaaggtgcaggctgctgtgggcacctctgctgccccagtgccctctgataaccactga

SEQ ID NO: 11 - DNA, 954 bp, synthetic construct
  source 1..954: mol_type = other DNA
atgaaggtgctgtgggctgccctgctggtgactttcctggctggctgccaggctaaggtggagcaggctgtggagactgagccagagcctgagctgaggcagcagactgaatggcagtctggccagaggtgggagctggccctgggcaggttctgggactacctgagatgggtgcagaccctgtctgagcaggtccaggaggagctgctgtcttctcaggtgacccaggagctgagggccctgatggatgagactatgaaggagctgaaggcttacaagtctgagctggaggagcagctgacccctgtggctgaggagaccagggccagactgagcaaggagctgcaggctgctcaggccaggctgggggctgacatggaggatgtgtgtggcaggctggtgcagtattctggggaggtgcaggctatgctgggccagagcactgaggaactgagggtgaggctggcctctcatctgaggaagctgaggaagaggctgctgagggatgctgatgacctgcagaagaggctggctgtgtatcaggctggggccagggagggggctgagagggggctgtctgctatcagggagaggctgggccccctggtggagcaggggagagtgagggctgccactgtggggtctctggctggccagcccctgcaggagagggcccaggcctggggggagaggctgagggctagaatggaggagatggggtctaggaccagggacaggctggatgaggtcaaggaacaggtggctgaggtcagggccaagctggaggagcaggcccagcagattaggctgcaggctgaggcctttcaggccaggctgaagagctggtttgagcccctggtggaggacatgcagaggcagtgggctggcctggtggagaaggtgcaggctgctgtgggcacttctgctgcccctgtgccctctgacaaccattga

SEQ ID NO: 12 - DNA, 954 bp, synthetic construct
  source 1..954: mol_type = other DNA
atgaaggtgctgtgggctgccctgctggtgaccttcctggctgggtgccaggctaaggtggagcaggctgtggagactgagcctgagcctgagctgaggcagcagactgagtggcagtctggccagaggtgggagctggccctgggcaggttttgggactacctgaggtgggtgcagaccctgtctgagcaggtgcaggaagagctgctgtctagccaggtgacccaggagctgagggccctgatggatgagaccatgaaggagctgaaggcctacaagtctgaactggaggagcagctgacccctgtggctgaggagactagggccaggctgagcaaggagctgcaggctgcccaggccagactgggggctgacatggaggatgtgtgtgggaggctggtgcagtactctggggaggtgcaggccatgctgggccagagcactgaggagctgagggtgagactggctagccatctgaggaagctgagaaagaggctgctgagggatgctgatgacctgcagaagaggctggctgtgtaccaggctggggccagggagggggctgagaggggcctgtctgccatcagggagaggctgggccccctggtggagcagggcagggtgagggctgctactgtgggcagcctggctggccagcccctgcaggagagggcccaggcttggggggagaggctgagggccaggatggaggagatgggcagcaggaccagggataggctggatgaggtgaaagagcaggtggctgaggtgagggccaagctggaggagcaggcccagcagatcaggctgcaggctgaggccttccaggctagactgaagagctggtttgagcccctggtggaggacatgcagaggcagtgggctggcctggtggagaaggtgcaggctgctgtgggcacctctgctgcccctgtgccttctgacaatcactga

SEQ ID NO: 13 - DNA, 954 bp, synthetic construct
  source 1..954: mol_type = other DNA
atgaaggtgctgtgggctgccctgctggtgaccttcctggctgggtgtcaggccaaggtggagcaggctgtggagactgagcctgagcctgaactgaggcagcagactgagtggcagtctggccagaggtgggagctggccctgggcaggttctgggattacctgaggtgggtgcagaccctgtctgagcaggtgcaggaggagctgctgtctagccaggtgacccaggagctgagggccctgatggatgagaccatgaaggagctgaaggcctacaagtctgagctggaggagcagctgactcctgtggctgaggagactagggccaggctgtccaaggagctgcaggctgcccaggccagactgggggctgacatggaagatgtgtgtgggaggctggtgcagtactctggggaggtgcaggccatgctggggcagagcactgaggagctgagggtgaggctggccagccacctgaggaagctgaggaagaggctgctgagggatgctgatgacctgcagaagaggctggctgtgtaccaggctggggccagggagggggctgaaagaggcctgtctgccatcagggagaggctggggcccctggtggagcagggcagggtgagggctgccactgtgggcagcctggctggccagcccctgcaggagagggcccaggcctggggggagaggctgagggccaggatggaggagatggggagcaggaccagggacaggctggatgaggtgaaggagcaggtggctgaggtgagggccaagctggaggagcaggcccagcagatcaggctgcaggctgaggctttccaggctaggctgaagagctggtttgagcccctggtggaggacatgcagaggcagtgggctggcctggtggagaaggtgcaggctgctgtggggacttctgctgccccagtgccctctgacaatcattga

SEQ ID NO: 14 - DNA, 954 bp, synthetic construct
  source 1..954: mol_type = other DNA
atgaaggtcctgtgggctgccctgctggtgaccttcctggctggctgccaggctaaggtggagcaggctgtggagactgagccagagcctgagctgaggcagcagactgagtggcagtctggccagaggtgggagctggccctgggcaggttctgggactacctgaggtgggtgcagactctgtctgagcaggtgcaggaggagctgctgtctagccaggtgacccaggagctgagggccctgatggatgagactatgaaggagctgaaggcttacaagtctgagctggaggagcagctgacccctgtggctgaggagaccagggctaggctgagcaaggagctgcaggctgcccaggccaggctgggggctgacatggaagatgtgtgtggcaggctggtgcagtactctggggaggtgcaggctatgctgggccagagcactgaggagctgagggtgaggctggcctctcacctgaggaagctgaggaagaggctgctgagggatgctgatgacctgcagaagaggctggctgtgtaccaggctggggccagggagggggctgagaggggcctgtctgctatcagggagaggctgggccccctggtggagcaggggagagtgagggctgccactgtgggctctctggctggccagcccctgcaggagagggctcaggcctggggggagaggctgagggccaggatggaagagatgggcagcaggaccagggacaggctggatgaggtgaaggagcaggtggctgaggtgagggctaagctggaggagcaggcccagcagattaggctgcaggctgaggccttccaggccaggctgaagagctggtttgagcccctggtggaggatatgcagaggcagtgggctgggctggtggagaaggtgcaggctgctgtgggcacttctgctgctcctgtgccctctgacaaccactga

SEQ ID NO: 15 - DNA, 954 bp, synthetic construct
  source 1..954: mol_type = other DNA
atgaaggtgctgtgggctgccctgctggtgacttttctggctggctgccaggccaaggtggagcaggctgtggagactgagcctgagcctgagctgaggcagcagactgagtggcagtctggccagaggtgggagctggccctggggaggttctgggactacctgaggtgggtgcagaccctgtctgagcaggtgcaggaggagctgctgagcagccaggtgactcaggagctgagggccctgatggatgagaccatgaaggaactgaaggcctacaagtctgaactggaggagcagctgacccctgtggctgaggagaccagggccaggctgtctaaggagctgcaggctgcccaggctaggctgggggctgatatggaggatgtgtgtggcagactggtgcagtactctggggaggtgcaggccatgctgggccagagcactgaggagctgagagtgaggctggcctctcacctgagaaagctgaggaagaggctgctgagggatgctgatgatctgcagaagaggctggctgtgtaccaggctggggccagggagggggctgagaggggcctgtctgccatcagggaaaggctgggccccctggtggagcagggcagagtgagggctgctactgtgggcagcctggctggccagcccctgcaggagagggcccaggcctggggggagaggctgagggccaggatggaggagatgggcagcaggactagggataggctggatgaggtgaaggagcaggtggctgaggtgagggccaagctggaggagcaggcccagcagatcagactgcaggctgaggccttccaggccaggctgaagagctggtttgagcccctggtggaggatatgcagaggcagtgggctggcctggtggagaaggtgcaggctgctgtgggcacctctgctgcccctgtgccctctgacaaccactga

SEQ ID NO: 16 - DNA, 954 bp, synthetic construct
  source 1..954: mol_type = other DNA
atgaaggtgctgtgggctgccctgctggtgaccttcctggctggctgccaggccaaggtggagcaggctgtggagactgagccagagcctgaactgaggcagcagactgaatggcagtctggccagaggtgggagctggccctggggaggttctgggactacctgaggtgggtgcagaccctgtctgagcaggtgcaggaggagctgctgagcagccaggtgactcaggagctgagggccctgatggatgagaccatgaaggagctgaaggcctataagtctgagctggaggagcagctgacccctgtggctgaggagaccagggccaggctgtctaaggagctgcaggctgctcaggccaggctgggggctgatatggaggatgtgtgtggcaggctggtgcagtactctggggaggtgcaggccatgctgggccagtctactgaggagctgagggtgaggctggccagccacctgaggaagctgaggaagaggctgctgagggatgctgatgacctgcagaagaggctggctgtgtaccaggctggggccagggagggggctgagaggggcctgtctgccattagggagaggctggggcccctggtggagcaggggagggtgagggctgccactgtggggagcctggctggccagcccctgcaggagagggcccaggcctggggggagagactgagagccaggatggaagagatgggcagcaggaccagggataggctggatgaggtgaaggagcaggtggctgaggtcagggccaagctggaggagcaggcccagcagatcaggctgcaggctgaggctttccaggctaggctgaagtcttggtttgaacctctggtggaagatatgcagaggcagtgggctggcctggtggagaaggtgcaggctgctgtgggcacttctgctgctcctgtgccctctgataaccactga

SEQ ID NO: 17 - DNA, 954 bp, synthetic construct
  source 1..954: mol_type = other DNA
atgaaggtgctgtgggctgctctgctggtgaccttcctggctggctgccaggccaaggtggagcaggctgtggagactgaacctgagcctgagctgaggcagcagactgagtggcagtctggccagaggtgggagctggccctgggcaggttctgggattacctgaggtgggtgcagactctgtctgagcaggtgcaggaggagctgctgtcttctcaggtgactcaggagctgagggccctgatggatgagaccatgaaggagctgaaggcctacaagtctgagctggaggagcagctgacccctgtggctgaggagaccagggccaggctgtctaaggagctgcaggctgcccaggccaggctgggggctgacatggaggatgtgtgtggcaggctggtgcagtattctggggaagtgcaggccatgctgggccagagcactgaggagctgagggtgaggctggccagccatctgaggaagctgaggaagaggctgctgagagatgctgatgacctgcagaagagactggctgtgtaccaggctggggccagggagggggctgagaggggcctgtctgctattagggaaaggctggggcctctggtggagcaggggagggtgagggctgccactgtgggcagcctggctggccagcccctgcaggaaagggcccaggcctggggggagaggctgagggccaggatggaggagatgggcagcaggactagggacaggctggatgaggtgaaggagcaggtggctgaggtcagggctaagctggaggagcaggcccagcagatcaggctgcaggctgaggccttccaggctaggctgaagtcttggtttgagcccctggtggaggacatgcagagacagtgggctggcctggtggagaaggtgcaggctgctgtgggcacctctgctgcccctgtgccctctgacaaccattga

SEQ ID NO: 18 - DNA, 954 bp, synthetic construct
  source 1..954: mol_type = other DNA
atgaaggtgctgtgggctgccctgctggtgaccttcctggctggctgccaggccaaggtggaacaggctgtggagactgagcctgagcctgagctgaggcagcagactgagtggcagtctggccagaggtgggagctggccctggggaggttttgggactacctgaggtgggtgcagaccctgtctgagcaggtccaggaggagctgctgagcagccaggtgactcaggagctgagagccctgatggatgagactatgaaggagctgaaggcctacaagtctgagctggaggagcagctgacccctgtggctgaggagaccagggccaggctgagcaaggagctgcaggctgctcaggccaggctgggggctgacatggaggatgtgtgtggcaggctggtgcagtacaggggggaggtgcaggccatgctgggccagagcactgaggagctgagggtgaggctggccagccacctgagaaagctgagaaagaggctgctgagggatgctgatgatctgcagaagaggctggctgtgtaccaggctggggccagggagggggctgagaggggcctgtctgccatcagggagaggctgggccccctggtggagcagggcagagtgagggctgccactgtgggcagcctggctggccagcccctgcaggagagggcccaggcctggggggagaggctgagagctaggatggaggagatgggcagcaggactagggacaggctggatgaggtgaaggagcaggtggctgaagtgagggccaagctggaggagcaggcccagcagatcaggctgcaggctgaggccttccaggccagactgaagtcttggtttgagcccctggtggaggacatgcagaggcagtgggctggcctggtggagaaggtgcaggctgctgtgggcacttctgctgcccctgtgccttctgacaaccactga

SEQ ID NO: 19 - DNA, 954 bp, synthetic construct
  source 1..954: mol_type = other DNA
atgaaggtgctgtgggctgctctgctggtcacctttctggctggctgtcaggccaaggtggagcaggctgtggagactgagcctgagcctgagctgaggcagcagactgagtggcagtctggccagaggtgggagctggccctgggcaggttttgggactacctgaggtgggtgcagaccctgtctgagcaggtgcaggaggagctgctgtctagccaggtgactcaggagctgagggctctgatggatgagaccatgaaggagctgaaggcctacaagtctgagctggaggagcagctgacccctgtggctgaggagaccagggccaggctgtctaaggagctgcaggctgctcaggccagactgggggctgatatggaggatgtgtgtggcaggctggtgcagtacagaggggaggtgcaggctatgctgggccagagcactgaggagctgagggtgaggctggccagccacctgaggaagctgaggaagaggctgctgagggatgctgatgacctgcagaagaggctggctgtgtaccaggctggggctagggagggggctgagaggggcctgtctgccatcagggagaggctggggcccctggtggagcaggggagggtgagggctgccactgtgggctctctggctggccagcccctgcaggagagggcccaggcttggggggagagactgagggccaggatggaggagatgggctctaggactagggataggctggatgaggtgaaggagcaggtggctgaggtgagggctaagctggaggagcaggcccagcagatcaggctgcaggctgaggccttccaggccaggctgaagagctggtttgagcctctggtggaggacatgcagaggcagtgggctgggctggtggagaaggtgcaggctgctgtgggcacttctgctgcccctgtgccctctgataaccactga

SEQ ID NO: 20 - DNA, 954 bp, synthetic construct
  source 1..954: mol_type = other DNA
atgaaggtgctgtgggctgccctgctggtgaccttcctggctggctgtcaggctaaagtggagcaggctgtggagactgagcctgagccagagctgaggcagcagactgagtggcagtctggccagagatgggagctggctctgggcaggttctgggactacctgaggtgggtgcagaccctgtctgagcaggtgcaggaggagctgctgtcttctcaggtgacccaggaactgagggccctgatggatgagactatgaaggagctgaaggcctacaagtctgagctggaggagcagctgacccctgtggctgaggagaccagggccaggctgagcaaggagctgcaggctgcccaggccagactgggggctgacatggaggatgtgtgtggcagactggtgcagtacaggggggaggtgcaggccatgctggggcagtctactgaggagctgagggtgaggctggccagccacctgaggaagctgaggaagaggctgctgagggatgctgatgacctgcagaagaggctggctgtgtatcaggctggggctagggagggggctgagaggggcctgtctgccatcagggagaggctggggcctctggtggagcagggcagggtgagggctgccactgtggggagcctggctggccagcctctgcaggagagggctcaggcctggggggagaggctgagggccaggatggaggagatggggagcaggaccagggacaggctggatgaggtgaaggagcaggtggctgaggtgagggccaagctggaagagcaggcccagcagatcaggctgcaggctgaggccttccaggccaggctgaagagctggtttgagcccctggtggaggacatgcagaggcagtgggctggcctggtggagaaggtgcaggctgctgtgggcacctctgctgcccctgtgccctctgacaatcactga

SEQ ID NO: 21 - DNA, 954 bp, synthetic construct
  source 1..954: mol_type = other DNA
atgaaggtgctgtgggctgccctgctggtgactttcctggctggctgccaggctaaggtggagcaggctgtggagactgaacctgagcctgagctgaggcagcagactgagtggcagtctggccagaggtgggagctggccctggggaggttctgggactacctgaggtgggtgcagactctgtctgagcaggtgcaggaggagctgctgagctctcaggtgacccaggagctgagggccctgatggatgaaactatgaaggagctgaaagcctacaagtctgagctggaggagcagctgacccctgtggctgaggagaccagggccaggctgtctaaggagctgcaggctgcccaggccaggctgggggctgatatggaagatgtgtgtggcaggctggtgcagtacaggggggaggtgcaggctatgctgggccagtctactgaggagctgagagtgaggctggccagccatctgaggaagctgaggaagaggctgctgagggatgctgatgatctgcagaagagactggctgtgtaccaggctggggctagggagggggctgagaggggcctgtctgccatcagggagaggctgggccccctggtggagcagggcagggtgagggctgccactgtgggctctctggctggccagcccctgcaggagagggcccaggcctggggggagaggctgagggctaggatggaagagatgggcagcaggaccagggacaggctggatgaagtgaaggagcaggtggctgaggtgagggccaagctggaggagcaggcccagcagattaggctgcaggctgaggccttccaggccaggctgaagtcttggtttgagccactggtggaggacatgcagaggcagtgggctggcctggtggagaaggtgcaggctgctgtgggcacctctgctgcccctgtgccttctgacaaccactga

SEQ ID NO: 22 - DNA, 954 bp, synthetic construct
  source 1..954: mol_type = other DNA
atgaaggtcctgtgggctgccctgctggtgaccttcctggctggctgccaggccaaggtggagcaggctgtggagactgagcctgagcctgagctgaggcagcagactgagtggcagtctgggcagaggtgggagctggccctgggcaggttctgggactacctgaggtgggtgcagactctgtctgaacaggtgcaggaggagctgctgagcagccaggtgacccaggagctgagggccctgatggatgagaccatgaaggagctgaaggcctataagtctgaactggaggagcagctgacccctgtggctgaggagactagggccaggctgagcaaggagctgcaggctgctcaggccagactgggggctgacatggaggatgtctgtggcagactggtgcagtacaggggggaggtgcaggctatgctggggcagagcactgaggagctgagggtgaggctggccagccacctgaggaagctgaggaagaggctgctgagggatgctgatgatctgcagaagagactggctgtgtaccaggctggggccagagagggggctgagagggggctgtctgccattagggagagactgggccccctggtggagcagggcagggtgagggctgccactgtgggctctctggctggccagcctctgcaggagagggctcaggcctggggggagaggctgagggctagaatggaggagatggggagcaggaccagggacaggctggatgaggtgaaagagcaggtggctgaggtcagggccaagctggaggagcaggcccagcagatcaggctgcaggctgaggctttccaggctaggctgaagagctggtttgagcccctggtggaggatatgcagaggcagtgggctggcctggtggagaaggtgcaggctgctgtgggcacttctgctgctcctgtgccctctgataaccactga

SEQ ID NO: 23 - DNA, 954 bp, synthetic construct
  source 1..954: mol_type = other DNA
atgaaggtgctgtgggctgccctgctggtgaccttcctggctggctgccaggccaaggtggagcaggctgtggagactgagcctgagcctgaactgaggcagcagactgaatggcagtctggccagaggtgggagctggccctgggcagattctgggattacctgaggtgggtgcagaccctgtctgagcaggtgcaggaggaactgctgagcagccaggtgactcaggagctgagggccctgatggatgagaccatgaaggagctgaaggcctataaatctgagctggaggagcagctgacccctgtggctgaggagactagggccagactgtctaaggagctgcaggctgcccaggccaggctgggggctgacatggaggatgtgtgtggcaggctggtgcagtacaggggggaggtgcaggccatgctgggccagtctactgaggagctgagggtgaggctggccagccacctgaggaagctgaggaagaggctgctgagggatgctgatgacctgcagaagaggctggctgtgtaccaggctggggccagggagggggctgagaggggcctgtctgccatcagagagaggctgggccccctggtggagcagggcagggtgagggctgccactgtgggctccctggctggccagcccctgcaggagagggcccaggcctggggggagaggctgagggccaggatggaggagatgggcagcaggaccagagacaggctggatgaggtcaaggagcaggtggctgaggtgagggccaagctggaggagcaggcccagcagatcagactgcaggctgaggccttccaggctaggctgaagtcttggtttgagccactggtggaggacatgcagaggcagtgggctggcctggtggaaaaggtgcaggctgctgtgggcacctctgctgcccctgtgccctctgacaatcactga

SEQ ID NO: 24 - DNA, 954 bp, synthetic construct
  source 1..954: mol_type = other DNA
atgaaggtgctgtgggctgccctgctggtgaccttcctggctggctgccaggctaaggtggagcaggctgtggagactgagcctgagcctgagctgaggcagcagactgagtggcagtctgggcagaggtgggagctggccctggggaggttctgggactacctgaggtgggtgcagactctgtctgagcaggtgcaggaggagctgctgagctctcaggtgactcaggagctgagggccctgatggatgagactatgaaggagctgaaggcttacaagtctgagctggaggagcagctgactcctgtggctgaggagaccagggccaggctgtctaaggagctgcaggctgcccaggccagactgggggctgacatggaggatgtgtgtggcaggctggtgcagtacaggggggaggtgcaggctatgctggggcagagcactgaggagctgagggtgaggctggccagccatctgaggaagctgaggaagaggctgctgagggatgctgatgacctgcagaagagactggctgtgtatcaggctggggccagggaaggggctgagaggggcctgtctgccatcagggagaggctgggccccctggtggaacaggggagggtgagggctgccactgtgggcagcctggctggccagcccctgcaggagagggctcaggcttggggggagaggctgagggccaggatggaggagatgggcagcaggaccagggataggctggatgaggtgaaggagcaggtggctgaggtgagggctaagctggaggagcaggctcagcagatcaggctgcaggctgaggctttccaggccaggctgaagagctggtttgagcccctggtggaggacatgcagaggcagtgggctggcctggtggagaaggtccaggctgctgtgggcacttctgctgcccctgtgccctctgacaaccattga

SEQ ID NO: 25 - DNA, 954 bp, synthetic construct
  source 1..954: mol_type = other DNA
atgaaggtgctgtgggctgctctgctggtgaccttcctggctggctgccaggccaaggtggagcaggctgtggagactgagcctgagcctgagctgaggcagcagactgagtggcagtctggccagaggtgggagctggccctggggaggttctgggactacctgaggtgggtgcagaccctgtctgagcaggtccaggaagagctgctgtcttctcaggtgacccaggaactgagggccctgatggatgagaccatgaaggaactgaaggcctacaagtctgagctggaggagcagctgacccctgtggctgaggagaccagggctaggctgagcaaggagctgcaggctgctcaggccaggctgggggctgacatggaggatgtgtgtgggaggctggtgcagtacaggggggaggtgcaggccatgctgggccagagcactgaggagctgagggtgagactggccagccatctgaggaagctgaggaagaggctgctgagggatgctgatgacctgcagaagaggctggctgtgtaccaggctggggccagggagggggctgagaggggcctgtctgctattagggagaggctgggccccctggtggagcaggggagggtgagggctgccactgtgggcagcctggctggccagcccctgcaggagagggcccaggcctggggggagaggctgagggccaggatggaggagatgggcagcaggaccagggacaggctggatgaggtgaaggagcaggtggctgaggtgagggccaagctggaggagcaggcccagcagatcaggctgcaggctgaggccttccaggccaggctgaagagctggtttgagcccctggtggaggacatgcagaggcagtgggctggcctggtggagaaggtgcaggctgctgtggggacctctgctgcccctgtgccttctgacaaccactga

SEQ ID NO: 26 - DNA, 954 bp, synthetic construct
  source 1..954: mol_type = other DNA
atgaaagtgctgtgggctgctctgctggtgaccttcctggctgggtgccaggccaaggtggagcaggctgtggagactgagcctgagcctgagctgaggcagcagactgagtggcagtctggccagaggtgggagctggccctggggaggttctgggactacctgaggtgggtgcagactctgtctgagcaggtgcaggaggagctgctgtctagccaggtgacccaggagctgagagccctgatggatgagaccatgaaggagctgaaggcctacaagtctgagctggaggagcagctgacccctgtggctgaggagaccagagctaggctgtctaaggagctgcaggctgctcaggccaggctgggggctgacatggaggatgtgtgtggcaggctggtgcagtataggggggaggtccaggccatgctggggcagagcactgaggagctgagggtgaggctggccagccatctgaggaagctgaggaagaggctgctgagggatgctgatgacctgcagaagagactggctgtgtaccaggctggggccagggagggggctgagaggggcctgtctgccatcagggagaggctggggcctctggtggagcagggcagggtgagggctgccactgtgggctctctggctggccagcccctgcaggagagggcccaggcttggggggagaggctgagagccagaatggaggagatgggcagcaggaccagggacaggctggatgaggtgaaggagcaggtggctgaggtgagggccaagctggaggagcaggcccagcagatcaggctgcaggctgaggcctttcaggccaggctgaagagctggtttgaacccctggtggaggacatgcagagacagtgggctggcctggtggagaaggtgcaggctgctgtgggcacctctgctgcccctgtgccctctgataaccactga

SEQ ID NO: 27 - DNA, 954 bp, synthetic construct
  source 1..954: mol_type = other DNA
atgaaggtgctgtgggctgccctgctggtgacctttctggctgggtgccaggctaaagtggagcaggctgtggagactgagcctgagcctgagctgaggcagcagactgagtggcagtctggccagaggtgggagctggctctgggcaggttctgggactatctgaggtgggtgcagactctgtctgaacaggtgcaggaggagctgctgagctctcaggtgacccaggagctgagggctctgatggatgagactatgaaggaactgaaggcctacaagtctgagctggaggagcagctgacccctgtggctgaggagactagggctaggctgagcaaggagctgcaggctgcccaggccagactgggggctgacatggaggatgtgtgtggcaggctggtgcagtacaggggggaggtgcaggctatgctggggcagagcactgaggagctgagggtgaggctggcctctcatctgaggaagctgaggaagaggctgctgagggatgctgatgacctgcagaagaggctggctgtgtaccaggctggggccagggagggggctgagaggggcctgtctgccatcagggagaggctgggccccctggtggagcaggggagggtgagggctgccactgtgggcagcctggctggccagcccctgcaggagagggctcaggcctggggggagagactgagggctagaatggaggagatggggagcaggaccagggataggctggatgaggtgaaggagcaggtggctgaggtgagggccaagctggaggagcaggcccagcagatcaggctgcaggctgaggcctttcaggccaggctgaagagctggtttgagcccctggtggaagatatgcagaggcagtgggctggcctggtggagaaggtgcaggctgctgtgggcacctctgctgcccctgtgccctctgacaaccactga

SEQ ID NO: 28 - DNA, 954 bp, synthetic construct
  source 1..954: mol_type = other DNA
atgaaggtgctgtgggctgccctgctggtgacctttctggctgggtgccaggctaaggtggagcaggctgtggagactgagcctgagcctgaactgaggcagcagactgagtggcagtctggccagaggtgggagctggccctgggcaggttctgggactatctgaggtgggtgcagaccctgtctgagcaggtgcaggaagagctgctgagctctcaggtgactcaggagctgagggccctgatggatgagaccatgaaggagctgaaggcctacaagtctgagctggaggagcagctgactcctgtggctgaagagaccagggccaggctgagcaaggagctgcaggctgcccaggctaggctgggggctgatatggaggatgtgtgtgggagactggtgcagtacaggggggaggtccaggctatgctgggccagagcactgaggagctgagggtgaggctggcctctcacctgaggaagctgaggaagaggctgctgagggatgctgatgacctgcagaagagactggctgtgtaccaggctggggctagggagggggctgagagggggctgtctgctatcagggagaggctggggcctctggtggagcagggcagggtgagggctgccactgtgggcagcctggctggccagcccctgcaggagagggcccaggcctggggggagaggctgagggccaggatggaggagatggggagcaggaccagggataggctggatgaggtgaaggagcaggtggctgaggtgagggctaagctggaggagcaggcccagcagattaggctgcaggctgaggcctttcaggctaggctgaagtcttggtttgagcccctggtggaggacatgcagaggcagtgggctggcctggtggagaaggtgcaggctgctgtgggcacctctgctgcccctgtgccctctgacaaccactga

SEQ ID NO: 29 - DNA, 954 bp, synthetic construct
  source 1..954: mol_type = other DNA
atgaaggtgctgtgggctgccctgctggtgactttcctggctggctgccaggccaaggtggagcaggctgtggagactgagcctgagcctgagctgaggcagcagactgagtggcagtctggccagaggtgggagctggccctgggcaggttctgggactacctgaggtgggtgcagaccctgtctgagcaggtgcaggaggagctgctgagcagccaggtgactcaggagctgagggccctgatggatgagaccatgaaggagctgaaggcctacaaatctgagctggaggagcagctgacccctgtggctgaggagaccagggccaggctgagcaaggagctgcaggctgctcaggccaggctgggggctgatatggaggatgtgtgtggcaggctggtgcagtacaggggggaggtgcaggccatgctgggccagagcactgaggagctgagggtgaggctggccagccatctgaggaagctgaggaagagactgctgagggatgctgatgatctgcagaagaggctggctgtgtaccaggctggggccagggagggggctgagaggggcctgtctgccatcagggagaggctgggccccctggtggagcagggcagggtgagggctgccactgtgggcagcctggctggccagcctctgcaggagagggcccaggcttggggggagagactgagggccaggatggaagagatggggtctaggaccagggataggctggatgaggtgaaggagcaggtggctgaggtgagggccaagctggaggaacaggctcagcagatcaggctgcaggctgaggccttccaggccagactgaagagctggtttgagcctctggtggaggacatgcagaggcagtgggctggcctggtggagaaagtgcaggctgctgtgggcacttctgctgcccctgtgccctctgacaaccactga

SEQ ID NO: 30 - DNA, 954 bp, synthetic construct
  source 1..954: mol_type = other DNA
atgaaggtgctgtgggctgccctgctggtgactttcctggctggctgccaggccaaggtggagcaggctgtggaaactgagcctgagcctgagctgaggcagcagactgagtggcagtctggccagaggtgggagctggctctgggcaggttttgggactacctgaggtgggtgcagaccctgtctgagcaggtgcaggaggagctgctgagcagccaggtgacccaggagctgagagccctgatggatgagaccatgaaggagctgaaggcttacaagtctgagctggaggagcagctgactcctgtggctgaggagaccagggctaggctgtctaaggagctgcaggctgctcaggctaggctgggggctgacatggaggatgtgtgtggcagactggtgcagtacaggggggaggtgcaggctatgctgggccagagcactgaggaactgagggtgaggctggccagccacctgaggaagctgaggaagaggctgctgagggatgctgatgacctgcagaagaggctggctgtgtaccaggctggggccagggagggggctgagaggggcctgtctgccatcagggagaggctggggcctctggtggagcagggcagggtcagggctgccactgtgggcagcctggctggccagcccctgcaggagagggcccaggcttggggggagaggctgagagctaggatggaggagatgggcagcaggactagggataggctggatgaggtgaaggagcaggtggctgaggtgagggctaagctggaggagcaggcccagcagatcaggctgcaggctgaggcctttcaggccaggctgaagagctggtttgagcctctggtggaggacatgcagaggcagtgggctggcctggtggagaaggtgcaggctgctgtggggacctctgctgcccctgtgccctctgacaaccattga

SEQ ID NO: 31 - DNA, 954 bp, synthetic construct
  source 1..954: mol_type = other DNA
atgaaggtgctgtgggctgctctgctggtgaccttcctggctggctgccaggctaaggtggagcaggctgtggagactgagcctgagcctgagctgaggcagcagactgagtggcagtctgggcagaggtgggagctggccctgggcaggttctgggactacctgaggtgggtgcagaccctgtctgagcaggtgcaggaggagctgctgtctagccaggtgacccaggaactgagggccctgatggatgagaccatgaaggagctgaaggcctacaagtctgagctggaggagcagctgacccctgtggctgaggagaccagggccaggctgtctaaggagctgcaggctgcccaggccaggctgggggctgacatggaggatgtgtgtggcaggctggtgcagtataggggggaggtgcaggctatgctgggccagagcactgaggagctgagggtcaggctggccagccacctgaggaagctgaggaagaggctgctgagggatgctgatgacctgcagaagaggctggctgtgtaccaggctggggccagggagggggctgagaggggcctgtctgccatcagggagaggctgggccctctggtggagcaggggagggtgagggctgccactgtgggcagcctggctggccagcccctgcaggaaagggcccaggcctggggggagagactgagggccaggatggaggagatgggcagcaggaccagagacaggctggatgaggtgaaggagcaggtggctgaggtgagggccaagctggaggagcaggcccagcagattaggctgcaggctgaggccttccaggccaggctgaagtcttggtttgagcccctggtggaagatatgcagaggcagtgggctggcctggtggagaaggtgcaggctgctgtggggacctctgctgctcctgtgccctctgacaaccactga

SEQ ID NO: 32 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 33 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYSGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 34 - protein, 317 aa, Homo sapiens [sequence omitted]

SEQ ID NO: 35 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYSGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 36 - protein, 18 aa, Homo sapiens
MAFLWLLSCWALLGTTFG

SEQ ID NO: 37 - DNA, 54 bp, synthetic construct
  source 1..54: mol_type = other DNA
atggccttcctgtggctgctgtcctgctgggcactgctgggcaccacatttggc

SEQ ID NO: 38 - DNA, 54 bp, synthetic construct
  source 1..54: mol_type = other DNA
atggcctttctgtggctgctgtcctgctgggccctgctggggaccacctttggc

SEQ ID NO: 39 - DNA, 54 bp, synthetic construct
  source 1..54: mol_type = other DNA
atggccttcctgtggctgctgagctgctgggccctgctggggaccacctttggg

SEQ ID NO: 40 - DNA, 54 bp, synthetic construct
  source 1..54: mol_type = other DNA
atggccttcctgtggctcctgagctgctgggccctcctggggaccacctttggg

SEQ ID NO: 41 - DNA, 54 bp, synthetic construct
  source 1..54: mol_type = other DNA
atggccttcctgtggctgctgagctgctgggccctgctggggaccacatttggc

SEQ ID NO: 42 - protein, 18 aa, Homo sapiens
MKSLVLLLCLAQLWGCHS

SEQ ID NO: 43 - DNA, 54 bp, synthetic construct
  source 1..54: mol_type = other DNA
atgaagtccctcgtcctgctcctttgtcttgctcagctctggggctgccactca

SEQ ID NO: 44 - protein, 17 aa, Homo sapiens
MWLPWALLLLWVPGCFA

SEQ ID NO: 45 - DNA, 51 bp, synthetic construct
  source 1..51: mol_type = other DNA
atgtggctgccttgggctctgttgcttctctgggtcccaggatgttttgct

SEQ ID NO: 46 - protein, 31 aa, Homo sapiens
MQLRNPELHLGCALALRFLALVSWDIPGARA

SEQ ID NO: 47 - DNA, 93 bp, synthetic construct
  source 1..93: mol_type = other DNA
atgcagctgaggaacccagaactacatctgggctgcgcgcttgcgcttcgcttcctggccctcgtttcctgggacatccctggggctagagca

SEQ ID NO: 48 - protein, 28 aa, Homo sapiens
MAAPGSARRPLLLLLLLLLLGLMHCASA

SEQ ID NO: 49 - DNA, 84 bp, synthetic construct
  source 1..84: mol_type = other DNA
atggcggcccccggcagcgcccggcgacccctgctgctgctactgctgttgctgctgctcggcctcatgcattgtgcgtcagca

SEQ ID NO: 50 - protein, 25 aa, Homo sapiens
MPALRPALLWALLALWLCCAAPAHA

SEQ ID NO: 51 - DNA, 75 bp, synthetic construct
  source 1..75: mol_type = other DNA
atgcccgccctgcgccccgctctgctgtgggcgctgctggcgctctggctgtgctgcgcggcccccgcgcatgca

SEQ ID NO: 52 - protein, 18 aa, Homo sapiens
MALSWVLTVLSLLPLLEA

SEQ ID NO: 53 - DNA, 54 bp, synthetic construct
  source 1..54: mol_type = other DNA
atggcgctgtcctgggttcttacagtcctgagcctcctacctctgctggaagcc

SEQ ID NO: 54 - protein, 19 aa, Homo sapiens
MRLAVGALLVCAVLGLCLA

SEQ ID NO: 55 - DNA, 57 bp, synthetic construct
  source 1..57: mol_type = other DNA
atgaggctcgccgtgggagccctgctggtctgcgccgtcctggggctgtgtctggct

SEQ ID NO: 56 - protein, 18 aa, Homo sapiens
MKVLWAALLVTFLAGCQA

SEQ ID NO: 57 - DNA, 54 bp, synthetic construct
  source 1..54: mol_type = other DNA
atgaaggttctgtgggctgcgttgctggtcacattcctggcaggatgccaggcc

SEQ ID NO: 58 - DNA, 321 bp, Homo sapiens
  source 1..321: mol_type = genomic DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 59 - DNA, 397 bp, Homo sapiens
  source 1..397: mol_type = genomic DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 60 - DNA, 543 bp, synthetic construct
  source 1..543: mol_type = other DNA
gatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaag

SEQ ID NO: 61 - DNA, 228 bp, synthetic construct
  source 1..228: mol_type = other DNA
ctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 62 - DNA, 228 bp, unidentified
  source 1..228: mol_type = other DNA; note = Bovinae
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 63 - DNA, 900 bp, synthetic construct
  source 1..900: mol_type = other DNA
aaagtggagcaggctgtggagactgagcctgagccagagctgaggcagcagactgagtggcagtctggccagagatgggagctggctctgggcaggttctgggactacctgaggtgggtgcagaccctgtctgagcaggtgcaggaggagctgctgtcttctcaggtgacccaggaactgagggccctgatggatgagactatgaaggagctgaaggcctacaagtctgagctggaggagcagctgacccctgtggctgaggagaccagggccaggctgagcaaggagctgcaggctgcccaggccagactgggggctgacatggaggatgtgtgtggcagactggtgcagtacaggggggaggtgcaggccatgctggggcagtctactgaggagctgagggtgaggctggccagccacctgaggaagctgaggaagaggctgctgagggatgctgatgacctgcagaagaggctggctgtgtatcaggctggggctagggagggggctgagaggggcctgtctgccatcagggagaggctggggcctctggtggagcagggcagggtgagggctgccactgtggggagcctggctggccagcctctgcaggagagggctcaggcctggggggagaggctgagggccaggatggaggagatggggagcaggaccagggacaggctggatgaggtgaaggagcaggtggctgaggtgagggccaagctggaagagcaggcccagcagatcaggctgcaggctgaggccttccaggccaggctgaagagctggtttgagcccctggtggaggacatgcagaggcagtgggctggcctggtggagaaggtgcaggctgctgtgggcacctctgctgcccctgtgccctctgacaatcactga

SEQ ID NO: 64 - DNA, 900 bp, synthetic construct
  source 1..900: mol_type = other DNA
aaggtggagcaggctgtggagactgagcctgagcctgagctgaggcagcagactgagtggcagtctggccagaggtgggagctggccctggggaggttctgggactacctgaggtgggtgcagactctgtctgagcaggtgcaggaggagctgctgtctagccaggtgacccaggagctgagagccctgatggatgagaccatgaaggagctgaaggcctacaagtctgagctggaggagcagctgacccctgtggctgaggagaccagagctaggctgtctaaggagctgcaggctgctcaggccaggctgggggctgacatggaggatgtgtgtggcaggctggtgcagtataggggggaggtccaggccatgctggggcagagcactgaggagctgagggtgaggctggccagccatctgaggaagctgaggaagaggctgctgagggatgctgatgacctgcagaagagactggctgtgtaccaggctggggccagggagggggctgagaggggcctgtctgccatcagggagaggctggggcctctggtggagcagggcagggtgagggctgccactgtgggctctctggctggccagcccctgcaggagagggcccaggcttggggggagaggctgagagccagaatggaggagatgggcagcaggaccagggacaggctggatgaggtgaaggagcaggtggctgaggtgagggccaagctggaggagcaggcccagcagatcaggctgcaggctgaggcctttcaggccaggctgaagagctggtttgaacccctggtggaggacatgcagagacagtgggctggcctggtggagaaggtgcaggctgctgtgggcacctctgctgcccctgtgccctctgataaccactga

SEQ ID NO: 65 - DNA, 900 bp, synthetic construct
  source 1..900: mol_type = other DNA
aaggtggagcaggctgtggagactgagcctgagcctgagctgaggcagcagactgagtggcagtctgggcagaggtgggagctggccctgggcaggttctgggactacctgaggtgggtgcagaccctgtctgagcaggtgcaggaggagctgctgtctagccaggtgacccaggaactgagggccctgatggatgagaccatgaaggagctgaaggcctacaagtctgagctggaggagcagctgacccctgtggctgaggagaccagggccaggctgtctaaggagctgcaggctgcccaggccaggctgggggctgacatggaggatgtgtgtggcaggctggtgcagtataggggggaggtgcaggctatgctgggccagagcactgaggagctgagggtcaggctggccagccacctgaggaagctgaggaagaggctgctgagggatgctgatgacctgcagaagaggctggctgtgtaccaggctggggccagggagggggctgagaggggcctgtctgccatcagggagaggctgggccctctggtggagcaggggagggtgagggctgccactgtgggcagcctggctggccagcccctgcaggaaagggcccaggcctggggggagagactgagggccaggatggaggagatgggcagcaggaccagagacaggctggatgaggtgaaggagcaggtggctgaggtgagggccaagctggaggagcaggcccagcagattaggctgcaggctgaggccttccaggccaggctgaagtcttggtttgagcccctggtggaagatatgcagaggcagtgggctggcctggtggagaaggtgcaggctgctgtggggacctctgctgctcctgtgccctctgacaaccactga

SEQ ID NO: 66 - DNA, 900 bp, synthetic construct
  source 1..900: mol_type = other DNA
aaggtggagcaggctgtggagactgagccagagcctgagctgaggcagcagactgaatggcagtctggccagaggtgggagctggccctgggcaggttctgggactacctgagatgggtgcagaccctgtctgagcaggtccaggaggagctgctgtcttctcaggtgacccaggagctgagggccctgatggatgagactatgaaggagctgaaggcttacaagtctgagctggaggagcagctgacccctgtggctgaggagaccagggccagactgagcaaggagctgcaggctgctcaggccaggctgggggctgacatggaggatgtgtgtggcaggctggtgcagtattctggggaggtgcaggctatgctgggccagagcactgaggaactgagggtgaggctggcctctcatctgaggaagctgaggaagaggctgctgagggatgctgatgacctgcagaagaggctggctgtgtatcaggctggggccagggagggggctgagagggggctgtctgctatcagggagaggctgggccccctggtggagcaggggagagtgagggctgccactgtggggtctctggctggccagcccctgcaggagagggcccaggcctggggggagaggctgagggctagaatggaggagatggggtctaggaccagggacaggctggatgaggtcaaggaacaggtggctgaggtcagggccaagctggaggagcaggcccagcagattaggctgcaggctgaggcctttcaggccaggctgaagagctggtttgagcccctggtggaggacatgcagaggcagtgggctggcctggtggagaaggtgcaggctgctgtgggcacttctgctgcccctgtgccctctgacaaccattga

SEQ ID NO: 67 - DNA, 900 bp, synthetic construct
  source 1..900: mol_type = other DNA
aaggtggagcaggctgtggagactgagcctgagcctgagctgaggcagcagactgagtggcagtctgggcagaggtgggagctggccctggggaggttctgggactacctgaggtgggtgcagactctgtctgagcaggtgcaggaggagctgctgtctagccaggtgacccaggagctgagggccctgatggatgagaccatgaaggagctgaaggcctacaagtctgagctggaagagcagctgacccctgtggctgaggagaccagggctaggctgagcaaggaactgcaggctgctcaggccaggctgggggctgacatggaagatgtgtgtgggaggctggtgcagtactctggggaggtgcaggccatgctgggccagagcactgaggagctgagggtgaggctggccagccacctgaggaagctgaggaagaggctgctgagggatgctgatgacctgcagaagaggctggctgtgtaccaggctggggccagggagggggctgagaggggcctgtctgctatcagggagaggctgggccccctggtggagcagggcagggtgagggctgccactgtgggcagcctggctgggcagcctctgcaggagagggcccaggcctggggggagaggctgagagccaggatggaggagatgggctctaggactagggacaggctggatgaggtgaaggagcaggtggctgaggtgagggccaagctggaggagcaggctcagcagatcaggctgcaggctgaggctttccaggccaggctgaagtcttggtttgagcccctggtggaggatatgcagaggcagtgggctggcctggtggagaaggtgcaggctgctgtgggcacctctgctgcccctgtgccctctgacaaccattga

SEQ ID NO: 68 - protein, 21 aa, synthetic construct
MKFALVAALALLLFLWSSCRA

SEQ ID NO: 69 - protein, 20 aa, synthetic construct
MKFALVAALALLLFLWGCHS

SEQ ID NO: 70 - protein, 21 aa, synthetic construct
MKWVTLLTLLLLLLFLWGCHS

SEQ ID NO: 71 - protein, 22 aa, synthetic construct
MKWVTLLTLLLLLLFLWSSCRA

SEQ ID NO: 72 - DNA, 63 bp, synthetic construct
  source 1..63: mol_type = other DNA
atgaagttcgccctggtggccgccctggccctgctgctgttcctgtggagcagctgcagggcc

SEQ ID NO: 73 - DNA, 60 bp, synthetic construct
  source 1..60: mol_type = other DNA
atgaagttcgccctggtggccgccctggccctgctgctgttcctgtggggctgccacagc

SEQ ID NO: 74 - DNA, 63 bp, synthetic construct
  source 1..63: mol_type = other DNA
atgaagtgggtgaccctgctgaccctgctgctgctgctgctgttcctgtggggctgccacagc

SEQ ID NO: 75 - DNA, 66 bp, synthetic construct
  source 1..66: mol_type = other DNA
atgaagtgggtgaccctgctgaccctgctgctgctgctgctgttcctgtggagcagctgcagggcc

SEQ ID NO: 76 - DNA, 2565 bp, synthetic construct
  source 1..2565: mol_type = other DNA
gcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgaaggtgctgtgggctgccctgctggtgaccttcctggctggctgtcaggctaaagtggagcaggctgtggagactgagcctgagccagagctgaggcagcagactgagtggcagtctggccagagatgggagctggctctgggcaggttctgggactacctgaggtgggtgcagaccctgtctgagcaggtgcaggaggagctgctgtcttctcaggtgacccaggaactgagggccctgatggatgagactatgaaggagctgaaggcctacaagtctgagctggaggagcagctgacccctgtggctgaggagaccagggccaggctgagcaaggagctgcaggctgcccaggccagactgggggctgacatggaggatgtgtgtggcagactggtgcagtacaggggggaggtgcaggccatgctggggcagtctactgaggagctgagggtgaggctggccagccacctgaggaagctgaggaagaggctgctgagggatgctgatgacctgcagaagaggctggctgtgtatcaggctggggctagggagggggctgagaggggcctgtctgccatcagggagaggctggggcctctggtggagcagggcagggtgagggctgccactgtggggagcctggctggccagcctctgcaggagagggctcaggcctggggggagaggctgagggccaggatggaggagatggggagcaggaccagggacaggctggatgaggtgaaggagcaggtggctgaggtgagggccaagctggaagagcaggcccagcagatcaggctgcaggctgaggccttccaggccaggctgaagagctggtttgagcccctggtggaggacatgcagaggcagtgggctggcctggtggagaaggtgcaggctgctgtgggcacctctgctgcccctgtgccctctgacaatcactgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgc

SEQ ID NO: 77 - DNA, 2565 bp, synthetic construct
  source 1..2565: mol_type = other DNA
gcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgaaagtgctgtgggctgctctgctggtgaccttcctggctgggtgccaggccaaggtggagcaggctgtggagactgagcctgagcctgagctgaggcagcagactgagtggcagtctggccagaggtgggagctggccctggggaggttctgggactacctgaggtgggtgcagactctgtctgagcaggtgcaggaggagctgctgtctagccaggtgacccaggagctgagagccctgatggatgagaccatgaaggagctgaaggcctacaagtctgagctggaggagcagctgacccctgtggctgaggagaccagagctaggctgtctaaggagctgcaggctgctcaggccaggctgggggctgacatggaggatgtgtgtggcaggctggtgcagtataggggggaggtccaggccatgctggggcagagcactgaggagctgagggtgaggctggccagccatctgaggaagctgaggaagaggctgctgagggatgctgatgacctgcagaagagactggctgtgtaccaggctggggccagggagggggctgagaggggcctgtctgccatcagggagaggctggggcctctggtggagcagggcagggtgagggctgccactgtgggctctctggctggccagcccctgcaggagagggcccaggcttggggggagaggctgagagccagaatggaggagatgggcagcaggaccagggacaggctggatgaggtgaaggagcaggtggctgaggtgagggccaagctggaggagcaggcccagcagatcaggctgcaggctgaggcctttcaggccaggctgaagagctggtttgaacccctggtggaggacatgcagagacagtgggctggcctggtggagaaggtgcaggctgctgtgggcacctctgctgcccctgtgccctctgataaccactgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgc

SEQ ID NO: 78 - DNA, 2565 bp, synthetic construct
  source 1..2565: mol_type = other DNA
gcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgaaggtgctgtgggctgctctgctggtgaccttcctggctggctgccaggctaaggtggagcaggctgtggagactgagcctgagcctgagctgaggcagcagactgagtggcagtctgggcagaggtgggagctggccctgggcaggttctgggactacctgaggtgggtgcagaccctgtctgagcaggtgcaggaggagctgctgtctagccaggtgacccaggaactgagggccctgatggatgagaccatgaaggagctgaaggcctacaagtctgagctggaggagcagctgacccctgtggctgaggagaccagggccaggctgtctaaggagctgcaggctgcccaggccaggctgggggctgacatggaggatgtgtgtggcaggctggtgcagtataggggggaggtgcaggctatgctgggccagagcactgaggagctgagggtcaggctggccagccacctgaggaagctgaggaagaggctgctgagggatgctgatgacctgcagaagaggctggctgtgtaccaggctggggccagggagggggctgagaggggcctgtctgccatcagggagaggctgggccctctggtggagcaggggagggtgagggctgccactgtgggcagcctggctggccagcccctgcaggaaagggcccaggcctggggggagagactgagggccaggatggaggagatgggcagcaggaccagagacaggctggatgaggtgaaggagcaggtggctgaggtgagggccaagctggaggagcaggcccagcagattaggctgcaggctgaggccttccaggccaggctgaagtcttggtttgagcccctggtggaagatatgcagaggcagtgggctggcctggtggagaaggtgcaggctgctgtggggacctctgctgctcctgtgccctctgacaaccactgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgc

SEQ ID NO: 79 - DNA, 2565 bp, synthetic construct
  source 1..2565: mol_type = other DNA
gcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgaaggtgctgtgggctgccctgctggtgactttcctggctggctgccaggctaaggtggagcaggctgtggagactgagccagagcctgagctgaggcagcagactgaatggcagtctggccagaggtgggagctggccctgggcaggttctgggactacctgagatgggtgcagaccctgtctgagcaggtccaggaggagctgctgtcttctcaggtgacccaggagctgagggccctgatggatgagactatgaaggagctgaaggcttacaagtctgagctggaggagcagctgacccctgtggctgaggagaccagggccagactgagcaaggagctgcaggctgctcaggccaggctgggggctgacatggaggatgtgtgtggcaggctggtgcagtattctggggaggtgcaggctatgctgggccagagcactgaggaactgagggtgaggctggcctctcatctgaggaagctgaggaagaggctgctgagggatgctgatgacctgcagaagaggctggctgtgtatcaggctggggccagggagggggctgagagggggctgtctgctatcagggagaggctgggccccctggtggagcaggggagagtgagggctgccactgtggggtctctggctggccagcccctgcaggagagggcccaggcctggggggagaggctgagggctagaatggaggagatggggtctaggaccagggacaggctggatgaggtcaaggaacaggtggctgaggtcagggccaagctggaggagcaggcccagcagattaggctgcaggctgaggcctttcaggccaggctgaagagctggtttgagcccctggtggaggacatgcagaggcagtgggctggcctggtggagaaggtgcaggctgctgtgggcacttctgctgcccctgtgccctctgacaaccattgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgc

SEQ ID NO: 80 - DNA, 2565 bp, synthetic construct
  source 1..2565: mol_type = other DNA
gcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgaaggtgctgtgggctgccctgctggtgacctttctggctgggtgccaggccaaggtggagcaggctgtggagactgagcctgagcctgagctgaggcagcagactgagtggcagtctgggcagaggtgggagctggccctggggaggttctgggactacctgaggtgggtgcagactctgtctgagcaggtgcaggaggagctgctgtctagccaggtgacccaggagctgagggccctgatggatgagaccatgaaggagctgaaggcctacaagtctgagctggaagagcagctgacccctgtggctgaggagaccagggctaggctgagcaaggaactgcaggctgctcaggccaggctgggggctgacatggaagatgtgtgtgggaggctggtgcagtactctggggaggtgcaggccatgctgggccagagcactgaggagctgagggtgaggctggccagccacctgaggaagctgaggaagaggctgctgagggatgctgatgacctgcagaagaggctggctgtgtaccaggctggggccagggagggggctgagaggggcctgtctgctatcagggagaggctgggccccctggtggagcagggcagggtgagggctgccactgtgggcagcctggctgggcagcctctgcaggagagggcccaggcctggggggagaggctgagagccaggatggaggagatgggctctaggactagggacaggctggatgaggtgaaggagcaggtggctgaggtgagggccaagctggaggagcaggctcagcagatcaggctgcaggctgaggctttccaggccaggctgaagtcttggtttgagcccctggtggaggatatgcagaggcagtgggctggcctggtggagaaggtgcaggctgctgtgggcacctctgctgcccctgtgccctctgacaaccattgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgc

SEQ ID NO: 81 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 82 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 83 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 84 - protein, 736 aa, synthetic construct
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 85 - DNA, 365 bp, unidentified
  source 1..365: mol_type = other DNA; note = cytomegalovirus
atctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 86 - DNA, 282 bp, Gallus gallus
  source 1..282: mol_type = genomic DNA
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 87 - DNA, 967 bp, Gallus gallus
  source 1..967: mol_type = genomic DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcg

SEQ ID NO: 88 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 89 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 90 - DNA, 119 bp, synthetic construct
  source 1..119: mol_type = other DNA
cctgcaggcagctgcgcgctcgctcgctcactgaggccgcccgggcaaagcccgggcggcctcagtgagcgagcgagcgcgcagagagggagtggccaactccatcactaggggttcct

SEQ ID NO: 91 - DNA, 119 bp, synthetic construct
  source 1..119: mol_type = other DNA
aggaacccctagtgatggagttggccactccctctctgcgcgctcgctcgctcactgaggccgcccgggctttgcccgggcggcctcagtgagcgagcgagcgcgcagctgcctgcagg

SEQ ID NO: 92 - DNA, 145 bp, synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 93 - DNA, 145 bp, unidentified
  source 1..145: mol_type = other DNA; note = Adeno-associated dependoparvovirus
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 94 - DNA, 145 bp, synthetic construct; identical to SEQ ID NO: 92
  source 1..145: mol_type = other DNA

SEQ ID NO: 95 - DNA, 954 bp, synthetic construct
  source 1..954: mol_type = other DNA
atgaaggtgttgtgggcagcactgctggtcactttcctggcaggctgccaggccaaggtggagcaggcagtagagactgaaccagaacctgaactgaggcaacagactgaatggcagagtggtcaaagatgggaactggcccttggaaggttttgggactacctgaggtgggtgcagaccctcagtgagcaggtgcaggaggagctgctgagctcccaggtgacccaagaactaagggctctgatggatgagaccatgaaggagctgaaggcctacaagtctgagctggaggaacagctcaccccagtggctgaagagactagagcaaggctcagcaaggagttgcaggctgcccaggccaggctgggggctgacatggaagatgtatgtggaaggcttgtgcagtacagtggtgaggtgcaggccatgctgggacagagcactgaagagctgagggtcaggctggcctcccacttaaggaagctaagaaaaagactgctcagagatgctgatgacctccagaagagactggctgtgtaccaggctggagccagagagggagcagagagaggactgagtgccattagggagagattgggtcccctggtagagcagggcagggtcagagctgccacagtgggctctttggcaggccagcctttgcaggaaagagctcaggcctggggagagagactgagagccaggatggaggagatgggatctaggaccagggacagactggatgaagtcaaagaacaagttgctgaggtgagggccaagctggaggagcaagcccagcagattaggctacaggcagaggccttccaggccaggctgaagagctggtttgagcctttggtagaggacatgcagagacagtgggctgggttggtggagaaggtccaggctgctgttggcacatcagcagcccctgtccccagtgacaaccactga

SEQ ID NO: 96 - DNA, 954 bp, synthetic construct
  source 1..954: mol_type = other DNA
atgaaggtcctctgggcagccctgctggtcacattcctggcaggctgtcaagccaaggtggaacaagctgtggagactgagccagagccagagctcaggcagcaaacagagtggcagtctggccagagatgggaattggcactgggaagattctgggactacctgaggtgggttcagaccctgtcagagcaggtgcaggaggagctcctgagctcacaagtcactcaagagctgagagcacttatggatgaaaccatgaaggaattaaaggcctacaaatcagaattggaagagcagctgacacctgtggctgaagagaccagggccagactgagtaaagaactgcaagcagcccaggccagacttggagctgacatggaggatgtttgtggcaggctggtgcagtacagtggtgaagtccaagccatgctgggacaatccacagaggagctgagggtcaggctggccagccacctaagaaaactgaggaagagactcctgagggatgctgatgatctccagaaaaggttggctgtgtaccaagctggagccagggaaggagctgaaaggggactctctgccatcagagaaagactgggtcccctggtggagcagggcagggtgagagcagccacagtgggaagcttggcaggacagccactccaggaaagggcacaggcatggggagaaaggctgagggccaggatggaggagatgggcagcaggaccagggacaggctggatgaggtgaaggaacaagtggcagaggtaagggcaaaactggaggagcaggcccagcagatcaggctccaggctgaagccttccaagcaaggttgaaaagctggtttgagcctcttgtggaagacatgcagagacagtgggctggccttgtggaaaaagtccaagctgcagtaggcaccagtgcagcacctgtcccatcagacaaccattga

SEQ ID NO: 97 - DNA, 954 bp, synthetic construct
  source 1..954: mol_type = other DNA
atgaaagtcctctgggctgccttgctggtaactttcttggctggctgccaggccaaggtggagcaagctgtggaaacagagccagagccagagctgaggcaacagacagaatggcagtcaggccagaggtgggagctggctctggggagattctgggattacctgaggtgggtccagacactctctgaacaagtgcaagaggagctgctgtcctcccaagtcacccaggagctgagagccttgatggatgaaaccatgaaagagctgaaagcttacaagtctgagctggaggaacagttgaccccagtggctgaggagaccagggccagattgtccaaggaactgcaggctgctcaggcaaggttgggggcagatatggaggatgtgtgtgggaggttggtgcagtacagtggtgaggtgcaggccatgctgggacagagcactgaagagctaagggtcagattagcaagtcatttgaggaagctgagaaagaggctgctgagagatgctgatgaccttcagaagagactggctgtctaccaagctggtgccagagaaggggctgaaagaggtctctcagccatcagggagaggctggggcctctggttgagcagggtagggtgagagcagccactgtgggatccctggctggtcaaccactccaagaaagagcacaggcctggggggagagactgagggccaggatggaggaaatgggcagcaggacaagagacaggctggatgaggtcaaggagcaagtagcagaagtgagggccaaactggaggaacaggcccagcagatcagactccaagctgaggccttccaggccaggctgaaatcttggtttgagcctttggtagaagacatgcagaggcagtgggcagggttggtagaaaaggtgcaggctgctgtggggacttcagcagcccctgtgccctcagacaatcactga

SEQ ID NO: 98 - DNA, 954 bp, synthetic construct
  source 1..954: mol_type = other DNA
atgaaggtcctctgggctgccctgctggtcacctttctagctgggtgccaggccaaggtggaacaagcagtggaaactgagcctgagcctgagctgaggcaacagacagagtggcagtctggtcagaggtgggagcttgcactggggaggttctgggattacctgaggtgggtccagaccctgagtgagcaggtgcaggaggagctccttagctcacaggttactcaggagttgagagcactcatggatgaaaccatgaaggagctgaaggcctacaaaagtgaactggaagaacagctgacccctgttgcagaagaaaccagggcaaggctctccaaagagctgcaagctgctcaggcaagactgggtgctgacatggaggatgtctgtggcaggctggtgcagtatagtggtgaggtgcaggcaatgctggggcagtccactgaagagctgagggtgaggctggcctctcatcttaggaagttaaggaagaggctgctcagggatgctgatgacctccagaagaggctggctgtgtatcaggctggggcaagggagggggcagagaggggcctctctgccataagggagagactgggtccactggtggaacagggcagggtgagagcagccacagtgggctctctggcagggcagcccctccaagaaagagcccaggcttggggagaaagactgagggccaggatggaggaaatgggatccaggacaagggacaggctggatgaggtgaaggagcaggtggctgaggtgagggcaaagctggaagagcaagcccagcagatcaggctccaggctgaagctttccaagcaaggctgaaaagctggtttgagcccctggtggaggacatgcagaggcagtgggcaggcctggtggagaaagttcaggcagctgtgggtacttcagctgcacctgtcccttctgacaatcactga

SEQ ID NO: 99 - DNA, 954 bp, synthetic construct
  source 1..954: mol_type = other DNA
atgaaagtcctgtgggcagccctcctggtcaccttcctggctggctgtcaagctaaagtggagcaggctgtggaaacagagcctgagccagaacttagacaacaaactgagtggcagagtgggcagaggtgggagctggccttgggcagattctgggactacctgaggtgggtccagactctttctgagcaggtgcaggaggagctcctgtccagtcaggtgacccaggagctgagggctctcatggatgagacaatgaaagagctgaaggcctacaaaagtgagctggaagagcagctgaccccagtggctgaagagacaagggccaggttaagcaaagagctccaggctgcccaggccaggctgggagcagacatggaggatgtctgtggcaggctggtgcagtatagtggggaggtgcaagccatgttaggccagagcactgaagagctgagggtgaggttagcttctcacctgagaaaattaaggaagaggctgctgagggatgctgatgacctgcagaagaggttggctgtttaccaggcaggggctagagagggagcagagaggggcctctctgccatcagagagaggctgggcccccttgtggagcagggcagggtgagagcagccactgttggctcacttgcagggcagcctctgcaggaaagagcccaggcatggggggagagactgagggcaagaatggaagagatgggctcaaggacaagagacaggctggatgaggtgaaggagcaggtggcagaagtgagggccaagctagaggaacaggcccagcagatcaggctccaggcagaagccttccaggccaggctcaagtcctggtttgagcccttagtggaggacatgcagaggcagtgggctggcctggtggagaaggtacaggctgctgtggggacttcagcagccccagtgcccagtgacaaccactga

SEQ ID NO: 100 - DNA, 954 bp, synthetic construct
  source 1..954: mol_type = other DNA
atgaaggtcctctgggcagctctcctggttaccttcttagctggctgtcaagccaaagtagaacaagcagtggaaacagagccagagccagagctcagacagcaaacagaatggcagtctggtcagagatgggaactggcactgggtaggttctgggactacctgaggtgggtccagaccctgtcagagcaggtccaggaagagctgctgagcagccaggtgactcaagagctgagggccctcatggatgaaaccatgaaggagctgaaggcctacaagagtgagttagaagagcagctcacacctgtggctgaagaaacaagggccaggctcagcaaggagctgcaagctgcacaggccaggctgggtgctgatatggaggatgtgtgtggcaggctggtgcagtatagtggtgaagtccaggccatgttgggccaatccacagaggagctcagggtcaggctggcctcccatctcaggaaactaagaaagagactgctcagggatgctgatgaccttcagaagaggctggctgtgtaccaggcaggagccagagagggggctgagaggggtctctcagccatcagggaaagacttgggcccttggttgagcagggcagggtgagagcagccacagttgggtctctggctggccagcccctccaggagagagctcaggcctggggtgagagactgagggccaggatggaggagatggggagcaggaccagggacaggttggatgaggtcaaggagcaggtggcagaagtgagagccaagctggaagagcaggcccagcaaatcaggctgcaagcagaggcatttcaggccaggctgaagtcatggtttgagcctctggtggaggacatgcagaggcagtgggctgggctggtggaaaaggtacaggctgctgtgggcaccagtgcagcacctgtcccatctgataaccattga

SEQ ID NO: 101 - DNA, 954 bp, synthetic construct
  source 1..954: mol_type = other DNA
atgaaagtcctgtgggcagccctccttgtgaccttcctggcaggctgtcaagcaaaggtggagcaggctgtagagactgagccagagcctgaactgaggcagcagacagaatggcagtctggccagagatgggagctggccctgggaagattttgggactacctgaggtgggtccagaccctgtcagaacaggttcaggaagagctgctttccagccaagtgacacaggagctgagagcccttatggatgagacaatgaaagaactgaaggcttataaaagtgagctagaggagcagctgaccccagtggctgaagagaccagggccaggctcagcaaagagctccaggctgcacaggccagattgggagctgacatggaggatgtgtgtgggagactagtgcagtacagtggtgaagtgcaggccatgctgggacaaagcacagaagagttgagagtcaggctggcatcccacctgaggaaactcaggaaaagactgctcagggatgctgatgacctccagaagagactggcagtctatcaggctggtgccagagagggggcagagaggggactctcagccatcagggagaggctggggcctctggttgagcagggcagagtcagagcagcaacagtggggagtctggcaggccagcccttacaagagagagctcaggcatggggagagaggctgagggccaggatggaagaaatgggatcaaggactagagacaggttggatgaagtaaaggagcaggtggcagaagtgagggccaagcttgaggagcaggcccagcagatcaggctacaggctgaggcatttcaggccaggctgaagagctggtttgagcccttggtggaggatatgcagaggcagtgggctggattggtggaaaaggtacaggctgctgtgggcacctcagcagctcctgtcccctctgataatcattga

SEQ ID NO: 102 - DNA, 954 bp, synthetic construct
  source 1..954: mol_type = other DNA
atgaaggtcctctgggcagccctgctggtcaccttcctggctggctgccaggccaaggtggagcaggctgtggagactgagccagagccagagctgaggcagcagacagagtggcagtctggccagagatgggagctggccctgggaagattctgggactacctgaggtgggtccagaccctgtcagagcaggtgcaggaggagctgctgagctcccaggtgacccaggagctgagggccctgatggatgaaaccatgaaggagctgaaggcctacaagagtgagctggaggagcagctgacccctgtggctgaagagaccagggccaggctcagcaaggagctgcaggctgcccaggccaggctgggagctgacatggaggatgtgtgtggcaggctggtgcagtacagtggtgaggtgcaggccatgctgggacagagcactgaagagctgagggtcaggctggcctcccacctgaggaagctgaggaagagactgctcagggatgctgatgacctccagaagaggctggctgtgtaccaggctggggccagagagggggcagagaggggactctcagccatcagggagaggctggggcctctggtggagcagggcagggtgagagcagccacagtgggctctctggcaggccagcctctccaggaaagagctcaggcctggggagagagactgagggccaggatggaggagatgggcagcaggaccagggacaggctggatgaggtgaaggagcaggtggctgaggtgagggccaagctggaggagcaggcccagcagatcaggctccaggcagaggccttccaggccaggctgaagagctggtttgagcccctggtggaggacatgcagaggcagtgggctggcctggtggagaaggtccaggctgctgtgggcacctcagcagcccctgtccccagtgacaaccactga

SEQ ID NO: 103 - DNA, 954 bp, synthetic construct
  source 1..954: mol_type = other DNA
atgaaagtgctgtgggctgccctgctggtcacattcctggctggctgccaggccaaggtggagcaggctgtggagacagagccagagccagaactgaggcagcagacagagtggcagtctggccaaagatgggagctagccctgggaagattctgggactacctgagatgggtgcagaccctctcagaacaagtccaggaggagctgctgtcctcacaggtgacccaggagctgagggccctgatggatgaaacaatgaaggagctgaaggcatataagtcagaattggaggagcagctgactcctgtggctgaggagaccagggctagactctccaaggagctccaggctgcccaggccagattaggagcagacatggaggatgtctgtgggaggctggtgcagtacagtggtgaggtccaggccatgctgggccaaagcacagaggagctgagggtcaggctggccagccatctgaggaagctgaggaaaaggctgctcagggatgctgatgacctgcagaagagactggcagtgtaccaggctggtgccagagagggggcagagagggggctctcagccatcagagagaggctggggcctctggtggagcagggcagagtgagggctgccactgtgggctccttggcaggacagcccctccaagagagagctcaggcctggggagagaggctcagggccaggatggaggaaatgggcagtaggaccagggacaggcttgatgaggtgaaggagcaggtggctgaggtcagagccaagctggaggagcaggctcagcagattaggctgcaggcagaagccttccaggccaggctgaaatcctggtttgagcccctggtggaagacatgcaaaggcagtgggcagggctggtggagaaggtgcaggctgctgtgggcacatcagctgcccctgtgcccagtgataaccactga

SEQ ID NO: 104 - DNA, 954 bp, synthetic construct
  source 1..954: mol_type = other DNA
atgaaagtgctatgggcagccctcttggtgaccttcctggctggctgccaggccaaggtggagcaggctgtggagacagagccagaaccagagctgagacagcaaactgagtggcagtcaggccagagatgggagctggccctggggagattttgggactacttaaggtgggtccagaccctcagtgagcaggtgcaggaagagctgctgagctcccaggtcacccaggagctgagggcccttatggatgaaaccatgaaggagctgaaggcctacaagtcagaactggaggagcagctcacccctgtggctgaggagaccagggcaaggctctccaaggagctacaggcagcccaggccaggctgggtgcagacatggaggatgtctgtgggagactggtgcagtacagtggtgaagtacaggccatgcttggccaatccacagaggagttgagggtgaggctggcctcccacctgaggaagctgagaaagagactcctcagggatgcagatgacctccagaagaggctggcagtttaccaggcaggggccagagagggtgcagaaagagggctgtcagccatcagggaaaggctgggccctctggtggagcagggcagagtgagagcagcaactgttggctccttggctgggcagcctctgcaggagagagctcaggcctggggggagagactcagggccaggatggaggagatgggctccaggacaagggacaggttggatgaagtgaaggagcaggtggctgaggtgagagcaaagttggaggagcaggcccagcagatcaggcttcaggcagaggccttccaggccaggctgaagtcctggtttgagccactggtggaggatatgcagagacagtgggcaggcctggtggagaaggtccaggcagcagtgggtacctctgcagccccagttcccagtgacaatcactga

SEQ ID NO: 105 - DNA, 954 bp, synthetic construct
  source 1..954: mol_type = other DNA
atgaaggtgctgtgggctgccctgctggtgaccttcctggctggctgtcaggctaaagtggagcaggctgtggagactgagcctgagccagagctgaggcagcagactgagtggcagtctggccagagatgggagctggctctgggcaggttctgggactacctgaggtgggtgcagaccctgtctgagcaggtgcaggaggagctgctgtcttctcaggtgacccaggaactgagggccctgatggatgagactatgaaggagctgaaggcctacaagtctgagctggaggagcagctgacccctgtggctgaggagaccagggccaggctgagcaaggagctgcaggctgcccaggccagactgggggctgacatggaggatgtgtgtggcagactggtgcagtacagtggggaggtgcaggccatgctggggcagtctactgaggagctgagggtgaggctggccagccacctgaggaagctgaggaagaggctgctgagggatgctgatgacctgcagaagaggctggctgtgtatcaggctggggctagggagggggctgagaggggcctgtctgccatcagggagaggctggggcctctggtggagcagggcagggtgagggctgccactgtggggagcctggctggccagcctctgcaggagagggctcaggcctggggggagaggctgagggccaggatggaggagatggggagcaggaccagggacaggctggatgaggtgaaggagcaggtggctgaggtgagggccaagctggaagagcaggcccagcagatcaggctgcaggctgaggccttccaggccaggctgaagagctggtttgagcccctggtggaggacatgcagaggcagtgggctggcctggtggagaaggtgcaggctgctgtgggcacctctgctgcccctgtgccctctgacaatcactga

SEQ ID NO: 106 - DNA, 2170 bp, synthetic construct
  source 1..2170: mol_type = other DNA
atgaaggtcctctgggcagccctgctggtcacattcctggcaggtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcaggctgtcaagccaaggtggaacaagctgtggagactgagccagagccagagctcaggcagcaaacagagtggcagtctggccagagatgggaattggcactgggaagattctgggactacctgaggtgggttcagaccctgtcagagcaggtgcaggaggagctcctgagctcacaagtcactcaagagctgaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagagcacttatggatgaaaccatgaaggaattaaaggcctacaaatcagaattggaagagcagctgacacctgtggctgaagagaccagggccagactgagtaaagaactgcaagcagcccaggccagacttggagctgacatggaggatgtttgtggcaggctggtgcagtacagtggtgaagtccaagccatgctgggacaatccacagaggagctgagggtcaggctggccagccacctaagaaaactgaggaagagactcctgagggatgctgatgatctccagaaaaggttggctgtgtaccaagctggagccagggaaggagctgaaaggggactctctgccatcagagaaagactgggtcccctggtggagcagggcagggtgagagcagccacagtgggaagcttggcaggacagccactccaggaaagggcacaggcatggggagaaaggctgagggccaggatggaggagatgggcagcaggaccagggacaggctggatgaggtgaaggaacaagtggcagaggtaagggcaaaactggaggagcaggcccagcagatcaggctccaggctgaagccttccaagcaaggttgaaaagctggtttgagcctcttgtggaagacatgcagagacagtgggctggccttgtggaaaaagtccaagctgcagtaggcaccagtgcagcacctgtcccatcagacaaccattga

SEQ ID NO: 107 - DNA, 2170 bp, synthetic construct
  source 1..2170: mol_type = other DNA
atgaaagtcctctgggctgccttgctggtaactttcttggctggtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcaggctgccaggccaaggtggagcaagctgtggaaacagagccagagccagagctgaggcaacagacagaatggcagtcaggccagaggtgggagctggctctggggagattctgggattacctgaggtgggtccagacactctctgaacaagtgcaagaggagctgctgtcctcccaagtcacccaggagctgaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagagccttgatggatgaaaccatgaaagagctgaaagcttacaagtctgagctggaggaacagttgaccccagtggctgaggagaccagggccagattgtccaaggaactgcaggctgctcaggcaaggttgggggcagatatggaggatgtgtgtgggaggttggtgcagtacagtggtgaggtgcaggccatgctgggacagagcactgaagagctaagggtcagattagcaagtcatttgaggaagctgagaaagaggctgctgagagatgctgatgaccttcagaagagactggctgtctaccaagctggtgccagagaaggggctgaaagaggtctctcagccatcagggagaggctggggcctctggttgagcagggtagggtgagagcagccactgtgggatccctggctggtcaaccactccaagaaagagcacaggcctggggggagagactgagggccaggatggaggaaatgggcagcaggacaagagacaggctggatgaggtcaaggagcaagtagcagaagtgagggccaaactggaggaacaggcccagcagatcagactccaagctgaggccttccaggccaggctgaaatcttggtttgagcctttggtagaagacatgcagaggcagtgggcagggttggtagaaaaggtgcaggctgctgtggggacttcagcagcccctgtgccctcagacaatcactga

SEQ ID NO: 108 - DNA, 2170 bp, synthetic construct
  source 1..2170: mol_type = other DNA
atgaaggtcctctgggctgccctgctggtcacctttctagctggtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcagggtgccaggccaaggtggaacaagcagtggaaactgagcctgagcctgagctgaggcaacagacagagtggcagtctggtcagaggtgggagcttgcactggggaggttctgggattacctgaggtgggtccagaccctgagtgagcaggtgcaggaggagctccttagctcacaggttactcaggagttgaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagagcactcatggatgaaaccatgaaggagctgaaggcctacaaaagtgaactggaagaacagctgacccctgttgcagaagaaaccagggcaaggctctccaaagagctgcaagctgctcaggcaagactgggtgctgacatggaggatgtctgtggcaggctggtgcagtatagtggtgaggtgcaggcaatgctggggcagtccactgaagagctgagggtgaggctggcctctcatcttaggaagttaaggaagaggctgctcagggatgctgatgacctccagaagaggctggctgtgtatcaggctggggcaagggagggggcagagaggggcctctctgccataagggagagactgggtccactggtggaacagggcagggtgagagcagccacagtgggctctctggcagggcagcccctccaagaaagagcccaggcttggggagaaagactgagggccaggatggaggaaatgggatccaggacaagggacaggctggatgaggtgaaggagcaggtggctgaggtgagggcaaagctggaagagcaagcccagcagatcaggctccaggctgaagctttccaagcaaggctgaaaagctggtttgagcccctggtggaggacatgcagaggcagtgggcaggcctggtggagaaagttcaggcagctgtgggtacttcagctgcacctgtcccttctgacaatcactga

SEQ ID NO: 109 - DNA, 2170 bp, synthetic construct
  source 1..2170: mol_type = other DNA
atgaaagtcctgtgggcagccctcctggtcaccttcctggctggtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcaggctgtcaagctaaagtggagcaggctgtggaaacagagcctgagccagaacttagacaacaaactgagtggcagagtgggcagaggtgggagctggccttgggcagattctgggactacctgaggtgggtccagactctttctgagcaggtgcaggaggagctcctgtccagtcaggtgacccaggagctgaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagggctctcatggatgagacaatgaaagagctgaaggcctacaaaagtgagctggaagagcagctgaccccagtggctgaagagacaagggccaggttaagcaaagagctccaggctgcccaggccaggctgggagcagacatggaggatgtctgtggcaggctggtgcagtatagtggggaggtgcaagccatgttaggccagagcactgaagagctgagggtgaggttagcttctcacctgagaaaattaaggaagaggctgctgagggatgctgatgacctgcagaagaggttggctgtttaccaggcaggggctagagagggagcagagaggggcctctctgccatcagagagaggctgggcccccttgtggagcagggcagggtgagagcagccactgttggctcacttgcagggcagcctctgcaggaaagagcccaggcatggggggagagactgagggcaagaatggaagagatgggctcaaggacaagagacaggctggatgaggtgaaggagcaggtggcagaagtgagggccaagctagaggaacaggcccagcagatcaggctccaggcagaagccttccaggccaggctcaagtcctggtttgagcccttagtggaggacatgcagaggcagtgggctggcctggtggagaaggtacaggctgctgtggggacttcagcagccccagtgcccagtgacaaccactga

SEQ ID NO: 110 - DNA, 2170 bp, synthetic construct
  source 1..2170: mol_type = other DNA
atgaaggtcctctgggcagctctcctggttaccttcttagctggtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcaggctgtcaagccaaagtagaacaagcagtggaaacagagccagagccagagctcagacagcaaacagaatggcagtctggtcagagatgggaactggcactgggtaggttctgggactacctgaggtgggtccagaccctgtcagagcaggtccaggaagagctgctgagcagccaggtgactcaagagctgaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagggccctcatggatgaaaccatgaaggagctgaaggcctacaagagtgagttagaagagcagctcacacctgtggctgaagaaacaagggccaggctcagcaaggagctgcaagctgcacaggccaggctgggtgctgatatggaggatgtgtgtggcaggctggtgcagtatagtggtgaagtccaggccatgttgggccaatccacagaggagctcagggtcaggctggcctcccatctcaggaaactaagaaagagactgctcagggatgctgatgaccttcagaagaggctggctgtgtaccaggcaggagccagagagggggctgagaggggtctctcagccatcagggaaagacttgggcccttggttgagcagggcagggtgagagcagccacagttgggtctctggctggccagcccctccaggagagagctcaggcctggggtgagagactgagggccaggatggaggagatggggagcaggaccagggacaggttggatgaggtcaaggagcaggtggcagaagtgagagccaagctggaagagcaggcccagcaaatcaggctgcaagcagaggcatttcaggccaggctgaagtcatggtttgagcctctggtggaggacatgcagaggcagtgggctgggctggtggaaaaggtacaggctgctgtgggcaccagtgcagcacctgtcccatctgataaccattga

SEQ ID NO: 111 - DNA, 2170 bp, synthetic construct
  source 1..2170: mol_type = other DNA
atgaaagtcctgtgggcagccctccttgtgaccttcctggcaggtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcaggctgtcaagcaaaggtggagcaggctgtagagactgagccagagcctgaactgaggcagcagacagaatggcagtctggccagagatgggagctggccctgggaagattttgggactacctgaggtgggtccagaccctgtcagaacaggttcaggaagagctgctttccagccaagtgacacaggagctgaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagagcccttatggatgagacaatgaaagaactgaaggcttataaaagtgagctagaggagcagctgaccccagtggctgaagagaccagggccaggctcagcaaagagctccaggctgcacaggccagattgggagctgacatggaggatgtgtgtgggagactagtgcagtacagtggtgaagtgcaggccatgctgggacaaagcacagaagagttgagagtcaggctggcatcccacctgaggaaactcaggaaaagactgctcagggatgctgatgacctccagaagagactggcagtctatcaggctggtgccagagagggggcagagaggggactctcagccatcagggagaggctggggcctctggttgagcagggcagagtcagagcagcaacagtggggagtctggcaggccagcccttacaagagagagctcaggcatggggagagaggctgagggccaggatggaagaaatgggatcaaggactagagacaggttggatgaagtaaaggagcaggtggcagaagtgagggccaagcttgaggagcaggcccagcagatcaggctacaggctgaggcatttcaggccaggctgaagagctggtttgagcccttggtggaggatatgcagaggcagtgggctggattggtggaaaaggtacaggctgctgtgggcacctcagcagctcctgtcccctctgataatcattga

SEQ ID NO: 112 - DNA, 2170 bp, synthetic construct
  source 1..2170: mol_type = other DNA
atgaaggtcctctgggcagccctgctggtcaccttcctggctggtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcaggctgccaggccaaggtggagcaggctgtggagactgagccagagccagagctgaggcagcagacagagtggcagtctggccagagatgggagctggccctgggaagattctgggactacctgaggtgggtccagaccctgtcagagcaggtgcaggaggagctgctgagctcccaggtgacccaggagctgaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagggccctgatggatgaaaccatgaaggagctgaaggcctacaagagtgagctggaggagcagctgacccctgtggctgaagagaccagggccaggctcagcaaggagctgcaggctgcccaggccaggctgggagctgacatggaggatgtgtgtggcaggctggtgcagtacagtggtgaggtgcaggccatgctgggacagagcactgaagagctgagggtcaggctggcctcccacctgaggaagctgaggaagagactgctcagggatgctgatgacctccagaagaggctggctgtgtaccaggctggggccagagagggggcagagaggggactctcagccatcagggagaggctggggcctctggtggagcagggcagggtgagagcagccacagtgggctctctggcaggccagcctctccaggaaagagctcaggcctggggagagagactgagggccaggatggaggagatgggcagcaggaccagggacaggctggatgaggtgaaggagcaggtggctgaggtgagggccaagctggaggagcaggcccagcagatcaggctccaggcagaggccttccaggccaggctgaagagctggtttgagcccctggtggaggacatgcagaggcagtgggctggcctggtggagaaggtccaggctgctgtgggcacctcagcagcccctgtccccagtgacaaccactga

SEQ ID NO: 113 - DNA, 2170 bp, synthetic construct
  source 1..2170: mol_type = other DNA
atgaaagtgctgtgggctgccctgctggtcacattcctggctggtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcaggctgccaggccaaggtggagcaggctgtggagacagagccagagccagaactgaggcagcagacagagtggcagtctggccaaagatgggagctagccctgggaagattctgggactacctgagatgggtgcagaccctctcagaacaagtccaggaggagctgctgtcctcacaggtgacccaggagctgaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagggccctgatggatgaaacaatgaaggagctgaaggcatataagtcagaattggaggagcagctgactcctgtggctgaggagaccagggctagactctccaaggagctccaggctgcccaggccagattaggagcagacatggaggatgtctgtgggaggctggtgcagtacagtggtgaggtccaggccatgctgggccaaagcacagaggagctgagggtcaggctggccagccatctgaggaagctgaggaaaaggctgctcagggatgctgatgacctgcagaagagactggcagtgtaccaggctggtgccagagagggggcagagagggggctctcagccatcagagagaggctggggcctctggtggagcagggcagagtgagggctgccactgtgggctccttggcaggacagcccctccaagagagagctcaggcctggggagagaggctcagggccaggatggaggaaatgggcagtaggaccagggacaggcttgatgaggtgaaggagcaggtggctgaggtcagagccaagctggaggagcaggctcagcagattaggctgcaggcagaagccttccaggccaggctgaaatcctggtttgagcccctggtggaagacatgcaaaggcagtgggcagggctggtggagaaggtgcaggctgctgtgggcacatcagctgcccctgtgcccagtgataaccactga

SEQ ID NO: 114 - DNA, 2170 bp, synthetic construct
  source 1..2170: mol_type = other DNA
atgaaagtgctatgggcagccctcttggtgaccttcctggctggtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcaggctgccaggccaaggtggagcaggctgtggagacagagccagaaccagagctgagacagcaaactgagtggcagtcaggccagagatgggagctggccctggggagattttgggactacttaaggtgggtccagaccctcagtgagcaggtgcaggaagagctgctgagctcccaggtcacccaggagctgaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagggcccttatggatgaaaccatgaaggagctgaaggcctacaagtcagaactggaggagcagctcacccctgtggctgaggagaccagggcaaggctctccaaggagctacaggcagcccaggccaggctgggtgcagacatggaggatgtctgtgggagactggtgcagtacagtggtgaagtacaggccatgcttggccaatccacagaggagttgagggtgaggctggcctcccacctgaggaagctgagaaagagactcctcagggatgcagatgacctccagaagaggctggcagtttaccaggcaggggccagagagggtgcagaaagagggctgtcagccatcagggaaaggctgggccctctggtggagcagggcagagtgagagcagcaactgttggctccttggctgggcagcctctgcaggagagagctcaggcctggggggagagactcagggccaggatggaggagatgggctccaggacaagggacaggttggatgaagtgaaggagcaggtggctgaggtgagagcaaagttggaggagcaggcccagcagatcaggcttcaggcagaggccttccaggccaggctgaagtcctggtttgagccactggtggaggatatgcagagacagtgggcaggcctggtggagaaggtccaggcagcagtgggtacctctgcagccccagttcccagtgacaatcactga

SEQ ID NO: 115 - DNA, 2170 bp, synthetic construct
  source 1..2170: mol_type = other DNA
atgaaggtgctgtgggctgccctgctggtgaccttcctggctggtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcaggctgtcaggctaaagtggagcaggctgtggagactgagcctgagccagagctgaggcagcagactgagtggcagtctggccagagatgggagctggctctgggcaggttctgggactacctgaggtgggtgcagaccctgtctgagcaggtgcaggaggagctgctgtcttctcaggtgacccaggaactgaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagggccctgatggatgagactatgaaggagctgaaggcctacaagtctgagctggaggagcagctgacccctgtggctgaggagaccagggccaggctgagcaaggagctgcaggctgcccaggccagactgggggctgacatggaggatgtgtgtggcagactggtgcagtacagtggggaggtgcaggccatgctggggcagtctactgaggagctgagggtgaggctggccagccacctgaggaagctgaggaagaggctgctgagggatgctgatgacctgcagaagaggctggctgtgtatcaggctggggctagggagggggctgagaggggcctgtctgccatcagggagaggctggggcctctggtggagcagggcagggtgagggctgccactgtggggagcctggctggccagcctctgcaggagagggctcaggcctggggggagaggctgagggccaggatggaggagatggggagcaggaccagggacaggctggatgaggtgaaggagcaggtggctgaggtgagggccaagctggaagagcaggcccagcagatcaggctgcaggctgaggccttccaggccaggctgaagagctggtttgagcccctggtggaggacatgcagaggcagtgggctggcctggtggagaaggtgcaggctgctgtgggcacctctgctgcccctgtgccctctgacaatcactga

SEQ ID NO: 116 - DNA, 43 bp, synthetic construct
  source 1..43: mol_type = other DNA
atgaaggtgctgtgggctgccctgctggtgaccttcctggctg

SEQ ID NO: 117 - DNA, 193 bp, synthetic construct
  source 1..193: mol_type = other DNA
gctgtcaggctaaagtggagcaggctgtggagactgagcctgagccagagctgaggcagcagactgagtggcagtctggccagagatgggagctggctctgggcaggttctgggactacctgaggtgggtgcagaccctgtctgagcaggtgcaggaggagctgctgtcttctcaggtgacccaggaactgag

SEQ ID NO: 118 - DNA, 718 bp, synthetic construct
  source 1..718: mol_type = other DNA
ggccctgatggatgagactatgaaggagctgaaggcctacaagtctgagctggaggagcagctgacccctgtggctgaggagaccagggccaggctgagcaaggagctgcaggctgcccaggccagactgggggctgacatggaggatgtgtgtggcagactggtgcagtacagtggggaggtgcaggccatgctggggcagtctactgaggagctgagggtgaggctggccagccacctgaggaagctgaggaagaggctgctgagggatgctgatgacctgcagaagaggctggctgtgtatcaggctggggctagggagggggctgagaggggcctgtctgccatcagggagaggctggggcctctggtggagcagggcagggtgagggctgccactgtggggagcctggctggccagcctctgcaggagagggctcaggcctggggggagaggctgagggccaggatggaggagatggggagcaggaccagggacaggctggatgaggtgaaggagcaggtggctgaggtgagggccaagctggaagagcaggcccagcagatcaggctgcaggctgaggccttccaggccaggctgaagagctggtttgagcccctggtggaggacatgcagaggcagtgggctggcctggtggagaaggtgcaggctgctgtgggcacctctgctgcccctgtgccctctgacaatcactga

SEQ ID NO: 119 - DNA, 1047 bp, synthetic construct
  source 1..1047: mol_type = other DNA
gtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcag

SEQ ID NO: 120 - DNA, 169 bp, synthetic construct
  source 1..169: mol_type = other DNA
gtcagtggcagccaggggcagctgcaccctttgcagctccagggttccccaagggccctgcctgctgatggccagtgggcattgtgaagggaagggagcaccaaatgggtggagggagggaggaagccctttgccctggcttggctgaggatccccttggcttttgcag

SEQ ID NO: 121 - DNA, 154 bp, synthetic construct
  source 1..154: mol_type = other DNA
gtaggcaatctgagacaaaaagcctactcttgaagataataatggaagattgatgattgtttagaatgaaaatatgttgaatgctgcacatctgtgttaccatgtttgctagttgtccacccatgtgattgaatactctgaaatattttttcag

SEQ ID NO: 122 - protein, 601 aa, synthetic construct
MAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 123 - protein, 535 aa, synthetic construct
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 124 - DNA, 954 bp, synthetic construct
  source 1..954: mol_type = other DNA
atgaaggtgttgtgggcagcactgctggtcactttcctggcaggctgccaggcgaaggtggagcaggcagtagagactgaaccagaacctgaactgaggcaacagactgaatggcagagtggtcaaagatgggaactggcccttggaaggttttgggactacctgaggtgggtgcagaccctcagtgagcaggtgcaggaggagctgctgagctcccaggtgacccaagaactaagggctctgatggatgagaccatgaaggagctgaaggcctacaagtctgagctggaggaacagctcaccccagtggctgaagagactagagcaaggctcagcaaggagttgcaggctgcccaggccaggctgggggctgacatggaagatgtatgtggaaggcttgtgcagtacaggggcgaggtgcaggccatgctgggacagagcactgaagagctgagggttcggctagcctcccacttaaggaagctaagaaaaagactgctcagagatgccgacgacctccagaagagactggctgtgtaccaggctggagccagagagggagcagagagaggactgagtgccattagggagagattgggtcccctggtagagcagggcagggtcagagctgccacagtgggctctttggcaggccagcctttgcaggaaagagctcaggcctggggagagagactgagagccaggatggaggagatgggatctaggaccagggacagactggatgaagtcaaagaacaagttgctgaggtccgggctaagctggaggagcaagcccagcagattaggctacaggcagaggccttccaggccaggctgaagagctggtttgagcctttggtagaggacatgcagagacagtgggctgggttggtggagaaggtccaggctgctgttggcacatcagcagcccctgtccccagtgacaaccactga

SEQ ID NO: 125 - DNA, 954 bp, synthetic construct
  source 1..954: mol_type = other DNA
atgaaggtcctctgggcagccctgctggtcacattcctggcaggctgtcaagccaaggtggaacaagctgtggagactgagccagagccagagctcaggcagcaaacagagtggcagtcggggcagagatgggaattggcactgggaagattctgggactacctgaggtgggttcagaccctgtcagagcaggtgcaggaggagctcctgagctcacaagtcactcaagagctgagagcacttatggatgaaaccatgaaggaattaaaggcctacaaatcagaattggaagagcagctgacacctgtggctgaagagaccagggccagactgagtaaagaactgcaagcagcccaggccagacttggagctgacatggaggatgtttgtggcaggctggtgcagtacagaggtgaagtccaagccatgctgggacaatccacagaggagctgcgtgtccggctagccagccacctaagaaaactgaggaagagactcctgagggatgctgatgatctccagaaaaggttggctgtgtaccaagctggagccagggaaggagctgaaaggggactctctgccatcagagaaagactgggtcccctggtggagcagggcagggtgcgggcagcaacagtgggaagcttggcaggacagccactccaggaaagggcacaggcatggggagaaaggctgagagcacggatggaagagatgggcagcaggaccagggacaggctggatgaggtgaaggaacaagtggcagaggtaagggcaaaactggaggagcaggcccagcagattcgcctccaggctgaagccttccaagcaaggttgaaaagctggtttgagcctcttgtggaagacatgcagagacagtgggctggccttgtggaaaaagtccaagctgcagtaggcaccagtgcagcacctgtcccatcagacaaccattga

SEQ ID NO: 126 - DNA, 954 bp, synthetic construct
  source 1..954: mol_type = other DNA
atgaaagtcctctgggctgccttgctggtaactttcttggctggctgccaggccaaggtggagcaagctgtggaaacagagcctgagccggagctgaggcaacagacagaatggcagtcaggccagaggtgggagctggctctggggagattctgggattaccttcgctgggtccagacactctctgaacaagtgcaagaggagctgctgtcctcccaagtcacccaggagctgagagccttgatggatgaaaccatgaaagagctgaaagcttacaagtctgagctggaggaacagttgaccccagtggctgaggagaccagggccagattgtccaaggaactgcaggctgctcaggcaaggttgggggcagatatggaggatgtgtgtgggaggttggtgcagtacagaggtgaggtgcaggccatgctgggacagagcactgaagagctaagggtcagattagcaagtcatttgaggaagctgagaaagaggctgctgagagacgcggatgaccttcagaagagactggctgtctaccaagctggtgccagagaaggggctgaaagaggtctctcagccatcagggagaggctggggcctctggttgagcagggtagggtgagggcggctactgtgggatccctggctggtcaaccactccaagaaagagcacaggcctggggggagagactccgtgccagaatggaggaaatgggcagcaggacaagagacaggctggatgaggtcaaggagcaagtagcagaagtgagggccaaactggaggaacaggcccagcagatcagactccaagctgaggccttccaggccaggctgaaatcttggtttgagcctttggtagaagacatgcagaggcagtgggcagggttggtagaaaaggtgcaggctgctgtggggacttcagcagcccctgtgccctcagacaatcactga

SEQ ID NO: 127 - DNA, 954 bp, synthetic construct
  source 1..954: mol_type = other DNA
atgaaggtcctctgggctgccctgctggtcacctttctagctgggtgccaggccaaggtggaacaagcagtggaaactgagcctgagcctgagctgaggcaacagacagagtggcagtctggtcagaggtgggagcttgcactggggaggttctgggattacctccgctgggtgcagaccctgagtgagcaggtgcaggaggagctccttagctcacaggttactcaggagttgagagcactcatggatgaaaccatgaaggagctgaaggcctacaaaagtgaactggaagaacagctgacccctgttgcagaagaaaccagggcaaggctctccaaagagctgcaagctgctcaggcaagactgggtgctgacatggaggatgtctgtggcaggctggtgcagtatcgtggcgaagtacaggcaatgctggggcagtccactgaagagctgagagtgcggctggcttctcatcttaggaagttaaggaagaggctgctccgagatgctgatgacctccagaagaggctggctgtgtatcaggctggggcaagggagggggcagagaggggcctctctgccataagggagagactgggtccactggtggaacaaggccgagtgagagcagccacagtcggctctctggcagggcagcccctccaagaaagagcccaggcttggggagaaagactgagggccaggatggaggaaatgggatccaggacaagagatcgcctggatgaggtgaaggagcaggtggctgaggtgagggcaaagctggaagagcaagcccagcagatcaggctccaggctgaagctttccaagcaaggctgaaaagctggtttgagcccctggtggaggacatgcagaggcagtgggcaggcctggtggagaaagttcaggcagctgtgggtacttcagctgcacctgtcccttctgacaatcactga

SEQ ID NO: 128 - DNA, 954 bp, synthetic construct
  source 1..954: mol_type = other DNA
atgaaagtcctgtgggcagcgctcctggtcaccttcctggctggctgtcaagctaaagtggagcaggctgtggaaacagagcctgagccagaacttagacaacaaactgagtggcagagtgggcagaggtgggagctggccttgggcagattctgggactacctgcgctgggtccagactctttctgagcaggtgcaggaggagctcctgtccagtcaggtgacccaggagctgagggctctcatggatgagacaatgaaagagctgaaggcctacaaaagtgagctggaagagcagctgaccccagtggctgaagagacaagggccaggttaagcaaagagctccaggctgcccaggccaggctgggagcagacatggaggatgtctgcggccgcctggtccagtatagaggggaggtgcaagccatgttaggccagagcactgaagagctgagggtgaggttagcttctcacctgagaaaattaaggaagaggctgctgagggatgctgatgacctgcagaagaggttggctgtttaccaggcaggggctagagagggagcagagaggggcctctctgccatcagagagaggctgggcccccttgtggagcagggccgtgtccgggcagctactgttggctcacttgcagggcagcctctgcaggaaagagcccaggcatggggggagcggttaagggcaagaatggaagagatgggctcaaggacaagagacaggctggacgaagtgaaggagcaggtggcagaagtgagggccaagctagaggaacaggcccagcagattcgcctccaggcagaagccttccaggccaggctcaagtcctggtttgagcccttagtggaggacatgcagaggcagtgggctggcctggtggagaaggtacaggctgctgtggggacttcagcagccccagtgcccagtgacaaccactga

SEQ ID NO: 129 - DNA, 954 bp, synthetic construct
  source 1..954: mol_type = other DNA
atgaaggtcctctgggcagctctcctggttaccttcttagctggctgtcaagccaaagtagaacaagcagtggaaacagagccggagccagagctcagacagcaaacagaatggcagtctggtcagagatgggaactggcactgggtaggttctgggactacctgaggtgggtccagaccctgtcagagcaggtccaggaagagctgctgagcagccaggtgactcaagagctgcgtgccctcatggatgaaaccatgaaggagctgaaggcctacaagagtgagttagaagagcagctcacacctgtggctgaagaaacaagggcccgcctcagcaaggagctgcaagctgcacaggccaggctgggtgctgatatggaggatgtgtgcggcaggctggtgcagtatcgtggggaagtccaggccatgttgggccaatccacagaggagctcagagttcgccttgcctcccatctcaggaaactaagaaagagactactgcgggatgctgatgaccttcagaagaggctggctgtgtaccaggcaggagccagagagggggctgagaggggtctctcagccatcagggaaagacttgggcccttggttgagcagggcagggtgcgggcagccacagttgggtctctggctggccagcccctccaggagagagctcaggcctggggtgagagacttcgagctaggatggaggagatggggagcaggaccagggacaggttggatgaggtcaaggagcaggtggcagaagtgagagccaagctggaagagcaggcccagcaaatcaggctgcaagcagaggcatttcaggcccggctcaagtcatggtttgagcctctggtggaggacatgcagaggcagtgggctgggctggtggaaaaggtacaggctgctgtgggcaccagtgcagcacctgtcccatctgataaccattga

SEQ ID NO: 130 - DNA, 954 bp, synthetic construct
  source 1..954: mol_type = other DNA
atgaaagtcctgtgggcagccctccttgtgaccttcctggcaggctgtcaagcaaaggtggagcaggctgtagagactgagccagagcctgaactgaggcagcagacagaatggcagtctggccagagatgggagctggccctgggaagattttgggactatctccgctgggtccagaccctgtcagaacaggttcaggaagagctgctttccagccaagtgacacaggagctgagagcccttatggatgagacaatgaaagaactgaaggcttataaaagtgagctagaggagcagctgaccccagtggctgaagagacccgggcccggctgagcaaagagctccaggctgcacaggccagattgggagctgacatggaggatgtgtgtgggagactagtgcagtacaggggtgaagtgcaggccatgctgggacaaagcacagaagagttgagagtcaggctggcatcccacctgaggaaactcaggaaaagactcctccgtgatgcagatgacctccagaagagactggcagtctatcaggctggtgccagagagggcgccgagagaggactctcagccatcagggagcggctggggcctctggttgagcagggcagagtcagagcagcaacagtggggagtctggccggccagcccttacaagagagagctcaggcatggggagagaggcttcgcgctaggatggaagaaatgggatcaaggactagagacaggttggatgaagtaaaggagcaggtggcagaagtgagggccaagcttgaggagcaggcccagcagatcaggctacaggctgaggcatttcaggccaggctgaagagctggtttgagcccttggtggaggatatgcagaggcagtgggctggattggtggaaaaggtacaggctgctgttgggacgagtgctgctcctgtcccctctgataatcattga

(19) United States
(12) Patent Application Publication
(10) Pub. No.: US 2026/0315626 A1
(43) Pub. Date: Oct. 15, 2026

(54) APOE GENE THERAPY

(71) Applicant: Spark Therapeutics, Inc., Philadelphia, PA (US)

(72) Inventors: Tyler Glen DEMAREST, Sicklerville, NJ (US); Daniel Cohen, Philadelphia, PA (US)

(21) Appl. No.: 19/119,166

(22) PCT Filed: Oct. 10, 2023
(86) PCT No.: PCT/US2023/076406

§ 371 Date: Apr. 8, 2025

Related U.S. Application Data
(60) Provisional application No. 63/378,960, filed on Oct. 10, 2022.

(51) Int. Cl. A61K 48/00; A61K 9/00; A61P 9/10; A61P 25/28; C07K 14/775; C12N 15/113; C12N 15/86; C12N 15/88

(52) U.S. Cl. CPC A61K 48/0058; A61K 9/0019; A61K 48/0075; A61P 9/10; A61P 25/28; C07K 14/775; C12N 15/113; C12N 15/86; C12N 15/88; C07K 2319/02; C12N 2310/14; C12N 2750/14143; C12N 2750/14152; C12N 2830/008; C12N 2830/42

(57) ABSTRACT

The present relates to polynucleotide constructs encoding an ApoE3 related protein optionally containing one or more intron. Potential uses of the different constructs include gene therapy targeting one or more disease or disorder, for example, diseases or disorders related to cholesterol levels, atherosclerosis, coronary heart disease, dementia, cerebral amyloid angiopathy, or Alzheimer's disease.

### CROSS REFERENCE TO RELATED APPLICATION

The present application claims priority to U.S. Provisional Application No. 63/378,960 filed on Oct. 10, 2022, the disclosure of which is incorporated herein by reference in its entirety.

### REFERENCE TO SEQUENCE LISTING SUBMITTED ELECTRONICALLY

The contents of the electronic sequence listing (065830_11WO1.xml; Size: 210,461 bytes; and Date of Creation: Oct. 5, 2023) is herein incorporated by reference in its entirety.

### BACKGROUND OF THE INVENTION

ApoE is synthesized as a precursor protein of 317 amino acids containing an 18 amino acid leader sequence. Cleavage of the leader sequence gives a mature form of ApoE containing 299 amino acids. (Khalil et al., Atherosclerosis (2021) 328:11-22 and Tudorache et al., (2017) Computational and Structural Biotechnology Journal 15:359-365.)

ApoE is mainly involved in lipid metabolism and provides for different activities including mediating hepatic and extrahepatic uptake of plasma lipoproteins and cholesterol efflux from lipid-laden macrophages. Processes indicated to involve ApoE include neuroprotection, anti-microbial defense, and oxidative stress. (Khalil et al., Atherosclerosis (2021) 328:11-22 and Tudorache et al., (2017) Computational and Structural Biotechnology Journal 15:359-365.)

Three major ApoE isoforms are present in humans: ApoE2, ApoE3 and ApoE4. The different isoforms are distinguished by amino acids in two locations. With respect to the mature ApoE, ApoE2 has a cysteine at amino acid 112 and a cysteine at amino acid 158, ApoE3 has a cysteine at amino acid 112 and an arginine at amino acid 158, and ApoE4 has an arginine at amino acid 112 and an arginine at amino acid 158. (Khalil et al., Atherosclerosis (2021) 328:11-22 and Tudorache et al., (2017) Computational and Structural Biotechnology Journal 15:359-365.)

Different ApoE isoforms, including minor forms, are associated with an increased risk of different diseases or disorders. (Zhou et al., (2021) Current Opinion is Neurobiology 69:58-67.) An approximately linear relationship of ApoE genotypes (when ordered ε2/ε2, ε2/ε3, ε2/ε4, ε3/ε3, ε3/ε4, ε4/ε4) with LDL-C and with coronary risk was identified. (Bennet et al., JAMA. (2007) 298(11):1300-1311). Additional correlations include ApoE2 homozygosity often resulting in type III hyperlipoproteinemia and ApoE4 being associated with Alzheimer's disease. (Khalil et al., Atherosclerosis (2021) 328:11-22 and Tudorache et al., (2017) Computational and Structural Biotechnology Journal 15:359-365.)

A substitution identified as the Christchurch substitution provides an ApoE 136 arginine to serine substitution. ApoE2 containing the Christchurch substitution appeared to significantly contribute to hyperlipemia. (Wardel et al., J. Clin. Invest. (1987) 80(2):483-490.) A patient having two copies of ApoE3 containing the Christchurch substitution (“ApoE3(ch)”) was indicated to be resistant to autosomal dominant Alzheimer's disease, and to have elevated triglycerides and total cholesterol. (Arboleda-Velasquez et al., Nature Medicine (2019) 25:1680-1683.)

International Patent Publication Nos. WO2022/115535, WO2021/108809, and WO2020/243346 mention treatment of Alzheimer's disease.

### BRIEF SUMMARY OF THE INVENTION

The present invention features polynucleotide constructs comprising a nucleotide sequence having a region of at least 85% sequence identity to the sequence of any of SEQ ID NOs: 63-67, or nucleotides 55-951 of SEQ ID NOs: 95-105 or 124-130; optionally comprising one or more intron; and CpG reduced introns related to any of SEQ ID NOs: 119-121. Preferred constructs are CpG reduced compared to the native ApoE3 sequence and may further comprise additional variations compared to the native sequence. The polynucleotide constructs can be used, for example, in gene therapy targeting one or more disease or disorder, for example, diseases or disorders related to cholesterol levels, atherosclerosis, coronary heart disease, dementia (e.g., vascular dementia or frontotemporal dementia), cerebral amyloid angiopathy, or Alzheimer's disease.

Thus, a first aspect of the present invention describes a polynucleotide comprising an ApoE encoding nucleotide sequence having at least 85% sequence identity to the sequence of any of SEQ ID NOs: 63-67 or nucleotides 55-951 of SEQ ID NOs: 95-105 or 124-130, wherein the polynucleotide encodes an ApoE3 related protein comprising an amino acid sequence at least 90% identical to SEQ ID NO: 32, where the protein comprises a cysteine in a location corresponding to amino acid 112 of SEQ ID NO: 32 and an arginine at a location corresponding to amino acid 158 of SEQ ID NO: 32, and wherein the ApoE encoding nucleotide sequence optionally comprises one or more introns. The at least 85% identical to any of SEQ ID NOs: 63-67 or nucleotides 55-951 of SEQ ID NOs: 95-105 or 124-130 is independent of any intron that may be present in the ApoE encoding nucleic acids.

Nucleotides 1-897 of SEQ ID NOs: 63-67 code for amino acids. Nucleotides 898-900 of SEQ ID NOs: 63-67 code for a stop codon.

Reference to an indicated percent identity to two or more reference sequences, and similar language throughout the specification providing for an indicated percent identity to two or more reference sequences, provides the indicated percent identity or percent identity range independently to each of the referenced sequences. In determining percent identity for a polynucleotide, RNA and the corresponding DNA are considered the same in the absence of a reference to the polynucleotide being RNA or DNA. Corresponding RNA and DNA include uracil for thymine and replacement of the ribose backbone for the deoxyribose backbone.

Different polynucleotide constructs are provided herein, including polynucleotides encoding for ApoE3 related protein, polynucleotides comprising an expression cassette comprising an ApoE3 related protein encoding nucleic acid sequence operably linked to one or more expression control elements, polynucleotides comprising recombinant viral vector nucleic acid wherein the 5′ and/or 3′ end of the polynucleotides have elements providing for packaging into a viral vector and/or viral replication, and vector genome plasmids.

Reference to one or more expression control elements “operably linked” or “operably coupled” to ApoE encoding nucleic acid indicates the expression control element(s) impacts ApoE3 related protein expression. ApoE3 related protein expression can be impacted in different ways, such as increased production of ApoE3 related protein mRNA transcripts, increased nuclear transport and stability of mRNA transcripts, and increased mRNA translation.

Another aspect of the present invention is directed to administering a polynucleotide construct described herein to achieve one or more of the following: reduce cholesterol, reduce LDL/VLDL, increase HDL or reduce total cholesterol/HDL ratio; or treating or reducing the likelihood of hypercholesteremia, Type III Familial hyperlipoproteinemia, Familial Hypercholesterolemia, Cerebral amyloid angiopathy, dementia, post-stent restenosis, atherosclerosis, coronary heart disease or Alzheimer's disease.

Additional aspects include polynucleotide constructs described herein for use in medicine or achieving one or more of the following: reduce cholesterol, reduce LDL/VLDL, increase HDL or reduce total cholesterol/HDL ratio; or treating or reducing the likelihood of hypercholesteremia, Type III Familial hyperlipoproteinemia, Familial Hypercholesterolemia, Cerebral amyloid angiopathy, dementia, post-stent restenosis, atherosclerosis, coronary heart disease or Alzheimer's disease; and the use of a construct described herein for the preparation of medicament for use in medicine or achieving one or more of the following: reduce cholesterol, reduce LDL/VLDL, increase HDL or reduce total cholesterol/HDL ratio; or treating or reducing the likelihood of hypercholesteremia, Type III Familial hyperlipoproteinemia, Familial Hypercholesterolemia, Cerebral amyloid angiopathy, dementia, post-stent restenosis, atherosclerosis, coronary heart disease or Alzheimer's disease.

Additional aspects include an AAV vector genome; a method of producing a rAAV vector; a method of obtaining an rAAV vector; and polynucleotides related to any SEQ ID NOs: 119-121, wherein the polynucleotide has 0-5 CpGs.

Other features and advantages of the present invention are apparent from additional descriptions provided herein, including different examples. The provided examples illustrate different components and methodology useful in practicing the present invention. Such examples do not limit the claimed invention. Based on the present disclosure, the skilled artisan can identify and employ other components and methodology useful for practicing the present invention.

### BRIEF DESCRIPTION OF THE DRAWINGS

FIG. 1 provides a schematic example of a recombinant adeno-associated viral (rAAV) polynucleotide cassette. The provided example provides the location of a 5′ ITR, an ApoE/hAAT promoter/enhancer, a HBB2 intron, a Kozak sequence, an ApoE3(ch) encoding nucleic acid, a polyA sequence and a 3′ ITR.

FIGS. 2A-2E illustrate ApoE3 and ApoE3ch transgene expression, and the impact of transgene expression on cholesterol, in ApoE knockout mice over the course of 36 weeks. Recombinant AAV comprising an ApoE3(ch) transgene were provided at a low dose of 3e12 vg/kg or a high dose of 1e13 vg/kg. Recombinant AAV comprising an ApoE3 transgene were provided at a low dose of 3e12 vg/kg or a high dose of 6e12 vg/kg rAAV. FIG. 2A illustrates hAPOE plasma levels, FIG. 2B illustrates total cholesterol, FIG. 2C illustrates LDL/VLDL, FIG. 2D illustrates HDL, and FIG. 2E illustrates the total cholesterol/HDL ratio. Week 0 timepoint indicates cholesterol levels prior to AAV administration.

FIGS. 3A-3E illustrate ApoE3ch transgene expression from different constructs, and the impact of transgene expression on cholesterol at 3 weeks post-treatment. FIG. 3A illustrates hAPOE levels (** p<0.01, *** p<0.001, **** p<0.0001 vs Excipient; +, equal to APOE3ch Native expression), FIG. 3B illustrates total cholesterol (#p<0.05, ##p<0.01, ###p<0.001, ####p<0.0001 vs. WT excipient; * p<0.05, **p<0.01, *** p<0.001, **** p<0.0001 vs. KO Excipient; percent decrease from KO Excipient animals is depicted on select bars); FIG. 3C illustrate LDL/VLDL (##p<0.01, ###p<0.001, ####p<0.0001 vs. WT Excipient; * p<0.05, **** p<0.0001 vs. KO Excipient; percent decrease from KO Excipient animals is depicted on select bars); FIG. 3D illustrates HDL cholesterol (#p<0.05, ##p<0.01, ###p<0.001 vs. WT Excipient); and FIG. 3E illustrates total cholesterol/HDL ratio (##p<0.01 vs. WT excipient; * p<0.05 vs. KO Excipient; percent increase from KO Excipient animals is depicted on select bars).

FIGS. 4A-4E illustrate ApoE3ch transgene expression from different constructs, and the impact of transgene expression on cholesterol at 6 weeks post-treatment. FIG. 4A illustrates hAPOE levels (** p<0.01, *** p<0.001, **** p<0.0001 vs Excipient; +, equal to APOE3ch Native expression); FIG. 4B illustrates total cholesterol (##p<0.01, ####p<0.0001 vs. WT Excipient; * p<0.05, *** p<0.001, **** p<0.0001 vs. KO Excipient; percent decrease from KO Excipient animals is depicted on select bars); FIG. 4C illustrates the LDL/VLDL (###p<0.001, ####p<0.0001 vs. WT Excipient; * p<0.01, **** p<0.0001 vs. KO Excipient; percent decrease from KO Excipient animals is depicted on select bars); FIG. 4D illustrates HDL cholesterol (#p<0.05, ####p<0.0001 vs. WT Excipient; * p<0.05, ** p<0.01 vs. KO Excipient; percent increase from KO Excipient animals is depicted on select bars); and FIG. 4E illustrates the total cholesterol/HDL ratio (##p<0.01 vs. WT Excipient; * p<0.05 vs. KO Excipient).

FIGS. 5A-5E illustrate ApoE3 transgene expression from different constructs, and the impact of transgene expression on cholesterol at 3 weeks. FIG. 5A illustrates hAPOE levels (*** p<0.001, **** p<0.0001 vs excipient; unless indicated by bracket); FIG. 5B illustrates total cholesterol (#p<0.05, ###p<0.01, ####p<0.0001 vs. WT Excipient; * p<0.05, **** p<0.0001 vs. Excipient; or comparison indicated by brackets; percent decrease from KO Excipient animals is depicted on select bars); FIG. 5C illustrates the LDL/VLDL (#p<0.05, ###p<0.01, ####p<0.0001 vs. WT Excipient; * p<0.05, **** p<0.0001 vs. KO Excipient; or comparison indicated by brackets; percent decrease from KO Excipient animals is depicted on select bars); FIG. 5D illustrates HDL cholesterol (#p<0.05, ###p<0.01, ####p<0.0001 vs. WT Excipient; * p<0.05, **** p<0.0001 vs. KO Excipient; or comparison indicated by brackets; percent increase from KO Excipient animals is depicted on select bars); and FIG. 5E illustrates total cholesterol/HDL ratio (##p<0.01 vs. WT Excipient; * p<0.05 vs. KO Excipient).

FIGS. 6A-6E illustrate ApoE3 transgene expression from different constructs, and the impact of transgene expression on cholesterol at 6 weeks. FIG. 6A illustrates hAPOE levels (*** p<0.001, **** p<0.0001 vs excipient, unless indicated by brackets); FIG. 6B illustrates total cholesterol (#p<0.05, ####p<0.0001 vs. WT excipient; **** p<0.0001 vs. KO Excipient; percent decrease from KO Excipient animals is depicted on select bars). FIG. 6C illustrates LDL/VLDL cholesterol (#p<0.05, ###p<0.001, ####p<0.0001 vs. WT Excipient; **** p<0.0001 vs. KO Excipient; percent decrease from KO Excipient animals is depicted on select bars). FIG. 6D illustrates HDL cholesterol (#p<0.05, ##p<0.01, ####p<0.0001 vs. WT Excipient; ** p<0.01 vs. KO Excipient; percent decrease from KO Excipient animals is depicted on select bars). FIG. 6E illustrates the total cholesterol/HDL ratio (##p<0.01 vs. WT Excipient; * p<0.05, **p<0.01 vs. KO Excipient).

FIGS. 7A and 7B illustrate liver tissue levels of hAPOE/total protein (FIG. 7A; p<0.05 vs ε3(ch) Native constructs) and vector genome copy number/μg gDNA (FIG. 7B), resulting from different constructs. Reference to “ns vs. native” in FIG. 7B, indicates the difference between ε3ch-Native and different CpG reduced constructs was not significant (*p<0.05 for constructs ε3-8, ε3-9, ε3-11, ε3-12 and ε3-15 vs. ε3-Native constructs).

FIG. 8 provides a black and white figure of atheroma fatty inclusions stained with Oil Red O. WT Excipient refers to wild type mouse (C57BL/6) administered excipient. KO Excipient refers to ApoE knockout mouse (B6.129P2-Apoetm1Unc/J, Jackson Labs, Strain #:002052) administered excipient. APOE3ch(low) refers to KO mouse provided a low dose of 3e12 vg/kg rAAV APOE3ch(high) refers to KO mouse provided a high dose of 1e13 vg/kg rAAV. APOE3(low) refers to KO mouse provided a low dose of 3e12 vg/kg rAAV. APOE3(high) refers to KO mouse provided a high dose of 6e12 vg/kg rAAV.

FIG. 9 illustrates percent aortic lesion areas. WT Excipient refers to wild type mouse (C57BL/6) administered excipient. KO Excipient refers to ApoE knockout mice (B6.129P2-Apoetm1Unc/J, Jackson Labs, Strain #:002052) administered excipient. APOEch(low) refers to KO mice provided a low dose of 3e12 vg/kg rAAV. APOEch(high) refers to KO mice provided a high dose of 1e13 vg/kg rAAV. APOE3(low) refers to KO mice provided a low dose of 3e12 vg/kg rAAV. APOE3(high) refers to KO mice provided a high dose of 6e12 vg/kg rAAV. **** p<0.0001 vs. KO Excipient; or comparison indicated by brackets.

FIGS. 10A-10F are bar diagrams illustrating the anti-inflammatory effects of rAAV comprising an APOE3ch or an APOE3 transgene in ApoE knockout (KO) mice (B6.129P2-Apoetm1Unc/J, Jackson Labs, Strain #:002052). APOE3ch L and APOE3-L refer to KO mice provided a low dose of 3e12 vg/kg rAAV APOE3ch-H and APOE3-H refers to KO mice provided a high dose of 1e13 vg/kg rAAV Anti-inflammatory effects were measured using a MesoScale Diagnostics Mouse Cytokine panel. FIG. 10A illustrates IL-5 levels, FIG. 10B illustrates IL-6 levels, FIG. 10C illustrates TNF-α levels, FIG. 10D illustrates IL-17A/F levels, FIG. 10E illustrates CCL2 levels, FIG. 10F illustrates CXCL2. One-way ANOVA, Dunnett's post-hoc test. #p<0.05, ##p<0.01 vs. WT Excipient, *p<0.05, **p<0.01 vs. KO Excipient.

FIGS. 11A-11D are bar diagrams illustrating the effect of rAAV comprising an APOEch or an APOE transgene in ApoE knockout (KO) mice (B6.129P2-Apoetm1Unc/J, Jackson Labs, Strain #:002052), on glial fibrillar acidic protein (GFAP) levels. APOE3ch L and APOE3-L refer to KO mice provided a low dose of 3e12 vg/kg rAAV. APOE3ch-H and APOE3-H refers to KO mice provided a high dose of 1e13 vg/kg rAAV FIG. 11A illustrates GFAP/total protein in the cortex determined by JESS capillary electrophoresis (ProteinSimple). FIG. 11B illustrates GFAP/total protein in the hippocampus determined by JESS capillary electrophoresis (ProteinSimple). FIG. 11C illustrates % area of GFAP in whole brain determined by immunofluorescence staining with Anti-GFAP Antibody (AB5541, Millipore; 1:500) and percent GFAP area quantified using HALO® image analysis software (IndicaLabs). FIG. 11D illustrates GFAP % area in the hippocampus determined by immunofluorescence quantification using HALO® image analysis software (IndicaLabs). One-way ANOVA followed by Dunn's or Fisher's posthoc tests. #p<0.05 vs. WT, *p<0.05, **p<0.01 vs. KO Excipient.

FIGS. 12A-12D are bar diagrams illustrating the effect of rAAV comprising an APOE3ch or an APOE3 transgene in ApoE knockout (KO) mice (B6.129P2-Apoetm1Unc/J, Jackson Labs, Strain #:002052), on pre and post-synaptic proteins in the cortex and hippocampus. APOE3ch (low) and APOE3 (low) refer to KO mice provided a low dose of 3e12 vg/kg rAAV. APOE3ch (high) and APOE3 (high) refer to KO mice provided a high dose of 1e13 vg/kg rAAV FIG. 12A illustrates synaptophysin/total protein in the hippocampus. FIG. 12B illustrates PSD-95/total protein in the hippocampus. FIG. 12C illustrates synaptophysin/total protein in the cortex. FIG. 12D illustrates PSD-95/total protein in the cortex. Synaptic proteins were determined by JESS capillary electrophoresis (ProteinSimple). Kruskal-Wallis One-way ANOVA followed by Dunn's posthoc test. #p<0.05 vs. WT, *p<0.05, **p<0.01 vs. KO Excipient

FIGS. 13A-13D are bar diagrams illustrating the effect of rAAV comprising an APOE3ch or APOE3 transgene in 1 year-old ApoE knockout (KO) mice (B6.129P2-Apoetm1Unc/J, Jackson Labs, Strain #:002052), on atherosclerosis reversal of pre-existing lesions. FIG. 13A illustrates % aortic lesion area in KO mice and mice administered different transgene sequences encoding for ApoE: ε3N (native ApoE3) 2e¹¹ vg/kg, ε3-3 (ApoE3-3) 2e¹¹ vg/kg, and ε3-3 (ApoE3-3) 2e¹² vg/kg. FIG. 13B illustrates the % change in atherosclerotic lesions in KO baseline from FIG. 13A and a combination of the ε3N (native ApoE3) 2e¹¹ vg/kg and ε3-3 (ApoE3-3) 2e¹¹ vg/kg groups from FIG. 13A. FIG. 13C illustrates % aortic lesion area in KO untreated mice and KO mice administered different transgene sequences encoding for ApoE3ch: ε3N (native ApoE3ch) 2e¹¹ vg/kg, ε3ch-9 (ApoE3ch-9) 2e¹¹ vg/kg, and ε3ch-9 (ApoE3ch-9) 2e¹² vg/kg. FIG. 13D illustrates the % change in atherosclerotic lesions in KO baseline from FIG. 13C and a combination of ε3N (native ApoE3ch) 2e¹¹ vg/kg and ε3ch-9 (ApoE3ch-9) 2e¹¹ vg/kg groups from FIG. 13C.

FIGS. 14A-14D illustrate the results of cognitive deficit testing in mice administered rAAVs comprising APOE3(ch) transgenes, in a Novel Object Recognition (NOR) memory test. The rAAV was administered at a low dose of 2e11 vg/mouse or a high dose of 2e12 total vg/mouse. FIG. 14A illustrates NOR results for 1-year old C57BL/6 and ApoE knockout mice (*p<0.05, unpaired 2-tailed t-test) indicating cognitive impairment in 1-year old ApoE knockout mice compared to age-matched C57BL/6 mice. FIG. 14B illustrates NOR results prior to gene therapy, and 5-weeks following treatment with gene therapy using native ApoE3 and ApoE3(ch) sequences in the same mice before and after gene therapy treatment. FIG. 14C illustrate NOR results with a low dose of rAAV encoding CpG-0 ApoE3 (ε3-3) and ApoE3ch (ε3ch-9) rAAV. FIG. 14D illustrate NOR results with a high dose of rAAV encoding CpG-0 ApoE3 (ε3-3) and ApoE3ch (ε3ch-9) rAAV

FIGS. 15A and 15B illustrate ApoE expression from different transgenes. FIG. 15A provides a bar graph showing the performance of codon-optimized, CpG-reduced cDNAs encoding ApoE3. Plasmids encoding either wild-type (wt) ApoE3 or codon-optimized ApoE3 cDNAs were transfected in triplicate into AML-12 cells and antigen levels were measured in cell culture supernatants at 72 hours post-transfection. ApoE3 levels were assayed by ELISA and plotted as the mean+/−the standard deviation. FIG. 15B provides a bar graph showing the performance of codon-optimized ApoE3 cDNA functionalized by the addition of intron sequences. Plasmids encoding either wild-type (wt) ApoE3 or intron-containing cDNA variants of ApoE3 were transfected in triplicate into AML-12 cells, and antigen levels were measured in cell culture supernatants at 72 hours post-transfection. For comparison purposes, the non-intron containing codon-optimized ApoE3-3 and H30 variants were included as benchmarks. ApoE3 levels were assayed by ELISA and plotted as the mean+/−the standard deviation.

### DETAILED DESCRIPTION OF THE INVENTION

The present invention features polynucleotide constructs comprising an ApoE encoding nucleotide sequence having at least 85% sequence identity to the sequence of any of SEQ ID NOs: 63-67 or nucleotides 55-951 of SEQ ID NOs: 95-105 or 124-130, wherein the polynucleotide encodes an ApoE3 related protein comprising an amino acid sequence at least 90% identical to SEQ ID NO: 32, where the protein comprises a cysteine in a location corresponding to amino acid 112 of SEQ ID NO: 32 and an arginine at a location corresponding to amino acid 158 of SEQ ID NO: 32, and wherein the ApoE encoding nucleotide sequence optionally comprises one or more introns. ApoE3(ch) is an ApoE3 related protein comprising a serine at a position corresponding to amino acid 136 of SEQ ID NO: 32.

The provided constructs can be used in different methods including gene therapy targeting different diseases or disorders related to cholesterol, lipoprotein levels, cardiovascular disease, dementia or Alzheimer's disease in a subject. The examples provided below illustrate, for example, the ability of different transgenes encoding for ApoE3(ch) and ApoE3 expression to reduce total cholesterol level, increase HDL, decrease LDL/VLDL, decrease total cholesterol/HDL ratio, decrease of atherosclerosis lesions (atheroma), reduce inflammatory proteins, increase synaptogenesis, and improve cognitive function in an Novel Object Recognition test in ApoE knockout mice. Additional uses of the constructs include studying the impact of ApoE gene therapy in animal models.

Reference to “subject” indicates a mammal, including a human; non-human primates such as apes, gibbons, gorillas, chimpanzees, orangutans, and macaques; domestic animals such as dogs and cats; farm animals such as poultry, ducks, horses, cows, goats, sheep and pigs; and experimental animals such as mice, rats, rabbits, and guinea pigs. A preferred subject is a human.

Polynucleotides encoding ApoE3 related protein can be delivered using non-viral or viral delivery. Viral vectors that can be used in gene therapy include retroviral vectors, adenovirus vectors, AAV vectors, and herpes simplex viral vectors. Non-viral delivery includes naked DNA and the use of nanoparticles.

Reference to a percent “identical”, “identity” and similar terminology are with respect to two sequences having maximal alignment in a particular area. The provided area is with respect to the indicated reference sequence. For example, sequence “identical” or “identity” to human mature ApoE3 protein can be calculated by determining the number of identical amino acids in aligned sequences, dividing by the total number of amino acids in SEQ ID NO: 32 (299 amino acids) and multiplying by 100. Percent “identical” or “identity” for nucleic acid sequences can be determined in an analogous manner where nucleotides to the reference sequence are aligned to achieve maximal alignment, dividing by the total number of nucleotides in the reference sequence and multiplying by 100. Percent “identical” or “identity” for an ApoE encoding sequence determined independently of any intron, indicates for calculating purposes, intron(s) are removed prior to the alignment.

The terms “nucleic acid” and “polynucleotide” are used interchangeably herein to refer to all forms of nucleic acid, oligonucleotides, including deoxyribonucleic acid (DNA) and ribonucleic acid (RNA). In discussing nucleic acids, a sequence or structure of a particular polynucleotide can be described herein according to the convention of providing the sequence in the 5′ to 3′ direction.

In certain embodiments, nucleic acids include genomic DNA, cDNA, antisense DNA/RNA, plasmid DNA, linear DNA, (poly- and oligo-nucleotide), chromosomal DNA, spliced or unspliced mRNA, rRNA, tRNA inhibitory DNA or RNA (RNAi, e.g., small or short hairpin (sh)RNA, microRNA (miRNA), small or short interfering (si)RNA, trans-splicing RNA, or antisense RNA), locked nucleic acid analogue (LNA), oligonucleotide DNA (ODN) single and double stranded, immunostimulating sequence (ISS), riboswitches and ribozymes.

In certain embodiments, nucleic acids include naturally occurring, synthetic, and intentionally modified or altered polynucleotides. Nucleic acids can be single, double, or triplex, quadruplex, linear or circular, and can be of any length.

According to certain embodiments, the polynucleotide is a single-stranded (ssDNA) or a double-stranded DNA (dsDNA) molecule. According to certain embodiments, the dsDNA molecule is a minicircle, a nanoplasmid, open linear duplex DNA or a closed-ended linear duplex DNA (CELiD/ceDNA/doggybone DNA). According to certain embodiments, the ssDNA molecule is a closed circular or an open linear DNA.

A “transgene” refers to a nucleic acid that is intended or has been introduced into a cell and operably linked to a promoter. Transgenes include heterologous polynucleotide sequence, such as nucleic acid encoding ApoE3 related protein and a heterologous promoter.

Preferred polynucleotide constructs are “CpG reduced” or “CpG depleted”. “CpG reduced” or “CpG depleted” refer to (i) a nucleotide sequence wherein one or more of the CpG dinucleotides (or motifs) are removed from a reference nucleic acid sequence; and/or (ii) the percentage of CpGs in a referred to polynucleotide is 0% to 15%. In different embodiments, the CpG percentage is 0%, about 0.5%, about 1.0%, about 2.0%, about 3.0%, about 4.0%, or about 5.0%, about 6%, about 7%, about 8%, about 9%, about 10%, about 11%, about 12%, about 13%, about 14%, or about 15% CpGs; and/or up to about 0.5%, up to about 1.0%, up to about 2.0%, up to about 3.0%, up to about 4.0%, up to about 5.0%, up to about 6%, up to about 7%, up to about 8%, up to about 9%, up to about 10%, up to about 11%, up to about 12%, up to about 13%, up to about 14%, or up to about 15% CpGs.

CpG motifs can be suitably reduced or eliminated in a nucleotide sequence encoding a ApoE3 related protein and in other sequences that be present in particular constructs (e.g., expression cassettes and viral vectors). Other sequences that may be present include non-coding sequences such as 5′ and 3′ untranslated regions (UTRs), stuffer sequences, promoter, enhancer, polyadenylation signal, ITRs, and intron(s).

The singular forms “a,” “an,” and “the” include plural reference unless the context clearly dictates otherwise.

The conjunctive term “and/or” between multiple recited elements encompasses both individual and combined options. For instance, where two elements are conjoined by “and/or”, a first option refers to the applicability of the first option without the second, a second option refers to the applicability of the second option without the first, and a third option refers to the applicability of the first and second options together. Any one of the options is understood to fall within the meaning, and therefore satisfy the requirement of the term “and/or”. Concurrent applicability of more than one of the options is also understood to fall within the meaning of the term “and/or.”

Unless clearly indicated otherwise by the context employed the terms “or” and “and” have the same meaning as “and/or”.

Reference to terms such as “including”, “for example”, “e.g.,” “such as” followed by different members or examples, are open-ended descriptions where the listed members or examples are illustrative and other members or examples can be provided or used.

The terms “polypeptide,” “protein” and “peptide” can be used interchangeably to refer to an amino acid sequence without regard to function. Polypeptides and peptides contain at least two amino acids, while proteins contain at least about 10 amino acid acids. The provided amino acids include naturally occurring amino acids and amino acids provided by cellular modification.

Reference to “comprise”, and variations such as “comprises” and “comprising”, used with respect to an element or group of elements is open-ended and does not exclude additional unrecited elements or method steps. Terms such as “including”, “containing” and “characterized by” are synonymous with comprising. In the different aspects and embodiments described herein reference to an open-ended term such as “comprising” can be replaced by “consisting” or “consisting essentially of”.

Reference to “consisting of” excludes any element, step, or ingredient not specified in the listed claim elements, where such element, step or ingredient is related to the claimed invention.

Reference to “consisting essentially of” limits the scope of a claim to the specified materials or steps and those that do not materially affect the basic and novel characteristic(s) of the claimed invention.

The term “about” refers to a value within 10% of the underlying parameter (i.e., plus or minus 10%). For example, “about 1:10” includes 1.1:10.1 or 0.9:9.9, and “about 5 hours” includes 4.5 hours or 5.5 hours. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100%, as well as 95.1%, 95.2%, 95.3%, 95.4%, 95.5%, etc., 96.1%, 96.2%, 96.3%, 96.4%, 96.5% and so forth and reference to a numerical range, such as “1-4” includes 1, 2, 3, 4 as well as 1.1, 1.2, 1.3, 1.4 and so forth. As a further illustration, “1 to 4 weeks” includes 7, 8, 9, 10, 11, 12, 13, 14, 15, 16, 17, 18, 19, 20, 21, 22, 23, 24, 25, 26, 27, or 28 days.

Further, reference to a numerical range, such as “0.01 to 10” includes 0.011, 0.012, 0.013 etc., as well as 9.5, 9.6, 9.7, 9.8, 9.9 and so forth. For example, a dosage of about “0.01 mg/kg to about 10 mg/kg” body weight of a subject includes 0.011 mg/kg, 0.012 mg/kg, 0.013 mg/kg, 0.014 mg/kg, 0.015 mg/kg etc., as well as 9.5 mg/kg, 9.6 mg/kg, 9.7 mg/kg, 9.8 mg/kg, 9.9 mg/kg and so forth.

Reference to an integer with more (greater) or less than includes numbers greater or less than the reference number, respectively. Thus, for example, reference to more than 2 includes 3, 4, 5, 6, 7, 8, 9, 10, 11, 12, 13, 14, 15 or more; and administration “two or more” times includes 2, 3, 4, 5, 6, 7, 8, 9, 10, 11, 12, 13, 14, 15, or more times.

Various references including articles and patent publications are cited or described in the background and throughout the specification. Each of these references is herein incorporated by reference in their entirety. None of the references are admitted to be prior art with respect to any inventions disclosed or claimed. In some cases, particular references are indicated to be incorporated by reference herein to highlight the incorporation.

The definitions provided herein, including those in the present section, and other sections of the application apply throughout the present application.

Unless defined otherwise, all technical and scientific terms used herein have the same meaning commonly understood to one of ordinary skill in the art to which this invention pertains.

The description has been separated into various sections and paragraphs, and provides examples of various embodiments. These separations should not be considered as disconnecting the substance of a paragraph or section or embodiments from the substance of another paragraph or section or embodiment. The provided descriptions have broad application and encompasses all the combinations of the various sections, paragraphs and sentences that can be contemplated. The discussion of any embodiment is meant only to be exemplary and is not intended to suggest the scope of the disclosure, including the claims (unless otherwise provided in the clams), is limited to these examples.

The instant invention is generally disclosed herein using affirmative language to describe the numerous embodiments of the instant invention. The instant invention also specifically includes embodiments in which particular subject matter is excluded, in full or in part, such as substances or materials, method steps and conditions, protocols, or procedures. For example, in certain embodiments of the instant invention, materials and/or method steps are excluded. Thus, even though the instant invention is generally not expressed herein in terms of what the instant invention does not include, embodiments that are not expressly excluded in the instant invention are nevertheless disclosed herein.

### I. ApoE Protein Encoding Polynucleotide

ApoE encoding nucleotide sequences encode an ApoE3 related protein comprising an amino acid sequence at least 90% identical to SEQ ID NO: 32, where the protein contains a cysteine in a location corresponding to amino acid 112 of SEQ ID NO: 32 and an arginine at a location corresponding to amino acid 158 of SEQ ID NO: 32. If intron(s) are present, the percent identity is determined independent of the intron(s). In certain embodiments, no intron is present in the ApoE encoding nucleic acid.

In different embodiments the ApoE3 related protein is at least 95%, at least 96% at least 97%, at least 98%, at least 99% identical to the amino acid sequence of SEQ ID NO: 32; differs from SEQ ID NO: 32 by 1, up to 2, up to 3, up to 4, up to 5, up to 6, up to 7, up to 8, up to 9, or up to 10 amino acids; or is provided by SEQ ID NO: 32.

In different embodiments the ApoE3 related protein further comprises a serine at a position corresponding to amino acid 136 of SEQ ID NOs: 32, and is at least 95%, at least 96%, at least 97%, at least 98%, at least 99% identical to the amino acid sequence of SEQ ID NO: 32; differs from ApoE3(ch) of SEQ ID NO: 33 by 1, up to 2, up to 3, up to 4, up to 5, up to 6, up to 7, up to 8, up to 9, or up to 10 amino acids; or is provided by SEQ ID NOs: 32 or 33. The serine at a position corresponding to amino acid 136 of SEQ ID NO: 32, provides for a Christchurch substitution.

Additional ApoE3 related proteins can be obtained using ApoE3 sequences of SEQ ID NO: 32 and SEQ ID NO: 33 as a starting construct based upon the existing knowledge of different ApoE domains and different ApoE sequences. Examples of references describing different domains and sequences include Khalil et al., Atherosclerosis (2021) 328:11-22 and Tudorache et al., (2017) Computational and Structural Biotechnology Journal 15:359-365.

In certain embodiments, the ApoE encoding sequence comprises a sequence having a sequence identity of least 85% to any of SEQ ID NOs: 63-67, and nucleotides 55-954 of any of SEQ ID NOs: 3-10, 12-14, 16-19, 21-25, 27-30, and SEQ ID NOs: 95-105 or 124-130. In further embodiments, the provided sequence identity is at least 86%, at least 87%, at least 88%, at least 89%, at least 90%, at least 91%, at least 92%, at least 93%, at least 94%, at least 95%, at least 96%, at least 97%, at least 98%, at least 99% or 100% to any of SEQ ID NO: 63, SEQ ID NO: 64, SEQ ID NO: 65, SEQ ID NO: 66, SEQ ID NO: 67, or nucleotides 1-897 of any of SEQ ID NO: 63, SEQ ID NO: 64, SEQ ID NO: 65, SEQ ID NO: 66, SEQ ID NO: 67; or nucleotides 55-951 or 55-954 of any of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 12, SEQ ID NO: 13, SEQ ID NO: 14, SEQ ID NO: 16, SEQ ID NO: 17, SEQ ID NO: 18, SEQ ID NO: 19, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 29, SEQ ID NO: 30, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104 and SEQ ID NO: 105, SEQ ID NO: 124, SEQ ID NO: 125, SEQ ID NO: 126, SEQ ID NO: 127, SEQ ID NO: 128: SEQ ID NO: 129, and SEQ ID NO: 130.

In certain embodiments, the ApoE encoding sequence is terminated by one, two, or more than two stop codons.

Reference to a sequences provided in the present application that include a stop codon, include embodiments where the stop codon is not present, multiply stop codons are present, and different stop codons are present.

The mature ApoE3 related protein can be formed intracellularly from a mature ApoE sequence further comprising a signal peptide. Signal peptides are short N-terminal amino acid sequences providing for protein secretion. Signal peptides direct proteins to or through the endoplasmic reticulum secretory pathway and are generally cleaved within the endoplasmic reticulum prior to secretion. Thus, a signal peptide enhances secretion of a polypeptide from the cell as compared to the secretion level of the corresponding polypeptide lacking a signal peptide.

In certain embodiments, the ApoE3 related protein comprises a signal peptide. Signal peptides can be derived in whole or in part from the secretory signal of a secreted polypeptide and/or can be in whole or in part synthetic. Generally, known signal peptides are from about 10-15 to 50-60 amino acids in length. Further, known secretory signals from secreted polypeptides can be altered or modified (e.g., by substitution, deletion, truncation or insertion of amino acids) as long as the resulting secretory signal sequence functions to enhance polypeptide secretion.

In certain embodiments, signal peptides comprise, consist essentially of or consist of a naturally occurring secretory signal sequence or a modification thereof. Examples of synthetic or artificial secretory signal peptides are provided in, for example, Barash et al., Biochem. Biophys. Res. Comm. (2002).

In certain embodiments, the signal peptide is selected from the ApoE3 signal peptide, a human chymotrypsinogen B2 signal peptide (“sp7”; 18 amino acid signal peptide of NCBI reference sequence NP_001020371), alpha 2-HS-glycoprotein (AHSG) signal peptide, CD300 signal peptide, lysosome-associated membrane glycoprotein 1 (LAMP1) signal peptide, Notch 2 signal peptide, orosomucoid 1 (ORM1) signal peptide, transferrin (TF) signal peptide, secrecon (artificial signal sequence described in Barash et al., Biochem Biophys Res Commun. 2002; 294: 835-842), mouse IgKVIII, human IgKVIII, CD33, tPA, a-1 antitrypsin signal peptide, and native secreted alkaline phosphatase (SEAP).

In certain embodiments the signal peptide has at least 90% identity to an amino acid sequence selected from the group consisting of SEQ ID NOs: 36, 42, 44, 46, 48, 50, 52, 54 and 56. In further embodiments the signal peptide has at least 95%, at least 96%, at least 97%, at least 98%, at least 99%, or 100% identity to any of SEQ ID NOs: 36, 42, 44, 46, 48, 50, 52, 54, 56 and 68-71; or differs from any of SEQ ID NOs: 36, 42, 44, 46, 48, 50, 52, 54, 56 and 68-71 by any of 1, 2, or 3 amino acids.

In different embodiments the ApoE3 related protein comprises a signal peptide and is at least 96%, at least 97%, at least 98%, at least 99% or 100% identical to the amino acid sequence of SEQ ID NOs: 34 or 35; differs from SEQ ID NOs: 34 or 35 by 1, up to 2, up to 3, up to 4, up to 5, up to 6, up to 7, up to 8, up to 9 or up to 10 amino acids; is provided by SEQ ID NO: 34; or is provided by SEQ ID NO: 35. SEQ ID NO: 34 provides the native ApoE3 sequence comprising the ApoE3 signal peptide, while SEQ ID NO: 35 provides an ApoE3(ch) comprising the ApoE3 signal peptide.

In certain embodiments the ApoE encoding nucleotide sequence comprises one or more introns. Introns are characterized by 5′ and 3′ splice consensus sequences providing the intron boundaries along with an adenosine at the branch point. The adenosine is involved in breaking the phosphodiester bond at the upstream exon-intron boundary. In certain embodiments, the 5′ and 3′ splice consensus sequences comprise 5′ GU . . . AG 3′. In certain embodiments, the 5′ and 3′ splice consensus sequences comprise 5′ AU . . . AC 3′. Consensus splice sites are described, for example, in Jurica and Roybal, RNA Splicing, Eds. Lennarz and Lane, Encyclopedia of Biological Chemistry (Second Edition), Academic Press, 2013, pages 185-190; and Qu et al., Front Genet. 2017 Apr. 11; 8:38; each of which are hereby incorporated by reference herein in their entirety.

The branch point is also typically located in a consensus sequence (e.g., haptamer sequence) around 18-40 nucleotides upstream of the 3′ acceptor site and a polypyrimidine tract generally separates the branch point from the 3′ splice site. In certain embodiments the branch point comprising the adenosine is about 18-40 nucleotides upstream the 3′ acceptor site and the polypyrimidine tract primarily comprising C and U residues is present. RNA splicing is described for example, by Jurica and Roybal, RNA Splicing, Eds. Lennarz and Lane, Encyclopedia of Biological Chemistry (Second Edition), Academic Press, 2013, pages 185-190; and Berger et al., Wiley Interdiscip Rev RNA. 2016 July; 7(4):487-98; each of which are hereby incorporated by reference herein in their entirety.

In further embodiments, the ApoE encoding nucleotide sequence has 1 or 2 introns and/or the introns are located in a naturally occurring position. Native intron splice sites in the coding sequence are located at codon 15, between nucleotides 43 and 44 of SEQ ID NO: 1 (split Gly G/GC); at codon 79 (split Arg AG/G) between nucleotides 236 and 237 of SEQ ID NO: 1.

In certain embodiments the ApoE encoding nucleotide sequence (ApoE transgene) comprises 5′ to 3′:
(a) a first exon corresponding to nucleotides 1-43 of SEQ ID NO: 1, wherein the first exon has a sequence identity of at least 85% to nucleotides 1-43 of any of SEQ ID NOs: 3-31 95-105 or 124-130, provided that the terminal 3′ nucleotide of the first exon is G, in further embodiments concerning the first exon, the first exon has a sequence identity of at least 90%, at least 95%, at least 96%, at least 97%, at least 98%, at least 99% or 100% to nucleotides 1-43 of any of SEQ ID NOs: 3-31, 95-105, and 124-130;
(b) a first intron at a position corresponding to between nucleotides 43 and 44 of SEQ ID NO: 1;
(c) a second exon corresponding to nucleotides 44-236 of SEQ ID NO: 1; wherein the second exon has a sequence identity of at least 85% to nucleotides 44-236 of any of SEQ ID NOs: 3-31, 95-105 or 124-130, provided that the terminal 5′ nucleotides of the second exon is G (in a further embodiment GC) and the terminal 3′ nucleotides of the second exon is AG, in further embodiments concerning the second exon, the second first exon has a sequence identity of at least 90%, at least 95%, at least 96%, at least 97%, at least 98%, at least 99% or 100% to nucleotides 44-236 any of SEQ ID NOs: 3-31, 95-105 or 124-130;
(d) a second intron at a position corresponding to between nucleotides 236 and 237 of SEQ ID NO: 1,
(e) a third exon corresponding to nucleotides 237-951 of SEQ ID NO: 1, wherein the third exon has a sequence identity of at least 85% to nucleotides 237-951 of any of SEQ ID NOs: 3-31, 95-105 or 124-130, provided that the terminal 5′ nucleotide of the third exon is G, in further embodiments concerning the third exon, the third exon has a sequence identity of at least 90%, at least 95%, at least 96%, at least 97%, at least 98%, at least 99% or 100% to nucleotides 237-951 any of SEQ ID NOs: 3-31, 95-105, or 124-130;
wherein the first, second and third exons together encode an ApoE3 related protein comprising an amino acid sequence at least 95% identical to SEQ ID NO: 35, in further embodiments, the ApoE related protein comprises an amino acid sequence identity at least 96%, at least 97%, at least 98%, or at least 99% to SEQ ID NO: 35, differs from SEQ ID NO: 35 by 1, 2, 3, 4, or 5 amino acids, or comprises SEQ ID NO: 34 or SEQ ID NO: 35.

Each of possibilities within (a), (b), (c), (d), and (e) can be combined independently, for each provided sequence and sequence identity. For example, (1) the first exon can independently of the second and third exons have a sequence identity of at least 85%, at least 90%, at least 95%, at least 96%, at least 97%, at least 98%, at least 99% or 100% to nucleotides 1-43 of any of SEQ ID NOs: 3-31, 95-105, or 124-130; (2) the second exon independently of the first and third exons can have a sequence identity of at least 85%, at least 90%, at least 95%, at least 96%, at least 97%, at least 98%, at least 99% or 100% to nucleotides 44-236 of any of SEQ ID NOs: 3-31, 95-105, or 124-130; and (3) the third exon independently of the first and second exons can have a sequence identity of at least 85%, at least 90%, at least 95%, at least 96%, at least 97%, at least 98%, at least 99% or 100% to nucleotides 44-236 any of SEQ ID NOs: 3-31, 95-105, or 124-130.

Reference to a “corresponding” nucleotide or nucleotide region to a reference sequence (e.g., SEQ ID NO: 1), indicates the corresponding nucleotide position or nucleotide region (e.g., exon or intron) is that position or region matching up with the indicated position or region of the reference sequence when a maximal alignment takes place. The maximal alignment takes into account any addition, deletion and/or substitution. Preferably, only substitutions are present.

In certain embodiments the first intron comprises a sequence with a sequence identity of at least 50%, at least 60%, at least 70%, at least 80%, at least 90%, at least 95%, at least 96%, at least 97%, at least 98%, at least 99%, or 100% to any of SEQ ID NOs: 119-122 and the second intron independently comprises a sequence with a sequence identity of at least 50%, at least 60%, at least 70%, at least 80%, at least 90%, at least 95%, at least 96%, at least 97%, at least 98%, at least 99%, or 100% identity to any of SEQ ID NOs: 119-122.

In certain embodiments the first intron consists of a sequence of any of SEQ ID NOs: 119-121 or a sequence differing from any of SEQ ID NOs: 119-121 by 1 to 10 nucleotides and the second intron independently consists of a sequence of any of SEQ ID NOs: 119-121 or a sequence differing from any of SEQ ID NOs: 119-121 by 1 to 10 nucleotides.

In certain embodiments the first exon has a sequence identity of at least 95%, at least 96%, at least 97%, at least 98%, at least 99% or 100% to nucleotides 1-43 of any of SEQ ID NOs: 11, 15, 20, 26, 31 or 95-105;
the second exon has a sequence identity of at least 95%, at least 96%, at least 97%, at least 98%, at least 99% or 100% to nucleotides 44-236 of any of SEQ ID NOs: 11, 15, 20, 26, 31 or 95-105; and
the third exon has a sequence identity of at least 95%, at least 96%, at least 97%, at least 98%, at least 99% or 100% to nucleotides 237-951 of any of SEQ ID NOs: 11, 15, 20, 26, 31 or 95-105, wherein the ApoE related protein comprises an amino acid sequence identity at least 98%, or at least 99% to SEQ ID NO: 35, differs from SEQ ID NO: 35 by 1, 2, 3, 4, or 5 amino acids, or comprises SEQ ID NO: 34 or SEQ ID NO: 35.

In certain embodiments the ApoE encoding nucleotide sequence comprises a sequence with a sequence identity of at least 85%, at least 95%, at least 96%, at least 97%, at least 98%, at least 99% or 100% identity to any of SEQ ID NOs: 106-115.

In certain embodiments, one or more intron is selected from the rabbit β-globin intron with splice donor/splice acceptor, SV40 intron with splice donor/splice acceptor, human β-globin introns, intron 2 of the human hemoglobin beta gene, hFIX int1 (intron 1 of the human coagulation factor IX gene), CBA-rHHB (synthetic intron derived from the fusion of the intron 1 of the chicken beta actin gene and intron 2 of the rabbit hemoglobin beta), CBA (intron 1 of the chicken beta actin gene), hGH (intron 1 of the human growth hormone gene), hFIX synth (synthetic intron derived from different portions of the human coagulation factor IX gene and present in the pLIVE vector, Mirus Bio, Madison, WI); human hemoglobin subunit beta (HBB2) synthetic intron, and optimized HBB2; and chimeric introns such as introns made up of the 5′-splice donor of the first human β-globin intron and the branch and 3′-acceptor site from the intron that is between the leader and the body of the immunoglobulin gene heavy chain variable region. (Buck et al., Int. J. Mol. Sci. (2020), 21, 4197; Ronzitti et al. Mol. Ther. Methods Clin Dev. (2016) July 20; 3:16049; and the HBB-IGG intron provided by the pCMVNT™ vector.)

In certain embodiments introns are 50 bases to 1,500 bases.

In certain embodiments, any of the ApoE encoding nucleotide sequences and introns provided herein contain 0-5, 0-10, or 0-15 CpGs; 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, and 82 CpGs; 0%, about 0.5%, about 1.0%, about 2.0%, about 3.0%, about 4.0%, or about 5.0% CpGs; and/or up to about 0.5%, up to about 1.0%, up to about 2.0%, up to about 3.0%, up to about 4.0%, or up to about 5.0% CpGs; preferably 0 CpGs.

### II. Expression Cassettes

Polynucleotide expression cassettes contain an ApoE encoding nucleic acid operably linked to one or more expression control elements. The “expression control element” is a nucleic acid sequence influencing expression of an ApoE encoding nucleic acid.

Expression control can be affected, for example, at the level of transcription, translation, splicing, and message stability. Expression control elements are typically located 5′ (“upstream”) or 3′ (“downstream”) of a transcribed nucleic acid. Expression control elements can also be located within the transcript (e.g., in an intron). Expression control elements can be located adjacent to or at a distance away from the transcribed sequence. One or more expression control elements may be present. Examples of expression control elements include a promoter, enhancer, an intron, polyadenylation signal, a Kozak sequence, post-transcriptional regulator elements and a termination sequence.

A promoter is a DNA region where transcription is initiated. In general, transcribed nucleic acid are located 3′ of a promoter sequence. In certain embodiments, a promoter sequence is coupled to an enhancer. Enhancers are DNA regions that increase promoter transcription. Enhancers can be adjacent to a promoter or can be distal. Typically, enhancers are located upstream of a promoter, but can be located downstream or within a promoter sequence.

Expression control elements such as a promoter and an enhancer can be chosen to preferentially drive expression in a particular cell or tissue type. Expression control elements are typically active in particular cells, tissues or organs because they are recognized by transcriptional activator proteins, or other regulators of transcription, that are unique to a specific cell, tissue or organ type. (See, e.g., Green, M. and Sambrook, J. (2012) Molecular Cloning: A Laboratory Manual. 4th Edition, Vol. II, Cold Spring Harbor Laboratory Press, New York; and Ausubel et al., (2010) Current protocols in molecular biology, John Wiley & Sons, New York).

The incorporation of tissue specific regulatory elements in the expression construct provides for at least partial tissue tropism for the expression of ApoE3 related protein. Reference to a promoter or enhancer specific for a particular cell type of tissue, indicates the promoter or enhancer provides higher levels of expression and/or secretion in the indicated cell or tissue type. Examples of promoters specific for liver are the transthyretin (TTR) gene promoter; human alpha 1-antitrypsin (hAAT) promoter; the apolipoprotein A-I promoter; albumin, Miyatake et al., J. Virol., 71:5124-32 (1997); hepatitis B virus core promoter, Sandig et al., Gene Ther. 3:1002-9 (1996); alpha-fetoprotein (AFP), Arbuthnot, et al., Hum. Gene. Ther., 7:1503-14 (1996); human Factor IX promoter; thyroxin binding globulin (TBG) promoter; TTR minimal enhancer/promoter; alpha-antitrypsin promoter; LSP (845 nt) (requires intronless scAAV); and LSP1 promoter. An example of an enhancer active in liver is apolipoprotein E (ApoE) HCR-1 and HCR-2 (Allan et al., J. Biol. Chem., 272:29113-19 (1997)).

Expression control elements also include ubiquitous or promiscuous promotors and promoters/enhancers capable of driving polynucleotide expression in many different cell types. Such elements include the cytomegalovirus (CMV) immediate early promoter/enhancer sequences, the Rous sarcoma virus (RSV) promoter/enhancer sequences, phosphoglycerate kinase (PKG) promoter, CAG (composite of the CMV enhancer, the chicken beta actin promoter (CBA) and the rabbit beta globin intron) (see, e.g., Boshart et al., (1985) Cell, 41:521-530), the SV40 promoter, the dihydrofolate reductase promoter, and the cytoplasmic b-actin promoter.

Examples of the CNS specific promoters include: neuron specific promoters such as the NSE (neuronal specific enolase), synapsin or NeuN, platelet-derived growth factor (PDGF), platelet-derived growth factor B-chain (PDGF-0), methyl-CpG binding protein 2 (MeCP2), Ca²/calmodulin-dependent protein kinase II (CaMKII), metabotropic glutamate receptor 2 (mGluR2), neurofilament light (NFL) or heavy (NFH), β-globin minigene nP2, preproenkephalin (PPE), enkephalin (Enk), and excitatory amino acid transporter 2 (EAAT2) promoters; astrocyte specific promoters such as the glial fibrillar acidic protein (GFAP) and EAAT2 promoters; oligodendrocyte specific promoters such as the myelin basic protein (MBP)/myelin-associated glycoprotein and oligodendrocyte transcription factor 2 promoter; neurons/hypothalamus specific promoters such as the proopiomelanocortin (POMC) promoter; and neurons/spinal cord specific promoters. (See, e.g., U.S. Patent Publication No. 2021/214749 and Adeno-Associated Virus Vectors (2019), Ed. Castle., 1^{st} Edition, Springer New York, New York, NY.; both of which are hereby incorporated by reference herein in their entirety.)

Additional promoters include the SV40 early promoter, superoxide dismutase 1 (SOD1) promotor, mouse mammary tumor virus LTR promoter, adenovirus major late promoter (Ad MLP), herpes simplex virus (HSV) promoter, SFFV promoter, rat insulin promoter, TBG promoter, the desmin promoter and similar muscle-specific promoters, the EF1-alpha promoter, synthetic promoters, hybrid promoters, and promoters with multi-tissue specificity.

Expression control elements also can impact expression in a manner that is regulatable by a signal or stimuli increasing or decreasing expression. A regulatable element increasing expression of transcribed nucleic acid in response to a signal or stimuli is also referred to as an “inducible element” (i.e., is induced by a signal). Typically, the amount of increase or decrease conferred by such elements is proportional to the amount of signal or stimuli present. Particular examples include zinc-inducible sheep metallothionine (MT) promoter; the steroid hormone-inducible mouse mammary tumor virus (MMTV) promoter; the T7 polymerase promoter system (International Patent Publication No. WO1998/10088); the tetracycline-repressible system (Gossen, et al., Proc. Natl. Acad. Sci. USA, 89:5547-5551 (1992)); the tetracycline-inducible system (Gossen et al., Science 268: 1766-1769 (1995); see also Harvey et al., Curr. Opin. Chem. Biol. 2:512-518 (1998)); the RU486-inducible system (Wang et al., Nat. Biotech. 15:239-243 (1997) and Wang et al., Gene Ther. 4:432-441 (1997); and the rapamycin-inducible system (Magari et al., J. Clin. Invest. 100:2865-2872 (1997); and Rivera et al., Nat. Medicine. 2:1028-1032 (1996)). Other examples of regulatable control elements include those regulated by a specific physiological state such as temperature, acute phase, or development.

In certain embodiments the expression cassette further comprises one or more introns independent of ApoE encoding nucleotide acid. A variety of different introns can be used to enhance gene expression. Examples of introns that may be used include the rabbit β-globin intron with splice donor/splice acceptor, SV40 intron with splice donor/splice acceptor, human β-globin introns, intron 2 of the human hemoglobin beta gene, hFIX int1 (intron 1 of the human coagulation factor IX gene), CBA-rHHB (synthetic intron derived from the fusion of the intron 1 of the chicken beta actin gene and intron 2 of the rabbit hemoglobin beta), CBA (intron 1 of the chicken beta actin gene), hGH (intron 1 of the human growth hormone gene), hFIX synth (synthetic intron derived from different portions of the human coagulation factor IX gene and present in the pLIVE vector, Mirus Bio, Madison, WI); human hemoglobin subunit beta (HBB2) synthetic intron, and optimized HBB2; and chimeric introns such as introns made up of the 5′-splice donor of the first human β-globin intron and the branch and 3′-acceptor site from the intron that is between the leader and the body of the immunoglobulin gene heavy chain variable region. (Buck et al., Int. J. Mol. Sci. (2020), 21, 4197; Ronzitti et al. Mol. Ther. Methods Clin Dev. (2016) July 20; 3:16049; and the HBB-IGG intron provided by the pCMVNT™ vector.)

In certain embodiments the expression cassette comprises a post-transcriptional regulatory element. Post-translational regulatory elements such as Woodchuck post-transcriptional regulatory element (WPRE) and Hepatitis B regulatory element can increase gene expression. (Buck et al., Int. J. Mol. Sci. (2020), 21, 4197).

Polyadenylation signal sequences provide for the formation of a polyA tail, which facilitates nuclear export, translation and/or mRNA stability, and may also be involved in transcription termination. Examples of polyadenylation signal sequences include SV40 late polyadenylation signal, bovine growth hormone polyA (bGHpA) signal sequence, synthetic polyA, mouse β-globin pA, rabbit β-globin pA, and H4-based pA, (Buck et al., Int. J. Mol. Sci. (2020), 21, 4197).

In certain embodiments, the expression cassette comprises a Kozak consensus sequence or a variation thereof. Kozak consensus sequences play an role in translation initiation. The Kozak consensus sequence and variations are provided in, for example, McClements et al., (2021) Molecular vision, 27, 233-242.

In certain embodiments the expression cassette comprises from 5′ to 3′ operatively coupled to the ApoE encoding sequence: a promoter or promoter/enhancer, an intron, a Kozak sequence, the ApoE encoding sequence and a polyadenylation signal.

In certain embodiments the expression cassette further comprises an miRNA target sequences, which in further embodiments is incorporated into the 3′ UTR of the expression cassette. An miRNA target sequence is recognized by miRNA present in particular cells or tissues leading to degradation of mRNA transcripts. Based on the presence of certain miRNA in particular cells, incorporating an miRNA target sequences can be used to reduce expression in certain cell or tissue types. Multiple tandem repeats of miRNA target sequences can be used to increase degradation. (Geisle et al., (2016) World Journal of Experimental Medicine 6(2): 37-54.)

In certain embodiments the expression cassettes comprises miRNA target sequence for the dorsal root ganglia, liver, or immune cells.

In certain embodiments, the expression cassette encoding nucleotide sequence contains any of 0-5, 0-10, 0-15, 0-50, or 0-100 CpGs; 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, and 82 CpGs; 0%, about 0.5%, about 1.0%, about 2.0%, about 3.0%, about 4.0%, about 5.0%, about 6%, about 7%, about 8%, about 9%, about 10%, about 11%, about 12%, about 13%, about 14%, or about 15% CpGs; and/or up to about 0.5%, up to about 1.0%, up to about 2.0%, up to about 3.0%, up to about 4.0%, up to about 5.0%, up to about 6%, up to about 7%, up to about 8%, up to about 9%, up to about 10%, up to about 11%, up to about 12%, up to about 13%, up to about 14%, or up to about 15% CpGs.

### III. Recombinant Viral Vector Nucleic Acid

Polynucleotide recombinant viral nucleic acid contain 5′ and/or 3′ viral elements providing for viral packaging and may provide for additional activities such as self-priming, DNA replication, promoter activity, genome integration, or episomal concatermerization. The 5′ and 3′ elements are general located at or near the 5′ and 3′ terminal end of the recombinant viral nucleic acid and can be naturally occurring or modified versions of naturally occurring sequences. Examples of 5′ and 3′ elements include adenovirus ITRs, adeno-associated virus ITRs and packaging sequence; and retrovirus 5′ and 3′ long terminal repeats (LTRs) and packaging sequence. (Naso et al., (2017) BioDrugs, 31(4), 317-334; Bulcha et al., (2021) Sig. Transduct. Target Ther. 6:53 (2021); and Liu and Seol (2020) BMB Reports; 53(11):565-575.)

The term “recombinant,” as a modifier of nucleic acid or a vector indicates a combination of elements that does not occur in nature. For example, recombinant viral vector nucleic acid provide 5′ and/or 3′ viral elements along with an expression cassette containing one or more elements not naturally associated with the 5′ and/or 3′ elements. Similarly, a viral vector, such as an rAAV vector may contain a naturally occurring or modified capsid, encapsidating recombinant viral vector nucleic acid.

Polynucleotides, expression cassettes and viral vector nucleic acid are compatible with the particular viral vector. For example, rAAV comprises ssDNA, adenovirus vectors comprising dsDNA, and retrovirus vectors comprising ssRNA.

In certain embodiments, the viral vector nucleic acid contains any of 0-5, 0-10, 0-15, 0-50, 0-100, or 0 to 150 CpGs; 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, and 82 CpGs; 0%, about 0.5%, about 1.0%, about 2.0%, about 3.0%, about 4.0%, about 5.0%, about 6%, about 7%, about 8%, about 9%, about 10%, about 11%, about 12%, about 13%, about 14%, or about 15% CpGs; and/or up to about 0.5%, up to about 1.0%, up to about 2.0%, up to about 3.0%, up to about 4.0%, up to about 5.0%, up to about 6%, up to about 7%, up to about 8%, up to about 9%, up to about 10%, up to about 11%, up to about 12%, up to about 13%, up to about 14% or up to about 15% CpGs.

### IV. Viral Vectors

In certain embodiments the gene delivery vehicle is a viral vector. Viral vectors comprises a protein capsid encapsidating recombinant viral nucleic acid and can deliver the nucleic acid to cells or tissues. Depending on the particular vector, the viral vector may further comprise a viral envelope. Examples of viral vectors that can be used for gene therapy include adenovirus vectors, rAAV, retrovirus vectors and herpes simplex vectors.

Different serotypes exist within different types of viruses. The different serotypes can provide for different activities, such as cell or tissue tropism and likelihood of generating a host immune response. The term “serotype” broadly refers to both serologically distinct viruses as well as viruses not serologically distinct that can be within a subgroup or a variant of a given serotype. Serologic distinctiveness can be determined based on the lack of cross-reactivity between antibodies to one capsid as compared to another capsid. Such cross-reactivity differences are usually due to differences in capsid protein sequences/antigenic determinants (e.g., due to VP1, VP2, and/or VP3 sequence differences of AAV serotypes).

As more naturally occurring virus isolates are discovered or capsid mutants generated, there may or may not be serological differences with any of the currently existing serotypes. Thus, in cases where the new virus has no serological difference, this new virus would be a subgroup or variant of the corresponding serotype.

### IV.A. Adenovirus Vectors

Adenoviruses are non-enveloped double-stranded DNA viruses. Recombinant adenovirus vectors comprise recombinant adenovirus nucleic acid lacking one or more protein involved in viral replication, and further comprise an adenoviral capsid. Recombinant adenovirus vectors can be produced containing different amounts of adenoviral DNA. The Ad genome is flanked by hairpin-like inverted terminal repeats (ITRs) varying in length from 30-371 bp at its termini. The ITRs serve as self-priming structures that promote primase-independent DNA replication. A packaging signal located at the left arm of the genome is required for viral genome packaging. (Liu and Seol (2020) BMB Reports; 53(11):565-575; and Bulcha et at, (2021) Sig. Transduct. Target Ther. 6:53.)

In certain embodiments, the recombinant adenovirus vector is a third-generation vector, which are also referred to as “gutless” or “helper-dependent”. Gutless vectors can be produced from recombinant adenoviral nucleic acid where all, or substantially all viral sequences, except for the ITRs and the packaging signal are not present. Gutless adenovirus vectors are high capacity vectors able to accommodate up to about 36 kb of DNA insert. Preferred recombinant adenovirus nucleic acid is about 27 kb to about 37 kb. Stuffer sequences can be added to recombinant adenovirus nucleic acid to increase nucleic acid size and capsid incorporation. Preferred stuffer sequences avoid coding sequences, repetitive sequences, recombination sequences, and immunogenic sequences. (Liu and Seol (2020) BMB Reports; 53(11):565-575; Bulcha et al., (2021) Sig. Transduct. Target Ther. 6:53; and Sandig et al., PNAS (2000) 97(3):1002-1007, each of which are hereby incorporated by reference herein in their entirety.)

In certain embodiments, recombinant adenovirus vectors can be produced based on rare human serotypes or chimpanzee serotypes. The use of chimpanzee and rare human serotypes may be helpful in reducing host immune response against recombinant adenovirus vectors due to preexisting immunity. (Guo et al., (2018) Human vaccines & immunotherapeutics, 14(7):1679-1685 and Bulcha et al., (2021) Sig. Transduct. Target Ther. 6:53)

Adenovirus vectors can be produced by supplying viral proteins needed for vector production in trans using, for example, appropriate helper viruses or plasmids and cell lines. (Liu and Seol (2020) BMB Reports; 53(11):565-575; and Bulcha et al., (2021) Sig. Transduct. Target Ther. 6:53.)

### IV.B. AAV Vectors

Recombinant adeno-associated viral vector (also referred to herein as “rAAV”) are based on the adeno-associated virus. The adeno-associated virus is a single-strand DNA virus containing a 4.7-kb genome flanked by 145-nt ITRs on both ends of the genome. ITR activity is important for self-priming and packaging, and may also provide additional activity such as promoter activity.

An rAAV contains AAV recombinant nucleic acid and a viral capsid. The rAAV recombinant nucleic acid lacks one or more AAV proteins involved in viral replication. In certain embodiments, the rAAV nucleic acid is at least about 2.5 kb. In certain embodiments, rAVV nucleic acid has a size range about 4 kb to about 5.2 kb. If needed, stuffer sequences can be used to increase rAAV nucleic acid size and packaging efficiency. In different embodiments, the rAAV nucleic acid including stuffer is 4-5.2 kb, 3.0-5.5 kb, 4.0-5.0 kb, 4.3-4.8 kb, about 4.2 kb, about 4.3 kb, about 4.4 kb about 4.5 kb, about 4.6 kb, or about 4.7 kb. Preferred stuffer sequences avoid coding sequences, repetitive sequences, recombination sequences, and immunogenic sequences.

In certain embodiments rAAV nucleic acid comprise a 5′ ITR and/or 3′ ITR independently selected from 5′ and 3′ ITRs provided in AAV1, AAV2, AAV3, AAV4, AAV5, AAV6, AAV7, AAV8, AAV9, AAV10, AAV11, AAV12, AAVrh.10, AAVrh.74 and AAV3B ITRs. In further embodiments 5′ and 3′ ITRs are present, and both ITRs are from the same serotype genome.

In further embodiments the 5′ ITR comprises a sequence with a sequence identity of at least 95%, at least 96%, at least 97%, at least 98%, at least 99% or 100% to any of SEQ ID NOs: 81, 88, 90, 92 and 94 and the 3′ ITR independently comprises a sequence with a sequence identity of at least 95%, at least 96%, at least 97%, at least 98%, at least 99% or 100% to any of SEQ ID NOs: 82, 89, 91 and 93.

In certain embodiments the rAAV is a self-complementary adeno-associated virus vector (scAAV) or short hairpin adeno-associated virus vector (shAAV). scAAV and shAAV provide for a double-stranded rAAV nucleic acid that can be incorporated into an AAV capsid. scAAV and shAAV comprise inverted dimeric repeats providing intramolecular double-stranded DNA. scAAV can be produced by mutating an ITR terminal resolution site so that rep fails to nick the terminal resolution site. shAAV can utilize a short hairpin to produce double-stranded AAV nucleic acid. scAAV and shAAV being double-stranded DNA provide an advantage in circumventing the DNA synthesis step required for single-stranded rAAV nucleic acid upon entry into a cell. A potential disadvantage of scAAV and shAAV is the size of DNA inserts that can be incorporated is reduced by about half compared to single-stranded rAAV nucleic acid. (U.S. Pat. No. 10,457,940; Xie et al., Mol Ther. (2017) 25(6):1363-1374; and McCarty Mol. Ther. (2008) 16(10):1648-1656; each of which are hereby incorporated by reference herein in their entirety.)

Naturally occurring AAV capsids contain viral protein VP1, VP2 and VP3 in a ratio of about 1:1:10. AAV vectors can be produced where all three viral proteins are based upon a particular serotypes or where one, two or all three viral protein are based on different serotypes or variants thereof.

In certain embodiments, AAV capsids are based on VP1, VP2 or VP3 having a sequence identity of at least 80% to a VP1, VP2 or VP3 of any of AAV1, AAV2, AAV3, AAV4, AAV5, AAV6, AAV7, AAV8, AAV9, AAV10, AAV11, AAV12, AAVrh.74, AAV3B, AAV-2i8, AAVrh.10, AAVrh.8, AAVHSC, AAV-B1, AAV-AS, AAV1/rh.10, SEQ ID NO: 83 and SEQ ID NO: 84; as well as variants (e.g., capsid variants, such as amino acid insertions, additions, substitutions and deletions) thereof. (See for example, U.S. Pat. Nos. 9,909,142 and 9,840,719 disclosing RHM4-1, RHM15-1, RHM15-2, RHM15-3/RHM15-5, RHM15-4 and RHM15-6; U.S. Patent Publication No. 2013/0059732 and U.S. Pat. No. 9,169,299, disclosing LK01, LK02, and LK03; and U.S. Pat. No. 11,110,153; the disclosures of which are herein incorporated in their entirety.)

Recombinant AAV capsid and nucleic acid can be based on the same serotype (or subgroup or variant), or can be different from each other. In certain embodiments, an rAAV nucleic acid has the same serotype genome (e.g., ITRs) as the encapsidating capsid protein.

In different embodiments, the rAAV capsid comprises a protein having a sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.9% or 100% identical to a VP1, VP2 or VP3 of any of AAV1, AAV2, AAV3, AAV4, AAV5, AAV6, AAV7, AAV8, AAV9, AAV10, AAV11, AAV12, AAVrh.74, AAV3B, AAV-2i8, AAVrh.10, AAVrh.8, AAVHSC, AAV-B1, AAV-AS, AAV1/rh.10; and VP1 of SEQ ID NO: 83 or SEQ ID NO: 84.

In certain embodiments, the rAAV capsid comprises a VP1 comprising SEQ ID NO: 83, a VP2 comprising SEQ ID NO: 122 and a VP3 comprising SEQ ID NO: 123.

In certain embodiments involving treatment of a CNS disease or disorder, the capsid provides for CNS expression. Examples of such rAAV capsids and the design of rAAV capsids able to provide for CNS expression are provided in Chen et al., (2021) Journal of Controlled Release 333, 129-138 (e.g., AAV9, AAVrh.10, AAVrh.8, AAVHSC, AAV-B1, AAV-AS, and AAV1/rh.10), U.S. Pat. No. 9,585,971, Goertsen et al., Nat. Neurosci. 25, 106-115 (2022), and Ittner et al., Br. J. Pharmacol. (2019) 176:3649-3655, each of which are incorporated by reference herein in its entirety.

Recombinant AAV can be produced by supplying viral proteins needed for vector production in trans using for example, appropriate helper viruses or plasmids and cell lines. In certain embodiments, rAAV is produced using a rAAV vector genome plasmid. The plasmid comprises that portion of the rAAV nucleic acid that is ultimately packaged or encapsidated to form a viral (e.g., rAAV) particle. The “plasmid backbone,” contains elements important for propagation and recombinant virus production. Except for possible 3′ ITR and/or 5′ ITR cloning remnants the plasmid backbone is not itself packaged or encapsidated into virus (e.g., AAV) particles.

Recombinant AAV can be produced from different types of cell lines. In certain embodiments human HEK293 cells are used (American Type Culture Collection Accession Number ATCC CRL1573). Other host cell lines appropriate for rAAV production are described in, for example, Robert et al., (2017) Biotechnol. J., 12: 1600193; and International Application PCT/US2017/024951, the disclosures of which are herein incorporated in its entirety.

The AAV genome contains two main genes: rep and cap. Transcription from the rep gene is initiated from two different promoters resulting in the production of nonstructural proteins designated Rep78, Rep68, Rep52, and Rep40. The rep proteins function in genome replication and/or encapsidation. The cap gene encodes for structural proteins making up the capsid (VP1, VP2 and Vp3); a non-structural assembly-activating protein (APP), which performs functions related to capsid assembly; and the membrane-associated accessory protein, which may be associated with production phases of the replication cycle. (Maurer and Weitzman (2020) Hum. Gene Ther. 31(9-10):499-511, hereby incorporated by reference herein in its entirety.)

AAV requires helper virus functions to complete it replication cycle. Helper virus functions can be supplied by different viruses in permissive cell lines. Permissive cell lines are cell lines able to support viral replication. Examples of helper viruses for AAV include adenovirus, HSV-1, HPV-16, and HBoV1 which can be used in conjunction with, for example, permissive primate cells; and baculovirus which can be used in conjunction with, for example, permissive insect cells such as sf9. (Maurer and Weitzman (2020) Hum. Gene Ther. (2020) 31(9-10):499-511 and Meier et al., (2020) Viruses 19; 12(6):662, both of which are herein incorporated by reference herein in their entirety.)

Recombinant AAV can be produced by supplying viral proteins needed for vector production in trans using, for example, appropriate helper viruses or plasmids and cell lines. In certain embodiments, rAAV is produced using a rAAV vector genome plasmid. The plasmid comprises that portion of the rAAV nucleic acid ultimately packaged or encapsidated to form a viral (e.g., rAAV) vector. The “plasmid backbone,” contains elements important for propagation and recombinant virus production. Except for possible 3′ ITR and/or 5′ ITR cloning remnants the plasmid backbone is not itself packaged or encapsidated into virus particles.

The vector genome plasmid may contain regions such an origin of replication and a selectable marker. Additional sites that may be present include cloning sites.

Recombinant AAV can be produced from different types of cell lines including HeLa, A549, BHK, Vero, and HEK293, or derivatives thereof. Other host cell lines appropriate for rAAV vector production are described in, for example, Robert et al., (2017) Biotechnol. J. (2017) 12(3), 1600193; and International Application No. PCT/US2017/024951; the disclosures of which are herein incorporated in its entirety.

Recombinant AAV can be cultured under a variety of different conditions suitable for providing cell growth and gene expression. References describing rAAV manufacturing include Clément and Grieger (2016) Mol. Ther. Methods Clin. Dev. 16; 3:16002; Robert et al., (2017) Biotechnol. J. 12(3), 1600193; and Adeno-Associated Virus Vectors (2019), Ed. Castle., 1^{st} Edition, Springer New York, New York, NY.; each of which are hereby incorporated by reference herein in their entirety.)

In certain embodiments, a rAAV vector is produced by a rAAV production cell comprising rAAV helper virus activity. The genome of the rAAV production cell comprises rAAV nucleic acid, the rep gene and the cap gene.

In certain embodiments, a rAAV vector is produced by culturing a rAAV permissive cell comprising an AAV genome plasmid, where the rAAV permissive cell further comprises rep and cap genes provided either as part of the cell genome and/or by one or more separate plasmids; and helper virus activity either as part of the cell genome and/or provided by one or more separate plasmids. In further embodiments, (a) the rAAV permissive cell line is a packaging cell, wherein the genome of the packaging cell comprises the cap gene and the rep gene; (b) the rep gene, cap gene, and helper activity are provided from the same plasmid; or (c) the rep gene and cap gene are provided by a rep cap plasmid and helper activity is provided by a helper plasmid.

In certain embodiments involving the use of HSV helper functions, the helper functions are provided by genes encoding for at least UL5, UL8, UL52, and ICP8.

In certain embodiments involving the use of adenovirus helper functions, the helper function are provided by genes encoding for at least E1A, E1B19K, E1B55K, E2A, E4orf6 and VA RNA. In certain embodiments E1, E2A and VR RNA functions are provided by a helper plasmid, where additional helper functions are provided by a host strain.

In certain embodiments, rAAV vector is obtained by producing rAAV using methods described herein and purifying the rAAV. Purification of rAAV can performed using techniques such as gradient-based purification, column-based, and combined methods. (See, e.g., Ayuso et al., (2010), Curr Gene Ther. (2010) 10(6):423-36, hereby incorporated by reference herein in its entirety.)

In certain embodiments, AAV helper functions are introduced into the host cell by transfecting the host cell with an AAV helper construct either prior to, or concurrently with, the transfection of an AAV expression vector. A host cell having AAV helper functions can be referred to as a “helper cell” or “packaging helper cell.” AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be, for example, in the form of a plasmid, phage, transposon, cosmid, virus, or virion. A number of AAV helper constructs have been described, such as plasmids pAAV/Ad and pIM29+45 which encode both Rep and Cap expression products. A number of other vectors are known which encode Rep and/or Cap expression products. Recombinant AAV can be produced, for example, as described in U.S. Pat. No. 9,408,904; and International Applications PCT/US2017/025396 and PCT/US2016/064414, the disclosures of which are herein incorporated in their entirety.

### IV.C. Retrovirus Vectors

Retroviruses are enveloped, single-stranded RNA viruses comprising 5′ and 3′ LTRs, and a signal packaging sequence located just outside of the LTR. Different types of retrovirus vectors can contain different amounts of viral genome. In certain embodiments, the retrovirus vector is a lentiviral vector based on HIV, retaining all cis-acting sequences needed for viral RNA packaging, reverse transcription and proviral DNA integration, while removing all HIV protein-coding genes. Lentiviral vectors have a packaging capacity of up to about 9 kb. If needed, stuffer sequence can be used to increase rAAV nucleic acid size and packaging efficiency. Lentiviral vectors can be produced by supplying viral proteins needed for vector production in trans using appropriate plasmids and cell lines. (Bulcha et al., (2021) Sig. Transduct. Target Ther. 6:53.)

### V. Non-Viral Vectors

In certain embodiments, the gene delivery vehicle is a non-viral vector. Non-viral vectors include nanoparticles and naked nucleic acid. Preferred non-viral vectors are nanoparticles. A variety of different nanoparticles can be employed including lipid nanoparticles (LNP), polymeric nanoparticles, lipid polymer nanoparticles (LPNP), protein and peptide-based nanoparticles, DNA dendrimers and DNA-based nanocarriers, carbon nanotubes, microparticles, microcapsules, inorganic nanoparticles, peptide cage nanoparticles, and exosomes. (See, e.g., Riley and Vermerris Nanomaterials (2017) 201, 7, 94; Thomas et al., Molecules (2019), 24, 3744; Bochicchio et al., (2021), 13, 198; Munagala et al., Cancer Letters (2021), 505, 58; Fu et al., (2020) NanoImpact 20, 100261; Neshat et al. (2020) Current Opin. Biotechnol. 66:1-10; Ouranidis et al., (2022) Biomedicines, 10, 50; and Qin et al., Signal Transduct Target Ther. (2022) May 21; 7(1):166, each of which are hereby incorporated by reference herein in their entirety.)

If desired, a nanoparticle can target a cell type using, for example, targeting ligands recognizing a target cell receptor. Examples of targeting ligands include carbohydrates (e.g., galactose, mannose, glucose, and galactomannan), endogenous ligands (e.g., folic acid and transferrin), antibodies and protein/peptides (e.g., RGD, epidermal growth factor, and low density lipoprotein) and peptides. (For example, Teo et al., Advanced Drug Delivery Reviews (2016), 98, 41.)

Nanoparticles can be used to deliver the ApoE encoding polynucleotide constructs described herein to a cell. In different embodiments, nanoparticles can deliver additional therapeutic compounds; and one or more additional compounds is provided in different nanoparticles. Reference to compound includes small molecules and large molecules (e.g., therapeutic proteins and antibodies).

The production of different nanoparticles and incorporation of nucleic acid and other compounds is well known in the art. Examples of publications illustrating incorporation of nucleic acid in a particular nanoparticle such as an LPNP and a LNP include Teo et al., Advanced Drug Delivery Reviews (2016) 98, 41; Bochicchio et al., Pharmaceutics (2021) 13, 198; Mahzabin and Das, IJPSR (2021) 12(1), 65; and Teixeira et al., (2017) Prog. Lipid Res. October; 68:1-11 (each of which are hereby incorporated by reference herein in their entirety). Factors that may impact small molecule incorporation into a nanoparticle include hydrophobicity and the presence of an ionizable moiety. (See, e.g., Nii and Ishii International Journal of Pharmaceutics (2005) 298, 198; and Chen et al., Journal of Controlled Release (2018) 286, 46.)

### V.A. Lipid-Based Delivery Systems

Lipid-based delivery systems include the use of a lipid as a component. Examples of lipid-based delivery systems include liposomes, LNPs, micelles, and extracellular vesicles.

A “lipid nanoparticle” or “LNP” refers to a lipid-based vesicle useful for delivery of nucleic acid molecules and having dimensions on the nanoscale. In different embodiments the nanoparticle is from about 10 nm to about 1000 nm, about 50 nm to about 500 nm, or about 50 nm to about 200 nm.

DNA is negatively charged. Thus, it can be beneficial for the LNP to comprise a cationic lipid such as, for example, an amino lipid. Exemplary amino lipids are described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, all of which are incorporated herein in their entirety. In certain embodiments, the LNP comprises amino lipids described in U.S. Pat. No. 9,512,073, hereby incorporated herein in its entirety.

The terms “cationic lipid” and “amino lipid” are used interchangeably herein to include lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipid can also be titratable cationic lipids. In certain embodiments, the cationic lipids comprise a protonatable tertiary amine (e.g., pH-titratable) group; C18 alkyl chains, wherein each alkyl chain independently can have one or more double bonds, one or more triple bonds; and ether, ester, or ketal linkages between the head group and alkyl chains.

Cationic lipids include 1,2-dilinoleyloxy-N,N-dimethylaminopropane (DLinDMA), 1,2-dilinolenyloxy-N,N-dimethylaminopropane (DLenDMA), 1,2-di-7-linolenyloxy-N,N-dimethylaminopropane (γ-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino)butanoate (DLin-M-C3-DMA, also known as MC3), salts thereof, and mixtures thereof. Other cationic lipids also include 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, γ-DLen-C2K-DMA, and (DLin-MP-DMA) (also known as 1-B11).

Still further cationic lipids include 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECTAMINE® (comprising DOSPA and DOPE, available from GIBCO/BRL).

Additional ionizable lipids that can be used include C12-200, 306Oi10, MC3, cKK-E12, bCKK-E12, Lipid 5, Lipid 9, ATX-002, ATX-003, and Merck-32. U.S. Patent Application Publication No. 2017/0367988, describes Merck-32.

In further embodiments, cationic lipid can be present in an amount from about 10% by molar ratio of the LNP to about 85% by molar ratio of the LNP, or from about 50% by molar ratio of the LNP to about 75% by molar ratio of the LNP.

LNP can comprise a neutral lipid. Neutral lipids can comprise a lipid species existing either in an uncharged or neutral zwitterionic form at physiological pH. Such lipids include diacylphosphatidylcholine, diacylphosphatidylethanolamine, ceramide, sphingomyelin, dihydrosphingomyelin, cephalin, and cerebrosides. The selection of neutral lipids is generally guided by considerations including particle size and stability. In certain embodiments, the neutral lipid component can be a lipid having two acyl groups (e.g., diacylphosphatidylcholine and diacylphosphatidylethanolamine).

Lipids having a variety of acyl chain groups of varying chain length and degree of saturation are available or can be isolated or synthesized. In certain embodiments, lipids containing saturated fatty acids with carbon chain lengths in the range of C14 to C22 can be used. In certain embodiments lipids with mono or di-unsaturated fatty acids with carbon chain lengths in the range of C14 to C22 are used. Additionally, lipids having mixtures of saturated and unsaturated fatty acid chains can be used. Exemplary neutral lipids include 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or a phosphatidylcholine. The neutral lipids can also be composed of sphingomyelin, dihydrosphingomyelin, or phospholipids with other head groups, such as serine and inositol.

In further embodiments, providing for neutral lipids, the neutral lipid can be present in an amount from about 0.1% by weight of the LNP to about 99% by weight of the LNP, or from about 5% by weight of the LNP to about 15% by weight of the LNP, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

LNP can contain additional components such as sterols and polyethylene glycol. Sterols can confer fluidity to the LNP. As used herein “sterol” refers to a naturally occurring sterol of plant (phytosterols) or animal (zoosterols) origin as well as non-naturally occurring synthetic sterols, all of which are characterized by the presence of a hydroxyl group at the 3-position of the steroid A-ring. Suitable sterols include those conventionally used in the field of liposome, lipid vesicle or lipid particle preparation, most commonly cholesterol. Phytosterols include campesterol, sitosterol, and stigmasterol. Sterols also include sterol-modified lipids, such as those described in U.S. Patent Application Publication No. 2011/0177156. In different embodiments providing for a sterol, the sterol is present in an amount from about 1% by weight of the LNP to about 80% by weight of the LNP or from about 10% by weight of the LNP to about 25% by weight of the LNP.

Polyethylene glycol (PEG) is a linear, water-soluble polymer of ethylene PEG repeating units with two terminal hydroxyl groups. PEGs are classified by their molecular weights, for example, PEG 2000 has an average molecular weight of about 2,000 daltons, and PEG 5000 has an average molecular weight of about 5,000 daltons. PEGs commercially available from Sigma Chemical Co. and other companies include monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments concerning PEG, PEG has an average molecular weight of about 550 to about 10,000 daltons and is optionally substituted by alkyl, alkoxy, acyl or aryl. In further embodiments, the PEG is substituted with methyl at the terminal hydroxyl position. In further embodiments, the PEG has an average molecular weight from about 750 to about 5,000 daltons, or from about 1,000 to about 5,000 daltons, or from about 1,500 to about 3,000 daltons, or from about 2,000 daltons, or from about 750 daltons.

PEG-modified lipids include the PEG-dialkyloxypropyl conjugates (PEG-DAA) described in U.S. Pat. Nos. 8,936,942 and 7,803,397. PEG-modified lipids (or lipid-polyoxyethylene conjugates) can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873, PEG-modified dialkylamines and PEG-modified 1,2-diacyloxypropan-3-amines. In certain embodiments, the PEG-modified lipid can be PEG-modified diacylglycerols and dialkylglycerols. In certain embodiments, the PEG can be in an amount from about 0.1% by weight of the LNP to about 50% by weight of the LNP, or from about 5% by weight of the LNP to about 15% by weight of the LNP.

In further embodiments concerning LNP size, prior to encapsulating nucleic acid, LNPs have a size range from about 10 nm to 500 nm, or from about 50 nm to about 200 nm, or from 75 nm to about 125 nm.

In certain embodiments concerning LNP, the LNP is described by Billingsley et al., Nano Lett. 2020, 20, 1578 or Billingsley et al., International Patent Publication No. WO 2021/077066 (both of which are hereby incorporated by reference herein in their entirety). Billingsley et al., and WO2021/077066 describe LNPs containing lipid-anchored PEG, cholesterol, phospholipid and ionizable lipids. In certain embodiments, the LNP contains a C14-4 polyamine core and/or has a particle size of about 70 nm. C14-4 has the following structure.

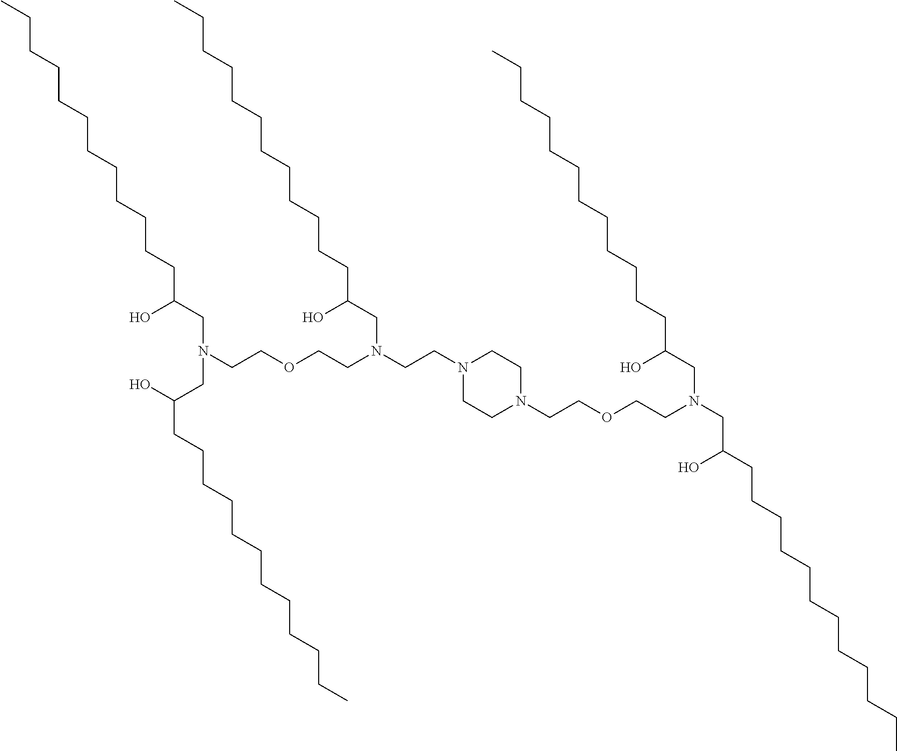 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

In certain embodiments the LNP is made up of a cationic lipid or lipopeptide described by U.S. Pat. Nos. 10,493,031, 10,682,374 or WO2021/077066 (each of which is hereby incorporated by reference herein in its entirety). In certain embodiments, the LNP contains a cationic lipid, a cholesterol-based lipid, and/or one or more PEG-modified lipids. In certain embodiments the LNP contains cKK-E12 (Dong et al., PNAS (2014) 111(11), 3955):

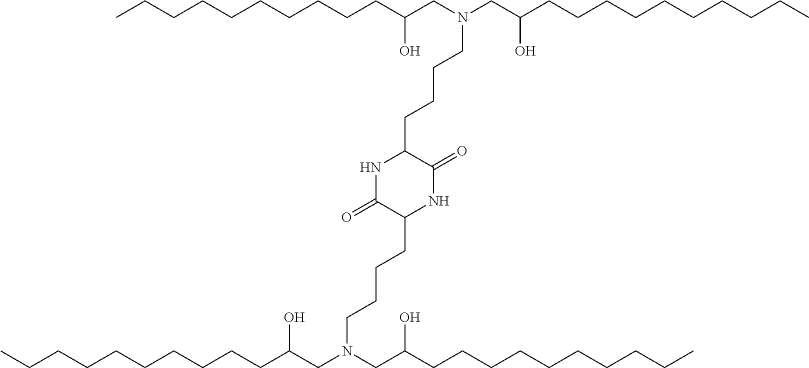 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

In certain embodiments the LNP comprises a modified form of cKK-E12 referred to herein as “bCKK-E12,” having the following structure:

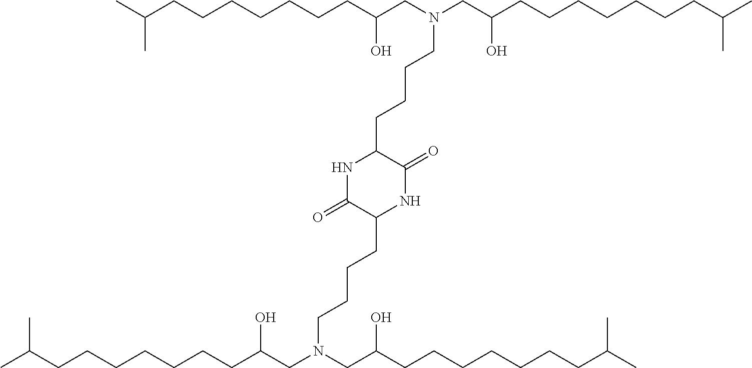 [SMILES: CC(C)CCCCCCCC(O)CN(CCCCC1NC(=O)C(CCCCN(CC(O)CCCCCCCC(C)C)CC(O)CCCCCCCC(C)C)NC1=O)CC(O)CCCCCCCC(C)C]

In certain embodiments the LNP comprises Lipid 1, 2, 3, 4, 5, 6, 7, 8, 9 or 10 as described by Sabnis et al., Molecular Therapy 2018, 26:6, 1509-1519 (hereby incorporated by reference herein in its entirety). In certain embodiments the LNP comprises Lipid 5, 8, 9, 10, or 11 described in Sabnis et al.

Lipid 5 of Sabnis et al. has the structure:

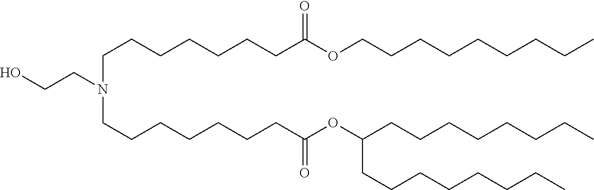 [SMILES: CCCCCCCCCOC(=O)CCCCCCCN(CCO)CCCCCCCC(=O)OC(CCCCCCCC)CCCCCCCC]

Lipid 9 of Sabnis et al. has the structure:

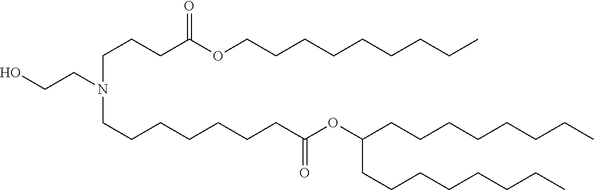 [SMILES: CCCCCCCCCOC(=O)CCCN(CCO)CCCCCCCC(=O)OC(CCCCCCCC)CCCCCCCC]

Additional lipids that may be utilized include those described by Roces et al., Pharmaceutics, 2020, 12, 1095; Jayaraman et al, Angew. Chem. Int. Ed., 2012, 51, 8529-8533; Maier et al., www.moleculartherapy.org, 2013, Vol. 21, No. 8, 1570-1578; Liu et al., Adv. Mater. 2019, 31, 1902575, e.g., BAMEA-016B; Cheng et al., Adv. Mater., 2018, 30, 1805308, e.g., 5A2-SC8; Hajj and Ball, Small, 2019 15, 1805097, e.g., 306Oi10; Du et al., U.S. Patent Application Publication No. 20160376224; and Tanaka et al., Adv. Funct. Mater., 2020, 30, 1910575; each of which are hereby incorporated by reference herein in their entirety.

In further embodiments, the nanoparticle is an LNP. In further embodiments the LPN in mol % comprises, consists essentially, or consists, of the following components: (1) one or more cationic lipids from about 20% to about 65%, one or more phospholipid lipids from about 1% to about 50%, one or more PEG-conjugated lipid from about 0.1% to about 10%, and cholesterol from about 0% to about 70%; and (2) one or more cationic lipids from about 20% to about 50%, one or more phospholipid lipids from about 5% to about 20%, one or more PEG-conjugated lipids from about 0.1% to about 5%, and cholesterol from about 20% to about 60%. In further embodiments the phospholipid lipid is a neutral lipid; and the phospholipid lipid is DOPE or DSPC.

In further embodiments the LNP, in mole %, comprises, consists essentially, or consists of the following components: (1) cKK-E12, about 35%; C14-PEG2000, about 2.5%; cholesterol, about 46.5%; and DOPE, about 16%; (2) bCKK-E12, about 35%; C14-PEG2000, about 2.5%; cholesterol, about 46.5%; and DOPE, about 16%; (3) Lipid 9 (further described in Sabnis et al.), about 50%; C14-PEG2000, about 1.5%; cholesterol, about 38.5%; and DSPC about 10%; or (4) Lipid 5 (further described in Sabnis et al.), about 50%; C14-PEG2000 about 1.5%; cholesterol about 38.5%; and DSPC about 10%; and (5) ionizable lipid, about 50%; DSPC, about 10%; cholesterol, about 37.5%; and stabilizer (PEG-Lipid), about 2.5%; or (6) is GenVoy-ILM™ LNP (Precision NanoSystems).

### V.B. Polymer-Based Nanoparticles

Polymer-based delivery systems can be made from a variety of different natural and synthetic materials. DNA and other compounds can be entrapped into the polymeric matrix of polymeric nanoparticles or can be adsorbed or conjugated on the surface of the nanoparticles. Examples of commonly used polymers for nucleic acid delivery include poly(lactic-co-glycolic acid) (PLGA), poly lactic acid (PLA), poly(ethylene imine) (PEI) and PEI derivatives, chitosan, dendrimers, polyanhydride, polycaprolactone, polymethacrylates, poly-L-lysine, pullulan, dextran, and hyaluronic acid, poly-β-aminoesters. (Thomas et al., (2019) Molecules 24, 3744.)

Polymeric-based nanoparticles can have different sizes, ranging from about 1 nm to about 1000 nm, from about 10 nm to about 500 nm, from about 50 nm to about 200 nm, from about 100 nm to about 150 nm, and from about 150 nm or less.

### V.C. Lipid Polymer Nanoparticles

Lipid polymer nanoparticles are hybrid nanoparticles providing both a lipid component and a polymer component, and as such can be considered to be an LNP or LPNP. The LPNP configuration can provide an outer polymer and inner lipid or an outer lipid and inner polymer. The presence of two different types of material facilitates designing nanoparticles to provide for delayed release of a component. Different lipid and polymer components can be selected taking into account the material be delivered. (For example, see Teo et al., Advanced Drug Delivery Reviews (2016) 98, 41; Bochicchio et al., Pharmaceutics (2021) 13, 198; Mahzabin and Das, IJPSR (2021) 12(1), 65; and Teixeira et al., (2017) Prog. Lipid Res. October; 68:1-11.)

### V.D. Protein and Peptide-Based Nanoparticles

Protein and peptide-based systems can employ a variety of different proteins and peptides. Examples of proteins that can be employed include gelatin and elastin. Peptide-based systems can employ, for example, CPPs.

CPPs are short peptides (6-30 amino acid residues) potentially capable of intracellular penetration to deliver therapeutic molecules. The majority of CPPs consists mainly of arginine and lysine residues, making them cationic and hydrophilic, but CPPs can also be amphiphilic, anionic, or hydrophobic. CPPs can be derived from natural biomolecules (e.g., HIV-1 Tat protein), or obtained by synthetic methods (e.g., poly-L-lysine, polyarginine) (Singh et al., Drug Deliv. 2018; 25(1):1996-2006). Examples of CPPs include cationic CPPs (highly positively charged) such as the Tat peptide, penetratin, protamine, poly-L-lysine, and polyarginine; amphipathic CPPs (chimeric or fused peptides, constructed from different sources, containing both positively and negatively charged amino acid sequences), such as transportan, VT5, bactenecin-7 (Bac7), proline-rich peptide (PPR), SAP (VRLPPP)₃, TP10, pep-1, and MPG); membranotropic CPPs (exhibit both hydrophobic and amphipathic nature simultaneously, and comprise both large aromatic residues and small residues) such as H625, SPIONs-PEG-CPP and NPs; and hydrophobic CPPs (contain only non-polar motifs or residues) such as SG3, PFVYLI, pep-7, and fibroblast growth factors.

Protein and peptide nanoparticles can be provided in different sizes, for example, ranging from about 1 nm to about 1000 nm, from about 10 nm to about 500 nm, from about 50 nm to about 200 nm, from about 100 nm to about 150 nm, or from about 150 nm or less.

### V.E. Peptide Cage Nanoparticles

Peptide cage-based delivery systems can be produced from proteinaceous material able to assemble into a cage-like structure forming a constrained internal environment. Peptide cages can comprise a proteinaceous shell that self-assembles to form a protein cage (e.g., a structure with an interior cavity that is either naturally accessible to the solvent or can be made so by altering solvent concentration, pH, or equilibria ratios). The monomers of the protein cages can be naturally occurring or variant forms, including amino acid substitutions, insertions, and deletions (e.g., fragments).

Different types of protein “shells” can be assembled and loaded with different types of materials. Protein cages can be produced using viral coat protein(s) (e.g., from the Cowpea Chlorotic Mottle Virus protein coat), as well non-viral proteins (e.g., U.S. Pat. Nos. 6,180,389 and 6,984,386, U.S. Patent Publication No. 20040028694, and U.S. Patent Publication No. 20090035389, each of which is incorporated by reference herein in their entity).

Examples of protein cages derived from non-viral proteins include: eukaryotic or prokaryotic derived ferritins and apoferritins such as 12 and 24 subunit ferritins; and heat shock proteins (HSPs), such as the class of 24 subunit heat shock proteins that form an internal core space, the small HSP of Methanococcus jannaschii, the dodecameric Dsp HSP of E. coli; and the MrgA protein.

Protein cages can have different core sizes, such as ranging from about 1 nm to about 1000 nm, from about 10 nm to about 500 nm, from about 50 nm to about 200 nm, from about 100 nm to about 150 nm, or from about 150 nm or less.

### V.F. Exosomes

Exosomes are small biological membrane vesicles and have been utilized to deliver various cargoes including small molecules, peptides, proteins and nucleic acids. Exosomes generally range in size from about 30 nm to 100 nm and can be taken up by a cell and deliver its cargo. Cargoes can be associated with exosome surface structure or may be encapsulated within the exosome bilayer.

Various modifications can be made to exosomes facilitating cargo delivery and cell targeting. Modifications for facilitating cargo delivery include structures for associating with cargoes such as protein scaffolds and polymers. Modifications for cell targeting include targeting ligands and modifying surface charge. Publications describing production, modification, and use of exosomes for delivery of different cargoes include Munagala et al., Cancer Letters (2021), 505, 58; Fu et al., (2020) NanoImpact 20, 100261; and Dooley et al., (2021) Molecular Therapy 29(5), 1729 (each of which is hereby incorporated by reference herein).

### VI. Pharmaceutical Compositions

Pharmaceutical compositions comprise a pharmaceutical acceptable carrier facilitating administration and/or storage of the polynucleotide constructs, viral vectors and non-viral vectors described herein. Reference to “pharmaceutically acceptable” indicates the components do not cause substantial undesirable biological effects at the amount utilized. Pharmaceutically acceptable carriers can contain different components such as one or more pharmaceutically acceptable excipients such as a salt, sugar, buffer, solvent, preservative, protein and surfactant. A particular excipient can have more than one function. Examples of pharmaceutically acceptable excipients and carriers that can be used for viral vectors are provided in, for example, International Patent Publication No. WO2021/071835.

Pharmaceutical compositions can be formulated to be compatible with a particular route of administration or delivery. Compositions suitable for parenteral administration include aqueous and non-aqueous solutions, suspensions or emulsions, which preparations are typically sterile and can be isotonic with the blood of the intended recipient. Illustrative examples include water, buffered saline, Hanks' solution, Ringer's solution, dextrose, fructose, ethanol, animal vegetable and synthetic oils. Aqueous injection suspensions can contain substances which increase the viscosity of the suspension, such as sodium carboxymethyl cellulose, sorbitol, or dextran.

In an embodiment, the pharmaceutical composition contains a formulation capable of injection into a subject. Examples of injectable formulation components include isotonic, sterile, saline solutions, salts (e.g., monosodium or disodium phosphate, sodium, potassium, calcium or magnesium chloride and mixtures of such salts), buffered saline, sugars (e.g., dextrose), and water for injection. Pharmaceutical compositions include dry, for example, freeze-dried compositions which upon addition of sterilized water or physiological saline, permit the constitution of solutions suitable for administration.

Additionally, suspensions can be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilizers or agents which increase compound solubility facilitating the preparation of concentrated solutions.

An “effective amount” or “sufficient amount” refers to an amount providing an indicated or desired effect. The effective amount can be administered, in single or multiple doses, alone or in combination, with one or more other compositions (e.g., additional therapeutic or immunosuppressive agents), treatments, protocols, or therapeutic regimens; and provide for a long or short term response.

Pharmaceutical compositions comprising transgenes encoding ApoE3 related protein can be delivered to a subject, so as to allow production of the encoded protein. Delivery can be in vivo or ex vivo. In certain embodiments, pharmaceutical compositions comprise sufficient genetic material to enable a recipient to produce a therapeutically effective amount of a protein in the subject.

A “therapeutically effective amount” refers to an amount of an active ingredient or component that elicits the desired or indicated biological or medicinal response in a subject. A therapeutically effective amount can be determined based on observed symptoms and/or through the use of biomarkers associated with a particular disease or disorder. Selection of a particular effective dose can be optimized taking into account different factors, including the disease to be treated or prevented, the symptoms involved, safety and effectiveness in animal models, the patient's body mass, and the patient's immune status. The optimal dose to be employed in the formulation will also depend on the route of administration, and the severity of the disease or disorder, and can be evaluated depending upon patient's circumstances. Effective doses can be extrapolated from dose-response curves derived from in vitro or animal model test systems.

In certain embodiments, a pharmaceutical composition comprising a rAAV vector comprises empty AAV capsids. In certain embodiments, in a pharmaceutical composition comprising rAAV vectors and empty AAV capsids, the ratio of the empty AAV capsids to the rAAV vector is within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100. In certain embodiments, the ratio of the empty AAV capsids to the rAAV vector is about 2:1, 3:1, 4:1, 5:1, 6:1, 7:1, 8:1, 9:1, or 10:1.

Additional guidance and examples of pharmaceutical compositions and delivery systems are provided in, for example, Remington: The Science and Practice of Pharmacy (2020) 23th ed., University of the Sciences in Philadelphia, published by Elsevier; The Merck Index (2013) 15th ed., Whitehouse, NJ; Pharmaceutical Principles of Solid Dosage Forms (1993), Technomic Publishing Co., Inc., Lancaster, Pa.; and Ansel and Stoklosa, Pharmaceutical Calculations (2001) 11th ed., Lippincott Williams & Wilkins, Baltimore, MD.

### VII. Administration and Treatment

The polynucleotide constructs, viral vectors and non-viral vectors described herein can be administered to a subject, preferably a human subject, to provide for prophylactic treatment reducing the likelihood or severity of a disease or order and/or treating a diagnosed disease or disorder. In certain embodiments, the polynucleotide components, vector selection, route of administration, and/or particular pharmaceutical composition is selected taking into account the particular disease or disorder being treated.

Subject having a particular disease or disorder, or at an increased risk of a particular disease or disorder can be identified, for example, based on symptoms, biomarkers and genetic markers. Examples of treatments include one or more of the following: reduce cholesterol, reduce LDL/VLDL, increase HDL or reduce total cholesterol/HDL ratio; or treating or reducing the likelihood of hypercholesteremia, Type III Familial hyperlipoproteinemia, Familial Hypercholesterolemia, Cerebral amyloid angiopathy, dementia, post-stent restenosis, atherosclerosis, coronary heart disease or Alzheimer's disease.

In certain embodiments, treatment results in a reduction of atherosclerotic lesions.

In certain embodiments, the subject is a human subject having one or two ApoE4 alleles, or two ApoE2 alleles.

In certain embodiments treatment is carried out by providing for peripheral ApoE3 related expression, for example, liver expression. High liver expression can be achieved, for example, using polynucleotide expression cassettes comprising promoter or promoter/enhances providing for high liver expression; viral vectors providing for liver tropism; and nanoparticles targeting the liver. In different embodiments, the promoter is the human alpha 1-antitrypsin (hAAT) promoter, apolipoprotein A-I promoter, albumin promoter, hepatitis B virus core promoter, alpha-fetoprotein (AFP), human Factor IX promoter, thyroxin binding globulin promoter, TTR minimal enhancer/promoter, alpha-antitrypsin promoter or LSP1 promoter; and/or the apolipoprotein E (apoE) HCR-1 and HCR-2 enhancer is provided. In further embodiments, rAAV has a serotype based on AAV2, AAV3B, or a VP1 based on SEQ ID NO: 83 or SEQ ID NO: 84.

In certain embodiments Alzheimer's disease is treated using nucleic acid expression components, vectors components and/or techniques providing for CNS expression of ApoE3 related proteins; subjects are selected based on biomarker or genetic makers associated with Alzheimer's disease; and/or subjects are diagnosed with Alzheimer's disease. In further embodiments the ApoE3 related protein contains the Christchurch substitution.

In certain embodiments Alzheimer's disease is treated using nucleic acid expression components, vectors components and/or techniques providing for peripheral ApoE3 related protein expression. Peripheral ApoE isoforms, separated from those in the brain by the blood brain barrier, have been indicated to differentially impact Alzheimer's disease pathogenesis and cognition. (Liu et al., (2022), Nature Neuroscience 25:1020-1033.) In further embodiments the ApoE3 related protein contains the Christchurch substitution.

Alzheimer's disease subjects can be identified based on an abnormal decrease in cognitive ability which can be carried out in conjunction with measuring amyloid plaques (e.g., PET scan or the Lumipulse G β-Amyloid Ratio (1-42/1-40) test). Subjects at a greater risk of developing Alzheimer's disease can be identified based upon genetic markers associated with Alzheimer's disease. In different embodiments, the subject is a human subject having one or two ApoE4 alleles, or is a PSEN1 (presenilin 1) mutation carrier.

Depending upon targeted disease or disorder, administration can be by different routes, such as, subcutaneously, epidermally, intradermally, intrathecally, intraorbitally, intramucosally, intranasally, intraperitoneally, intravenously, intra-pleurally, intraarterially, intracavitary, orally, intrahepatically, via the portal vein, intramuscularly, intraparenchymal, intracisternal or intraventricular administration. In certain embodiments viral or non-viral vectors are administered to a patient via infusion in a pharmaceutically carrier.

CNS administration can also be carried out using techniques facilitating transport across the blood brain barrier including disruption of the blood brain barrier and making use blood brain barrier carriers. (Chen et al., (2021) Journal of Controlled Release 333, 129-138 and Bellettato and Scrapa, Italian Journal of Pediatrics (2018) 44(Suppl 2):131; Haumann et al., (2020) CNS Drugs 34, 1121-1131; and Cammalleri et al., J. Clin. Neurophysiol. (2020) March; 37(2):104-117, each of which are hereby incorporated by reference herein in their entirety.) Techniques facilitating crossing the blood brain barrier can be utilized on the gene delivery vehicle and/or the ApoE3 related proteins.

In certain embodiments, treatment of a CNS disease or disorder, such as Alzheimer's disease, is carried out using expression systems providing for expression outside of the CNS (e.g., liver expression) in combination with techniques facilitating ApoE related protein transport across the blood brain barrier.

In certain embodiments, treatment of a CNS disease or disorder, such as Alzheimer's disease is carried out using techniques facilitating the gene delivery vehicle crossing the blood brain barrier. In a further embodiment crossing of the blood brain barrier is facilitated using focused ultrasound in combination with microbubbles. (Cammalleri et al., J Clin Neurophysiol. (2020) March; 37(2):104-117, hereby incorporated by reference herein in its entirety.)

In certain embodiments components providing for CNS expression comprise the PGK promoter, CBh promoter, or EF1 alpha promoter.

In certain embodiments, an AAV capsid providing for CNS entry is used. Examples of such capsids are provided in Chen et al., (2021) Journal of Controlled Release 333, 129-138 (e.g., AAV9, AAVrh.10, AAVrh.8, AAVHSC, AAV-B1, AAV-AS, and AAV1/rh.10), U.S. Pat. No. 9,585,971, and U.S. Patent Publication No. US202/1214749, each of which are hereby incorporated by reference herein in their entirety.

CNS administration can also be carried out, for example, by direct administration to the brain using needles or catheter. (For example, International Publication No. WO 2021/108809, Cohen-Pferrer et al., Pediatric Neurology 67 (2017) 23-35; and U.S. patent Ser. No. 10/369,329; each of which are hereby incorporated by reference herein in their entirety.)

Another example of a technique for CNS administration is convection enhanced delivery. Convection enhanced deliver comprises surgical exposure of brain followed by placement of a catheter directly into the target area, followed by infusion of a therapeutic agent. (U.S. Patent Publication No. 2022/010001; and Debinski et al (2009) Expert Rev Neurother. 9(10): 1519-27; both of which are hereby incorporated by reference herein in their entirety.)

CNS delivery devices, systems and techniques also include those described in, for example, U.S. Pat. No. 8,128,600, U.S. Patent Publication No. 2020/0324089, U.S. patent Ser. No. 11/129,643, U.S. patent Ser. No. 11/154,377, U.S. Patent Publication No. 2021/0343397, U.S. Patent Publication No. 2021/0282866, U.S. Pat. Nos. 9,572,928, 8,337,458, U.S. patent Ser. No. 10/722,265, and U.S. Patent publication No. 2021/214749, each of which are incorporated by reference herein in their entirety.

Optimal doses can vary depending upon different factors such as the particular therapeutic, and desired endpoint. The dose amount, number, frequency or duration can be proportionally increased or reduced, taking into account adverse side effects, complications or other risk factors of the treatment or therapy and the status of the subject.

A “unit dosage form” refers to a physically discrete unit containing a predetermined effective amount of active ingredient in combination with a pharmaceutically acceptable carrier. Unit dosage forms can be provided within, for example, ampules and vials, which can include a pharmaceutically acceptable carrier, or a composition in a freeze-dried or lyophilized state. In the case of a freeze-dried or lyophilized state, a sterile liquid carrier can be added prior to administration. Individual unit dosage forms can be included in multi-dose kits or containers.

An “effective amount” achieves the desired or indicated effect. For example, an effective amount for treatment decreases one or more adverse symptoms, reduces the likelihood of one or more symptoms associated with a disease or disorder, or reduces disease or disorder progression. Preferred effective amounts for treatment are effective to decrease multiple or all adverse symptoms.

In certain embodiments, pharmaceutical compositions comprising a viral or non-viral vector is administered to a subject at a dose suitable to reduce cholesterol, reduce LDL/VLDL, increase HDL or reduce total cholesterol/HDL ratio; or treating or reducing the likelihood of hypercholesteremia, Type III Familial hyperlipoproteinemia, Familial Hypercholesterolemia, Cerebral amyloid angiopathy, dementia (e.g., vascular dementia or frontotemporal dementia), post-stent restenosis, atherosclerosis, coronary heart disease or Alzheimer's disease. In different embodiments a suitable dosage is from about 0.01 mg/kg to about 10 mg/kg of vector per kg body weight of a subject, about 0.01 mg/kg to about 0.1 mg/kg of vector per kg body weight of a subject, about 0.1 mg/kg to about 1.0 mg/kg of vector per kg body weight of a subject, or about 1.0 mg/kg to about 10 mg/kg of vector per body weight of a subject.

Generally, rAAV doses range from at least 1×10⁸ vector genomes per kilogram (vg/kg) of the weight of the subject, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect. In different embodiments the rAAV dose is about 5×10¹¹ rAAV vg/kg or greater than about 5×10¹¹ rAAV vg/kg; about 1×10¹² rAAV vg/kg or greater than about 1×10¹² rAAV vg/kg; about 2×10¹² rAAV vg/kg or greater than about 2×10¹² rAAV vg/kg; about 3×10¹² rAAV vg/kg or greater than about 3×10¹² rAAV vg/kg; about 4×10¹² rAAV vg/kg or greater than about 4×10¹² rAAV vg/kg; about 5×10¹² rAAV vg/kg or greater than about 5×10¹² rAAV vg/kg; about 1×10¹³ rAAV vg/kg or greater than about 1×10¹³ rAAV vg/kg; about 2×10¹³ rAAV vg/kg or greater than about 2×10¹³ rAAV vg/kg; about 3×10¹³ rAAV vg/kg or greater than about 3×10¹³ rAAV vg/kg; about 4×10¹³ rAAV vg/kg or greater than about 4×10¹³ rAAV vg/kg; about 5×10¹³ rAAV vg/kg or greater than about 5×10¹³ rAAV vg/kg; about 6×10¹³ rAAV vg/kg or greater than about 6×10¹³ rAAV vg/kg.

Examples of dose ranges of rAAV vg/kg include a dose range from about 5×10¹¹ to about 6×10¹³ rAAV vg/kg; a dose range from about 5×10¹¹ to about 5.5×10¹¹ rAAV vg/kg; a dose range from about 5.5×10¹¹ to about 6×10¹¹ rAAV vg/kg; a dose range from about 6×10¹¹ to about 6.5×10¹¹ rAAV vg/kg; a dose range from about 6.5×10¹¹ to about 7×10¹¹ rAAV vg/kg; a dose range from about 7×10¹¹ to about 7.5×10¹¹ rAAV vg/kg; a dose range from about 7.5×10¹¹ to about 8×10¹¹ rAAV vg/kg; a dose range from about 8×10¹¹ to about 8.5×10¹¹ rAAV vg/kg; a dose range from about 8.5×10¹¹ to about 9×10¹¹ rAAV vg/kg; a dose range from about 9×10¹¹ to about 9.5×10¹¹ rAAV vg/kg; a dose range from about 9.5×10¹¹ to about 1×10¹² rAAV vg/kg; a dose range from about 1×10¹² to about 1.5×10¹² rAAV vg/kg; a dose range from about 1.5×10¹² to about 2×10¹² rAAV vg/kg; a dose range from about 2×10¹² to about 2.5×10¹² rAAV vg/kg; a dose range from about 2.5×10¹² to about 3×10¹² rAAV vg/kg; a dose range from about 3×10¹² to about 3.5×10¹² rAAV vg/kg; a dose range from about 3.5×10¹² to about 4×10¹² rAAV vg/kg; a dose range from about 4×10¹² to about 4.5×10¹² rAAV vg/kg; a dose range from about 4.5×10¹² to about 5×10¹² rAAV vg/kg; a dose range from about 5×10¹² to about 5.5×10¹² rAAV vg/kg; a dose range from about 5.5×10¹² to about 6×10¹² rAAV vg/kg; a dose range from about 6×10¹² to about 6.5×10¹² rAAV vg/kg; a dose range from about 6.5×10¹² to about 7×10¹² rAAV vg/kg; a dose range from about 7×10¹² to about 7.5×10¹² rAAV vg/kg; a dose range from about 7.5×10¹² to about 8×10¹² rAAV vg/kg; a dose range from about 8×10¹² to about 8.5×10¹² rAAV vg/kg; a dose range from about 8.5×10¹² to about 9×10¹² rAAV vg/kg; a dose range from about 9×10¹² to about 9.5×10¹² rAAV vg/kg; a dose range from about 9.5×10¹² to about 1×10¹³ rAAV vg/kg; a dose range from about 1×10¹³ to about 1.5×10¹³ rAAV vg/kg; a dose range from about 1.5×10¹³ to about 2×10¹³ rAAV vg/kg; a dose range from about 2×10¹³ to about 2.5×10¹³ rAAV vg/kg; a dose range from about 2.5×10¹³ to about 3×10¹³ rAAV vg/kg; a dose range from about 3×10¹³ to about 3.5×10¹³ rAAV vg/kg; a dose range from about 3.5×10¹³ to about 4×10¹³ rAAV vg/kg; a dose range from about 4×10¹³ to about 4.5×10¹³ rAAV vg/kg; a dose range from about 4.5×10¹³ to about 5×10¹³ rAAV vg/kg; a dose range from about 5×10¹³ to about 5.5×10¹³ rAAV vg/kg; a dose range from about 5.5×10¹³ to about 6×10¹³ rAAV vg/kg; a dose range from about 6×10¹³ to about 1×10¹⁴ rAAV vg/kg.

In certain embodiments, rAAV vg/kg are administered at a dose of about 5×10¹¹ vg/kg, about 6×10¹¹ vg/kg, about 7×10¹¹ vg/kg, about 8×10¹¹ vg/kg, about 9×10¹¹ vg/kg, about 1×10¹² vg/kg, about 2×10¹² vg/kg, about 3×10¹² vg/kg, about 4×10¹² vg/kg, about 5×10¹² vg/kg, about 6×10¹² vg/kg, about 7×10¹² vg/kg, about 8×10¹² vg/kg, about 9×10¹² vg/kg, about 1×10¹³ vg/kg, about 2×10¹³ vg/kg, about 3×10¹³ vg/kg, about 4×10¹³ vg/kg, about 5×10¹³ vg/kg, or about 6×10¹³ vg/kg.

In certain embodiments doses and dose ranges for other viral vectors is as provided herein with respect to rAAV. For example, in certain embodiments the dose and dose range for recombinant adenovirus vectors, recombinant retrovirus vectors (e.g., lentivirus), and recombinant herpes simplex virus vectors is the same as illustrated above with respect to rAAV.

In certain embodiments, the polynucleotide constructs, viral vectors and non-viral vectors described herein are administered in combination with additional compounds or treatments for a particular disease of disorder; and/or in combination with a compound decreasing an immune response generated against the polynucleotides, delivery vehicle and/or produced protein. Additional compounds or treatments can be provided in different modalities such as administered separately; and administered or performed prior to, substantially contemporaneously with or following administration the polynucleotide constructs, viral vectors and non-viral vectors described herein.

In certain embodiments, administration of polynucleotide constructs, viral vectors and non-viral vectors described herein is in combination with an immunosuppressive agent or regimen. Such agents and regimens can be utilized, as needed, to achieve immune tolerance or mitigate the immune response to the produced ApoE3 related protein, the provided polynucleotides, or the provided delivery vehicles. Examples of immunosuppressive agents and regimens include methotrexate, rituximab, intravenous gamma globulin (IVIG), omalizumab, ImmTOR® (synthetic vaccine particle (SVP)-rapamycin (rapamycin encapsulated in a biodegradable nanoparticle)), ImmTOR-IL™ (ImmTOR with Treg-selective IL-2 agonist), B-cell depletion, immunoadsorption, and plasmapheresis.

In certain embodiments, the viral vector or non-viral vector is administered in conjunction with one or more immunosuppressive agents, where one or more immunosuppressive agent is administered prior to, substantially at the same time as, or after, administering the vector or non-viral vector. In certain embodiments, the one or more immunosuppressive agent is administered concomitantly with the vector or non-viral vector. In certain embodiments, the one or more immunosuppressive agent is administered 1-12, 12-24 or 24-48 hours; or 2-4, 4-6, 6-8, 8-10, 10-14, 14-20, 20-25, 25-30, 30-50 days, or more than 50 days prior to viral or non-viral vector administration. In certain embodiments, the one or more immunosuppressive agent is administered 1-12, 12-24 or 24-48 hours; or 2-4, 4-6, 6-8, 8-10, 10-14, 14-20, 20-25, 25-30, 30-50 days, or more than 50 days; following viral or non-viral vector administration. Administration of immunosuppressive agents after a period of time following administering vector or non-viral vector can be done, for example, if there is a decrease in the encoded protein after the initial expression levels for a period of time, e.g., 20-25, 25-30, 30-50, 50-75, 75-100, 100-150, 150-200 or more than 200 days following vector or non-viral vector administration.

In certain embodiments, the immunosuppressive agent is an anti-inflammatory agent. In certain embodiments, the immunosuppressive agent is a steroid, e.g., a corticosteroid. In certain embodiments, the immunosuppressive agent is prednisone, prednisolone, calcineurin inhibitor (e.g., cyclosporine, tacrolimus), MMF (mycophenolic acid, e.g. CellCept®, Myfortic®), CD52 inhibitor (e.g., alemtuzumab), CTLA4-Ig (e.g., abatacept, belatacept), anti-CD3 mAb, anti-LFA-1 mAb (e.g., efalizumab), anti-CD40 mAb (e.g., ASKP1240), anti-CD22 mAb (e.g., epratuzumab), anti-CD20 mAb (e.g., rituximab, orelizumab, ofatumumab, veltuzumab), proteasome inhibitor (e.g., bortezomib), TACI-Ig (e.g., atacicept), anti-C5 mAb (e.g., eculizumab), mycophenolate, azathioprine, sirolimus everolimus, TNFR-Ig, anti-TNF mAb, tofacitinib, anti-IL-2R (e.g., basiliximab), anti-IL-17 mAb (e.g., secukinumab), anti-IL-6 mAb (e.g., anti-IL-6 antibody sirukumab, anti-IL-6 receptor antibody tocilizumab (Actemra®), IL-10 inhibitor, TGF-beta inhibitor, a B cell targeting antibody (e.g., rituximab), a mammalian target of rapamycin (mTOR) inhibitor (e.g., rapamycin), synthetic vaccine particle (SVP™)-rapamycin (rapamycin encapsulated in a biodegradable nanoparticle), intravenous gamma globulin (IVIG), omalizumab, methotrexate, a tyrosine kinase inhibitor (e.g., ibrutinib), cyclophosphamide, fingolimod, an inhibitor of B-cell activating factor (BAFF) (e.g., anti-BAFF mAb, e.g., belimumab), an inhibitor of a proliferation-inducing ligand (APRIL), anti-IL-1b mAb (e.g., canakinumab (Haris®)), a C3a inhibitor, a Tregitope (see, e.g., U.S. Pat. No. 10,213,496), or a combination and/or derivative thereof.

Immune-suppression protocols, including the use of rapamycin, alone or in combination with IL-10, can be used to decrease, reduce, inhibit, prevent or block humoral and cellular immune responses to ApoE3 related protein. Hepatic gene transfer with viral vector (e.g., rAAV) and non-viral vector can be used to induce immune tolerance to ApoE3 related protein through induction of regulatory T cells (Tregs).

Strategies to reduce (overcome) or avoid humoral immunity to viral vectors, such as rAAV in systemic gene transfer include, administering high vector doses; use of AAV empty capsids as decoys to adsorb anti-AAV antibodies; administration of immunosuppressive drugs to decrease, reduce, inhibit, prevent or eradicate the humoral immune response to rAAV; changing the rAAV capsid serotype or engineering the rAAV capsid to be less susceptible to neutralizing antibodies; use of plasma exchange cycles to adsorb anti-AAV immunoglobulins, thereby reducing anti-AAV antibody titer; and use of delivery techniques such as balloon catheters followed by saline flushing. Such strategies are described in Mingozzi et al., (2013) Blood, 122:23-36. Additional strategies include using AAV-specific plasmapheresis columns to selectively deplete anti-AAV antibodies without depleting the total immunoglobulin pool from plasma, as described in Bertin et al., 2020, Sci. Rep. 10:864. Similar techniques and strategies can be used for other types of viral vectors.

Empty capsids used as decoy probes can be provided in different ratios to viral vectors. Amounts of empty capsids administered can be calibrated based upon the amount (titer) of antibodies produced in a particular subject. In certain embodiments, the ratio of the empty AAV capsids to the rAAV vector is within or between about 100:1-50:1, from about 50:1 to 25 to 1, from about 25:1 to 10:1, from about 10:1 to 1:1, from about 1:1 to 1:10, from about 1:10 to 1:25, from about 1:25 to 1:50, or from about 1:50 to 1:100. In particular aspects, the ratio of the administered empty AAV capsids to rAAV vector is about 2:1, 3:1, 4:1, 5:1, 6:1, 7:1, 8:1, 9:1, or 10:1. Preferably, the serotype of the empty capsids is the same as the rAAV serotype.

In certain embodiments, viral vehicles are delivered using methods bypassing the bloodstream and viral antibodies. Examples of such techniques include deliver to the liver via the hepatic artery; and endoscopic retrograde cholangiopancreatography (ERCP) deliver to the liver. Delivery to the CNS via the carotid artery. Other ductal systems, such as the ducts of the submandibular gland, can also be used as portals for delivering viral vectors into a subject that develops or has preexisting anti-antibodies to the viral vector.

Additional strategies to reduce humoral immunity to rAAV (which can be applied to other viral vectors) include methods to remove, deplete, capture, and/or inactivate AAV antibodies, commonly referred to as apheresis and more particularly, plasmapheresis where blood products are involved. Apheresis or plasmapheresis, is a process in which a human subject's plasma is circulated ex vivo (extracorporal) through a device that modifies the plasma through addition, removal and/or replacement of components before its return to the patient. Plasmapheresis can be used to remove human immunoglobulins (e.g., IgG, IgE, IgA, IgD) from a blood product (e.g., plasma). This procedure can be employed to deplete, capture, inactivate, reduce or remove immunoglobulins (antibodies) that bind AAV thereby reducing the titer of AAV antibodies in the treated subject that can contribute to rAAV neutralization. An example is using a device composed of an AAV capsid affinity matrix column, and passing blood product (e.g., plasma) through an AAV capsid affinity matrix resulting in binding of AAV antibodies of different isotypes. (See, e.g., Bertin et al., 2020, Sci. Rep. 10, 864, hereby incorporated by reference herein in its entirety.)

In certain embodiments the polynucleotide constructs, viral vectors and non-viral vectors can be used in combination with an agent that blocks, inhibits, or reduces the interaction of IgG with the neonatal Fc receptor (FcRn), such as an anti-FcRn antibody, to reduce IgG recycling and enhance IgG clearance in vivo: and/or an agent that decreases the circulating antibodies that bind to a recombinant viral vector, or that binds to a nucleic acid or a polypeptide, protein or peptide encoded by a polynucleotide encapsidated by a recombinant viral vector, or that bind to the polynucleotide. In certain embodiments, antibody binding to a viral vector is reduced or inhibited by an agent that reduces interaction of IgG with FcRn, a protease or a glycosidase.

In certain embodiments, the polynucleotide constructs, viral vectors and non-viral vectors described herein can be used in combination with an endopeptidase (e.g., IdeS from Streptococcus pyogenes) or a modified variant thereof, or an endoglycosidase (e.g., S. pyogenes EndoS) or a modified variant thereof. Such treatment can, for example, be carried out to reduce or clear neutralizing antibodies against the gene delivery vehicle (e.g., viral vector capsid) and enable treatment of patients previously viewed as not eligible for gene therapy or that develop antibodies resulting from gene therapy. Such strategies are described in, for example, Leborgne et al., (2020) Nat. Med., 26:1096-1101.

In certain embodiments, method of treatment in a subject is carried out in combination with a compound reducing native subject ApoE expression. Native ApoE expression can be inhibited, for example, using inhibitory nucleic acid selectively targeting native ApoE encoding sequences. Reference to “selectively targeting” native ApoE sequence indicates that expression of the polynucleotide encoded ApoE3 related protein is not significant effected. The inhibitory nucleic acid can be provided on the same polynucleotide and/or vector that encodes for the ApoE3 related protein or using a separate viral or non-viral vector. Examples of inhibitory nucleic acid include a short hairpin RNA (shRNA), a small interfering RNA (siRNA), a microRNA (miRNA), a RNAi, a ribozyme, and an antisense RNA.

In certain embodiments an expression cassette further comprises an inhibitory nucleic acid selectively targeting one or two of naturally occurring ApoE2, ApoE3, and ApoE4 encoding nucleic acid. In further embodiments ApoE2 is targeted or ApoE4 is targeted. In certain embodiments, the subject has one ApoE4 allele or has two ApoE4 alleles and the inhibitory nucleic acid targets ApoE4 encoding nucleic acid; and the subject has two ApoE2 alleles and the inhibitory nucleic acid targets ApoE2. In certain embodiments, native ApoE3 is targeted in conjunction with using an ApoE3 related protein comprising a Christchurch mutation.

In certain embodiments, the polynucleotide constructs, viral vectors and non-viral vectors described herein are used in combination with one or more additional treatments, such as a treatment for hyperlipidemia, atherosclerosis, cardiovascular diseases and/or dementia. In different embodiments, the additional treatment comprises a statin (e.g., atorvastatin, fluvastatin, lovastatin, pitavastatin, pravastatin, rosuvastatin, and simvastatin), a PCSK9 inhibitor, and/or ezetimibe. In certain embodiments, the additional treatment comprises donepezil, galantamine, rivastigmine, or memantine. In certain embodiments, the additional dementia (e.g., Alzheimer's disease) treatment comprises a statin (e.g., atorvastatin, fluvastatin, lovastatin, pitavastatin, pravastatin, rosuvastatin, and simvastatin), a PCSK9 inhibitor, and/or ezetimibe.

### VIII. Kits

The present invention includes kits with packaging material and one or more components therein. A kit typically includes a label or packaging insert including a description of the components or instructions for use in vitro, in vivo, or ex vivo, of the components therein. A kit can contain a collection of such components, e.g., a viral or a non-viral vector, and optionally a second active, such as another compound, agent, drug or composition.

A kit refers to a physical structure housing one or more components. Packaging material can maintain the components sterilely, and can be made of material commonly used for such purposes such as paper, corrugated fiber, glass, plastic, foil, ampules, vials, and tubes.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacture location and date, and expiration dates. Labels or inserts can include information on a disease for which a kit component can be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on one or more benefits a component can provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that can be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

### IX. mRNA Therapeutics

In certain embodiments RNA versions of nucleic acid encoding for ApoE related proteins described herein is provided as an mRNA construct able to express the encoded protein inside a cell. The mRNA constructs comprises a 5′-cap, a 5′UTR, the encoding RNA, 3′UTR, and a poly(A) tail. The UTRs and poly(a) tail can provide for different functions such as participating in mRNA subcellular localization, regulating translation efficiency and mRNA stability. The design and production of mRNA constructs, including different modifications, are illustrated in different publications such as Ouranidis et al., (2022) Biomedicines, 10, 50, Qin et al., Signal Transduct Target Ther. (2022) May 21; 7(1):166, and U.S. Patent Publication No. U.S. 2013/0259924, each of which are hereby incorporated by reference herein in their entirety.

In certain embodiments, the mRNA construct is delivered to a cell or subject using nanoparticles. Examples of nanoparticle includes those provided in Sections V.A.-V.E. supra., Ouranidis et al., (2022) Biomedicines, 10, 50, and U.S. Patent Publication No. U.S. 2013/0259924.

Guidance for therapeutic administration and targeted diseases or disorders is provided, for example, in Section VII. supra.

### X. Additional Aspects and Embodiments

Additional aspects, embodiments, and combinations thereof include the following:

A first aspect is directed to a polynucleotide comprising an ApoE encoding nucleotide sequence having at least 85% sequence identity to the sequence any of SEQ ID NOs: 63-67 or nucleotides 55-951 of any of SEQ ID NOs: 95-105 and 124-130, wherein the polynucleotide encodes an ApoE3 related protein comprising an amino acid sequence at least 90% identical to SEQ ID NO: 32, where the protein comprises a cysteine in a location corresponding to amino acid 112 of SEQ ID NO: 32 and an arginine at a location corresponding to amino acid 158 of SEQ ID NO: 32; wherein the ApoE encoding nucleotide sequence optionally comprises one or more introns. The percent identical is determined independent of any intron present in the ApoE encoding nucleotide sequence.

Embodiment E1a further describes the first aspect, wherein the ApoE encoding nucleic acid sequence comprises one or more introns.

Embodiment E1b further describes the first aspect, wherein the ApoE encoding nucleic acid sequence does not comprise any introns. Introns may be present in other parts of the polynucleotide.

Embodiment E2a further describes the first aspect, Embodiments E1a and E1b, wherein the ApoE encoding nucleotide sequence has at least 95% sequence identity to nucleotides 55-951 of any of SEQ ID NOs: 3-10, 12-14, 16-19, 21-25, and 27-30. In further embodiments the ApoE encoding nucleic acid sequence has at least 96%, at least 97%, at least 98%, at least 99%, or 100% identity to nucleotides 55-951 or 55-954, of any of SEQ ID NOs: 3-10, 12-14, 16-19, 21-25, and 27-30; or differs from any of nucleotides 55-951 or 55-954 of any of SEQ ID NOs: 3-10, 12-14, 16-19, 21-25, and 27-30 by 1-40 nucleotides, 1-20 nucleotides, or 1-10 nucleotides.

Embodiment E2b further describes the first aspect, and Embodiments E1a and E1b, wherein the ApoE encoding nucleotide sequence has at least 95% sequence identity to nucleotides 55-951 of any of SEQ ID NOs: 95-105 and 124-130. In further embodiments the ApoE encoding nucleic acid sequence has at least 96%, at least 97%, at least 98%, at least 99%, or 100% identity to nucleotides 55-951 or 55-954, of any of SEQ ID NOs: 95-105 and 124-130; or differs from any of nucleotides 55-951 or 55-954 of any of SEQ ID NOs: 95-105 and 124-130 by 1-40 nucleotides, 1-20 nucleotides, or 1-10 nucleotides. Preferably, the sequence identity is with respect to any of SEQ ID NOs: 95-105.

Embodiment E3 further describes the first aspect, Embodiments E1a and E1b, wherein the ApoE encoding nucleotide sequence has at least 95% sequence identity to any of SEQ ID NOs: 63-67. In further embodiments the ApoE encoding nucleic acid sequence has at least 96%, at least 97%, at least 98%, at least 99%, or 100% identity to any of SEQ ID NOs: 63-67 or nucleotides 1-897 of any of SEQ ID NOs: 63-67; or differs from any of SEQ ID NOs: 63-67 by 1-40 nucleotides, 1-20 nucleotides, or 1-10 nucleotides.

Embodiment E4 further describes the first aspect, Embodiments E1a, E1b, E2a, E2b, and E3, wherein the ApoE3 related protein comprises a serine at a position corresponding to amino acid 136 of SEQ ID NO: 32.

Embodiment E5 further describes the first aspect, Embodiments E1a, E1b, E2a, E2b, and E3, wherein the ApoE3 related protein has at least 95% identity to the sequence of SEQ ID NO: 32. In further embodiments the protein has at least 96%, at least 97%, at least 98%, at least 99%, or 100% identity to the sequence of SEQ ID NO: 32; or differs from SEQ ID NO: 32 by 1-10 amino acids, or any of 1, 2, 3, 4, 5, 6, 7, 8, 9, or 10 amino acids.

Embodiment E6 further the describes the first aspect, Embodiments E1a, E1b, E2a, E2b, and E3, wherein the ApoE3 related protein has at least 95% identity to the sequence of SEQ ID NO: 33. In further embodiments the protein has at least 96%, at least 97%, at least 98%, at least 99%, or 100% identity to the sequence of SEQ ID NO: 33; or differs from SEQ ID NO: 33 by 1-10 amino acids, or any of 1, 2, 3, 4, 5, 6, 7, 8, 9, or 10 amino acids.

Embodiment E7 further the describes the first aspect, Embodiments E1a, E1b, E2a, E2b, E3, E4, E5, and E6 wherein the ApoE3 related protein further comprises a 5′ signal peptide and the ApoE3 encoding nucleic acid sequence further comprises a nucleotide sequence encoding the signal peptide.

Embodiment E8 further the describes Embodiment E7, wherein the signal peptide has at least 90% identity to an amino acid sequence selected from the group consisting of SEQ ID NOs: 36, 42, 44, 46, 48, 50, 52, 54, 56, and 68-71. In further embodiments the signal peptide has at least 95%, at least 96%, at least 97%, at least 98%, at least 99%, or 100% identity to any of SEQ ID NOs: 36, 42, 44, 46, 48, 50, 52, 54, 56, and 68-71; or differs from any of SEQ ID NOs: 36, 42, 44, 46, 48, 50, 52, 54, 56, and 68-71 by any of 1, 2, or 3 amino acids.

Embodiment E9 further describes Embodiment E8, wherein the nucleotide sequence encoding the signal peptide sequence comprises at least 90% identity to a sequence selected from the group consisting of SEQ ID NOs: 37-41, 43, 45, 47, 49, 51, 53, 55, 57 and 72-75. In further embodiments, the nucleotide sequence encoding the signal peptide has at least 96%, at least 97%, at least 98%, at least 99%, or 100% identity to the sequence of any of SEQ ID NOs: 37-41, 43, 45, 47, 49, 51, 53, 55, 57 and 72-75; or differs from any of SEQ ID NOs: 37-41, 43, 45, 47, 49, 51, 53, 55, 57 and 72-75 by 1-10 nucleotides, or any of 1, 2, 3, 4, 5, 6, 7, 8, 9, or 10 nucleotides.

Embodiment E10a further describes the first aspect, Embodiments E1a, E1b, E2a, E2b, E3, E4, E5, E6, E7, E8, and E9, wherein the ApoE encoding nucleic acid sequence has a sequence identity of at least 90% to the sequence of any of SEQ ID NOs: 3-31. In further embodiments the ApoE encoding nucleic acid sequence has at least 91%, at least 92%, at least 93%, at least 94%, at least 95%, at least 96%, at least 97%, at least 98%, at least 99%, or 100% to any of SEQ ID NOs: 3-31; or differs from any of SEQ ID NOs: 3-31 by 1-40 nucleotides, 1-20 nucleotides, or 1-10 nucleotides. In further embodiments, the percent identity is with respect to SEQ ID NOs: 20, 26, 31, 11 or 15; or with respect to SEQ NOs: 11 and 20.

Embodiment E10b further describes the first aspect, Embodiments E1a, E1b, E2a, E2b, E3, E4, E5, E6, E7, E8, and E9, wherein the ApoE encoding nucleic acid sequence has a sequence identity of at least 90% to the sequence of any of SEQ ID NOs: 95-105 and 124-130. In further embodiments the ApoE encoding nucleic acid sequence has at least 91%, at least 92%, at least 93%, at least 94%, at least 95%, at least 96%, at least 97%, at least 98%, at least 99%, or 100% to any of SEQ ID NOs: 95-105 and 124-130; or differs from any of SEQ ID NOs: 95-105 and 124-130 by 1-40 nucleotides, 1-20 nucleotides, or 1-10 nucleotides. In further embodiments, the percent identity is with respect to SEQ ID NOs: 95-105.

Embodiment E11 further the describes the first aspect, Embodiments E1a, E1b, E2a, E2b, E3, E4, E5, E6, E7, E8, E9, E10a and E10b, wherein the ApoE3 related sequence comprises the sequence of SEQ ID NOs: 34 or 35.

Embodiment E12a further the describes the first aspect, Embodiments E1a, E1b, E2a, E2b E3, E4, E5, E6, E7, E8, E9, E10a, E10b and E11, wherein the ApoE encoding nucleotide sequence comprises a sequence of (i) nucleotides 55 to 951 of any of SEQ ID NOs: 3-10, 12-14, 16-19, 21-25 and 27-30; (ii) nucleotides 55 to 954 of any of SEQ ID NOs: 3-10, 12-14, 16-19, 21-25 and 27-30; (iii) nucleotides 1-897 of any of SEQ ID NOs: 63-67; (iv) any of SEQ ID NOs: 63-67; (v) nucleotides 1 to 951 of any of SEQ ID NOs: 3-31; or (vi) any of SEQ ID NOs: 3-31.

Embodiment E12b further the describes the first aspect, Embodiments E1a, E1b, E2a, E2b, E3, E4, E5, E6, E7, E8, E9, E10a, E10b and E11 wherein the ApoE encoding nucleotide sequence comprises a sequence of (i) nucleotides 55 to 951 of any of SEQ ID NOs: 95-105 and 124-130; (ii) nucleotides 55 to 954 of any SEQ ID NOs: 95-105 and 124-130; (iii) nucleotides 1 to 951 of any of SEQ ID NOs: 95-105 and 124-130; or (iv) any of SEQ ID NOs: 95-105 and 124-130.

Embodiment 12c further the describes the first aspect, Embodiments E1a, E1b, E2a, E2b, E3, E4, E5, E6, E7, E8, E9, E10a, E10b and E11, wherein the ApoE encoding nucleotide sequence comprises a sequence at least 95% identical, at least 96% identical, at least 97% identical, at least 98% identical, at least 99% identical or 100% identical to (i) nucleotides 55 to 951 of any of SEQ ID NOs: 3-10, 12-14, 16-19, 21-25, 27-30, 95-105 and 124-130; (ii) nucleotides 55 to 954 of any of SEQ ID NOs: 3-10, 12-14, 16-19, 21-25, 27-30, 95-105 and 124-130; (iii) nucleotides 1-897 of any of SEQ ID NOs: 63-67; (iv) any of SEQ ID NOs: 63-67; (v) nucleotides 1 to 951 of any of SEQ ID NOs: 3-31, 95-105 and 124-130; or (vi) any of SEQ ID NOs: 3-31, 95-105 and 124-130; and the ApoE3 related protein comprises an amino acid sequence of SEQ ID NOs: 34 or 35.

Embodiment 12d further describes Embodiment 1a wherein the ApoE encoding nucleotide sequence comprises 5′ to 3′:
(a) a first exon corresponding to nucleotides 1-43 of SEQ ID NO: 1, wherein the first exon has a sequence identity of at least 85% to nucleotides 1-43 of any of SEQ ID NOs: 3-31 95-105, or 124-130, provided that the terminal 3′ nucleotide of the first exon is G. In further embodiments concerning the first exon, the first exon has a sequence identity of at least 90%, at least 95%, at least 96%, at least 97%, at least 98%, at least 99% or 100% to nucleotides 1-43 of any of SEQ ID NOs: 3-31, 95-105, and 124-130; and in further embodiments the sequence identity is with respect to nucleotides 1-43 of any of SEQ ID NOs: 11, 20, and 95-105;
(b) a first intron at a position corresponding to between nucleotides 43 and 44 of SEQ ID NO: 1;
(c) a second exon corresponding to nucleotides 44-236 of SEQ ID NO: 1; wherein the second exon has a sequence identity of at least 85% to nucleotides 44-236 of any of SEQ ID NOs: 3-31, 95-105 and 124-130, provided that the terminal 5′ nucleotides of the second exon is G (in a further embodiment GC) and the terminal 3′ nucleotides of the second exon is AG. In further embodiments concerning the second exon, the second exon has a sequence identity of at least 90%, at least 95%, at least 96%, at least 97%, at least 98%, at least 99% or 100% to nucleotides 44-236 of any of SEQ ID NOs: 3-31, 95-105, and 124-130; and in further embodiment the sequence identity is with respect to nucleotides 44-236 of any of SEQ ID NOs: 11, 20, 95-105 and 124-130;
(d) a second intron at a position corresponding to between nucleotides 236 and 237 of SEQ ID NO: 1;
(e) a third exon corresponding to nucleotides 237-951 of SEQ ID NO: 1, wherein the third exon has a sequence identity of at least 85% to nucleotides 237-951 of any of SEQ ID NOs: 3-31, 95-105, and 124-130, provided that the terminal 5′ nucleotide of the third exon is G, wherein the first, second and third exons together encode the ApoE3 related protein. In further embodiments concerning the third exon, the third exon has a sequence identity of at least 90%, at least 95%, at least 96%, at least 97%, at least 98%, at least 99% or 100% to nucleotides 237-951 any of SEQ ID NOs: 3-31, 95-105, and 124-130; and in further embodiments the sequence identity is with respect to nucleotides 237-951 of any of SEQ ID NOs: 11, 20, and 95-105;
wherein the ApoE related protein comprises an amino acid sequence at least 95% identical to SEQ ID NO: 35, in further embodiments, the ApoE related protein comprises an amino acid sequence at least 96% identical, at least 97% identical, at least 98% identical, at least 99% identical to SEQ ID NO: 35, differing from SEQ ID NO: 35 by 1, 2, 3, 4 or 5 amino acids, or comprises SEQ ID NO: 34 or SEQ ID NO: 35.

Each of possibilities with (a), (b), (c), (d), and (e) can be combined independently, for each sequence. For example, (1) the first exon can independently of the second and third exons have a sequence identity of at least 85%, at least 90%, at least 95%, at least 96%, at least 97%, at least 98%, at least 99% or 100% to nucleotides 1-43 of any of SEQ ID NOs: 3-31, 95-105, and 124-130; (2) the second exon independently of the first and third exons, can have a sequence identity of at least 85%, at least 90%, at least 95%, at least 96%, at least 97%, at least 98%, at least 99% or 100% to nucleotides 44-236 any of SEQ ID NOs: 3-31, 95-105, and 124-130; and (3) the third exon independently of the first and second exons can have a sequence identity of at least 85%, at least 90%, at least 95%, at least 96%, at least 97%, at least 98%, at least 99% or 100% to nucleotides 44-236 any of SEQ ID NOs: 3-31, 95-105, and 124-130.

Embodiment 12e further describes Embodiment 12d, wherein the first intron comprises a sequence with a sequence identity of at least 50%, at least 60%, at least 70%, at least 80%, at least 90%, at least 95%, at least 96%, at least 97%, at least 98%, at least 99%, or 100% to any of SEQ ID NOs: 119-122 and the second intron independently comprises a sequence with a sequence identity of at least 50%, at least 60%, at least 70%, at least 80%, at least 90%, at least 95%, at least 96%, at least 97%, at least 98%, at least 99%, or 100% identity to any of SEQ ID NOs: 119-122.

Embodiment 12f further describe Embodiment 12e, wherein the first intron consists of a sequence of any of SEQ ID NOs: 119-121 or a sequence differing from any of SEQ ID NOs: 119-121 by 1 to 10 nucleotides and the second intron independently consists of a sequence of any of SEQ ID NOs: 119-121 or a sequence differing from any of SEQ ID NOs: 119-121 by 1 to 10 nucleotides.

Embodiment 12g further describes Embodiments 12d, 12e, and 12f, wherein,
the first exon has a sequence identity of at least 95%, at least 96%, at least 97%, at least 98%, at least 99% or 100% to nucleotides 1-43 of any of SEQ ID NOs: 11, 20, and 95-105;
the second exon has a sequence identity of at least 95%, at least 96%, at least 97%, at least 98%, at least 99% or 100% to nucleotides 44-236 of any of SEQ ID NOs: 11, 20, and 95-105; and
the third exon has a sequence identity of at least 95%, at least 96%, at least 97%, at least 98%, at least 99% or 100% to nucleotides 237-951 of any of SEQ ID NOs: 11, 20, and 95-105, wherein the ApoE related protein has a sequence identity of at least 98% to SEQ ID NO: 35.

Embodiment 12 h further describes Embodiment 12g, wherein the ApoE encoding nucleotide sequence comprises a sequence with a sequence identity of at least 95%, at least 96%, at least 97%, at least 98%, at least 99% or 100% to any of SEQ ID NOs: 106-115.

Embodiment 12i further describes Embodiment E12d, E12e, E12f, E12g, and E12h, wherein the ApoE related protein comprises the sequence of SEQ ID NOs: 34 or 35.

Embodiment E13 further the describes the first aspect, Embodiments E1a, E1b, E2a, E2b, E3, E4, E5, E6, E7, E8, E9, E10a, E10b, E11, E12a, E12b, E12c, E12d, E12e, E12f, E12g, E12h, and E12i, wherein the ApoE encoding nucleic acid sequence contains any of 0-5, 0-10, or 0-15 CpGs; 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, and 82 CpGs; 0%, about 0.5%, about 1.0%, about 2.0%, about 3.0%, about 4.0%, or about 5.0% CpGs; and/or up to about 0.5%, up to about 1.0%, up to about 2.0%, up to about 3.0%, up to about 4.0%, or up to about 5.0% CpGs. Preferably, 0-10, 0-5, or 0 CpG.

Embodiment E14 further describes Embodiments E1a, E1b, E2a, E2b, E3, E4, E5, E6, E7, E8, E9, E10a, E10b, E11, E12a, E12b, E12c, E12d, E12e, E12f, E12g, E12h, E12i, and E13 wherein the polynucleotide is an expression cassette further comprising one or more expression control elements operably linked to the ApoE encoding nucleic sequence. In further embodiments, one or more expression control elements selected from a promoter, promoter/enhances, intron, polyadenylation signal and Kozak sequence are present. In further embodiments, the expression cassette comprises a promoter operably linked 5′ to the ApoE encoding nucleic sequence, and a polyadenylation site operably linked 3′ to the ApoE encoding nucleic acid; or the expression cassette comprises 5′ to 3′ operably linked to the ApoE encoding nucleic sequence, a promoter, an intron, a Kozak sequence, the ApoE encoding nucleic sequence and a polyadenylation signal.

Embodiment E15 further describes Embodiment E14, wherein the promoter is a liver specific promoter. In a further embodiment, the promoter is hAAT.

Embodiment E16 further describes Embodiments E14 and E15, wherein the promoter is operatively coupled to an HCR1 based enhancer having a sequence identity of at least 95%, at least about 96%, at least about 97%, at least about 98%, at least about 99% or 100% with SEQ ID NO: 58.

Embodiment E17 further describes Embodiment E14, wherein the promoter provides for expression in CNS cells. In further embodiments, the promoter is either a CAG promoter, a CBh promoter, a EF1α promoter or a hSynapsin promoter. In further embodiments, the expression cassette further comprises an miRNA target sequence to inhibit dorsal root ganglion, liver, and/or immune cell expression.

Embodiment E18 further describes Embodiments E14, E15, E16, and E17, wherein the intron comprises the sequence of SEQ ID NO: 60. In a further embodiment, the polyadenylation signal comprises the sequence of SEQ ID NO: 61 or SEQ ID NO: 62.

Embodiment E19 further describes Embodiments E14, E15, E16, E17, and E18, wherein the expression cassette further comprises an inhibitory nucleic acid selectively targeting one or two of ApoE2, ApoE3, and ApoE4 encoding nucleic acid. In further embodiments ApoE2 is targeted or ApoE4 is targeted.

Embodiment E20 further describes Embodiments E14, E15, E16, E17, E18, and E19, wherein the expression cassette contains any of 0-5, 0-10, 0-15, 0-50, or 0-100 CpGs; 0, 1,2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, and 82 CpGs; 0%, about 0.5%, about 1.0%, about 2.0%, about 3.0%, about 4.0%, about 5.0%, about 6%, about 7%, about 8%, about 9%, about 10%, about 11%, about 12%, about 13%, about 14%, or about 15% CpGs; and/or up to about 0.5%, up to about 1.0%, up to about 2.0%, up to about 3.0%, up to about 4.0%, up to about 5.0%, up to about 6%, up to about 7%, up to about 8%, up to about 9%, up to about 10%, up to about 11%, up to about 12%, up to about 13%, up to about 14%, or up to about 15% CpGs.

Embodiment E21, further describes Embodiments E14, E15, E16, E17, E18, E19, and E20 wherein the expression cassette comprises a sequence having at least 95%, at least 96%, at least 97%, at least 98%, at least 99%, or 100% identity to any of SEQ ID NOs: 76-80; comprises a sequence differing from any of SEQ ID NOs: 76-80 by 1-40 nucleotides, 1-20 nucleotides, or 1-10 nucleotides; or comprises a sequence having a sequence identity of at least 95%, at least 96%, at least 97%, at least 98%, at least 99%, or 100% identity to a modified SEQ ID NO: 76, where SEQ ID NO: 76 is modified by replacing nucleotides 1295-2248 with the sequence of any of SEQ ID NOs: 3-10, 12-14, 16-19, 21-25, 27-30, 95-115 and 124-130.

Embodiment E22 further describes E14, E15, E16, E17, E18, E19, E20, and E21, wherein the polynucleotide is a recombinant viral vector nucleic acid comprising the expression cassette and 5′ and/or 3′ viral elements associated elements providing for viral packaging and/or replication. In further embodiments, the recombinant viral vector nucleic acid is DNA; and/or is based on the AAV genome and comprises AAV 5′ and 3′ ITRs; the recombinant viral vector nucleic acid is DNA and is based on the adenovirus genome and comprises 5′ and 3′ ITRs and a packaging signal; and the recombinant viral vector nucleic acid is RNA and is based a retrovirus genome (e.g., lentivirus) and comprises 5′ and 3′ LTRs and a packaging signal. Reference to “based” on a viral genome indicates the ability replicate and be packaged in a capsid of a referenced virus.

Embodiment E23, further describes E22, wherein the polynucleotide is a rAAV nucleic acid comprising ITRs flanking the 5′ terminus of polynucleotide and/or the 3′ terminus of the polynucleotide. In a further embodiment, ITRs flank the 5′ and 3′ terminus of the polynucleotide.

Embodiment E24 further describes E23, wherein the 5′ and/or 3′ viral elements are each selected from the group consisting of AAV1, AAV2, AAV3, AAV4, AAV5, AAV6, AAV7, AAV8, AAV9, AAV10, AAV11, AAV12, AAVrh.10, AAVrh.74 and AAV3B.

Embodiment E25, further describes E23 wherein the 5′ ITR comprises a sequence with a sequence identity of at least 95%, at least 96%, at least 97%, at least 98%, at least 99% or 100% identity to any one of SEQ ID NOs: 81, 88, 90, 92 and 94 and the 3′ ITR independently comprises a sequence with a sequence identity of at least 95%, at least 96%, at least 97%, at least 98%, at least 99% or 100% to SEQ ID NOs: 82, 89, 91 and 93.

Embodiment E26 further describes E22, E23, E24, and E25 wherein the recombinant viral vector nucleic acid contains any of 0-5, 0-10, 0-15, 0-50, 0-100, or 0 to 150 CpGs; 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, and 82 CpGs; 0%, about 0.5%, about 1.0%, about 2.0%, about 3.0%, about 4.0%, about 5.0%, about 6%, about 7%, about 8%, about 9%, about 10%, about 11%, about 12%, about 13%, about 14%, or about 15% CpGs; and/or up to about 0.5%, up to about 1.0%, up to about 2.0%, up to about 3.0%, up to about 4.0%, up to about 5.0%, up to about 6%, up to about 7%, up to about 8%, up to about 9%, up to about 10%, up to about 11%, up to about 12%, up to about 13%, up to about 14%, or up to about 15% CpGs.

Embodiment E27 is directed to a gene delivery vehicle, wherein the gene delivery vehicle is a viral or a non-viral vector comprising the polynucleotide of any of the first aspect, E1-E21 or the recombinant viral vector of any of E22-E26.

Embodiment E28, further describes E27, wherein the gene delivery vehicle is a viral vector. In further embodiments, the vehicle is a rAAV vector, a recombinant retrovirus (e.g., lentivirus) vector, or a recombinant adenovirus vector.

Embodiment E29 further describes E28, wherein the viral vector is a rAAV, and the rAAV vector comprises a capsid comprising a VP1, VP2 or VP3 having a sequence identity of at least 90% to any of AAV1, AAV2, AAV3, AAV4, AAV5, AAV6, AAV7, AAV8, AAV9, AAV10, AAV11, AAV12, AAVrh.74, AAV3B, AAV-2i8, AAVrh.10, AAVrh.8, AAVHSC, AAV-B1, AAV-AS, AAV1/rh.10, SEQ ID NO: 83 and SEQ ID NO: 84. In further embodiments, the recombinant AAV vector capsid comprises a VP1, VP2 or VP3 having a sequence identity of at least 95%, at least 96%, at least 97%, at least 98%, at least 99% or 100% to any of AAV1, AAV2, AAV3, AAV4, AAV5, AAV6, AAV7, AAV8, AAV9, AAV10, AAV11, AAV12, AAVrh.74, AAV3B, AAV-2i8, AAVrh.10, AAVrh.8, AAVHSC, AAV-B1, AAV-AS, AAV1/rh.10, SEQ ID NO: 83 and SEQ ID NO: 84. In a further embodiment, the capsid comprises a VP1 comprising the sequence of SEQ ID NO: 83, a VP2 comprising the sequence of SEQ ID NO:122, and a VP3 comprising the sequence of SEQ ID NO: 123.

Embodiment E30 further describes E27, wherein the gene delivery vehicle is anon-viral vector. In further embodiments, the non-viral vector is a nanoparticle; is a nanoparticle selected from the group consisting of a lipid nanoparticle (LNP), a polymeric nanoparticle, a lipid polymer nanoparticle (LPNP), a protein or peptide-based nanoparticle, a DNA dendrimer or DNA-based nanocarrier, a carbon nanotube, a microparticle, a microcapsule, an inorganic nanoparticle, a peptide cage nanoparticle, and an exosome; is an LPN; or is an LPNP.

Embodiment E31 is directed to a pharmaceutical composition comprising the polynucleotide of any of the first aspect, E1-E21, the recombinant viral vector of any of E22-E26, or the gene delivery vehicle of any of E27-E30; and a pharmaceutically acceptable carrier.

Embodiment E32 further describes E31, wherein the composition comprises rAAV and empty AAV capsids, and the ratio of the empty AAV capsids to the rAAV is 100:1 to 1:100. In further embodiments the ratio of the empty AAV capsids to the rAAV is about 100:1 to 50:1, about 50:1 to 25:1, about 25:1 to 10:1, about 10:1 to 1:1, about 1:1 to 1:10, about 1:10 to 1:25, about 1:25 to 1:50, or about 1:50 to 1:100. In further embodiments, the ratio of empty AAV capsids to the rAAV is about 2:1, 3:1, 4:1, 5:1, 6:1, 7:1, 8:1, 9:1, or 10:1.

A second aspect of the present invention is directed a method of reducing cholesterol, reducing LDL/VLDL, increasing HDL or reducing total cholesterol/HDL ratio; or treating or reducing the likelihood of hypercholesteremia, Type III Familial hyperlipoproteinemia, Familial Hypercholesterolemia, Cerebral amyloid angiopathy, dementia, post-stent restenosis, atherosclerosis, coronary heart disease or Alzheimer's disease; comprising administering to a subject an effective amount of the polynucleotide of any of the first aspect, any of E1-E21, the recombinant viral vector of any of E22-E26, or the gene delivery vehicle of any of E27-E30, or the pharmaceutical composition of E31 or E32.

Embodiment 33, further describes the second aspect wherein the method reduces cholesterol, reduces the LDL/VLDL, increases HDL or reduces the total cholesterol/HDL ratio in a subject in need thereof.

Embodiment 34, further describes the second aspect or Embodiment E33, wherein the subject has hypercholesteremia.

Embodiment 35, further describes the second aspect, Embodiments E33 and E34, wherein the method treats or reduces the likelihood of hypercholesteremia, Type III Familial hyperlipoproteinemia, Familial Hypercholesterolemia, Cerebral amyloid angiopathy, dementia, post-stent restenosis, atherosclerosis, coronary heart disease or Alzheimer's disease in a subject.

Embodiment 36 further describes the second aspect, Embodiments E33, E34, and E35, wherein the subject is a statin hyporesponder or is statin intolerant.

Embodiment 37, further describes the second aspect, wherein the method is directed to treating or reducing the likelihood of Alzheimer's disease in a subject.

Embodiment 38, further describes the second aspect, wherein the method is directed to treating or reducing the likelihood of vascular dementia or frontotemporal dementia in a subject.

Embodiment 39a, further describes Embodiment E37, wherein administering comprises intraparenchymal, intracisternal or intraventricular administration.

Embodiment 39b, further describes the second aspect and Embodiments E37, E36, E37 and E38 wherein administering comprises intravenous administration.

Embodiment 40 further describes the second aspect, Embodiments E33, E34, E35, E37, E38, E39a and E39b wherein the subject has at least one ApoE4 allele, is an EpoE4 homozygote, or is an PSEN1 mutation carrier.

Embodiment 41, further describes the second aspect, Embodiments E33, E34, E35, E36, E37, E38, E39a, E39b, and E40, wherein native ApoE expression is inhibited. In further embodiments native ApoE expression is inhibited using inhibitory nucleic acid; and the inhibitory nucleic acid is selected from a short hairpin RNA (shRNA), a small interfering RNA (siRNA), a microRNA (miRNA), a RNAi, a ribozyme, and an antisense RNA.

Embodiment 42, further describes the second aspect, Embodiments E33, E34, E35, E36, E37, E38, E39a, E39b, E40, and E41 wherein the subject is a human.

A third aspect is directed to the polynucleotide of any of the first aspect, any of Embodiments E1-E21, the recombinant viral vector of any of Embodiments E22-E26, the gene delivery vehicle of any of Embodiments E27-E30, or the pharmaceutical composition of Embodiments E31 or E32 for use in medicine, and in any of the methods provided in the second aspect and any of Embodiments E33-E42.

A fourth aspect is directed to use of the polypeptide of any of the first aspect, any of Embodiments E1-E21, the recombinant viral vector of any of Embodiments E22-E26, the gene delivery vehicle of any of Embodiments E27-E30, or the pharmaceutical composition of Embodiments E31 or E32 for preparation of a medicament for use in medicine or as provided in the second aspect and any of Embodiments E33-42.

A fifth aspect is directed to an AAV vector genome plasmid comprising recombinant viral nucleic acid of any Embodiments E22-26.

Embodiment 43 further described the fifth aspect, wherein the plasmid lacks rep and cap genes.

A sixth aspect is directed to a method of producing a rAAV vector comprising the step of culturing an rAAV production cell line comprising rAAV helper virus activity, wherein the genome of the production cell comprises the recombinant viral vector nucleic acid of any one of Embodiments E22-26, a rep gene and a cap gene, wherein the rAAV vector is produced.

Embodiment 44 is directed to a method of producing rAAV vector comprising the step of culturing an rAAV permissive cell comprising the AAV genome plasmid of sixth aspect or Embodiment 43, wherein the rAAV permissive cell further comprises (a) rep and cap genes provided either as part of the cell genome and/or by one or more separate plasmids, and (b) helper virus activity provided by the cell genome and/or provided by one or more separate plasmids.

Embodiment 45 further describes Embodiment 44, wherein the rAAV permissive cell is a packaging cell, wherein the genome of the packaging comprises a cap gene and a rep gene.

Embodiment 46 further describes embodiment 44, wherein either (a) the rep gene, the cap gene and the helper activity is provided in a single plasmid or (b) the rep gene and said cap gene is provided by a rep cap plasmid and the helper activity is provided by a helper plasmid.

A seventh aspect is directed to a method of obtaining an rAAV vector comprising the steps of (a) producing the rAAV using the method of the sixth aspect or any of Embodiments 44-46 and (b) purifying the rAAV.

An eight aspect is directed to a polynucleotide comprising a sequence with at least 50%, at least 60%, at least 70%, at least 80%, at least 90%, at least 95%, at least 96%, at least 97%, at least 98%, at least 99%, or 100% sequence identity with the sequence of any of SEQ ID NOs: 119-121, wherein the polynucleotide has 0-5 CpGs. Preferably, the polynucleotide is an intron that can be excised from a pre-mRNA transcript mediated by a spliceosome.

Embodiment 47 further described the eight aspect, where the polynucleotide comprise the sequence of any of SEQ ID NOs: 119-121, or a sequence differing from any of SEQ ID NOs: 119-121 by 1-10 nucleotides, wherein the polynucleotide has no CpGs. In a further embodiment, the polynucleotide consists of the sequence of any of SEQ ID NOs: 119-121.

### XI. Sequences

Table 1 provides different nucleic acid and amino acid sequences. Sequences indicated in bold provide a codon.

In different embodiments (1) a polynucleotide comprises a nucleotide sequence having a sequence identity of at least 95%, at least 96%, at least 97%, at least 98%, at least 99%, or 100% to any of the nucleic acid sequences provided in Table 1; (2) a polynucleotide comprises a nucleotide sequence having a sequence identity of at least 95%, at least 96%, at least 97%, at least 98%, at least 99%, or 100% to any of the nucleic acid sequences provided in Table 1, wherein the stop codon shown in bold is not present and/or is replaced with a different stop codon; or (3) a polypeptide comprises an amino acid sequence having a sequence identity of at least 95%, at least 96%, at least 97%, at least 98%, at least 99%, or 100% to any of the amino acid sequences provided in Table 1.

TABLE 1
ApoE Encoding Sequences

Native ApoE3 (SEQ ID NO: 1)
ATGAAGGTTCTGTGGGCTGCGTTGCTGGTCACATTCCTGGCAGGATGCCAGGCCAAGGTGGAGCAAGCGGTGG
AGACAGAGCCGGAGCCCGAGCTGCGCCAGCAGACCGAGTGGCAGAGCGGCCAGCGCTGGGAACTGGCACTGGG
TCGCTTTTGGGATTACCTGCGCTGGGTGCAGACACTGTCTGAGCAGGTGCAGGAGGAGCTGCTCAGCTCCCAG
GTCACCCAGGAACTGAGGGCGCTGATGGACGAGACCATGAAGGAGTTGAAGGCCTACAAATCGGAACTGGAGG
AACAACTGACCCCGGTGGCGGAGGAGACGCGGGCACGGCTGTCCAAGGAGCTGCAGGCGGCGCAGGCCCGGCT
GGGCGCGGACATGGAGGACGTGTGCGGCCGCCTGGTGCAGTACCGCGGCGAGGTGCAGGCCATGCTCGGCCAG
AGCACCGAGGAGCTGCGGGTGCGCCTCGCCTCCCACCTGCGCAAGCTGCGTAAGCGGCTCCTCCGCGATGCCG
ATGACCTGCAGAAGCGCCTGGCAGTGTACCAGGCCGGGGCCCGCGAGGGCGCCGAGCGCGGCCTCAGCGCCAT
CCGCGAGCGCCTGGGGCCCCTGGTGGAACAGGGCCGCGTGCGGGCCGCCACTGTGGGCTCCCTGGCCGGCCAG
CCGCTACAGGAGCGGGCCCAGGCCTGGGGCGAGCGGCTGCGCGCGCGGATGGAGGAGATGGGCAGCCGGACCC
GCGACCGCCTGGACGAGGTGAAGGAGCAGGTGGCGGAGGTGCGCGCCAAGCTGGAGGAGCAGGCCCAGCAGAT
ACGCCTGCAGGCCGAGGCCTTCCAGGCCCGCCTCAAGAGCTGGTTCGAGCCCCTGGTGGAAGACATGCAGCGC
CAGTGGGCCGGGCTGGTGGAGAAGGTGCAGGCTGCCGTGGGCACCAGCGCCGCCCCTGTGCCCAGCGACAATC
ACTGA
Native ApoE3ch (SEQ ID NO: 2)
ATGAAGGTTCTGTGGGCTGCGTTGCTGGTCACATTCCTGGCAGGATGCCAGGCCAAGGTGGAGCAAGCGGTGG
AGACAGAGCCGGAGCCCGAGCTGCGCCAGCAGACCGAGTGGCAGAGCGGCCAGCGCTGGGAACTGGCACTGGG
TCGCTTTTGGGATTACCTGCGCTGGGTGCAGACACTGTCTGAGCAGGTGCAGGAGGAGCTGCTCAGCTCCCAG
GTCACCCAGGAACTGAGGGCGCTGATGGACGAGACCATGAAGGAGTTGAAGGCCTACAAATCGGAACTGGAGG
AACAACTGACCCCGGTGGCGGAGGAGACGCGGGCACGGCTGTCCAAGGAGCTGCAGGCGGCGCAGGCCCGGCT
GGGCGCGGACATGGAGGACGTGTGCGGCCGCCTGGTGCAGTACAGCGGCGAGGTGCAGGCCATGCTCGGCCAG
AGCACCGAGGAGCTGCGGGTGCGCCTCGCCTCCCACCTGCGCAAGCTGCGTAAGCGGCTCCTCCGCGATGCCG
ATGACCTGCAGAAGCGCCTGGCAGTGTACCAGGCCGGGGCCCGCGAGGGCGCCGAGCGCGGCCTCAGCGCCAT
CCGCGAGCGCCTGGGGCCCCTGGTGGAACAGGGCCGCGTGCGGGCCGCCACTGTGGGCTCCCTGGCCGGCCAG
CCGCTACAGGAGCGGGCCCAGGCCTGGGGCGAGCGGCTGCGCGCGCGGATGGAGGAGATGGGCAGCCGGACCC
GCGACCGCCTGGACGAGGTGAAGGAGCAGGTGGCGGAGGTGCGCGCCAAGCTGGAGGAGCAGGCCCAGCAGAT
ACGCCTGCAGGCCGAGGCCTTCCAGGCCCGCCTCAAGAGCTGGTTCGAGCCCCTGGTGGAAGACATGCAGCGC
CAGTGGGCCGGGCTGGTGGAGAAGGTGCAGGCTGCCGTGGGCACCAGCGCCGCCCCTGTGCCCAGCGACAATC
ACTGA
ApoE3ch-1 (SEQ ID NO: 3)
ATGAAGGTGCTGTGGGCTGCCCTGCTGGTGACTTTCCTGGCTGGCTGCCAGGCTAAGGTGGAGCAGGCTGTGG
AGACTGAGCCTGAGCCTGAGCTGAGGCAGCAGACTGAGTGGCAGTCTGGGCAGAGGTGGGAGCTGGCCCTGGG
CAGGTTCTGGGACTATCTGAGGTGGGTGCAGACCCTGTCTGAGCAGGTGCAGGAGGAGCTGCTGAGCTCTCAG
GTGACTCAGGAGCTGAGGGCCCTGATGGATGAGACCATGAAGGAGCTGAAGGCTTACAAGTCTGAGCTGGAGG
AGCAGCTGACCCCTGTGGCTGAGGAGACCAGAGCCAGGCTGAGCAAGGAGCTGCAGGCTGCTCAGGCTAGGCT
GGGGGCTGACATGGAGGATGTGTGTGGCAGGCTGGTGCAGTACTCTGGGGAGGTGCAGGCCATGCTGGGCCAG
AGCACTGAGGAGCTGAGGGTGAGGCTGGCTTCTCACCTGAGGAAGCTGAGAAAGAGGCTGCTGAGGGATGCTG
ATGATCTGCAGAAGAGGCTGGCTGTGTACCAGGCTGGGGCCAGGGAAGGGGCTGAGAGGGGCCTGTCTGCCAT
TAGGGAGAGGCTGGGGCCCCTGGTGGAGCAGGGGAGGGTGAGGGCTGCCACTGTGGGCTCTCTGGCTGGCCAG
CCCCTGCAGGAGAGAGCCCAGGCTTGGGGGGAGAGGCTGAGGGCCAGGATGGAGGAGATGGGCTCTAGGACCA
GGGATAGGCTGGATGAGGTGAAGGAGCAGGTGGCTGAGGTCAGGGCTAAGCTGGAGGAGCAGGCCCAGCAGAT
CAGGCTGCAGGCTGAGGCCTTCCAGGCCAGGCTGAAGAGCTGGTTTGAGCCCCTGGTGGAGGACATGCAGAGG
CAGTGGGCTGGGCTGGTGGAGAAGGTGCAGGCTGCTGTGGGGACCTCTGCTGCCCCTGTGCCCTCTGACAATC
ACTGA
ApoE3ch-2 (SEQ ID NO: 4)
ATGAAGGTGCTGTGGGCTGCCCTGCTGGTGACCTTCCTGGCTGGCTGCCAGGCCAAGGTGGAGCAGGCTGTGG
AGACTGAGCCTGAGCCTGAGCTGAGGCAGCAGACTGAGTGGCAGTCTGGCCAGAGGTGGGAGCTGGCCCTGGG
CAGATTCTGGGACTACCTGAGGTGGGTGCAGACCCTGTCTGAGCAGGTGCAGGAGGAGCTGCTGAGCAGCCAG
GTGACCCAGGAGCTGAGGGCCCTGATGGATGAGACCATGAAGGAGCTGAAGGCCTATAAGTCTGAGCTGGAGG
AGCAGCTGACCCCTGTGGCTGAGGAGACCAGGGCTAGGCTGAGCAAGGAGCTGCAGGCTGCCCAGGCTAGGCT
GGGGGCTGACATGGAGGATGTGTGTGGGAGGCTGGTGCAGTACTCTGGGGAGGTGCAGGCCATGCTGGGCCAG
AGCACTGAGGAGCTGAGGGTGAGGCTGGCCAGCCACCTGAGGAAGCTGAGAAAGAGGCTGCTGAGGGATGCTG
ATGATCTGCAGAAGAGACTGGCTGTGTACCAGGCTGGGGCCAGGGAGGGGGCTGAAAGGGGCCTGTCTGCTAT
CAGAGAGAGGCTGGGCCCCCTGGTGGAGCAGGGCAGGGTGAGGGCTGCCACTGTGGGCAGCCTGGCTGGGCAG
CCCCTGCAGGAAAGAGCCCAGGCTTGGGGGGAGAGACTGAGGGCCAGGATGGAGGAGATGGGCAGCAGGACTA
GGGACAGGCTGGATGAGGTGAAGGAGCAGGTGGCTGAGGTGAGAGCCAAGCTGGAGGAGCAGGCCCAGCAGAT
CAGGCTGCAGGCTGAGGCCTTCCAGGCCAGGCTGAAGAGCTGGTTTGAGCCCCTGGTGGAGGATATGCAGAGA
CAGTGGGCTGGCCTGGTGGAGAAGGTGCAGGCTGCTGTGGGCACCTCTGCTGCCCCTGTGCCTTCTGACAACC
ATTGA
ApoE3ch-3 (SEQ ID NO: 5)
ATGAAGGTGCTGTGGGCTGCCCTGCTGGTGACCTTTCTGGCTGGGTGCCAGGCCAAGGTGGAGCAGGCTGTGG
AGACTGAGCCTGAGCCTGAGCTGAGGCAGCAGACTGAGTGGCAGTCTGGGCAGAGGTGGGAGCTGGCCCTGGG
GAGGTTCTGGGACTACCTGAGGTGGGTGCAGACTCTGTCTGAGCAGGTGCAGGAGGAGCTGCTGTCTAGCCAG
GTGACCCAGGAGCTGAGGGCCCTGATGGATGAGACCATGAAGGAGCTGAAGGCCTACAAGTCTGAGCTGGAAG
AGCAGCTGACCCCTGTGGCTGAGGAGACCAGGGCTAGGCTGAGCAAGGAACTGCAGGCTGCTCAGGCCAGGCT
GGGGGCTGACATGGAAGATGTGTGTGGGAGGCTGGTGCAGTACTCTGGGGAGGTGCAGGCCATGCTGGGCCAG
AGCACTGAGGAGCTGAGGGTGAGGCTGGCCAGCCACCTGAGGAAGCTGAGGAAGAGGCTGCTGAGGGATGCTG
ATGACCTGCAGAAGAGGCTGGCTGTGTACCAGGCTGGGGCCAGGGAGGGGGCTGAGAGGGGCCTGTCTGCTAT
CAGGGAGAGGCTGGGCCCCCTGGTGGAGCAGGGCAGGGTGAGGGCTGCCACTGTGGGCAGCCTGGCTGGGCAG
CCTCTGCAGGAGAGGGCCCAGGCCTGGGGGGAGAGGCTGAGAGCCAGGATGGAGGAGATGGGCTCTAGGACTA
GGGACAGGCTGGATGAGGTGAAGGAGCAGGTGGCTGAGGTGAGGGCCAAGCTGGAGGAGCAGGCTCAGCAGAT
CAGGCTGCAGGCTGAGGCTTTCCAGGCCAGGCTGAAGTCTTGGTTTGAGCCCCTGGTGGAGGATATGCAGAGG
CAGTGGGCTGGCCTGGTGGAGAAGGTGCAGGCTGCTGTGGGCACCTCTGCTGCCCCTGTGCCCTCTGACAACC
ATTGA
ApoE3ch-4 (SEQ ID NO: 6)
ATGAAGGTGCTGTGGGCTGCCCTGCTGGTGACCTTCCTGGCTGGCTGTCAGGCCAAGGTGGAGCAGGCTGTGG
AGACTGAGCCTGAGCCAGAGCTGAGGCAGCAGACTGAGTGGCAGTCTGGGCAGAGGTGGGAGCTGGCTCTGGG
GAGGTTCTGGGACTACCTGAGATGGGTGCAGACCCTGTCTGAACAGGTGCAGGAGGAGCTGCTGAGCAGCCAG
GTGACCCAGGAGCTGAGAGCCCTGATGGATGAGACCATGAAGGAGCTGAAGGCCTACAAGTCTGAGCTGGAGG
AGCAGCTGACTCCTGTGGCTGAGGAGACTAGGGCCAGGCTGTCTAAGGAGCTGCAGGCTGCCCAGGCCAGGCT
GGGGGCTGACATGGAGGATGTGTGTGGCAGGCTGGTGCAGTACTCTGGGGAGGTCCAGGCCATGCTGGGCCAG
AGCACTGAGGAGCTGAGGGTGAGGCTGGCCAGCCACCTGAGGAAGCTGAGGAAGAGGCTGCTGAGGGATGCTG
ATGACCTGCAGAAAAGGCTGGCTGTGTACCAGGCTGGGGCCAGGGAGGGGGCTGAGAGAGGGCTGTCTGCCAT
TAGAGAGAGGCTGGGCCCCCTGGTGGAGCAGGGCAGGGTGAGGGCTGCTACTGTGGGCAGCCTGGCTGGGCAG
CCTCTGCAGGAGAGGGCCCAGGCCTGGGGGGAGAGGCTGAGGGCTAGGATGGAGGAGATGGGCTCTAGGACCA
GGGACAGGCTGGATGAGGTGAAGGAGCAGGTGGCTGAGGTGAGGGCCAAGCTGGAGGAGCAGGCCCAGCAGAT
CAGGCTGCAGGCTGAGGCCTTCCAGGCTAGGCTGAAGAGCTGGTTTGAGCCCCTGGTGGAGGACATGCAGAGG
CAGTGGGCTGGGCTGGTGGAGAAGGTGCAGGCTGCTGTGGGGACCTCTGCTGCTCCTGTGCCCTCTGACAACC
ACTGA
ApoE3ch-5 (SEQ ID NO: 7)
ATGAAGGTGCTGTGGGCTGCCCTGCTGGTGACCTTCCTGGCTGGGTGCCAGGCTAAGGTGGAGCAGGCTGTGG
AGACTGAGCCTGAACCTGAACTGAGGCAGCAGACTGAGTGGCAGTCTGGCCAGAGGTGGGAGCTGGCTCTGGG
GAGGTTCTGGGACTACCTGAGGTGGGTGCAGACTCTGTCTGAGCAGGTGCAGGAGGAGCTGCTGAGCAGCCAG
GTGACCCAGGAGCTGAGGGCCCTGATGGATGAGACCATGAAGGAGCTGAAGGCCTACAAGTCTGAGCTGGAGG
AGCAGCTGACCCCTGTGGCTGAGGAAACCAGGGCCAGGCTGTCTAAGGAGCTGCAGGCTGCTCAGGCTAGGCT
GGGGGCTGACATGGAGGATGTGTGTGGGAGACTGGTGCAGTATTCTGGGGAGGTGCAGGCCATGCTGGGCCAG
AGCACTGAGGAGCTGAGGGTGAGGCTGGCTAGCCACCTGAGAAAGCTGAGGAAGAGGCTGCTGAGGGATGCTG
ATGATCTGCAGAAGAGGCTGGCTGTGTATCAGGCTGGGGCCAGGGAGGGGGCTGAGAGGGGCCTGTCTGCCAT
CAGAGAGAGGCTGGGGCCTCTGGTGGAGCAGGGCAGGGTGAGGGCTGCTACTGTGGGCAGCCTGGCTGGCCAG
CCCCTGCAGGAGAGGGCCCAGGCCTGGGGGGAGAGGCTGAGGGCTAGGATGGAGGAGATGGGCAGCAGAACCA
GGGACAGGCTGGATGAGGTGAAGGAGCAGGTGGCTGAGGTGAGAGCCAAGCTGGAGGAGCAGGCTCAGCAGAT
CAGGCTGCAGGCTGAGGCTTTCCAGGCTAGGCTGAAGTCTTGGTTTGAGCCCCTGGTGGAAGATATGCAGAGG
CAGTGGGCTGGCCTGGTGGAGAAGGTGCAGGCTGCTGTGGGCACCTCTGCTGCCCCTGTGCCCTCTGACAATC
ATTGA
ApoE3ch-6 (SEQ ID NO: 8)
ATGAAGGTGCTGTGGGCTGCTCTGCTGGTGACCTTCCTGGCTGGGTGCCAGGCCAAGGTGGAGCAGGCTGTGG
AGACTGAGCCAGAGCCTGAGCTGAGGCAGCAGACTGAGTGGCAGTCTGGCCAGAGGTGGGAGCTGGCCCTGGG
CAGGTTCTGGGACTACCTGAGGTGGGTGCAGACCCTGTCTGAGCAGGTGCAGGAGGAGCTGCTGAGCTCTCAG
GTGACTCAGGAGCTGAGGGCCCTGATGGATGAGACCATGAAGGAGCTGAAGGCCTACAAGTCTGAGCTGGAGG
AGCAGCTGACCCCTGTGGCTGAGGAGACCAGGGCCAGGCTGAGCAAGGAGCTGCAGGCTGCCCAGGCCAGGCT
GGGGGCTGACATGGAGGATGTGTGTGGCAGGCTGGTGCAGTACTCTGGGGAGGTGCAGGCCATGCTGGGCCAG
TCTACTGAGGAGCTGAGGGTGAGGCTGGCCAGCCATCTGAGGAAGCTGAGGAAGAGGCTGCTGAGGGATGCTG
ATGATCTGCAGAAGAGGCTGGCTGTGTACCAGGCTGGGGCCAGGGAGGGGGCTGAGAGGGGCCTGTCTGCCAT
TAGGGAGAGGCTGGGGCCCCTGGTGGAGCAGGGCAGGGTGAGGGCTGCCACTGTGGGGTCTCTGGCTGGCCAG
CCCCTGCAGGAGAGGGCTCAGGCCTGGGGGGAGAGGCTGAGGGCCAGAATGGAGGAAATGGGCTCTAGGACCA
GGGACAGGCTGGATGAGGTGAAGGAGCAGGTGGCTGAGGTGAGGGCTAAGCTGGAGGAGCAGGCTCAGCAGAT
CAGGCTGCAGGCTGAGGCCTTCCAGGCCAGGCTGAAGAGCTGGTTTGAGCCCCTGGTGGAAGACATGCAGAGG
CAGTGGGCTGGCCTGGTGGAGAAGGTGCAGGCTGCTGTGGGCACCTCTGCTGCCCCTGTGCCCTCTGATAACC
ACTGA
ApoE3ch-7 (SEQ ID NO: 9)
ATGAAGGTGCTGTGGGCTGCCCTGCTGGTGACTTTCCTGGCTGGCTGTCAGGCCAAGGTGGAGCAGGCTGTGG
AGACTGAGCCTGAGCCTGAGCTGAGGCAGCAGACTGAGTGGCAGTCTGGCCAGAGGTGGGAGCTGGCCCTGGG
CAGGTTCTGGGATTACCTGAGGTGGGTGCAGACCCTGTCTGAGCAGGTGCAGGAGGAGCTGCTGAGCTCTCAG
GTGACCCAGGAGCTGAGGGCCCTGATGGATGAGACCATGAAGGAGCTGAAGGCCTACAAGTCTGAGCTGGAGG
AGCAGCTGACCCCTGTGGCTGAGGAGACCAGAGCTAGGCTGAGCAAGGAGCTGCAGGCTGCTCAGGCCAGGCT
GGGGGCTGACATGGAGGATGTGTGTGGCAGACTGGTGCAGTACTCTGGGGAGGTGCAGGCTATGCTGGGGCAG
AGCACTGAGGAGCTGAGAGTGAGGCTGGCCTCTCACCTGAGGAAGCTGAGGAAGAGGCTGCTGAGGGATGCTG
ATGACCTGCAGAAGAGGCTGGCTGTCTACCAGGCTGGGGCCAGGGAGGGGGCTGAGAGGGGCCTGTCTGCCAT
CAGGGAGAGGCTGGGCCCCCTGGTGGAGCAGGGCAGGGTGAGAGCTGCCACTGTGGGGAGCCTGGCTGGCCAG
CCCCTGCAGGAGAGGGCCCAGGCCTGGGGGGAGAGGCTGAGGGCTAGGATGGAGGAGATGGGCAGCAGGACCA
GGGACAGGCTGGATGAGGTGAAGGAGCAGGTGGCTGAGGTGAGGGCCAAGCTGGAGGAGCAGGCCCAGCAGAT
TAGGCTGCAGGCTGAGGCCTTCCAGGCCAGGCTGAAGAGCTGGTTTGAGCCTCTGGTGGAGGACATGCAGAGA
CAGTGGGCTGGCCTGGTGGAGAAGGTGCAGGCTGCTGTGGGGACCTCTGCTGCTCCAGTGCCCTCTGATAATC
ACTGA
ApoE3ch-8 (SEQ ID NO: 10)
ATGAAGGTGCTGTGGGCTGCCCTGCTGGTGACCTTCCTGGCTGGGTGCCAGGCTAAGGTGGAGCAGGCTGTGG
AGACTGAGCCTGAGCCTGAGCTGAGACAGCAGACTGAGTGGCAGTCTGGCCAGAGGTGGGAGCTGGCCCTGGG
GAGGTTCTGGGACTACCTGAGGTGGGTGCAGACCCTGTCTGAGCAGGTGCAGGAGGAGCTGCTGTCTTCTCAG
GTGACCCAGGAGCTGAGGGCCCTGATGGATGAGACCATGAAGGAGCTGAAGGCCTACAAGTCTGAGCTGGAGG
AGCAGCTGACTCCTGTGGCTGAGGAGACCAGGGCCAGGCTGTCTAAGGAGCTGCAGGCTGCTCAGGCCAGGCT
GGGGGCTGACATGGAGGATGTGTGTGGCAGGCTGGTGCAGTACTCTGGGGAGGTGCAGGCCATGCTGGGGCAG
AGCACTGAGGAGCTGAGGGTGAGGCTGGCCAGCCATCTGAGGAAGCTGAGGAAGAGGCTGCTGAGGGATGCTG
ATGACCTGCAGAAGAGGCTGGCTGTGTATCAGGCTGGGGCCAGGGAGGGGGCTGAGAGGGGCCTGTCTGCCAT
TAGGGAGAGGCTGGGCCCCCTGGTGGAGCAGGGGAGGGTGAGGGCTGCCACTGTGGGGAGCCTGGCTGGGCAG
CCCCTGCAGGAGAGGGCCCAGGCTTGGGGGGAGAGGCTGAGGGCCAGGATGGAGGAGATGGGGAGCAGGACCA
GGGATAGGCTGGATGAGGTCAAGGAGCAGGTGGCTGAGGTGAGGGCTAAGCTGGAGGAACAGGCCCAGCAGAT
CAGGCTGCAGGCTGAGGCTTTCCAGGCCAGACTGAAGAGCTGGTTTGAGCCCCTGGTGGAGGACATGCAGAGG
CAGTGGGCTGGCCTGGTGGAGAAGGTGCAGGCTGCTGTGGGCACCTCTGCTGCCCCAGTGCCCTCTGATAACC
ACTGA
ApoE3ch-9 (SEQ ID NO: 11)
ATGAAGGTGCTGTGGGCTGCCCTGCTGGTGACTTTCCTGGCTGGCTGCCAGGCTAAGGTGGAGCAGGCTGTGG
AGACTGAGCCAGAGCCTGAGCTGAGGCAGCAGACTGAATGGCAGTCTGGCCAGAGGTGGGAGCTGGCCCTGGG
CAGGTTCTGGGACTACCTGAGATGGGTGCAGACCCTGTCTGAGCAGGTCCAGGAGGAGCTGCTGTCTTCTCAG
GTGACCCAGGAGCTGAGGGCCCTGATGGATGAGACTATGAAGGAGCTGAAGGCTTACAAGTCTGAGCTGGAGG
AGCAGCTGACCCCTGTGGCTGAGGAGACCAGGGCCAGACTGAGCAAGGAGCTGCAGGCTGCTCAGGCCAGGCT
GGGGGCTGACATGGAGGATGTGTGTGGCAGGCTGGTGCAGTATTCTGGGGAGGTGCAGGCTATGCTGGGCCAG
AGCACTGAGGAACTGAGGGTGAGGCTGGCCTCTCATCTGAGGAAGCTGAGGAAGAGGCTGCTGAGGGATGCTG
ATGACCTGCAGAAGAGGCTGGCTGTGTATCAGGCTGGGGCCAGGGAGGGGGCTGAGAGGGGGCTGTCTGCTAT
CAGGGAGAGGCTGGGCCCCCTGGTGGAGCAGGGGAGAGTGAGGGCTGCCACTGTGGGGTCTCTGGCTGGCCAG
CCCCTGCAGGAGAGGGCCCAGGCCTGGGGGGAGAGGCTGAGGGCTAGAATGGAGGAGATGGGGTCTAGGACCA
GGGACAGGCTGGATGAGGTCAAGGAACAGGTGGCTGAGGTCAGGGCCAAGCTGGAGGAGCAGGCCCAGCAGAT
TAGGCTGCAGGCTGAGGCCTTTCAGGCCAGGCTGAAGAGCTGGTTTGAGCCCCTGGTGGAGGACATGCAGAGG
CAGTGGGCTGGCCTGGTGGAGAAGGTGCAGGCTGCTGTGGGCACTTCTGCTGCCCCTGTGCCCTCTGACAACC
ATTGA
ApoE3ch-10 (SEQ ID NO: 12)
ATGAAGGTGCTGTGGGCTGCCCTGCTGGTGACCTTCCTGGCTGGGTGCCAGGCTAAGGTGGAGCAGGCTGTGG
AGACTGAGCCTGAGCCTGAGCTGAGGCAGCAGACTGAGTGGCAGTCTGGCCAGAGGTGGGAGCTGGCCCTGGG
CAGGTTTTGGGACTACCTGAGGTGGGTGCAGACCCTGTCTGAGCAGGTGCAGGAAGAGCTGCTGTCTAGCCAG
GTGACCCAGGAGCTGAGGGCCCTGATGGATGAGACCATGAAGGAGCTGAAGGCCTACAAGTCTGAACTGGAGG
AGCAGCTGACCCCTGTGGCTGAGGAGACTAGGGCCAGGCTGAGCAAGGAGCTGCAGGCTGCCCAGGCCAGACT
GGGGGCTGACATGGAGGATGTGTGTGGGAGGCTGGTGCAGTACTCTGGGGAGGTGCAGGCCATGCTGGGCCAG
AGCACTGAGGAGCTGAGGGTGAGACTGGCTAGCCATCTGAGGAAGCTGAGAAAGAGGCTGCTGAGGGATGCTG
ATGACCTGCAGAAGAGGCTGGCTGTGTACCAGGCTGGGGCCAGGGAGGGGGCTGAGAGGGGCCTGTCTGCCAT
CAGGGAGAGGCTGGGCCCCCTGGTGGAGCAGGGCAGGGTGAGGGCTGCTACTGTGGGCAGCCTGGCTGGCCAG
CCCCTGCAGGAGAGGGCCCAGGCTTGGGGGGAGAGGCTGAGGGCCAGGATGGAGGAGATGGGCAGCAGGACCA
GGGATAGGCTGGATGAGGTGAAAGAGCAGGTGGCTGAGGTGAGGGCCAAGCTGGAGGAGCAGGCCCAGCAGAT
CAGGCTGCAGGCTGAGGCCTTCCAGGCTAGACTGAAGAGCTGGTTTGAGCCCCTGGTGGAGGACATGCAGAGG
CAGTGGGCTGGCCTGGTGGAGAAGGTGCAGGCTGCTGTGGGCACCTCTGCTGCCCCTGTGCCTTCTGACAATC
ACTGA
ApoE3ch-11 (SEQ ID NO: 13)
ATGAAGGTGCTGTGGGCTGCCCTGCTGGTGACCTTCCTGGCTGGGTGTCAGGCCAAGGTGGAGCAGGCTGTGG
AGACTGAGCCTGAGCCTGAACTGAGGCAGCAGACTGAGTGGCAGTCTGGCCAGAGGTGGGAGCTGGCCCTGGG
CAGGTTCTGGGATTACCTGAGGTGGGTGCAGACCCTGTCTGAGCAGGTGCAGGAGGAGCTGCTGTCTAGCCAG
GTGACCCAGGAGCTGAGGGCCCTGATGGATGAGACCATGAAGGAGCTGAAGGCCTACAAGTCTGAGCTGGAGG
AGCAGCTGACTCCTGTGGCTGAGGAGACTAGGGCCAGGCTGTCCAAGGAGCTGCAGGCTGCCCAGGCCAGACT
GGGGGCTGACATGGAAGATGTGTGTGGGAGGCTGGTGCAGTACTCTGGGGAGGTGCAGGCCATGCTGGGGCAG
AGCACTGAGGAGCTGAGGGTGAGGCTGGCCAGCCACCTGAGGAAGCTGAGGAAGAGGCTGCTGAGGGATGCTG
ATGACCTGCAGAAGAGGCTGGCTGTGTACCAGGCTGGGGCCAGGGAGGGGGCTGAAAGAGGCCTGTCTGCCAT
CAGGGAGAGGCTGGGGCCCCTGGTGGAGCAGGGCAGGGTGAGGGCTGCCACTGTGGGCAGCCTGGCTGGCCAG
CCCCTGCAGGAGAGGGCCCAGGCCTGGGGGGAGAGGCTGAGGGCCAGGATGGAGGAGATGGGGAGCAGGACCA
GGGACAGGCTGGATGAGGTGAAGGAGCAGGTGGCTGAGGTGAGGGCCAAGCTGGAGGAGCAGGCCCAGCAGAT
CAGGCTGCAGGCTGAGGCTTTCCAGGCTAGGCTGAAGAGCTGGTTTGAGCCCCTGGTGGAGGACATGCAGAGG
CAGTGGGCTGGCCTGGTGGAGAAGGTGCAGGCTGCTGTGGGGACTTCTGCTGCCCCAGTGCCCTCTGACAATC
ATTGA
ApoE3ch-12 (SEQ ID NO: 14)
ATGAAGGTCCTGTGGGCTGCCCTGCTGGTGACCTTCCTGGCTGGCTGCCAGGCTAAGGTGGAGCAGGCTGTGG
AGACTGAGCCAGAGCCTGAGCTGAGGCAGCAGACTGAGTGGCAGTCTGGCCAGAGGTGGGAGCTGGCCCTGGG
CAGGTTCTGGGACTACCTGAGGTGGGTGCAGACTCTGTCTGAGCAGGTGCAGGAGGAGCTGCTGTCTAGCCAG
GTGACCCAGGAGCTGAGGGCCCTGATGGATGAGACTATGAAGGAGCTGAAGGCTTACAAGTCTGAGCTGGAGG
AGCAGCTGACCCCTGTGGCTGAGGAGACCAGGGCTAGGCTGAGCAAGGAGCTGCAGGCTGCCCAGGCCAGGCT
GGGGGCTGACATGGAAGATGTGTGTGGCAGGCTGGTGCAGTACTCTGGGGAGGTGCAGGCTATGCTGGGCCAG
AGCACTGAGGAGCTGAGGGTGAGGCTGGCCTCTCACCTGAGGAAGCTGAGGAAGAGGCTGCTGAGGGATGCTG
ATGACCTGCAGAAGAGGCTGGCTGTGTACCAGGCTGGGGCCAGGGAGGGGGCTGAGAGGGGCCTGTCTGCTAT
CAGGGAGAGGCTGGGCCCCCTGGTGGAGCAGGGGAGAGTGAGGGCTGCCACTGTGGGCTCTCTGGCTGGCCAG
CCCCTGCAGGAGAGGGCTCAGGCCTGGGGGGAGAGGCTGAGGGCCAGGATGGAAGAGATGGGCAGCAGGACCA
GGGACAGGCTGGATGAGGTGAAGGAGCAGGTGGCTGAGGTGAGGGCTAAGCTGGAGGAGCAGGCCCAGCAGAT
TAGGCTGCAGGCTGAGGCCTTCCAGGCCAGGCTGAAGAGCTGGTTTGAGCCCCTGGTGGAGGATATGCAGAGG
CAGTGGGCTGGGCTGGTGGAGAAGGTGCAGGCTGCTGTGGGCACTTCTGCTGCTCCTGTGCCCTCTGACAACC
ACTGA
ApoE3ch-13 (SEQ ID NO: 15)
ATGAAGGTGCTGTGGGCTGCCCTGCTGGTGACTTTTCTGGCTGGCTGCCAGGCCAAGGTGGAGCAGGCTGTGG
AGACTGAGCCTGAGCCTGAGCTGAGGCAGCAGACTGAGTGGCAGTCTGGCCAGAGGTGGGAGCTGGCCCTGGG
GAGGTTCTGGGACTACCTGAGGTGGGTGCAGACCCTGTCTGAGCAGGTGCAGGAGGAGCTGCTGAGCAGCCAG
GTGACTCAGGAGCTGAGGGCCCTGATGGATGAGACCATGAAGGAACTGAAGGCCTACAAGTCTGAACTGGAGG
AGCAGCTGACCCCTGTGGCTGAGGAGACCAGGGCCAGGCTGTCTAAGGAGCTGCAGGCTGCCCAGGCTAGGCT
GGGGGCTGATATGGAGGATGTGTGTGGCAGACTGGTGCAGTACTCTGGGGAGGTGCAGGCCATGCTGGGCCAG
AGCACTGAGGAGCTGAGAGTGAGGCTGGCCTCTCACCTGAGAAAGCTGAGGAAGAGGCTGCTGAGGGATGCTG
ATGATCTGCAGAAGAGGCTGGCTGTGTACCAGGCTGGGGCCAGGGAGGGGGCTGAGAGGGGCCTGTCTGCCAT
CAGGGAAAGGCTGGGCCCCCTGGTGGAGCAGGGCAGAGTGAGGGCTGCTACTGTGGGCAGCCTGGCTGGCCAG
CCCCTGCAGGAGAGGGCCCAGGCCTGGGGGGAGAGGCTGAGGGCCAGGATGGAGGAGATGGGCAGCAGGACTA
GGGATAGGCTGGATGAGGTGAAGGAGCAGGTGGCTGAGGTGAGGGCCAAGCTGGAGGAGCAGGCCCAGCAGAT
CAGACTGCAGGCTGAGGCCTTCCAGGCCAGGCTGAAGAGCTGGTTTGAGCCCCTGGTGGAGGATATGCAGAGG
CAGTGGGCTGGCCTGGTGGAGAAGGTGCAGGCTGCTGTGGGCACCTCTGCTGCCCCTGTGCCCTCTGACAACC
ACTGA
ApoE3ch-14 (SEQ ID NO: 16)
ATGAAGGTGCTGTGGGCTGCCCTGCTGGTGACCTTCCTGGCTGGCTGCCAGGCCAAGGTGGAGCAGGCTGTGG
AGACTGAGCCAGAGCCTGAACTGAGGCAGCAGACTGAATGGCAGTCTGGCCAGAGGTGGGAGCTGGCCCTGGG
GAGGTTCTGGGACTACCTGAGGTGGGTGCAGACCCTGTCTGAGCAGGTGCAGGAGGAGCTGCTGAGCAGCCAG
GTGACTCAGGAGCTGAGGGCCCTGATGGATGAGACCATGAAGGAGCTGAAGGCCTATAAGTCTGAGCTGGAGG
AGCAGCTGACCCCTGTGGCTGAGGAGACCAGGGCCAGGCTGTCTAAGGAGCTGCAGGCTGCTCAGGCCAGGCT
GGGGGCTGATATGGAGGATGTGTGTGGCAGGCTGGTGCAGTACTCTGGGGAGGTGCAGGCCATGCTGGGCCAG
TCTACTGAGGAGCTGAGGGTGAGGCTGGCCAGCCACCTGAGGAAGCTGAGGAAGAGGCTGCTGAGGGATGCTG
ATGACCTGCAGAAGAGGCTGGCTGTGTACCAGGCTGGGGCCAGGGAGGGGGCTGAGAGGGGCCTGTCTGCCAT
TAGGGAGAGGCTGGGGCCCCTGGTGGAGCAGGGGAGGGTGAGGGCTGCCACTGTGGGGAGCCTGGCTGGCCAG
CCCCTGCAGGAGAGGGCCCAGGCCTGGGGGGAGAGACTGAGAGCCAGGATGGAAGAGATGGGCAGCAGGACCA
GGGATAGGCTGGATGAGGTGAAGGAGCAGGTGGCTGAGGTCAGGGCCAAGCTGGAGGAGCAGGCCCAGCAGAT
CAGGCTGCAGGCTGAGGCTTTCCAGGCTAGGCTGAAGTCTTGGTTTGAACCTCTGGTGGAAGATATGCAGAGG
CAGTGGGCTGGCCTGGTGGAGAAGGTGCAGGCTGCTGTGGGCACTTCTGCTGCTCCTGTGCCCTCTGATAACC
ACTGA
ApoE3ch-15 (SEQ ID NO: 17)
ATGAAGGTGCTGTGGGCTGCTCTGCTGGTGACCTTCCTGGCTGGCTGCCAGGCCAAGGTGGAGCAGGCTGTGG
AGACTGAACCTGAGCCTGAGCTGAGGCAGCAGACTGAGTGGCAGTCTGGCCAGAGGTGGGAGCTGGCCCTGGG
CAGGTTCTGGGATTACCTGAGGTGGGTGCAGACTCTGTCTGAGCAGGTGCAGGAGGAGCTGCTGTCTTCTCAG
GTGACTCAGGAGCTGAGGGCCCTGATGGATGAGACCATGAAGGAGCTGAAGGCCTACAAGTCTGAGCTGGAGG
AGCAGCTGACCCCTGTGGCTGAGGAGACCAGGGCCAGGCTGTCTAAGGAGCTGCAGGCTGCCCAGGCCAGGCT
GGGGGCTGACATGGAGGATGTGTGTGGCAGGCTGGTGCAGTATTCTGGGGAAGTGCAGGCCATGCTGGGCCAG
AGCACTGAGGAGCTGAGGGTGAGGCTGGCCAGCCATCTGAGGAAGCTGAGGAAGAGGCTGCTGAGAGATGCTG
ATGACCTGCAGAAGAGACTGGCTGTGTACCAGGCTGGGGCCAGGGAGGGGGCTGAGAGGGGCCTGTCTGCTAT
TAGGGAAAGGCTGGGGCCTCTGGTGGAGCAGGGGAGGGTGAGGGCTGCCACTGTGGGCAGCCTGGCTGGCCAG
CCCCTGCAGGAAAGGGCCCAGGCCTGGGGGGAGAGGCTGAGGGCCAGGATGGAGGAGATGGGCAGCAGGACTA
GGGACAGGCTGGATGAGGTGAAGGAGCAGGTGGCTGAGGTCAGGGCTAAGCTGGAGGAGCAGGCCCAGCAGAT
CAGGCTGCAGGCTGAGGCCTTCCAGGCTAGGCTGAAGTCTTGGTTTGAGCCCCTGGTGGAGGACATGCAGAGA
CAGTGGGCTGGCCTGGTGGAGAAGGTGCAGGCTGCTGTGGGCACCTCTGCTGCCCCTGTGCCCTCTGACAACC
ATTGA
APOE3-1: (SEQ ID NO: 18)
ATGAAGGTGCTGTGGGCTGCCCTGCTGGTGACCTTCCTGGCTGGCTGCCAGGCCAAGGTGGAACAGGCTGTGG
AGACTGAGCCTGAGCCTGAGCTGAGGCAGCAGACTGAGTGGCAGTCTGGCCAGAGGTGGGAGCTGGCCCTGGG
GAGGTTTTGGGACTACCTGAGGTGGGTGCAGACCCTGTCTGAGCAGGTCCAGGAGGAGCTGCTGAGCAGCCAG
GTGACTCAGGAGCTGAGAGCCCTGATGGATGAGACTATGAAGGAGCTGAAGGCCTACAAGTCTGAGCTGGAGG
AGCAGCTGACCCCTGTGGCTGAGGAGACCAGGGCCAGGCTGAGCAAGGAGCTGCAGGCTGCTCAGGCCAGGCT
GGGGGCTGACATGGAGGATGTGTGTGGCAGGCTGGTGCAGTACAGGGGGGAGGTGCAGGCCATGCTGGGCCAG
AGCACTGAGGAGCTGAGGGTGAGGCTGGCCAGCCACCTGAGAAAGCTGAGAAAGAGGCTGCTGAGGGATGCTG
ATGATCTGCAGAAGAGGCTGGCTGTGTACCAGGCTGGGGCCAGGGAGGGGGCTGAGAGGGGCCTGTCTGCCAT
CAGGGAGAGGCTGGGCCCCCTGGTGGAGCAGGGCAGAGTGAGGGCTGCCACTGTGGGCAGCCTGGCTGGCCAG
CCCCTGCAGGAGAGGGCCCAGGCCTGGGGGGAGAGGCTGAGAGCTAGGATGGAGGAGATGGGCAGCAGGACTA
GGGACAGGCTGGATGAGGTGAAGGAGCAGGTGGCTGAAGTGAGGGCCAAGCTGGAGGAGCAGGCCCAGCAGAT
CAGGCTGCAGGCTGAGGCCTTCCAGGCCAGACTGAAGTCTTGGTTTGAGCCCCTGGTGGAGGACATGCAGAGG
CAGTGGGCTGGCCTGGTGGAGAAGGTGCAGGCTGCTGTGGGCACTTCTGCTGCCCCTGTGCCTTCTGACAACC
ACTGA
APOE3-2: (SEQ ID NO: 19)
ATGAAGGTGCTGTGGGCTGCTCTGCTGGTCACCTTTCTGGCTGGCTGTCAGGCCAAGGTGGAGCAGGCTGTGG
AGACTGAGCCTGAGCCTGAGCTGAGGCAGCAGACTGAGTGGCAGTCTGGCCAGAGGTGGGAGCTGGCCCTGGG
CAGGTTTTGGGACTACCTGAGGTGGGTGCAGACCCTGTCTGAGCAGGTGCAGGAGGAGCTGCTGTCTAGCCAG
GTGACTCAGGAGCTGAGGGCTCTGATGGATGAGACCATGAAGGAGCTGAAGGCCTACAAGTCTGAGCTGGAGG
AGCAGCTGACCCCTGTGGCTGAGGAGACCAGGGCCAGGCTGTCTAAGGAGCTGCAGGCTGCTCAGGCCAGACT
GGGGGCTGATATGGAGGATGTGTGTGGCAGGCTGGTGCAGTACAGAGGGGAGGTGCAGGCTATGCTGGGCCAG
AGCACTGAGGAGCTGAGGGTGAGGCTGGCCAGCCACCTGAGGAAGCTGAGGAAGAGGCTGCTGAGGGATGCTG
ATGACCTGCAGAAGAGGCTGGCTGTGTACCAGGCTGGGGCTAGGGAGGGGGCTGAGAGGGGCCTGTCTGCCAT
CAGGGAGAGGCTGGGGCCCCTGGTGGAGCAGGGGAGGGTGAGGGCTGCCACTGTGGGCTCTCTGGCTGGCCAG
CCCCTGCAGGAGAGGGCCCAGGCTTGGGGGGAGAGACTGAGGGCCAGGATGGAGGAGATGGGCTCTAGGACTA
GGGATAGGCTGGATGAGGTGAAGGAGCAGGTGGCTGAGGTGAGGGCTAAGCTGGAGGAGCAGGCCCAGCAGAT
CAGGCTGCAGGCTGAGGCCTTCCAGGCCAGGCTGAAGAGCTGGTTTGAGCCTCTGGTGGAGGACATGCAGAGG
CAGTGGGCTGGGCTGGTGGAGAAGGTGCAGGCTGCTGTGGGCACTTCTGCTGCCCCTGTGCCCTCTGATAACC
ACTGA
APOE3-3: (SEQ ID NO: 20)
ATGAAGGTGCTGTGGGCTGCCCTGCTGGTGACCTTCCTGGCTGGCTGTCAGGCTAAAGTGGAGCAGGCTGTGG
AGACTGAGCCTGAGCCAGAGCTGAGGCAGCAGACTGAGTGGCAGTCTGGCCAGAGATGGGAGCTGGCTCTGGG
CAGGTTCTGGGACTACCTGAGGTGGGTGCAGACCCTGTCTGAGCAGGTGCAGGAGGAGCTGCTGTCTTCTCAG
GTGACCCAGGAACTGAGGGCCCTGATGGATGAGACTATGAAGGAGCTGAAGGCCTACAAGTCTGAGCTGGAGG
AGCAGCTGACCCCTGTGGCTGAGGAGACCAGGGCCAGGCTGAGCAAGGAGCTGCAGGCTGCCCAGGCCAGACT
GGGGGCTGACATGGAGGATGTGTGTGGCAGACTGGTGCAGTACAGGGGGGAGGTGCAGGCCATGCTGGGGCAG
TCTACTGAGGAGCTGAGGGTGAGGCTGGCCAGCCACCTGAGGAAGCTGAGGAAGAGGCTGCTGAGGGATGCTG
ATGACCTGCAGAAGAGGCTGGCTGTGTATCAGGCTGGGGCTAGGGAGGGGGCTGAGAGGGGCCTGTCTGCCAT
CAGGGAGAGGCTGGGGCCTCTGGTGGAGCAGGGCAGGGTGAGGGCTGCCACTGTGGGGAGCCTGGCTGGCCAG
CCTCTGCAGGAGAGGGCTCAGGCCTGGGGGGAGAGGCTGAGGGCCAGGATGGAGGAGATGGGGAGCAGGACCA
GGGACAGGCTGGATGAGGTGAAGGAGCAGGTGGCTGAGGTGAGGGCCAAGCTGGAAGAGCAGGCCCAGCAGAT
CAGGCTGCAGGCTGAGGCCTTCCAGGCCAGGCTGAAGAGCTGGTTTGAGCCCCTGGTGGAGGACATGCAGAGG
CAGTGGGCTGGCCTGGTGGAGAAGGTGCAGGCTGCTGTGGGCACCTCTGCTGCCCCTGTGCCCTCTGACAATC
ACTGA
APOE3-4 (SEQ ID NO: 21)
ATGAAGGTGCTGTGGGCTGCCCTGCTGGTGACTTTCCTGGCTGGCTGCCAGGCTAAGGTGGAGCAGGCTGTGG
AGACTGAACCTGAGCCTGAGCTGAGGCAGCAGACTGAGTGGCAGTCTGGCCAGAGGTGGGAGCTGGCCCTGGG
GAGGTTCTGGGACTACCTGAGGTGGGTGCAGACTCTGTCTGAGCAGGTGCAGGAGGAGCTGCTGAGCTCTCAG
GTGACCCAGGAGCTGAGGGCCCTGATGGATGAAACTATGAAGGAGCTGAAAGCCTACAAGTCTGAGCTGGAGG
AGCAGCTGACCCCTGTGGCTGAGGAGACCAGGGCCAGGCTGTCTAAGGAGCTGCAGGCTGCCCAGGCCAGGCT
GGGGGCTGATATGGAAGATGTGTGTGGCAGGCTGGTGCAGTACAGGGGGGAGGTGCAGGCTATGCTGGGCCAG
TCTACTGAGGAGCTGAGAGTGAGGCTGGCCAGCCATCTGAGGAAGCTGAGGAAGAGGCTGCTGAGGGATGCTG
ATGATCTGCAGAAGAGACTGGCTGTGTACCAGGCTGGGGCTAGGGAGGGGGCTGAGAGGGGCCTGTCTGCCAT
CAGGGAGAGGCTGGGCCCCCTGGTGGAGCAGGGCAGGGTGAGGGCTGCCACTGTGGGCTCTCTGGCTGGCCAG
CCCCTGCAGGAGAGGGCCCAGGCCTGGGGGGAGAGGCTGAGGGCTAGGATGGAAGAGATGGGCAGCAGGACCA
GGGACAGGCTGGATGAAGTGAAGGAGCAGGTGGCTGAGGTGAGGGCCAAGCTGGAGGAGCAGGCCCAGCAGAT
TAGGCTGCAGGCTGAGGCCTTCCAGGCCAGGCTGAAGTCTTGGTTTGAGCCACTGGTGGAGGACATGCAGAGG
CAGTGGGCTGGCCTGGTGGAGAAGGTGCAGGCTGCTGTGGGCACCTCTGCTGCCCCTGTGCCTTCTGACAACC
ACTGA
APOE3-5 (SEQ ID NO: 22)
ATGAAGGTCCTGTGGGCTGCCCTGCTGGTGACCTTCCTGGCTGGCTGCCAGGCCAAGGTGGAGCAGGCTGTGG
AGACTGAGCCTGAGCCTGAGCTGAGGCAGCAGACTGAGTGGCAGTCTGGGCAGAGGTGGGAGCTGGCCCTGGG
CAGGTTCTGGGACTACCTGAGGTGGGTGCAGACTCTGTCTGAACAGGTGCAGGAGGAGCTGCTGAGCAGCCAG
GTGACCCAGGAGCTGAGGGCCCTGATGGATGAGACCATGAAGGAGCTGAAGGCCTATAAGTCTGAACTGGAGG
AGCAGCTGACCCCTGTGGCTGAGGAGACTAGGGCCAGGCTGAGCAAGGAGCTGCAGGCTGCTCAGGCCAGACT
GGGGGCTGACATGGAGGATGTCTGTGGCAGACTGGTGCAGTACAGGGGGGAGGTGCAGGCTATGCTGGGGCAG
AGCACTGAGGAGCTGAGGGTGAGGCTGGCCAGCCACCTGAGGAAGCTGAGGAAGAGGCTGCTGAGGGATGCTG
ATGATCTGCAGAAGAGACTGGCTGTGTACCAGGCTGGGGCCAGAGAGGGGGCTGAGAGGGGGCTGTCTGCCAT
TAGGGAGAGACTGGGCCCCCTGGTGGAGCAGGGCAGGGTGAGGGCTGCCACTGTGGGCTCTCTGGCTGGCCAG
CCTCTGCAGGAGAGGGCTCAGGCCTGGGGGGAGAGGCTGAGGGCTAGAATGGAGGAGATGGGGAGCAGGACCA
GGGACAGGCTGGATGAGGTGAAAGAGCAGGTGGCTGAGGTCAGGGCCAAGCTGGAGGAGCAGGCCCAGCAGAT
CAGGCTGCAGGCTGAGGCTTTCCAGGCTAGGCTGAAGAGCTGGTTTGAGCCCCTGGTGGAGGATATGCAGAGG
CAGTGGGCTGGCCTGGTGGAGAAGGTGCAGGCTGCTGTGGGCACTTCTGCTGCTCCTGTGCCCTCTGATAACC
ACTGA
APOE3-6 (SEQ ID NO: 23)
ATGAAGGTGCTGTGGGCTGCCCTGCTGGTGACCTTCCTGGCTGGCTGCCAGGCCAAGGTGGAGCAGGCTGTGG
AGACTGAGCCTGAGCCTGAACTGAGGCAGCAGACTGAATGGCAGTCTGGCCAGAGGTGGGAGCTGGCCCTGGG
CAGATTCTGGGATTACCTGAGGTGGGTGCAGACCCTGTCTGAGCAGGTGCAGGAGGAACTGCTGAGCAGCCAG
GTGACTCAGGAGCTGAGGGCCCTGATGGATGAGACCATGAAGGAGCTGAAGGCCTATAAATCTGAGCTGGAGG
AGCAGCTGACCCCTGTGGCTGAGGAGACTAGGGCCAGACTGTCTAAGGAGCTGCAGGCTGCCCAGGCCAGGCT
GGGGGCTGACATGGAGGATGTGTGTGGCAGGCTGGTGCAGTACAGGGGGGAGGTGCAGGCCATGCTGGGCCAG
TCTACTGAGGAGCTGAGGGTGAGGCTGGCCAGCCACCTGAGGAAGCTGAGGAAGAGGCTGCTGAGGGATGCTG
ATGACCTGCAGAAGAGGCTGGCTGTGTACCAGGCTGGGGCCAGGGAGGGGGCTGAGAGGGGCCTGTCTGCCAT
CAGAGAGAGGCTGGGCCCCCTGGTGGAGCAGGGCAGGGTGAGGGCTGCCACTGTGGGCTCCCTGGCTGGCCAG
CCCCTGCAGGAGAGGGCCCAGGCCTGGGGGGAGAGGCTGAGGGCCAGGATGGAGGAGATGGGCAGCAGGACCA
GAGACAGGCTGGATGAGGTCAAGGAGCAGGTGGCTGAGGTGAGGGCCAAGCTGGAGGAGCAGGCCCAGCAGAT
CAGACTGCAGGCTGAGGCCTTCCAGGCTAGGCTGAAGTCTTGGTTTGAGCCACTGGTGGAGGACATGCAGAGG
CAGTGGGCTGGCCTGGTGGAAAAGGTGCAGGCTGCTGTGGGCACCTCTGCTGCCCCTGTGCCCTCTGACAATC
ACTGA
APOE3-7 (SEQ ID NO: 24)
ATGAAGGTGCTGTGGGCTGCCCTGCTGGTGACCTTCCTGGCTGGCTGCCAGGCTAAGGTGGAGCAGGCTGTGG
AGACTGAGCCTGAGCCTGAGCTGAGGCAGCAGACTGAGTGGCAGTCTGGGCAGAGGTGGGAGCTGGCCCTGGG
GAGGTTCTGGGACTACCTGAGGTGGGTGCAGACTCTGTCTGAGCAGGTGCAGGAGGAGCTGCTGAGCTCTCAG
GTGACTCAGGAGCTGAGGGCCCTGATGGATGAGACTATGAAGGAGCTGAAGGCTTACAAGTCTGAGCTGGAGG
AGCAGCTGACTCCTGTGGCTGAGGAGACCAGGGCCAGGCTGTCTAAGGAGCTGCAGGCTGCCCAGGCCAGACT
GGGGGCTGACATGGAGGATGTGTGTGGCAGGCTGGTGCAGTACAGGGGGGAGGTGCAGGCTATGCTGGGGCAG
AGCACTGAGGAGCTGAGGGTGAGGCTGGCCAGCCATCTGAGGAAGCTGAGGAAGAGGCTGCTGAGGGATGCTG
ATGACCTGCAGAAGAGACTGGCTGTGTATCAGGCTGGGGCCAGGGAAGGGGCTGAGAGGGGCCTGTCTGCCAT
CAGGGAGAGGCTGGGCCCCCTGGTGGAACAGGGGAGGGTGAGGGCTGCCACTGTGGGCAGCCTGGCTGGCCAG
CCCCTGCAGGAGAGGGCTCAGGCTTGGGGGGAGAGGCTGAGGGCCAGGATGGAGGAGATGGGCAGCAGGACCA
GGGATAGGCTGGATGAGGTGAAGGAGCAGGTGGCTGAGGTGAGGGCTAAGCTGGAGGAGCAGGCTCAGCAGAT
CAGGCTGCAGGCTGAGGCTTTCCAGGCCAGGCTGAAGAGCTGGTTTGAGCCCCTGGTGGAGGACATGCAGAGG
CAGTGGGCTGGCCTGGTGGAGAAGGTCCAGGCTGCTGTGGGCACTTCTGCTGCCCCTGTGCCCTCTGACAACC
ATTGA
APOE3-8 (SEQ ID NO: 25)
ATGAAGGTGCTGTGGGCTGCTCTGCTGGTGACCTTCCTGGCTGGCTGCCAGGCCAAGGTGGAGCAGGCTGTGG
AGACTGAGCCTGAGCCTGAGCTGAGGCAGCAGACTGAGTGGCAGTCTGGCCAGAGGTGGGAGCTGGCCCTGGG
GAGGTTCTGGGACTACCTGAGGTGGGTGCAGACCCTGTCTGAGCAGGTCCAGGAAGAGCTGCTGTCTTCTCAG
GTGACCCAGGAACTGAGGGCCCTGATGGATGAGACCATGAAGGAACTGAAGGCCTACAAGTCTGAGCTGGAGG
AGCAGCTGACCCCTGTGGCTGAGGAGACCAGGGCTAGGCTGAGCAAGGAGCTGCAGGCTGCTCAGGCCAGGCT
GGGGGCTGACATGGAGGATGTGTGTGGGAGGCTGGTGCAGTACAGGGGGGAGGTGCAGGCCATGCTGGGCCAG
AGCACTGAGGAGCTGAGGGTGAGACTGGCCAGCCATCTGAGGAAGCTGAGGAAGAGGCTGCTGAGGGATGCTG
ATGACCTGCAGAAGAGGCTGGCTGTGTACCAGGCTGGGGCCAGGGAGGGGGCTGAGAGGGGCCTGTCTGCTAT
TAGGGAGAGGCTGGGCCCCCTGGTGGAGCAGGGGAGGGTGAGGGCTGCCACTGTGGGCAGCCTGGCTGGCCAG
CCCCTGCAGGAGAGGGCCCAGGCCTGGGGGGAGAGGCTGAGGGCCAGGATGGAGGAGATGGGCAGCAGGACCA
GGGACAGGCTGGATGAGGTGAAGGAGCAGGTGGCTGAGGTGAGGGCCAAGCTGGAGGAGCAGGCCCAGCAGAT
CAGGCTGCAGGCTGAGGCCTTCCAGGCCAGGCTGAAGAGCTGGTTTGAGCCCCTGGTGGAGGACATGCAGAGG
CAGTGGGCTGGCCTGGTGGAGAAGGTGCAGGCTGCTGTGGGGACCTCTGCTGCCCCTGTGCCTTCTGACAACC
ACTGA
APOE3-9 (SEQ ID NO: 26)
ATGAAAGTGCTGTGGGCTGCTCTGCTGGTGACCTTCCTGGCTGGGTGCCAGGCCAAGGTGGAGCAGGCTGTGG
AGACTGAGCCTGAGCCTGAGCTGAGGCAGCAGACTGAGTGGCAGTCTGGCCAGAGGTGGGAGCTGGCCCTGGG
GAGGTTCTGGGACTACCTGAGGTGGGTGCAGACTCTGTCTGAGCAGGTGCAGGAGGAGCTGCTGTCTAGCCAG
GTGACCCAGGAGCTGAGAGCCCTGATGGATGAGACCATGAAGGAGCTGAAGGCCTACAAGTCTGAGCTGGAGG
AGCAGCTGACCCCTGTGGCTGAGGAGACCAGAGCTAGGCTGTCTAAGGAGCTGCAGGCTGCTCAGGCCAGGCT
GGGGGCTGACATGGAGGATGTGTGTGGCAGGCTGGTGCAGTATAGGGGGGAGGTCCAGGCCATGCTGGGGCAG
AGCACTGAGGAGCTGAGGGTGAGGCTGGCCAGCCATCTGAGGAAGCTGAGGAAGAGGCTGCTGAGGGATGCTG
ATGACCTGCAGAAGAGACTGGCTGTGTACCAGGCTGGGGCCAGGGAGGGGGCTGAGAGGGGCCTGTCTGCCAT
CAGGGAGAGGCTGGGGCCTCTGGTGGAGCAGGGCAGGGTGAGGGCTGCCACTGTGGGCTCTCTGGCTGGCCAG
CCCCTGCAGGAGAGGGCCCAGGCTTGGGGGGAGAGGCTGAGAGCCAGAATGGAGGAGATGGGCAGCAGGACCA
GGGACAGGCTGGATGAGGTGAAGGAGCAGGTGGCTGAGGTGAGGGCCAAGCTGGAGGAGCAGGCCCAGCAGAT
CAGGCTGCAGGCTGAGGCCTTTCAGGCCAGGCTGAAGAGCTGGTTTGAACCCCTGGTGGAGGACATGCAGAGA
CAGTGGGCTGGCCTGGTGGAGAAGGTGCAGGCTGCTGTGGGCACCTCTGCTGCCCCTGTGCCCTCTGATAACC
ACTGA
APOE3-10 (SEQ ID NO: 27)
ATGAAGGTGCTGTGGGCTGCCCTGCTGGTGACCTTTCTGGCTGGGTGCCAGGCTAAAGTGGAGCAGGCTGTGG
AGACTGAGCCTGAGCCTGAGCTGAGGCAGCAGACTGAGTGGCAGTCTGGCCAGAGGTGGGAGCTGGCTCTGGG
CAGGTTCTGGGACTATCTGAGGTGGGTGCAGACTCTGTCTGAACAGGTGCAGGAGGAGCTGCTGAGCTCTCAG
GTGACCCAGGAGCTGAGGGCTCTGATGGATGAGACTATGAAGGAACTGAAGGCCTACAAGTCTGAGCTGGAGG
AGCAGCTGACCCCTGTGGCTGAGGAGACTAGGGCTAGGCTGAGCAAGGAGCTGCAGGCTGCCCAGGCCAGACT
GGGGGCTGACATGGAGGATGTGTGTGGCAGGCTGGTGCAGTACAGGGGGGAGGTGCAGGCTATGCTGGGGCAG
AGCACTGAGGAGCTGAGGGTGAGGCTGGCCTCTCATCTGAGGAAGCTGAGGAAGAGGCTGCTGAGGGATGCTG
ATGACCTGCAGAAGAGGCTGGCTGTGTACCAGGCTGGGGCCAGGGAGGGGGCTGAGAGGGGCCTGTCTGCCAT
CAGGGAGAGGCTGGGCCCCCTGGTGGAGCAGGGGAGGGTGAGGGCTGCCACTGTGGGCAGCCTGGCTGGCCAG
CCCCTGCAGGAGAGGGCTCAGGCCTGGGGGGAGAGACTGAGGGCTAGAATGGAGGAGATGGGGAGCAGGACCA
GGGATAGGCTGGATGAGGTGAAGGAGCAGGTGGCTGAGGTGAGGGCCAAGCTGGAGGAGCAGGCCCAGCAGAT
CAGGCTGCAGGCTGAGGCCTTTCAGGCCAGGCTGAAGAGCTGGTTTGAGCCCCTGGTGGAAGATATGCAGAGG
CAGTGGGCTGGCCTGGTGGAGAAGGTGCAGGCTGCTGTGGGCACCTCTGCTGCCCCTGTGCCCTCTGACAACC
ACTGA
APOE3-11 (SEQ ID NO: 28)
ATGAAGGTGCTGTGGGCTGCCCTGCTGGTGACCTTTCTGGCTGGGTGCCAGGCTAAGGTGGAGCAGGCTGTGG
AGACTGAGCCTGAGCCTGAACTGAGGCAGCAGACTGAGTGGCAGTCTGGCCAGAGGTGGGAGCTGGCCCTGGG
CAGGTTCTGGGACTATCTGAGGTGGGTGCAGACCCTGTCTGAGCAGGTGCAGGAAGAGCTGCTGAGCTCTCAG
GTGACTCAGGAGCTGAGGGCCCTGATGGATGAGACCATGAAGGAGCTGAAGGCCTACAAGTCTGAGCTGGAGG
AGCAGCTGACTCCTGTGGCTGAAGAGACCAGGGCCAGGCTGAGCAAGGAGCTGCAGGCTGCCCAGGCTAGGCT
GGGGGCTGATATGGAGGATGTGTGTGGGAGACTGGTGCAGTACAGGGGGGAGGTCCAGGCTATGCTGGGCCAG
AGCACTGAGGAGCTGAGGGTGAGGCTGGCCTCTCACCTGAGGAAGCTGAGGAAGAGGCTGCTGAGGGATGCTG
ATGACCTGCAGAAGAGACTGGCTGTGTACCAGGCTGGGGCTAGGGAGGGGGCTGAGAGGGGGCTGTCTGCTAT
CAGGGAGAGGCTGGGGCCTCTGGTGGAGCAGGGCAGGGTGAGGGCTGCCACTGTGGGCAGCCTGGCTGGCCAG
CCCCTGCAGGAGAGGGCCCAGGCCTGGGGGGAGAGGCTGAGGGCCAGGATGGAGGAGATGGGGAGCAGGACCA
GGGATAGGCTGGATGAGGTGAAGGAGCAGGTGGCTGAGGTGAGGGCTAAGCTGGAGGAGCAGGCCCAGCAGAT
TAGGCTGCAGGCTGAGGCCTTTCAGGCTAGGCTGAAGTCTTGGTTTGAGCCCCTGGTGGAGGACATGCAGAGG
CAGTGGGCTGGCCTGGTGGAGAAGGTGCAGGCTGCTGTGGGCACCTCTGCTGCCCCTGTGCCCTCTGACAACC
ACTGA
APOE3-12 (SEQ ID NO: 29)
ATGAAGGTGCTGTGGGCTGCCCTGCTGGTGACTTTCCTGGCTGGCTGCCAGGCCAAGGTGGAGCAGGCTGTGG
AGACTGAGCCTGAGCCTGAGCTGAGGCAGCAGACTGAGTGGCAGTCTGGCCAGAGGTGGGAGCTGGCCCTGGG
CAGGTTCTGGGACTACCTGAGGTGGGTGCAGACCCTGTCTGAGCAGGTGCAGGAGGAGCTGCTGAGCAGCCAG
GTGACTCAGGAGCTGAGGGCCCTGATGGATGAGACCATGAAGGAGCTGAAGGCCTACAAATCTGAGCTGGAGG
AGCAGCTGACCCCTGTGGCTGAGGAGACCAGGGCCAGGCTGAGCAAGGAGCTGCAGGCTGCTCAGGCCAGGCT
GGGGGCTGATATGGAGGATGTGTGTGGCAGGCTGGTGCAGTACAGGGGGGAGGTGCAGGCCATGCTGGGCCAG
AGCACTGAGGAGCTGAGGGTGAGGCTGGCCAGCCATCTGAGGAAGCTGAGGAAGAGACTGCTGAGGGATGCTG
ATGATCTGCAGAAGAGGCTGGCTGTGTACCAGGCTGGGGCCAGGGAGGGGGCTGAGAGGGGCCTGTCTGCCAT
CAGGGAGAGGCTGGGCCCCCTGGTGGAGCAGGGCAGGGTGAGGGCTGCCACTGTGGGCAGCCTGGCTGGCCAG
CCTCTGCAGGAGAGGGCCCAGGCTTGGGGGGAGAGACTGAGGGCCAGGATGGAAGAGATGGGGTCTAGGACCA
GGGATAGGCTGGATGAGGTGAAGGAGCAGGTGGCTGAGGTGAGGGCCAAGCTGGAGGAACAGGCTCAGCAGAT
CAGGCTGCAGGCTGAGGCCTTCCAGGCCAGACTGAAGAGCTGGTTTGAGCCTCTGGTGGAGGACATGCAGAGG
CAGTGGGCTGGCCTGGTGGAGAAAGTGCAGGCTGCTGTGGGCACTTCTGCTGCCCCTGTGCCCTCTGACAACC
ACTGA
APOE3-13 (SEQ ID NO: 30)
ATGAAGGTGCTGTGGGCTGCCCTGCTGGTGACTTTCCTGGCTGGCTGCCAGGCCAAGGTGGAGCAGGCTGTGG
AAACTGAGCCTGAGCCTGAGCTGAGGCAGCAGACTGAGTGGCAGTCTGGCCAGAGGTGGGAGCTGGCTCTGGG
CAGGTTTTGGGACTACCTGAGGTGGGTGCAGACCCTGTCTGAGCAGGTGCAGGAGGAGCTGCTGAGCAGCCAG
GTGACCCAGGAGCTGAGAGCCCTGATGGATGAGACCATGAAGGAGCTGAAGGCTTACAAGTCTGAGCTGGAGG
AGCAGCTGACTCCTGTGGCTGAGGAGACCAGGGCTAGGCTGTCTAAGGAGCTGCAGGCTGCTCAGGCTAGGCT
GGGGGCTGACATGGAGGATGTGTGTGGCAGACTGGTGCAGTACAGGGGGGAGGTGCAGGCTATGCTGGGCCAG
AGCACTGAGGAACTGAGGGTGAGGCTGGCCAGCCACCTGAGGAAGCTGAGGAAGAGGCTGCTGAGGGATGCTG
ATGACCTGCAGAAGAGGCTGGCTGTGTACCAGGCTGGGGCCAGGGAGGGGGCTGAGAGGGGCCTGTCTGCCAT
CAGGGAGAGGCTGGGGCCTCTGGTGGAGCAGGGCAGGGTCAGGGCTGCCACTGTGGGCAGCCTGGCTGGCCAG
CCCCTGCAGGAGAGGGCCCAGGCTTGGGGGGAGAGGCTGAGAGCTAGGATGGAGGAGATGGGCAGCAGGACTA
GGGATAGGCTGGATGAGGTGAAGGAGCAGGTGGCTGAGGTGAGGGCTAAGCTGGAGGAGCAGGCCCAGCAGAT
CAGGCTGCAGGCTGAGGCCTTTCAGGCCAGGCTGAAGAGCTGGTTTGAGCCTCTGGTGGAGGACATGCAGAGG
CAGTGGGCTGGCCTGGTGGAGAAGGTGCAGGCTGCTGTGGGGACCTCTGCTGCCCCTGTGCCCTCTGACAACC
ATTGA
APOE3-15 (SEQ ID NO: 31)
ATGAAGGTGCTGTGGGCTGCTCTGCTGGTGACCTTCCTGGCTGGCTGCCAGGCTAAGGTGGAGCAGGCTGTGG
AGACTGAGCCTGAGCCTGAGCTGAGGCAGCAGACTGAGTGGCAGTCTGGGCAGAGGTGGGAGCTGGCCCTGGG
CAGGTTCTGGGACTACCTGAGGTGGGTGCAGACCCTGTCTGAGCAGGTGCAGGAGGAGCTGCTGTCTAGCCAG
GTGACCCAGGAACTGAGGGCCCTGATGGATGAGACCATGAAGGAGCTGAAGGCCTACAAGTCTGAGCTGGAGG
AGCAGCTGACCCCTGTGGCTGAGGAGACCAGGGCCAGGCTGTCTAAGGAGCTGCAGGCTGCCCAGGCCAGGCT
GGGGGCTGACATGGAGGATGTGTGTGGCAGGCTGGTGCAGTATAGGGGGGAGGTGCAGGCTATGCTGGGCCAG
AGCACTGAGGAGCTGAGGGTCAGGCTGGCCAGCCACCTGAGGAAGCTGAGGAAGAGGCTGCTGAGGGATGCTG
ATGACCTGCAGAAGAGGCTGGCTGTGTACCAGGCTGGGGCCAGGGAGGGGGCTGAGAGGGGCCTGTCTGCCAT
CAGGGAGAGGCTGGGCCCTCTGGTGGAGCAGGGGAGGGTGAGGGCTGCCACTGTGGGCAGCCTGGCTGGCCAG
CCCCTGCAGGAAAGGGCCCAGGCCTGGGGGGAGAGACTGAGGGCCAGGATGGAGGAGATGGGCAGCAGGACCA
GAGACAGGCTGGATGAGGTGAAGGAGCAGGTGGCTGAGGTGAGGGCCAAGCTGGAGGAGCAGGCCCAGCAGAT
TAGGCTGCAGGCTGAGGCCTTCCAGGCCAGGCTGAAGTCTTGGTTTGAGCCCCTGGTGGAAGATATGCAGAGG
CAGTGGGCTGGCCTGGTGGAGAAGGTGCAGGCTGCTGTGGGGACCTCTGCTGCTCCTGTGCCCTCTGACAACC
ACTGA
ApoE Polypeptide
Mature APOE3 SEQ ID NO: 32 (amino acid sequence)
KVEQAVETEPEPELRQQTEWQSGQRWELALGREWDYLRWVQTLSEQVQEELLSSQVTQELRALMDETMKELKA
YKSELEEQLTPVAEETRARLSKELQAAQARLGADMEDVCGRLVQYRGEVQAMLGQSTEELRVRLASHLRKLRK
RLLRDADDLQKRLAVYQAGAREGAERGLSAIRERLGPLVEQGRVRAATVGSLAGQPLQERAQAWGERLRARME
EMGSRTRDRLDEVKEQVAEVRAKLEEQAQQIRLQAEAFQARLKSWFEPLVEDMQRQWAGLVEKVQAAVGTSAA
PVPSDNH
Mature APOE3ch (SEQ ID NO: 33) (amino acid sequence)
KVEQAVETEPEPELRQQTEWQSGQRWELALGRFWDYLRWVQTLSEQVQEELLSSQVTQELRALMDETMKELKA
YKSELEEQLTPVAEETRARLSKELQAAQARLGADMEDVCGRLVQYSGEVQAMLGQSTEELRVRLASHLRKLRK
RLLRDADDLQKRLAVYQAGAREGAERGLSAIRERLGPLVEQGRVRAATVGSLAGQPLQERAQAWGERLRARME
EMGSRTRDRLDEVKEQVAEVRAKLEEQAQQIRLQAEAFQARLKSWFEPLVEDMQRQWAGLVEKVQAAVGTSAA
PVPSDNH
Full length APOE (SEQ ID NO: 34) (amino acid sequence)
MKVLWAALLVTFLAGCQAKVEQAVETEPEPELRQQTEWQSGQRWELALGRFWDYLRWVQTLSEQVQEELLSSQ
VTQELRALMDETMKELKAYKSELEEQLTPVAEETRARLSKELQAAQARLGADMEDVCGRLVQYRGEVQAMLGQ
STEELRVRLASHLRKLRKRLLRDADDLQKRLAVYQAGAREGAERGLSAIRERLGPLVEQGRVRAATVGSLAGQ
PLQERAQAWGERLRARMEEMGSRTRDRLDEVKEQVAEVRAKLEEQAQQIRLQAEAFQARLKSWFEPLVEDMQR
QWAGLVEKVQAAVGTSAAPVPSDNH
Full length APOE3ch (SEQ ID NO: 35) (amino acid sequence)
MKVLWAALLVTFLAGCQAKVEQAVETEPEPELRQQTEWQSGQRWELALGRFWDYLRWVQTLSEQVQEELLSSQ
VTQELRALMDETMKELKAYKSELEEQLTPVAEETRARLSKELQAAQARLGADMEDVCGRLVQYSGEVQAMLGQ
STEELRVRLASHLRKLRKRLLRDADDLQKRLAVYQAGAREGAERGLSAIRERLGPLVEQGRVRAATVGSLAGQ
PLQERAQAWGERLRARMEEMGSRTRDRLDEVKEQVAEVRAKLEEQAQQIRLQAEAFQARLKSWFEPLVEDMQR
QWAGLVEKVQAAVGTSAAPVPSDNH
Signal
Sp7 (SEQ ID NO: 36)
MAFLWLLSCWALLGTTFG
Sp7_1 (SEQ ID NO: 37)
ATGGCCTTCCTGTGGCTGCTGTCCTGCTGGGCACTGCTGGGCACCACATTTGGC
Sp7_2 (SEQ ID NO: 38)
ATGGCCTTTCTGTGGCTGCTGTCCTGCTGGGCCCTGCTGGGGACCACCTTTGGC
Sp7_3 (SEQ ID NO: 39)
ATGGCCTTCCTGTGGCTGCTGAGCTGCTGGGCCCTGCTGGGGACCACCTTTGGG
Sp7_4 (SEQ ID NO: 40)
ATGGCCTTCCTGTGGCTCCTGAGCTGCTGGGCCCTCCTGGGGACCACCTTTGGG
Sp7_5 (SEQ ID NO: 41)
ATGGCCTTCCTGTGGCTGCTGAGCTGCTGGGCCCTGCTGGGGACCACATTTGGC
AHSG (SEQ ID NO: 42)
MKSLVLLLCLAQLWGCHS
AHSG (SEQ ID NO: 43)
ATGAAGTCCCTCGTCCTGCTCCTTTGTCTTGCTCAGCTCTGGGGCTGCCACTCA
CD300 (SEQ ID NO: 44)
MWLPWALLLLWVPGCFA
CD300 (SEQ ID NO: 45)
ATGTGGCTGCCTTGGGCTCTGTTGCTTCTCTGGGTCCCAGGATGTTTTGCT
GLA (SEQ ID NO: 46)
MQLRNPELHLGCALALRFLALVSWDIPGARA
GLA (SEQ ID NO: 47)
ATGCAGCTGAGGAACCCAGAACTACATCTGGGCTGCGCGCTTGCGCTTCGCTTCCTGGCCCTCGTTTCCTGGG
ACATCCCTGGGGCTAGAGCA
LAMP1 (SEQ ID NO: 48)
MAAPGSARRPLLLLLLLLLLGLMHCASA
LAMP1 (SEQ ID NO: 49)
ATGGCGGCCCCCGGCAGCGCCCGGCGACCCCTGCTGCTGCTACTGCTGTTGCTGCTGCTCGGCCTCATGCATT
GTGCGTCAGCA
Notch2 (SEQ ID NO: 50)
MPALRPALLWALLALWLCCAAPAHA
Notch2 (SEQ ID NO: 51)
ATGCCCGCCCTGCGCCCCGCTCTGCTGTGGGCGCTGCTGGCGCTCTGGCTGTGCTGCGCGGCCCCCGCGCATG
CA
ORM1 (SEQ ID NO: 52)
MALSWVLTVLSLLPLLEA
ORM1 (SEQ ID NO: 53)
ATGGCGCTGTCCTGGGTTCTTACAGTCCTGAGCCTCCTACCTCTGCTGGAAGCC
TF (SEQ ID NO: 54)
MRLAVGALLVCAVLGLCLA
TF (SEQ ID NO: 55)
ATGAGGCTCGCCGTGGGAGCCCTGCTGGTCTGCGCCGTCCTGGGGCTGTGTCTGGCT
ApoE3 signal amino acid (SEQ ID NO: 56)
MKVLWAALLVTFLAGCQA
ApoE3 signal nucleotide (SEQ ID NO: 57)
ATGAAGGTTCTGTGGGCTGCGTTGCTGGTCACATTCCTGGCAGGATGCCAGGCC
Vector Components
ApoE HCR-1 Enhancer (SEQ ID NO: 58)
AGGCTCAGAGGCACACAGGAGTTTCTGGGCTCACCCTGCCCCCTTCCAACCCCTCAGTTCCCATCCTCCAGCA
GCTGTTTGTGTGCTGCCTCTGAAGTCCACACTGAACAAACTTCAGCCTACTCATGTCCCTAAAATGGGCAAAC
ATTGCAAGCAGCAAACAGCAAACACACAGCCCTCCCTGCCTGCTGACCTTGGAGCTGGGGCAGAGGTCAGAGA
CCTCTCTGGGCCCATGCCACCTCCAACATCCACTCGACCCCTTGGAATTTCGGTGGAGAGGAGCAGAGGTTGT
CCTGGCGTGGTTTAGGTAGTGTGAGAGGG
hAAT Promoter (SEQ ID NO: 59)
GATCTTGCTACCAGTGGAACAGCCACTAAGGATTCTGCAGTGAGAGCAGAGGGCCAGCTAAGTGGTACTCTCC
CAGAGACTGTCTGACTCACGCCACCCCCTCCACCTTGGACACAGGACGCTGTGGTTTCTGAGCCAGGTACAAT
GACTCCTTTCGGTAAGTGCAGTGGAAGCTGTACACTGCCCAGGCAAAGCGTCCGGGCAGCGTAGGCGGGCGAC
TCAGATCCCAGCCAGTGGACTTAGCCCCTGTTTGCTCCTCCGATAACTGGGGTGACCTTGGTTAATATTCACC
AGCAGCCTCCCCCGTTGCCCCTCTGGATCCACTGCTTAAATACGGACGAGGACAGGGCCCTGTCTCCTCAGCT
TCAGGCACCACCACTGACCTGGGACAGTGAAT
modified HBB2 Intron: (SEQ ID NO: 60)
GATCCTGAGAACTTCAGGGTGAGTCTATGGGACCCTTGATGTTTTCTTTCCCCTTCTTTTCTATGGTTAAGTT
CATGTCATAGGAAGGGGAGAAGTAACAGGGTACACATATTGACCAAATCAGGGTAATTTTGCATTTGTAATTT
TAAAAAATGCTTTCTTCTTTTAATATACTTTTTTGTTTATCTTATTTCTAATACTTTCCCTAATCTCTTTCTT
TCAGGGCAATAATGATACAATGTATCATGCCTCTTTGCACCATTCTAAAGAATAACAGTGATAATTTCTGGGT
TAAGGCAATAGCAATATTTCTGCATATAAATATTTCTGCATATAAATTGTAACTGATGTAAGAGGTTTCATAT
TGCTAATAGCAGCTACAATCCAGCTACCATTCTGCTTTTATTTTCTGGTTGGGATAAGGCTGGATTATTCTGA
GTCCAAGCTAGGCCCTTTTGCTAATCTTGTTCATACCTCTTATCTTCCTCCCACAGCTCCTGGGCAACCTGCT
GGTCTCTCTGCTGGCCCATCACTTTGGCAAAG
Syn-PolyA: (SEQ ID NO: 61)
CTACTGTGCCTTCTAGTTGCCAGCCATCTGTTGTTTGCCCCTCCCCCTTGCCTTCCTTGACCCTGGAAGGTGC
CACTCCCACTGTCCTTTCCTAATAAAATGAGGAAATTGCATCACATTGTCTGAGTAGGTGTCATTCTATTCTG
GGGGGTGGGGTGGGGCAGGACAGCAAGGGGGAGGATTGGGAAGACAATAGCAGGCATGCTGGGGATGCAGTGG
GCTCTATGG
BGH-PolyA: (SEQ ID NO: 62)
CGACTGTGCCTTCTAGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAGGTGTCATTCTATTCTG
GGGGGTGGGGTGGGGCAGGACAGCAAGGGGGAGGATTGGGAAGACAATAGCAGGCATGCTGGGGATGCGGTGG
GCTCTATGG
Mature ApoE
Mature encoding sequence for ApoE3-3 (SEQ ID NO: 63)
AAAGTGGAGCAGGCTGTGGAGACTGAGCCTGAGCCAGAGCTGAGGCAGCAGACTGAGTGGCAGTCTGGCCAGA
GATGGGAGCTGGCTCTGGGCAGGTTCTGGGACTACCTGAGGTGGGTGCAGACCCTGTCTGAGCAGGTGCAGGA
GGAGCTGCTGTCTTCTCAGGTGACCCAGGAACTGAGGGCCCTGATGGATGAGACTATGAAGGAGCTGAAGGCC
TACAAGTCTGAGCTGGAGGAGCAGCTGACCCCTGTGGCTGAGGAGACCAGGGCCAGGCTGAGCAAGGAGCTGC
AGGCTGCCCAGGCCAGACTGGGGGCTGACATGGAGGATGTGTGTGGCAGACTGGTGCAGTACAGGGGGGAGGT
GCAGGCCATGCTGGGGCAGTCTACTGAGGAGCTGAGGGTGAGGCTGGCCAGCCACCTGAGGAAGCTGAGGAAG
AGGCTGCTGAGGGATGCTGATGACCTGCAGAAGAGGCTGGCTGTGTATCAGGCTGGGGCTAGGGAGGGGGCTG
AGAGGGGCCTGTCTGCCATCAGGGAGAGGCTGGGGCCTCTGGTGGAGCAGGGCAGGGTGAGGGCTGCCACTGT
GGGGAGCCTGGCTGGCCAGCCTCTGCAGGAGAGGGCTCAGGCCTGGGGGGAGAGGCTGAGGGCCAGGATGGAG
GAGATGGGGAGCAGGACCAGGGACAGGCTGGATGAGGTGAAGGAGCAGGTGGCTGAGGTGAGGGCCAAGCTGG
AAGAGCAGGCCCAGCAGATCAGGCTGCAGGCTGAGGCCTTCCAGGCCAGGCTGAAGAGCTGGTTTGAGCCCCT
GGTGGAGGACATGCAGAGGCAGTGGGCTGGCCTGGTGGAGAAGGTGCAGGCTGCTGTGGGCACCTCTGCTGCC
CCTGTGCCCTCTGACAATCACTGA
Mature encoding sequence for ApoE3-9 (SEQ ID NO: 64)
AAGGTGGAGCAGGCTGTGGAGACTGAGCCTGAGCCTGAGCTGAGGCAGCAGACTGAGTGGCAGTCTGGCCAGA
GGTGGGAGCTGGCCCTGGGGAGGTTCTGGGACTACCTGAGGTGGGTGCAGACTCTGTCTGAGCAGGTGCAGGA
GGAGCTGCTGTCTAGCCAGGTGACCCAGGAGCTGAGAGCCCTGATGGATGAGACCATGAAGGAGCTGAAGGCC
TACAAGTCTGAGCTGGAGGAGCAGCTGACCCCTGTGGCTGAGGAGACCAGAGCTAGGCTGTCTAAGGAGCTGC
AGGCTGCTCAGGCCAGGCTGGGGGCTGACATGGAGGATGTGTGTGGCAGGCTGGTGCAGTATAGGGGGGAGGT
CCAGGCCATGCTGGGGCAGAGCACTGAGGAGCTGAGGGTGAGGCTGGCCAGCCATCTGAGGAAGCTGAGGAAG
AGGCTGCTGAGGGATGCTGATGACCTGCAGAAGAGACTGGCTGTGTACCAGGCTGGGGCCAGGGAGGGGGCTG
AGAGGGGCCTGTCTGCCATCAGGGAGAGGCTGGGGCCTCTGGTGGAGCAGGGCAGGGTGAGGGCTGCCACTGT
GGGCTCTCTGGCTGGCCAGCCCCTGCAGGAGAGGGCCCAGGCTTGGGGGGAGAGGCTGAGAGCCAGAATGGAG
GAGATGGGCAGCAGGACCAGGGACAGGCTGGATGAGGTGAAGGAGCAGGTGGCTGAGGTGAGGGCCAAGCTGG
AGGAGCAGGCCCAGCAGATCAGGCTGCAGGCTGAGGCCTTTCAGGCCAGGCTGAAGAGCTGGTTTGAACCCCT
GGTGGAGGACATGCAGAGACAGTGGGCTGGCCTGGTGGAGAAGGTGCAGGCTGCTGTGGGCACCTCTGCTGCC
CCTGTGCCCTCTGATAACCACTGA
Mature encoding sequence for ApoE3-15 (SEQ ID NO: 65)
AAGGTGGAGCAGGCTGTGGAGACTGAGCCTGAGCCTGAGCTGAGGCAGCAGACTGAGTGGCAGTCTGGGCAGA
GGTGGGAGCTGGCCCTGGGCAGGTTCTGGGACTACCTGAGGTGGGTGCAGACCCTGTCTGAGCAGGTGCAGGA
GGAGCTGCTGTCTAGCCAGGTGACCCAGGAACTGAGGGCCCTGATGGATGAGACCATGAAGGAGCTGAAGGCC
TACAAGTCTGAGCTGGAGGAGCAGCTGACCCCTGTGGCTGAGGAGACCAGGGCCAGGCTGTCTAAGGAGCTGC
AGGCTGCCCAGGCCAGGCTGGGGGCTGACATGGAGGATGTGTGTGGCAGGCTGGTGCAGTATAGGGGGGAGGT
GCAGGCTATGCTGGGCCAGAGCACTGAGGAGCTGAGGGTCAGGCTGGCCAGCCACCTGAGGAAGCTGAGGAAG
AGGCTGCTGAGGGATGCTGATGACCTGCAGAAGAGGCTGGCTGTGTACCAGGCTGGGGCCAGGGAGGGGGCTG
AGAGGGGCCTGTCTGCCATCAGGGAGAGGCTGGGCCCTCTGGTGGAGCAGGGGAGGGTGAGGGCTGCCACTGT
GGGCAGCCTGGCTGGCCAGCCCCTGCAGGAAAGGGCCCAGGCCTGGGGGGAGAGACTGAGGGCCAGGATGGAG
GAGATGGGCAGCAGGACCAGAGACAGGCTGGATGAGGTGAAGGAGCAGGTGGCTGAGGTGAGGGCCAAGCTGG
AGGAGCAGGCCCAGCAGATTAGGCTGCAGGCTGAGGCCTTCCAGGCCAGGCTGAAGTCTTGGTTTGAGCCCCT
GGTGGAAGATATGCAGAGGCAGTGGGCTGGCCTGGTGGAGAAGGTGCAGGCTGCTGTGGGGACCTCTGCTGCT
CCTGTGCCCTCTGACAACCACTGA
Mature encoding sequence for ApoE3ch-9 (SEQ ID NO: 66)
AAGGTGGAGCAGGCTGTGGAGACTGAGCCAGAGCCTGAGCTGAGGCAGCAGACTGAATGGCAGTCTGGCCAGA
GGTGGGAGCTGGCCCTGGGCAGGTTCTGGGACTACCTGAGATGGGTGCAGACCCTGTCTGAGCAGGTCCAGGA
GGAGCTGCTGTCTTCTCAGGTGACCCAGGAGCTGAGGGCCCTGATGGATGAGACTATGAAGGAGCTGAAGGCT
TACAAGTCTGAGCTGGAGGAGCAGCTGACCCCTGTGGCTGAGGAGACCAGGGCCAGACTGAGCAAGGAGCTGC
AGGCTGCTCAGGCCAGGCTGGGGGCTGACATGGAGGATGTGTGTGGCAGGCTGGTGCAGTATTCTGGGGAGGT
GCAGGCTATGCTGGGCCAGAGCACTGAGGAACTGAGGGTGAGGCTGGCCTCTCATCTGAGGAAGCTGAGGAAG
AGGCTGCTGAGGGATGCTGATGACCTGCAGAAGAGGCTGGCTGTGTATCAGGCTGGGGCCAGGGAGGGGGCTG
AGAGGGGGCTGTCTGCTATCAGGGAGAGGCTGGGCCCCCTGGTGGAGCAGGGGAGAGTGAGGGCTGCCACTGT
GGGGTCTCTGGCTGGCCAGCCCCTGCAGGAGAGGGCCCAGGCCTGGGGGGAGAGGCTGAGGGCTAGAATGGAG
GAGATGGGGTCTAGGACCAGGGACAGGCTGGATGAGGTCAAGGAACAGGTGGCTGAGGTCAGGGCCAAGCTGG
AGGAGCAGGCCCAGCAGATTAGGCTGCAGGCTGAGGCCTTTCAGGCCAGGCTGAAGAGCTGGTTTGAGCCCCT
GGTGGAGGACATGCAGAGGCAGTGGGCTGGCCTGGTGGAGAAGGTGCAGGCTGCTGTGGGCACTTCTGCTGCC
CCTGTGCCCTCTGACAACCATTGA
Mature encoding sequence for ApoE3ch-3 (SEQ ID NO: 67)
AAGGTGGAGCAGGCTGTGGAGACTGAGCCTGAGCCTGAGCTGAGGCAGCAGACTGAGTGGCAGTCTGGGCAGA
GGTGGGAGCTGGCCCTGGGGAGGTTCTGGGACTACCTGAGGTGGGTGCAGACTCTGTCTGAGCAGGTGCAGGA
GGAGCTGCTGTCTAGCCAGGTGACCCAGGAGCTGAGGGCCCTGATGGATGAGACCATGAAGGAGCTGAAGGCC
TACAAGTCTGAGCTGGAAGAGCAGCTGACCCCTGTGGCTGAGGAGACCAGGGCTAGGCTGAGCAAGGAACTGC
AGGCTGCTCAGGCCAGGCTGGGGGCTGACATGGAAGATGTGTGTGGGAGGCTGGTGCAGTACTCTGGGGAGGT
GCAGGCCATGCTGGGCCAGAGCACTGAGGAGCTGAGGGTGAGGCTGGCCAGCCACCTGAGGAAGCTGAGGAAG
AGGCTGCTGAGGGATGCTGATGACCTGCAGAAGAGGCTGGCTGTGTACCAGGCTGGGGCCAGGGAGGGGGCTG
AGAGGGGCCTGTCTGCTATCAGGGAGAGGCTGGGCCCCCTGGTGGAGCAGGGCAGGGTGAGGGCTGCCACTGT
GGGCAGCCTGGCTGGGCAGCCTCTGCAGGAGAGGGCCCAGGCCTGGGGGGAGAGGCTGAGAGCCAGGATGGAG
GAGATGGGCTCTAGGACTAGGGACAGGCTGGATGAGGTGAAGGAGCAGGTGGCTGAGGTGAGGGCCAAGCTGG
AGGAGCAGGCTCAGCAGATCAGGCTGCAGGCTGAGGCTTTCCAGGCCAGGCTGAAGTCTTGGTTTGAGCCCCT
GGTGGAGGATATGCAGAGGCAGTGGGCTGGCCTGGTGGAGAAGGTGCAGGCTGCTGTGGGCACCTCTGCTGCC
CCTGTGCCCTCTGACAACCATTGA
Signal
SNY1 Amino Acid (SEQ ID NO: 68)
MKFALVAALALLLFLWSSCRA
SNY2 Amino Acid (SEQ ID NO: 69)
MKFALVAALALLLFLWGCHS
SNY3 Amino Acid (SEQ ID NO: 70)
MKWVTLLTLLLLLLFLWGCHS
SNY4 Amino Acid (SEQ ID NO: 71)
MKWVTLLTLLLLLLFLWSSCRA
SNY1 encoding nuclei acid (SEQ ID NO: 72)
ATGAAGTTCGCCCTGGTGGCCGCCCTGGCCCTGCTGCTGTTCCTGTGGAGCAGCTGCAGGGCC
SNY2 encoding nuclei acid (SEQ ID NO: 73)
ATGAAGTTCGCCCTGGTGGCCGCCCTGGCCCTGCTGCTGTTCCTGTGGGGCTGCCACAGC
SNY3 encoding nuclei acid (SEQ ID NO: 74)
ATGAAGTGGGTGACCCTGCTGACCCTGCTGCTGCTGCTGCTGTTCCTGTGGGGCTGCCACAGC
SNY4 encoding nuclei acid (SEQ ID NO: 75)
ATGAAGTGGGTGACCCTGCTGACCCTGCTGCTGCTGCTGCTGTTCCTGTGGAGCAGCTGCAGGGCC
Expression cassettes
ApoE3-3 Expression cassette (SEQ ID NO: 76)
GCGGCCTAGTAGGCTCAGAGGCACACAGGAGTTTCTGGGCTCACCCTGCCCCCTTCCAACCCCTCAGTTCCCA
TCCTCCAGCAGCTGTTTGTGTGCTGCCTCTGAAGTCCACACTGAACAAACTTCAGCCTACTCATGTCCCTAAA
ATGGGCAAACATTGCAAGCAGCAAACAGCAAACACACAGCCCTCCCTGCCTGCTGACCTTGGAGCTGGGGCAG
AGGTCAGAGACCTCTCTGGGCCCATGCCACCTCCAACATCCACTCGACCCCTTGGAATTTCGGTGGAGAGGAG
CAGAGGTTGTCCTGGCGTGGTTTAGGTAGTGTGAGAGGGGTACCCGGGGATCTTGCTACCAGTGGAACAGCCA
CTAAGGATTCTGCAGTGAGAGCAGAGGGCCAGCTAAGTGGTACTCTCCCAGAGACTGTCTGACTCACGCCACC
CCCTCCACCTTGGACACAGGACGCTGTGGTTTCTGAGCCAGGTACAATGACTCCTTTCGGTAAGTGCAGTGGA
AGCTGTACACTGCCCAGGCAAAGCGTCCGGGCAGCGTAGGCGGGCGACTCAGATCCCAGCCAGTGGACTTAGC
CCCTGTTTGCTCCTCCGATAACTGGGGTGACCTTGGTTAATATTCACCAGCAGCCTCCCCCGTTGCCCCTCTG
GATCCACTGCTTAAATACGGACGAGGACAGGGCCCTGTCTCCTCAGCTTCAGGCACCACCACTGACCTGGGAC
AGTGAATAGATCCTGAGAACTTCAGGGTGAGTCTATGGGACCCTTGATGTTTTCTTTCCCCTTCTTTTCTATG
GTTAAGTTCATGTCATAGGAAGGGGAGAAGTAACAGGGTACACATATTGACCAAATCAGGGTAATTTTGCATT
TGTAATTTTAAAAAATGCTTTCTTCTTTTAATATACTTTTTTGTTTATCTTATTTCTAATACTTTCCCTAATC
TCTTTCTTTCAGGGCAATAATGATACAATGTATCATGCCTCTTTGCACCATTCTAAAGAATAACAGTGATAAT
TTCTGGGTTAAGGCAATAGCAATATTTCTGCATATAAATATTTCTGCATATAAATTGTAACTGATGTAAGAGG
TTTCATATTGCTAATAGCAGCTACAATCCAGCTACCATTCTGCTTTTATTTTCTGGTTGGGATAAGGCTGGAT
TATTCTGAGTCCAAGCTAGGCCCTTTTGCTAATCTTGTTCATACCTCTTATCTTCCTCCCACAGCTCCTGGGC
AACCTGCTGGTCTCTCTGCTGGCCCATCACTTTGGCAAAGCACGCGTGCCACCATGAAGGTGCTGTGGGCTGC
CCTGCTGGTGACCTTCCTGGCTGGCTGTCAGGCTAAAGTGGAGCAGGCTGTGGAGACTGAGCCTGAGCCAGAG
CTGAGGCAGCAGACTGAGTGGCAGTCTGGCCAGAGATGGGAGCTGGCTCTGGGCAGGTTCTGGGACTACCTGA
GGTGGGTGCAGACCCTGTCTGAGCAGGTGCAGGAGGAGCTGCTGTCTTCTCAGGTGACCCAGGAACTGAGGGC
CCTGATGGATGAGACTATGAAGGAGCTGAAGGCCTACAAGTCTGAGCTGGAGGAGCAGCTGACCCCTGTGGCT
GAGGAGACCAGGGCCAGGCTGAGCAAGGAGCTGCAGGCTGCCCAGGCCAGACTGGGGGCTGACATGGAGGATG
TGTGTGGCAGACTGGTGCAGTACAGGGGGGAGGTGCAGGCCATGCTGGGGCAGTCTACTGAGGAGCTGAGGGT
GAGGCTGGCCAGCCACCTGAGGAAGCTGAGGAAGAGGCTGCTGAGGGATGCTGATGACCTGCAGAAGAGGCTG
GCTGTGTATCAGGCTGGGGCTAGGGAGGGGGCTGAGAGGGGCCTGTCTGCCATCAGGGAGAGGCTGGGGCCTC
TGGTGGAGCAGGGCAGGGTGAGGGCTGCCACTGTGGGGAGCCTGGCTGGCCAGCCTCTGCAGGAGAGGGCTCA
GGCCTGGGGGGAGAGGCTGAGGGCCAGGATGGAGGAGATGGGGAGCAGGACCAGGGACAGGCTGGATGAGGTG
AAGGAGCAGGTGGCTGAGGTGAGGGCCAAGCTGGAAGAGCAGGCCCAGCAGATCAGGCTGCAGGCTGAGGCCT
TCCAGGCCAGGCTGAAGAGCTGGTTTGAGCCCCTGGTGGAGGACATGCAGAGGCAGTGGGCTGGCCTGGTGGA
GAAGGTGCAGGCTGCTGTGGGCACCTCTGCTGCCCCTGTGCCCTCTGACAATCACTGAAGATCTAGAGCTGAA
TTCCTGCAGCCAGGGGGATCAGCCTCTACTGTGCCTTCTAGTTGCCAGCCATCTGTTGTTTGCCCCTCCCCCT
TGCCTTCCTTGACCCTGGAAGGTGCCACTCCCACTGTCCTTTCCTAATAAAATGAGGAAATTGCATCACATTG
TCTGAGTAGGTGTCATTCTATTCTGGGGGGTGGGGTGGGGCAGGACAGCAAGGGGGAGGATTGGGAAGACAAT
AGCAGGCATGCTGGGGATGCAGTGGGCTCTATGGCTTCTGAGGCAGAAAGAACCAGCTGGGGCTCGAGATCCA
CTAGGGCCGC
ApoE3-9 Expression cassette (SEQ ID NO: 77)
GCGGCCTAGTAGGCTCAGAGGCACACAGGAGTTTCTGGGCTCACCCTGCCCCCTTCCAACCCCTCAGTTCCCA
TCCTCCAGCAGCTGTTTGTGTGCTGCCTCTGAAGTCCACACTGAACAAACTTCAGCCTACTCATGTCCCTAAA
ATGGGCAAACATTGCAAGCAGCAAACAGCAAACACACAGCCCTCCCTGCCTGCTGACCTTGGAGCTGGGGCAG
AGGTCAGAGACCTCTCTGGGCCCATGCCACCTCCAACATCCACTCGACCCCTTGGAATTTCGGTGGAGAGGAG
CAGAGGTTGTCCTGGCGTGGTTTAGGTAGTGTGAGAGGGGTACCCGGGGATCTTGCTACCAGTGGAACAGCCA
CTAAGGATTCTGCAGTGAGAGCAGAGGGCCAGCTAAGTGGTACTCTCCCAGAGACTGTCTGACTCACGCCACC
CCCTCCACCTTGGACACAGGACGCTGTGGTTTCTGAGCCAGGTACAATGACTCCTTTCGGTAAGTGCAGTGGA
AGCTGTACACTGCCCAGGCAAAGCGTCCGGGCAGCGTAGGCGGGCGACTCAGATCCCAGCCAGTGGACTTAGC
CCCTGTTTGCTCCTCCGATAACTGGGGTGACCTTGGTTAATATTCACCAGCAGCCTCCCCCGTTGCCCCTCTG
GATCCACTGCTTAAATACGGACGAGGACAGGGCCCTGTCTCCTCAGCTTCAGGCACCACCACTGACCTGGGAC
AGTGAATAGATCCTGAGAACTTCAGGGTGAGTCTATGGGACCCTTGATGTTTTCTTTCCCCTTCTTTTCTATG
GTTAAGTTCATGTCATAGGAAGGGGAGAAGTAACAGGGTACACATATTGACCAAATCAGGGTAATTTTGCATT
TGTAATTTTAAAAAATGCTTTCTTCTTTTAATATACTTTTTTGTTTATCTTATTTCTAATACTTTCCCTAATC
TCTTTCTTTCAGGGCAATAATGATACAATGTATCATGCCTCTTTGCACCATTCTAAAGAATAACAGTGATAAT
TTCTGGGTTAAGGCAATAGCAATATTTCTGCATATAAATATTTCTGCATATAAATTGTAACTGATGTAAGAGG
TTTCATATTGCTAATAGCAGCTACAATCCAGCTACCATTCTGCTTTTATTTTCTGGTTGGGATAAGGCTGGAT
TATTCTGAGTCCAAGCTAGGCCCTTTTGCTAATCTTGTTCATACCTCTTATCTTCCTCCCACAGCTCCTGGGC
AACCTGCTGGTCTCTCTGCTGGCCCATCACTTTGGCAAAGCACGCGTGCCACCATGAAAGTGCTGTGGGCTGC
TCTGCTGGTGACCTTCCTGGCTGGGTGCCAGGCCAAGGTGGAGCAGGCTGTGGAGACTGAGCCTGAGCCTGAG
CTGAGGCAGCAGACTGAGTGGCAGTCTGGCCAGAGGTGGGAGCTGGCCCTGGGGAGGTTCTGGGACTACCTGA
GGTGGGTGCAGACTCTGTCTGAGCAGGTGCAGGAGGAGCTGCTGTCTAGCCAGGTGACCCAGGAGCTGAGAGC
CCTGATGGATGAGACCATGAAGGAGCTGAAGGCCTACAAGTCTGAGCTGGAGGAGCAGCTGACCCCTGTGGCT
GAGGAGACCAGAGCTAGGCTGTCTAAGGAGCTGCAGGCTGCTCAGGCCAGGCTGGGGGCTGACATGGAGGATG
TGTGTGGCAGGCTGGTGCAGTATAGGGGGGAGGTCCAGGCCATGCTGGGGCAGAGCACTGAGGAGCTGAGGGT
GAGGCTGGCCAGCCATCTGAGGAAGCTGAGGAAGAGGCTGCTGAGGGATGCTGATGACCTGCAGAAGAGACTG
GCTGTGTACCAGGCTGGGGCCAGGGAGGGGGCTGAGAGGGGCCTGTCTGCCATCAGGGAGAGGCTGGGGCCTC
TGGTGGAGCAGGGCAGGGTGAGGGCTGCCACTGTGGGCTCTCTGGCTGGCCAGCCCCTGCAGGAGAGGGCCCA
GGCTTGGGGGGAGAGGCTGAGAGCCAGAATGGAGGAGATGGGCAGCAGGACCAGGGACAGGCTGGATGAGGTG
AAGGAGCAGGTGGCTGAGGTGAGGGCCAAGCTGGAGGAGCAGGCCCAGCAGATCAGGCTGCAGGCTGAGGCCT
TTCAGGCCAGGCTGAAGAGCTGGTTTGAACCCCTGGTGGAGGACATGCAGAGACAGTGGGCTGGCCTGGTGGA
GAAGGTGCAGGCTGCTGTGGGCACCTCTGCTGCCCCTGTGCCCTCTGATAACCACTGAAGATCTAGAGCTGAA
TTCCTGCAGCCAGGGGGATCAGCCTCTACTGTGCCTTCTAGTTGCCAGCCATCTGTTGTTTGCCCCTCCCCCT
TGCCTTCCTTGACCCTGGAAGGTGCCACTCCCACTGTCCTTTCCTAATAAAATGAGGAAATTGCATCACATTG
TCTGAGTAGGTGTCATTCTATTCTGGGGGGTGGGGTGGGGCAGGACAGCAAGGGGGAGGATTGGGAAGACAAT
AGCAGGCATGCTGGGGATGCAGTGGGCTCTATGGCTTCTGAGGCAGAAAGAACCAGCTGGGGCTCGAGATCCA
CTAGGGCCGC
ApoE3-15 Expression cassette (SEQ ID NO: 78)
GCGGCCTAGTAGGCTCAGAGGCACACAGGAGTTTCTGGGCTCACCCTGCCCCCTTCCAACCCCTCAGTTCCCA
TCCTCCAGCAGCTGTTTGTGTGCTGCCTCTGAAGTCCACACTGAACAAACTTCAGCCTACTCATGTCCCTAAA
ATGGGCAAACATTGCAAGCAGCAAACAGCAAACACACAGCCCTCCCTGCCTGCTGACCTTGGAGCTGGGGCAG
AGGTCAGAGACCTCTCTGGGCCCATGCCACCTCCAACATCCACTCGACCCCTTGGAATTTCGGTGGAGAGGAG
CAGAGGTTGTCCTGGCGTGGTTTAGGTAGTGTGAGAGGGGTACCCGGGGATCTTGCTACCAGTGGAACAGCCA
CTAAGGATTCTGCAGTGAGAGCAGAGGGCCAGCTAAGTGGTACTCTCCCAGAGACTGTCTGACTCACGCCACC
CCCTCCACCTTGGACACAGGACGCTGTGGTTTCTGAGCCAGGTACAATGACTCCTTTCGGTAAGTGCAGTGGA
AGCTGTACACTGCCCAGGCAAAGCGTCCGGGCAGCGTAGGCGGGCGACTCAGATCCCAGCCAGTGGACTTAGC
CCCTGTTTGCTCCTCCGATAACTGGGGTGACCTTGGTTAATATTCACCAGCAGCCTCCCCCGTTGCCCCTCTG
GATCCACTGCTTAAATACGGACGAGGACAGGGCCCTGTCTCCTCAGCTTCAGGCACCACCACTGACCTGGGAC
AGTGAATAGATCCTGAGAACTTCAGGGTGAGTCTATGGGACCCTTGATGTTTTCTTTCCCCTTCTTTTCTATG
GTTAAGTTCATGTCATAGGAAGGGGAGAAGTAACAGGGTACACATATTGACCAAATCAGGGTAATTTTGCATT
TGTAATTTTAAAAAATGCTTTCTTCTTTTAATATACTTTTTTGTTTATCTTATTTCTAATACTTTCCCTAATC
TCTTTCTTTCAGGGCAATAATGATACAATGTATCATGCCTCTTTGCACCATTCTAAAGAATAACAGTGATAAT
TTCTGGGTTAAGGCAATAGCAATATTTCTGCATATAAATATTTCTGCATATAAATTGTAACTGATGTAAGAGG
TTTCATATTGCTAATAGCAGCTACAATCCAGCTACCATTCTGCTTTTATTTTCTGGTTGGGATAAGGCTGGAT
TATTCTGAGTCCAAGCTAGGCCCTTTTGCTAATCTTGTTCATACCTCTTATCTTCCTCCCACAGCTCCTGGGC
AACCTGCTGGTCTCTCTGCTGGCCCATCACTTTGGCAAAGCACGCGTGCCACCATGAAGGTGCTGTGGGCTGC
TCTGCTGGTGACCTTCCTGGCTGGCTGCCAGGCTAAGGTGGAGCAGGCTGTGGAGACTGAGCCTGAGCCTGAG
CTGAGGCAGCAGACTGAGTGGCAGTCTGGGCAGAGGTGGGAGCTGGCCCTGGGCAGGTTCTGGGACTACCTGA
GGTGGGTGCAGACCCTGTCTGAGCAGGTGCAGGAGGAGCTGCTGTCTAGCCAGGTGACCCAGGAACTGAGGGC
CCTGATGGATGAGACCATGAAGGAGCTGAAGGCCTACAAGTCTGAGCTGGAGGAGCAGCTGACCCCTGTGGCT
GAGGAGACCAGGGCCAGGCTGTCTAAGGAGCTGCAGGCTGCCCAGGCCAGGCTGGGGGCTGACATGGAGGATG
TGTGTGGCAGGCTGGTGCAGTATAGGGGGGAGGTGCAGGCTATGCTGGGCCAGAGCACTGAGGAGCTGAGGGT
CAGGCTGGCCAGCCACCTGAGGAAGCTGAGGAAGAGGCTGCTGAGGGATGCTGATGACCTGCAGAAGAGGCTG
GCTGTGTACCAGGCTGGGGCCAGGGAGGGGGCTGAGAGGGGCCTGTCTGCCATCAGGGAGAGGCTGGGCCCTC
TGGTGGAGCAGGGGAGGGTGAGGGCTGCCACTGTGGGCAGCCTGGCTGGCCAGCCCCTGCAGGAAAGGGCCCA
GGCCTGGGGGGAGAGACTGAGGGCCAGGATGGAGGAGATGGGCAGCAGGACCAGAGACAGGCTGGATGAGGTG
AAGGAGCAGGTGGCTGAGGTGAGGGCCAAGCTGGAGGAGCAGGCCCAGCAGATTAGGCTGCAGGCTGAGGCCT
TCCAGGCCAGGCTGAAGTCTTGGTTTGAGCCCCTGGTGGAAGATATGCAGAGGCAGTGGGCTGGCCTGGTGGA
GAAGGTGCAGGCTGCTGTGGGGACCTCTGCTGCTCCTGTGCCCTCTGACAACCACTGAAGATCTAGAGCTGAA
TTCCTGCAGCCAGGGGGATCAGCCTCTACTGTGCCTTCTAGTTGCCAGCCATCTGTTGTTTGCCCCTCCCCCT
TGCCTTCCTTGACCCTGGAAGGTGCCACTCCCACTGTCCTTTCCTAATAAAATGAGGAAATTGCATCACATTG
TCTGAGTAGGTGTCATTCTATTCTGGGGGGTGGGGTGGGGCAGGACAGCAAGGGGGAGGATTGGGAAGACAAT
AGCAGGCATGCTGGGGATGCAGTGGGCTCTATGGCTTCTGAGGCAGAAAGAACCAGCTGGGGCTCGAGATCCA
CTAGGGCCGC
ApoE3ch-9 Expression cassette (SEQ ID NO: 79)
GCGGCCTAGTAGGCTCAGAGGCACACAGGAGTTTCTGGGCTCACCCTGCCCCCTTCCAACCCCTCAGTTCCCA
TCCTCCAGCAGCTGTTTGTGTGCTGCCTCTGAAGTCCACACTGAACAAACTTCAGCCTACTCATGTCCCTAAA
ATGGGCAAACATTGCAAGCAGCAAACAGCAAACACACAGCCCTCCCTGCCTGCTGACCTTGGAGCTGGGGCAG
AGGTCAGAGACCTCTCTGGGCCCATGCCACCTCCAACATCCACTCGACCCCTTGGAATTTCGGTGGAGAGGAG
CAGAGGTTGTCCTGGCGTGGTTTAGGTAGTGTGAGAGGGGTACCCGGGGATCTTGCTACCAGTGGAACAGCCA
CTAAGGATTCTGCAGTGAGAGCAGAGGGCCAGCTAAGTGGTACTCTCCCAGAGACTGTCTGACTCACGCCACC
CCCTCCACCTTGGACACAGGACGCTGTGGTTTCTGAGCCAGGTACAATGACTCCTTTCGGTAAGTGCAGTGGA
AGCTGTACACTGCCCAGGCAAAGCGTCCGGGCAGCGTAGGCGGGCGACTCAGATCCCAGCCAGTGGACTTAGC
CCCTGTTTGCTCCTCCGATAACTGGGGTGACCTTGGTTAATATTCACCAGCAGCCTCCCCCGTTGCCCCTCTG
GATCCACTGCTTAAATACGGACGAGGACAGGGCCCTGTCTCCTCAGCTTCAGGCACCACCACTGACCTGGGAC
AGTGAATAGATCCTGAGAACTTCAGGGTGAGTCTATGGGACCCTTGATGTTTTCTTTCCCCTTCTTTTCTATG
GTTAAGTTCATGTCATAGGAAGGGGAGAAGTAACAGGGTACACATATTGACCAAATCAGGGTAATTTTGCATT
TGTAATTTTAAAAAATGCTTTCTTCTTTTAATATACTTTTTTGTTTATCTTATTTCTAATACTTTCCCTAATC
TCTTTCTTTCAGGGCAATAATGATACAATGTATCATGCCTCTTTGCACCATTCTAAAGAATAACAGTGATAAT
TTCTGGGTTAAGGCAATAGCAATATTTCTGCATATAAATATTTCTGCATATAAATTGTAACTGATGTAAGAGG
TTTCATATTGCTAATAGCAGCTACAATCCAGCTACCATTCTGCTTTTATTTTCTGGTTGGGATAAGGCTGGAT
TATTCTGAGTCCAAGCTAGGCCCTTTTGCTAATCTTGTTCATACCTCTTATCTTCCTCCCACAGCTCCTGGGC
AACCTGCTGGTCTCTCTGCTGGCCCATCACTTTGGCAAAGCACGCGTGCCACCATGAAGGTGCTGTGGGCTGC
CCTGCTGGTGACTTTCCTGGCTGGCTGCCAGGCTAAGGTGGAGCAGGCTGTGGAGACTGAGCCAGAGCCTGAG
CTGAGGCAGCAGACTGAATGGCAGTCTGGCCAGAGGTGGGAGCTGGCCCTGGGCAGGTTCTGGGACTACCTGA
GATGGGTGCAGACCCTGTCTGAGCAGGTCCAGGAGGAGCTGCTGTCTTCTCAGGTGACCCAGGAGCTGAGGGC
CCTGATGGATGAGACTATGAAGGAGCTGAAGGCTTACAAGTCTGAGCTGGAGGAGCAGCTGACCCCTGTGGCT
GAGGAGACCAGGGCCAGACTGAGCAAGGAGCTGCAGGCTGCTCAGGCCAGGCTGGGGGCTGACATGGAGGATG
TGTGTGGCAGGCTGGTGCAGTATTCTGGGGAGGTGCAGGCTATGCTGGGCCAGAGCACTGAGGAACTGAGGGT
GAGGCTGGCCTCTCATCTGAGGAAGCTGAGGAAGAGGCTGCTGAGGGATGCTGATGACCTGCAGAAGAGGCTG
GCTGTGTATCAGGCTGGGGCCAGGGAGGGGGCTGAGAGGGGGCTGTCTGCTATCAGGGAGAGGCTGGGCCCCC
TGGTGGAGCAGGGGAGAGTGAGGGCTGCCACTGTGGGGTCTCTGGCTGGCCAGCCCCTGCAGGAGAGGGCCCA
GGCCTGGGGGGAGAGGCTGAGGGCTAGAATGGAGGAGATGGGGTCTAGGACCAGGGACAGGCTGGATGAGGTC
AAGGAACAGGTGGCTGAGGTCAGGGCCAAGCTGGAGGAGCAGGCCCAGCAGATTAGGCTGCAGGCTGAGGCCT
TTCAGGCCAGGCTGAAGAGCTGGTTTGAGCCCCTGGTGGAGGACATGCAGAGGCAGTGGGCTGGCCTGGTGGA
GAAGGTGCAGGCTGCTGTGGGCACTTCTGCTGCCCCTGTGCCCTCTGACAACCATTGAAGATCTAGAGCTGAA
TTCCTGCAGCCAGGGGGATCAGCCTCTACTGTGCCTTCTAGTTGCCAGCCATCTGTTGTTTGCCCCTCCCCCT
TGCCTTCCTTGACCCTGGAAGGTGCCACTCCCACTGTCCTTTCCTAATAAAATGAGGAAATTGCATCACATTG
TCTGAGTAGGTGTCATTCTATTCTGGGGGGTGGGGTGGGGCAGGACAGCAAGGGGGAGGATTGGGAAGACAAT
AGCAGGCATGCTGGGGATGCAGTGGGCTCTATGGCTTCTGAGGCAGAAAGAACCAGCTGGGGCTCGAGATCCA
CTAGGGCCGC
ApoE3ch-3 Expression cassette (SEQ ID NO: 80)
GCGGCCTAGTAGGCTCAGAGGCACACAGGAGTTTCTGGGCTCACCCTGCCCCCTTCCAACCCCTCAGTTCCCA
TCCTCCAGCAGCTGTTTGTGTGCTGCCTCTGAAGTCCACACTGAACAAACTTCAGCCTACTCATGTCCCTAAA
ATGGGCAAACATTGCAAGCAGCAAACAGCAAACACACAGCCCTCCCTGCCTGCTGACCTTGGAGCTGGGGCAG
AGGTCAGAGACCTCTCTGGGCCCATGCCACCTCCAACATCCACTCGACCCCTTGGAATTTCGGTGGAGAGGAG
CAGAGGTTGTCCTGGCGTGGTTTAGGTAGTGTGAGAGGGGTACCCGGGGATCTTGCTACCAGTGGAACAGCCA
CTAAGGATTCTGCAGTGAGAGCAGAGGGCCAGCTAAGTGGTACTCTCCCAGAGACTGTCTGACTCACGCCACC
CCCTCCACCTTGGACACAGGACGCTGTGGTTTCTGAGCCAGGTACAATGACTCCTTTCGGTAAGTGCAGTGGA
AGCTGTACACTGCCCAGGCAAAGCGTCCGGGCAGCGTAGGCGGGCGACTCAGATCCCAGCCAGTGGACTTAGC
CCCTGTTTGCTCCTCCGATAACTGGGGTGACCTTGGTTAATATTCACCAGCAGCCTCCCCCGTTGCCCCTCTG
GATCCACTGCTTAAATACGGACGAGGACAGGGCCCTGTCTCCTCAGCTTCAGGCACCACCACTGACCTGGGAC
AGTGAATAGATCCTGAGAACTTCAGGGTGAGTCTATGGGACCCTTGATGTTTTCTTTCCCCTTCTTTTCTATG
GTTAAGTTCATGTCATAGGAAGGGGAGAAGTAACAGGGTACACATATTGACCAAATCAGGGTAATTTTGCATT
TGTAATTTTAAAAAATGCTTTCTTCTTTTAATATACTTTTTTGTTTATCTTATTTCTAATACTTTCCCTAATC
TCTTTCTTTCAGGGCAATAATGATACAATGTATCATGCCTCTTTGCACCATTCTAAAGAATAACAGTGATAAT
TTCTGGGTTAAGGCAATAGCAATATTTCTGCATATAAATATTTCTGCATATAAATTGTAACTGATGTAAGAGG
TTTCATATTGCTAATAGCAGCTACAATCCAGCTACCATTCTGCTTTTATTTTCTGGTTGGGATAAGGCTGGAT
TATTCTGAGTCCAAGCTAGGCCCTTTTGCTAATCTTGTTCATACCTCTTATCTTCCTCCCACAGCTCCTGGGC
AACCTGCTGGTCTCTCTGCTGGCCCATCACTTTGGCAAAGCACGCGTGCCACCATGAAGGTGCTGTGGGCTGC
CCTGCTGGTGACCTTTCTGGCTGGGTGCCAGGCCAAGGTGGAGCAGGCTGTGGAGACTGAGCCTGAGCCTGAG
CTGAGGCAGCAGACTGAGTGGCAGTCTGGGCAGAGGTGGGAGCTGGCCCTGGGGAGGTTCTGGGACTACCTGA
GGTGGGTGCAGACTCTGTCTGAGCAGGTGCAGGAGGAGCTGCTGTCTAGCCAGGTGACCCAGGAGCTGAGGGC
CCTGATGGATGAGACCATGAAGGAGCTGAAGGCCTACAAGTCTGAGCTGGAAGAGCAGCTGACCCCTGTGGCT
GAGGAGACCAGGGCTAGGCTGAGCAAGGAACTGCAGGCTGCTCAGGCCAGGCTGGGGGCTGACATGGAAGATG
TGTGTGGGAGGCTGGTGCAGTACTCTGGGGAGGTGCAGGCCATGCTGGGCCAGAGCACTGAGGAGCTGAGGGT
GAGGCTGGCCAGCCACCTGAGGAAGCTGAGGAAGAGGCTGCTGAGGGATGCTGATGACCTGCAGAAGAGGCTG
GCTGTGTACCAGGCTGGGGCCAGGGAGGGGGCTGAGAGGGGCCTGTCTGCTATCAGGGAGAGGCTGGGCCCCC
TGGTGGAGCAGGGCAGGGTGAGGGCTGCCACTGTGGGCAGCCTGGCTGGGCAGCCTCTGCAGGAGAGGGCCCA
GGCCTGGGGGGAGAGGCTGAGAGCCAGGATGGAGGAGATGGGCTCTAGGACTAGGGACAGGCTGGATGAGGTG
AAGGAGCAGGTGGCTGAGGTGAGGGCCAAGCTGGAGGAGCAGGCTCAGCAGATCAGGCTGCAGGCTGAGGCTT
TCCAGGCCAGGCTGAAGTCTTGGTTTGAGCCCCTGGTGGAGGATATGCAGAGGCAGTGGGCTGGCCTGGTGGA
GAAGGTGCAGGCTGCTGTGGGCACCTCTGCTGCCCCTGTGCCCTCTGACAACCATTGAAGATCTAGAGCTGAA
TTCCTGCAGCCAGGGGGATCAGCCTCTACTGTGCCTTCTAGTTGCCAGCCATCTGTTGTTTGCCCCTCCCCCT
TGCCTTCCTTGACCCTGGAAGGTGCCACTCCCACTGTCCTTTCCTAATAAAATGAGGAAATTGCATCACATTG
TCTGAGTAGGTGTCATTCTATTCTGGGGGGTGGGGTGGGGCAGGACAGCAAGGGGGAGGATTGGGAAGACAAT
AGCAGGCATGCTGGGGATGCAGTGGGCTCTATGGCTTCTGAGGCAGAAAGAACCAGCTGGGGCTCGAGATCCA
CTAGGGCCGC
ITRs
5′ ITR (SEQ ID NO: 81)
CTGCGCGCTCGCTCGCTCACTGAGGCCGCCCGGGCAAAGCCCGGGCGTCGGGCGACCTTTGGTCGCCCGGCCT
CAGTGAGCGAGCGAGCGCGCAGAGAGGGAGTGGCCAACTCCATCACTAGGGGTTCCT
3′ ITR (SEQ ID NO: 82)
AGGAACCCCTAGTGATGGAGTTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCGCGCAG
Capsid
VP1: (SEQ ID NO: 83)
MAADGYLPDWLEDNLSEGIREWWDLKPGAPKPKANQQKQDNGRGLVLPGYKYLGPENGLDKGEPVNAADAAAL
EHDKAYDQQLQAGDNPYLRYNHADAEFQERLQEDTSFGGNLGRAVFQAKKRVLEPLGLVESPVKTAPGKKRPV
EPSPQRSPDSSTGIGKKGQQPAKKRLNFGQTGDSESVPDPQPIGEPPAAPSGVGPNTMAAGGGAPMADNNEGA
DGVGSSSGNWHCDSTWLGDRVITTSTRTWALPTYNNHLYKQISNGTSGGSTNDNTYFGYSTPWGYFDFNRFHC
HFSPRDWQRLINNNWGFRPKRLNFKLENIQVKEVTQNEGTKTIANNLTSTIQVFTDSEYQLPYVLGSAHQGCL
PPFPADVFMIPQYGYLTLNNGSQAVGRSSFYCLEYFPSQMLRTGNNFEFSYNFEDVPFHSSYAHSQSLDRLMN
PLIDQYLYYLSRTQSTGGTAGTQQLLFSQAGPNNMSAQAKNWLPGPCYRQQRVSTTLSQNNNSNFAWTGATKY
HLNGRDSLVNPGVAMATHKDDEERFFPSSGVLMFGKQGAGKDNVDYSSVMLTSEEEIKTTNPVATEQYGVVAD
NLQQQNAAPIVGAVNSQGALPGMVWQNRDVYLQGPIWAKIPHTDGNFHPSPLMGGFGLKHPPPQILIKNTPVP
ADPPTTFNQAKLASFITQYSTGQVSVEIEWELQKENSKRWNPEIQYTSNYYKSTNVDFAVNTEGTYSEPRPIG
TRYLTRNL
VP1 (SEQ ID NO: 84)
MAADGYLPDWLEDNLSEGIREWWALQPGAPKPKANQQHQDNARGLVLPGYKYLGPGNGLDKGEPVNAADAAAL
EHDKAYDQQLKAGDNPYLKYNHADAEFQERLKEDTSFGGNLGRAVFQAKKRLLEPLGLVEEAAKTAPGKKRPV
DQSPQEPDSSSGVGKSGKQPARKRLNFGQTGDSESVPDPQPLGEPPAAPTSLGSNTMASGGGAPMADNNEGAD
GVGNSSGNWHCDSQWLGDRVITTSTRTWALPTYNNHLYKQISSQSGASNDNHYFGYSTPWGYFDENRFHCHFS
PRDWQRLINNNWGFRPKKLSFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFQFSYTFEDVPFHSSYAHSQSLDRLMNPLI
DQYLYYLNRTQGTTSGTTNQSRLLFSQAGPQSMSLQARNWLPGPCYRQQRLSKTANDNNNSNFPWTAASKYHL
NGRDSLVNPGPAMASHKDDEEKFFPMHGNLIFGKEGTTASNAELDNVMITDEEEIRTTNPVATEQYGTVANNL
QSSNTAPTTRTVNDQGALPGMVWQDRDVYLQGPIWAKIPHTDGHFHPSPLMGGFGLKHPPPQIMIKNTPVPAN
PPTTFSPAKFASFITQYSTGQVSVEIEWELQKENSKRWNPEIQYTSNYNKSVNVDFTVDTNGVYSEPRPIGTR
YLTRPL
VP2 (SEQ ID NO: 122)
MAPGKKRPVEPSPQRSPDSSTGIGKKGQQPAKKRLNFGQTGDSESVPDPQPIGEPPAAPSGVGPNTMAAGGGA
PMADNNEGADGVGSSSGNWHCDSTWLGDRVITTSTRTWALPTYNNHLYKQISNGTSGGSTNDNTYFGYSTPWG
YFDFNRFHCHFSPRDWQRLINNNWGFRPKRLNFKLFNIQVKEVTQNEGTKTIANNLTSTIQVFTDSEYQLPYV
LGSAHQGCLPPFPADVFMIPQYGYLTLNNGSQAVGRSSFYCLEYFPSQMLRTGNNFEFSYNFEDVPFHSSYAH
SQSLDRLMNPLIDQYLYYLSRTQSTGGTAGTQQLLFSQAGPNNMSAQAKNWLPGPCYRQQRVSTTLSQNNNSN
FAWTGATKYHLNGRDSLVNPGVAMATHKDDEERFFPSSGVLMFGKQGAGKDNVDYSSVMLTSEEEIKTTNPVA
TEQYGVVADNLQQQNAAPIVGAVNSQGALPGMVWQNRDVYLQGPIWAKIPHTDGNFHPSPLMGGFGLKHPPPQ
ILIKNTPVPADPPTTFNQAKLASFITQYSTGQVSVEIEWELQKENSKRWNPEIQYTSNYYKSTNVDFAVNTEG
TYSEPRPIGTRYLTRNL
VP3 (SEQ ID NO: 123)
MAAGGGAPMADNNEGADGVGSSSGNWHCDSTWLGDRVITTSTRTWALPTYNNHLYKQISNGTSGGSTNDNTYF
GYSTPWGYFDFNRFHCHESPRDWQRLINNNWGFRPKRLNFKLFNIQVKEVTQNEGTKTIANNLTSTIQVFTDS
EYQLPYVLGSAHQGCLPPFPADVEMIPQYGYLTLNNGSQAVGRSSFYCLEYFPSQMLRTGNNFEFSYNFEDVP
FHSSYAHSQSLDRLMNPLIDQYLYYLSRTQSTGGTAGTQQLLFSQAGPNNMSAQAKNWLPGPCYRQQRVSTTL
SQNNNSNFAWTGATKYHLNGRDSLVNPGVAMATHKDDEERFFPSSGVLMFGKQGAGKDNVDYSSVMLTSEEEI
KTTNPVATEQYGVVADNLQQQNAAPIVGAVNSQGALPGMVWQNRDVYLQGPIWAKIPHTDGNFHPSPLMGGFG
LKHPPPQILIKNTPVPADPPTTFNQAKLASFITQYSTGQVSVEIEWELQKENSKRWNPEIQYTSNYYKSTNVD
FAVNTEGTYSEPRPIGTRYLTRNL
Vector components and ITRs
CMV enhancer: (SEQ ID NO: 85)
ATCTAGTTATTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGACGTATGTTC
CCATAGTAACGCCAATAGGGACTTTCCATTGACGTCAATGGGTGGAGTATTTACGGTAAACTGCCCACTTGGC
AGTACATCAAGTGTATCATATGCCAAGTACGCCCCCTATTGACGTCAATGACGGTAAATGGCCCGCCTGGCAT
TATGCCCAGTACATGACCTTATGGGACTTTCCTACTTGGCAGTACATCTACGTATTAGTCATCGCTATTACCA
chicken beta-actin promoter (SEQ ID NO: 86)
TGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGG
CGGGGCGAGGGGCGGGGCGGGGCGAGGCGGAGAGGTGCGGCGGCAGCCAATCAGAGCGGCGCGCTCCGAAAGT
TTCCTTTTATGGCGAGGCGGCGGCGGCGGCGGCCCTATAAAAAGCGAAGCGCGCGGCGGGCGG
chicken beta-actin intron (SEQ ID NO: 87)
GGAGTCGCTGCGCGCTGCCTTCGCCCCGTGCCCCGCTCCGCCGCCGCCTCGCGCCGCCCGCCCCGGCTCTGAC
TGACCGCGTTACTCCCACAGGTGAGCGGGCGGGACGGCCCTTCTCCTCCGGGCTGTAATTAGCGCTTGGTTTA
ATGACGGCTTGTTTCTTTTCTGTGGCTGCGTGAAAGCCTTGAGGGGCTCCGGGAGGGCCCTTTGTGCGGGGGG
AGCGGCTCGGGGGGTGCGTGCGTGTGTGTGTGCGTGGGGAGCGCCGCGTGCGGCTCCGCGCTGCCCGGCGGCT
GTGAGCGCTGCGGGCGCGGCGCGGGGCTTTGTGCGCTCCGCAGTGTGCGCGAGGGGAGCGCGGCCGGGGGCGG
TGCCCCGCGGTGCGGGGGGGGCTGCGAGGGGAACAAAGGCTGCGTGCGGGGTGTGTGCGTGGGGGGGTGAGCA
GGGGGTGTGGGCGCGTCGGTCGGGCTGCAACCCCCCCTGCACCCCCCTCCCCGAGTTGCTGAGCACGGCCCGG
CTTCGGGTGCGGGGCTCCGTACGGGGCGTGGCGCGGGGCTCGCCGTGCCGGGCGGGGGGTGGCGGCAGGTGGG
GGTGCCGGGCGGGGGGGGGCCGCCTCGGGCCGGGGAGGGCTCGGGGGAGGGGCGCGGCGGCCCCCGGAGCGCC
GGCGGCTGTCGAGGCGCGGCGAGCCGCAGCCATTGCCTTTTATGGTAATCGTGCGAGAGGGCGCAGGGACTTC
CTTTGTCCCAAATCTGTGCGGAGCCGAAATCTGGGAGGCGCCGCCGCACCCCCTCTAGCGGGCGCGGGGCGAA
GCGGTGCGGCGCCGGCAGGAAGGAAATGGGCGGGGAGGGCCTTCGTGCGTCGCCGCGCCGCCGTCCCCTTCTC
CCTCTCCAGCCTCGGGGCTGTCCGCGGGGGGACGGCTGCCTTCGGGGGGGACGGGGCAGGGCGGGGTTCGGCT
TCTGGCGTGTGACCGGCG
5′ ITR 141bp (SEQ ID NO: 88)
CCTGCAGGCAGCTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGGGTTCCT
3′ ITR 141bp (SEQ ID NO: 89)
AGGAACCCCTAGTGATGGAGTTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCGCGCAGCTGCCTGCAGG
5′ ITR 119bp (SEQ ID NO: 90)
CCTGCAGGCAGCTGCGCGCTCGCTCGCTCACTGAGGCCGCCCGGGCAAAGCCCGGGCGGCCTCAGTGAGCGAG
CGAGCGCGCAGAGAGGGAGTGGCCAACTCCATCACTAGGGGTTCCT
3′ ITR 119bp (SEQ ID NO: 91)
AGGAACCCCTAGTGATGGAGTTGGCCACTCCCTCTCTGCGCGCTCGCTCGCTCACTGAGGCCGCCCGGGCTTT
GCCCGGGCGGCCTCAGTGAGCGAGCGAGCGCGCAGCTGCCTGCAGG
5′ ITR 145bp FLOP (SEQ ID NO: 92)
TTGGCCACTCCCTCTCTGCGCGCTCGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGCCAACTCCATCACTAGGGGTTCCT
3′ ITR AAV2 (SEQ ID NO: 93)
AGGAACCCCTAGTGATGGAGTTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCGCGCAGAGAGGGAGTGGCCAA
5′ ITR 145bp FLIP (SEQ ID NO: 94)
TTGGCCACTCCCTCTCTGCGCGCTCGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGCCAACTCCATCACTAGGGGTTCCT
Mature ApoE (CpG free)
ApoE3ch-40 (SEQ ID NO: 95)
ATGAAGGTGTTGTGGGCAGCACTGCTGGTCACTTTCCTGGCAGGCTGCCAGGCCAAGGTGGAGCAGGCAGTAG
AGACTGAACCAGAACCTGAACTGAGGCAACAGACTGAATGGCAGAGTGGTCAAAGATGGGAACTGGCCCTTGG
AAGGTTTTGGGACTACCTGAGGTGGGTGCAGACCCTCAGTGAGCAGGTGCAGGAGGAGCTGCTGAGCTCCCAG
GTGACCCAAGAACTAAGGGCTCTGATGGATGAGACCATGAAGGAGCTGAAGGCCTACAAGTCTGAGCTGGAGG
AACAGCTCACCCCAGTGGCTGAAGAGACTAGAGCAAGGCTCAGCAAGGAGTTGCAGGCTGCCCAGGCCAGGCT
GGGGGCTGACATGGAAGATGTATGTGGAAGGCTTGTGCAGTACAGTGGTGAGGTGCAGGCCATGCTGGGACAG
AGCACTGAAGAGCTGAGGGTCAGGCTGGCCTCCCACTTAAGGAAGCTAAGAAAAAGACTGCTCAGAGATGCTG
ATGACCTCCAGAAGAGACTGGCTGTGTACCAGGCTGGAGCCAGAGAGGGAGCAGAGAGAGGACTGAGTGCCAT
TAGGGAGAGATTGGGTCCCCTGGTAGAGCAGGGCAGGGTCAGAGCTGCCACAGTGGGCTCTTTGGCAGGCCAG
CCTTTGCAGGAAAGAGCTCAGGCCTGGGGAGAGAGACTGAGAGCCAGGATGGAGGAGATGGGATCTAGGACCA
GGGACAGACTGGATGAAGTCAAAGAACAAGTTGCTGAGGTGAGGGCCAAGCTGGAGGAGCAAGCCCAGCAGAT
TAGGCTACAGGCAGAGGCCTTCCAGGCCAGGCTGAAGAGCTGGTTTGAGCCTTTGGTAGAGGACATGCAGAGA
CAGTGGGCTGGGTTGGTGGAGAAGGTCCAGGCTGCTGTTGGCACATCAGCAGCCCCTGTCCCCAGTGACAACC
ACTGA
ApoE3ch-41 (SEQ ID NO: 96)
ATGAAGGTCCTCTGGGCAGCCCTGCTGGTCACATTCCTGGCAGGCTGTCAAGCCAAGGTGGAACAAGCTGTGG
AGACTGAGCCAGAGCCAGAGCTCAGGCAGCAAACAGAGTGGCAGTCTGGCCAGAGATGGGAATTGGCACTGGG
AAGATTCTGGGACTACCTGAGGTGGGTTCAGACCCTGTCAGAGCAGGTGCAGGAGGAGCTCCTGAGCTCACAA
GTCACTCAAGAGCTGAGAGCACTTATGGATGAAACCATGAAGGAATTAAAGGCCTACAAATCAGAATTGGAAG
AGCAGCTGACACCTGTGGCTGAAGAGACCAGGGCCAGACTGAGTAAAGAACTGCAAGCAGCCCAGGCCAGACT
TGGAGCTGACATGGAGGATGTTTGTGGCAGGCTGGTGCAGTACAGTGGTGAAGTCCAAGCCATGCTGGGACAA
TCCACAGAGGAGCTGAGGGTCAGGCTGGCCAGCCACCTAAGAAAACTGAGGAAGAGACTCCTGAGGGATGCTG
ATGATCTCCAGAAAAGGTTGGCTGTGTACCAAGCTGGAGCCAGGGAAGGAGCTGAAAGGGGACTCTCTGCCAT
CAGAGAAAGACTGGGTCCCCTGGTGGAGCAGGGCAGGGTGAGAGCAGCCACAGTGGGAAGCTTGGCAGGACAG
CCACTCCAGGAAAGGGCACAGGCATGGGGAGAAAGGCTGAGGGCCAGGATGGAGGAGATGGGCAGCAGGACCA
GGGACAGGCTGGATGAGGTGAAGGAACAAGTGGCAGAGGTAAGGGCAAAACTGGAGGAGCAGGCCCAGCAGAT
CAGGCTCCAGGCTGAAGCCTTCCAAGCAAGGTTGAAAAGCTGGTTTGAGCCTCTTGTGGAAGACATGCAGAGA
CAGTGGGCTGGCCTTGTGGAAAAAGTCCAAGCTGCAGTAGGCACCAGTGCAGCACCTGTCCCATCAGACAACC
ATTGA
ApoE3ch-42 (SEQ ID NO: 97)
ATGAAAGTCCTCTGGGCTGCCTTGCTGGTAACTTTCTTGGCTGGCTGCCAGGCCAAGGTGGAGCAAGCTGTGG
AAACAGAGCCAGAGCCAGAGCTGAGGCAACAGACAGAATGGCAGTCAGGCCAGAGGTGGGAGCTGGCTCTGGG
GAGATTCTGGGATTACCTGAGGTGGGTCCAGACACTCTCTGAACAAGTGCAAGAGGAGCTGCTGTCCTCCCAA
GTCACCCAGGAGCTGAGAGCCTTGATGGATGAAACCATGAAAGAGCTGAAAGCTTACAAGTCTGAGCTGGAGG
AACAGTTGACCCCAGTGGCTGAGGAGACCAGGGCCAGATTGTCCAAGGAACTGCAGGCTGCTCAGGCAAGGTT
GGGGGCAGATATGGAGGATGTGTGTGGGAGGTTGGTGCAGTACAGTGGTGAGGTGCAGGCCATGCTGGGACAG
AGCACTGAAGAGCTAAGGGTCAGATTAGCAAGTCATTTGAGGAAGCTGAGAAAGAGGCTGCTGAGAGATGCTG
ATGACCTTCAGAAGAGACTGGCTGTCTACCAAGCTGGTGCCAGAGAAGGGGCTGAAAGAGGTCTCTCAGCCAT
CAGGGAGAGGCTGGGGCCTCTGGTTGAGCAGGGTAGGGTGAGAGCAGCCACTGTGGGATCCCTGGCTGGTCAA
CCACTCCAAGAAAGAGCACAGGCCTGGGGGGAGAGACTGAGGGCCAGGATGGAGGAAATGGGCAGCAGGACAA
GAGACAGGCTGGATGAGGTCAAGGAGCAAGTAGCAGAAGTGAGGGCCAAACTGGAGGAACAGGCCCAGCAGAT
CAGACTCCAAGCTGAGGCCTTCCAGGCCAGGCTGAAATCTTGGTTTGAGCCTTTGGTAGAAGACATGCAGAGG
CAGTGGGCAGGGTTGGTAGAAAAGGTGCAGGCTGCTGTGGGGACTTCAGCAGCCCCTGTGCCCTCAGACAATC
ACTGA
ApoE3ch-43 (SEQ ID NO: 98)
ATGAAGGTCCTCTGGGCTGCCCTGCTGGTCACCTTTCTAGCTGGGTGCCAGGCCAAGGTGGAACAAGCAGTGG
AAACTGAGCCTGAGCCTGAGCTGAGGCAACAGACAGAGTGGCAGTCTGGTCAGAGGTGGGAGCTTGCACTGGG
GAGGTTCTGGGATTACCTGAGGTGGGTCCAGACCCTGAGTGAGCAGGTGCAGGAGGAGCTCCTTAGCTCACAG
GTTACTCAGGAGTTGAGAGCACTCATGGATGAAACCATGAAGGAGCTGAAGGCCTACAAAAGTGAACTGGAAG
AACAGCTGACCCCTGTTGCAGAAGAAACCAGGGCAAGGCTCTCCAAAGAGCTGCAAGCTGCTCAGGCAAGACT
GGGTGCTGACATGGAGGATGTCTGTGGCAGGCTGGTGCAGTATAGTGGTGAGGTGCAGGCAATGCTGGGGCAG
TCCACTGAAGAGCTGAGGGTGAGGCTGGCCTCTCATCTTAGGAAGTTAAGGAAGAGGCTGCTCAGGGATGCTG
ATGACCTCCAGAAGAGGCTGGCTGTGTATCAGGCTGGGGCAAGGGAGGGGGCAGAGAGGGGCCTCTCTGCCAT
AAGGGAGAGACTGGGTCCACTGGTGGAACAGGGCAGGGTGAGAGCAGCCACAGTGGGCTCTCTGGCAGGGCAG
CCCCTCCAAGAAAGAGCCCAGGCTTGGGGAGAAAGACTGAGGGCCAGGATGGAGGAAATGGGATCCAGGACAA
GGGACAGGCTGGATGAGGTGAAGGAGCAGGTGGCTGAGGTGAGGGCAAAGCTGGAAGAGCAAGCCCAGCAGAT
CAGGCTCCAGGCTGAAGCTTTCCAAGCAAGGCTGAAAAGCTGGTTTGAGCCCCTGGTGGAGGACATGCAGAGG
CAGTGGGCAGGCCTGGTGGAGAAAGTTCAGGCAGCTGTGGGTACTTCAGCTGCACCTGTCCCTTCTGACAATC
ACTGA
ApoE3ch-44 (SEQ ID NO: 99)
ATGAAAGTCCTGTGGGCAGCCCTCCTGGTCACCTTCCTGGCTGGCTGTCAAGCTAAAGTGGAGCAGGCTGTGG
AAACAGAGCCTGAGCCAGAACTTAGACAACAAACTGAGTGGCAGAGTGGGCAGAGGTGGGAGCTGGCCTTGGG
CAGATTCTGGGACTACCTGAGGTGGGTCCAGACTCTTTCTGAGCAGGTGCAGGAGGAGCTCCTGTCCAGTCAG
GTGACCCAGGAGCTGAGGGCTCTCATGGATGAGACAATGAAAGAGCTGAAGGCCTACAAAAGTGAGCTGGAAG
AGCAGCTGACCCCAGTGGCTGAAGAGACAAGGGCCAGGTTAAGCAAAGAGCTCCAGGCTGCCCAGGCCAGGCT
GGGAGCAGACATGGAGGATGTCTGTGGCAGGCTGGTGCAGTATAGTGGGGAGGTGCAAGCCATGTTAGGCCAG
AGCACTGAAGAGCTGAGGGTGAGGTTAGCTTCTCACCTGAGAAAATTAAGGAAGAGGCTGCTGAGGGATGCTG
ATGACCTGCAGAAGAGGTTGGCTGTTTACCAGGCAGGGGCTAGAGAGGGAGCAGAGAGGGGCCTCTCTGCCAT
CAGAGAGAGGCTGGGCCCCCTTGTGGAGCAGGGCAGGGTGAGAGCAGCCACTGTTGGCTCACTTGCAGGGCAG
CCTCTGCAGGAAAGAGCCCAGGCATGGGGGGAGAGACTGAGGGCAAGAATGGAAGAGATGGGCTCAAGGACAA
GAGACAGGCTGGATGAGGTGAAGGAGCAGGTGGCAGAAGTGAGGGCCAAGCTAGAGGAACAGGCCCAGCAGAT
CAGGCTCCAGGCAGAAGCCTTCCAGGCCAGGCTCAAGTCCTGGTTTGAGCCCTTAGTGGAGGACATGCAGAGG
CAGTGGGCTGGCCTGGTGGAGAAGGTACAGGCTGCTGTGGGGACTTCAGCAGCCCCAGTGCCCAGTGACAACC
ACTGA
ApoE3ch-45 (SEQ ID NO: 100)
ATGAAGGTCCTCTGGGCAGCTCTCCTGGTTACCTTCTTAGCTGGCTGTCAAGCCAAAGTAGAACAAGCAGTGG
AAACAGAGCCAGAGCCAGAGCTCAGACAGCAAACAGAATGGCAGTCTGGTCAGAGATGGGAACTGGCACTGGG
TAGGTTCTGGGACTACCTGAGGTGGGTCCAGACCCTGTCAGAGCAGGTCCAGGAAGAGCTGCTGAGCAGCCAG
GTGACTCAAGAGCTGAGGGCCCTCATGGATGAAACCATGAAGGAGCTGAAGGCCTACAAGAGTGAGTTAGAAG
AGCAGCTCACACCTGTGGCTGAAGAAACAAGGGCCAGGCTCAGCAAGGAGCTGCAAGCTGCACAGGCCAGGCT
GGGTGCTGATATGGAGGATGTGTGTGGCAGGCTGGTGCAGTATAGTGGTGAAGTCCAGGCCATGTTGGGCCAA
TCCACAGAGGAGCTCAGGGTCAGGCTGGCCTCCCATCTCAGGAAACTAAGAAAGAGACTGCTCAGGGATGCTG
ATGACCTTCAGAAGAGGCTGGCTGTGTACCAGGCAGGAGCCAGAGAGGGGGCTGAGAGGGGTCTCTCAGCCAT
CAGGGAAAGACTTGGGCCCTTGGTTGAGCAGGGCAGGGTGAGAGCAGCCACAGTTGGGTCTCTGGCTGGCCAG
CCCCTCCAGGAGAGAGCTCAGGCCTGGGGTGAGAGACTGAGGGCCAGGATGGAGGAGATGGGGAGCAGGACCA
GGGACAGGTTGGATGAGGTCAAGGAGCAGGTGGCAGAAGTGAGAGCCAAGCTGGAAGAGCAGGCCCAGCAAAT
CAGGCTGCAAGCAGAGGCATTTCAGGCCAGGCTGAAGTCATGGTTTGAGCCTCTGGTGGAGGACATGCAGAGG
CAGTGGGCTGGGCTGGTGGAAAAGGTACAGGCTGCTGTGGGCACCAGTGCAGCACCTGTCCCATCTGATAACC
ATTGA
ApoE3ch-46 (SEQ ID NO: 101)
ATGAAAGTCCTGTGGGCAGCCCTCCTTGTGACCTTCCTGGCAGGCTGTCAAGCAAAGGTGGAGCAGGCTGTAG
AGACTGAGCCAGAGCCTGAACTGAGGCAGCAGACAGAATGGCAGTCTGGCCAGAGATGGGAGCTGGCCCTGGG
AAGATTTTGGGACTACCTGAGGTGGGTCCAGACCCTGTCAGAACAGGTTCAGGAAGAGCTGCTTTCCAGCCAA
GTGACACAGGAGCTGAGAGCCCTTATGGATGAGACAATGAAAGAACTGAAGGCTTATAAAAGTGAGCTAGAGG
AGCAGCTGACCCCAGTGGCTGAAGAGACCAGGGCCAGGCTCAGCAAAGAGCTCCAGGCTGCACAGGCCAGATT
GGGAGCTGACATGGAGGATGTGTGTGGGAGACTAGTGCAGTACAGTGGTGAAGTGCAGGCCATGCTGGGACAA
AGCACAGAAGAGTTGAGAGTCAGGCTGGCATCCCACCTGAGGAAACTCAGGAAAAGACTGCTCAGGGATGCTG
ATGACCTCCAGAAGAGACTGGCAGTCTATCAGGCTGGTGCCAGAGAGGGGGCAGAGAGGGGACTCTCAGCCAT
CAGGGAGAGGCTGGGGCCTCTGGTTGAGCAGGGCAGAGTCAGAGCAGCAACAGTGGGGAGTCTGGCAGGCCAG
CCCTTACAAGAGAGAGCTCAGGCATGGGGAGAGAGGCTGAGGGCCAGGATGGAAGAAATGGGATCAAGGACTA
GAGACAGGTTGGATGAAGTAAAGGAGCAGGTGGCAGAAGTGAGGGCCAAGCTTGAGGAGCAGGCCCAGCAGAT
CAGGCTACAGGCTGAGGCATTTCAGGCCAGGCTGAAGAGCTGGTTTGAGCCCTTGGTGGAGGATATGCAGAGG
CAGTGGGCTGGATTGGTGGAAAAGGTACAGGCTGCTGTGGGCACCTCAGCAGCTCCTGTCCCCTCTGATAATC
ATTGA
ApoE3ch-47 (SEQ ID NO: 102)
ATGAAGGTCCTCTGGGCAGCCCTGCTGGTCACCTTCCTGGCTGGCTGCCAGGCCAAGGTGGAGCAGGCTGTGG
AGACTGAGCCAGAGCCAGAGCTGAGGCAGCAGACAGAGTGGCAGTCTGGCCAGAGATGGGAGCTGGCCCTGGG
AAGATTCTGGGACTACCTGAGGTGGGTCCAGACCCTGTCAGAGCAGGTGCAGGAGGAGCTGCTGAGCTCCCAG
GTGACCCAGGAGCTGAGGGCCCTGATGGATGAAACCATGAAGGAGCTGAAGGCCTACAAGAGTGAGCTGGAGG
AGCAGCTGACCCCTGTGGCTGAAGAGACCAGGGCCAGGCTCAGCAAGGAGCTGCAGGCTGCCCAGGCCAGGCT
GGGAGCTGACATGGAGGATGTGTGTGGCAGGCTGGTGCAGTACAGTGGTGAGGTGCAGGCCATGCTGGGACAG
AGCACTGAAGAGCTGAGGGTCAGGCTGGCCTCCCACCTGAGGAAGCTGAGGAAGAGACTGCTCAGGGATGCTG
ATGACCTCCAGAAGAGGCTGGCTGTGTACCAGGCTGGGGCCAGAGAGGGGGCAGAGAGGGGACTCTCAGCCAT
CAGGGAGAGGCTGGGGCCTCTGGTGGAGCAGGGCAGGGTGAGAGCAGCCACAGTGGGCTCTCTGGCAGGCCAG
CCTCTCCAGGAAAGAGCTCAGGCCTGGGGAGAGAGACTGAGGGCCAGGATGGAGGAGATGGGCAGCAGGACCA
GGGACAGGCTGGATGAGGTGAAGGAGCAGGTGGCTGAGGTGAGGGCCAAGCTGGAGGAGCAGGCCCAGCAGAT
CAGGCTCCAGGCAGAGGCCTTCCAGGCCAGGCTGAAGAGCTGGTTTGAGCCCCTGGTGGAGGACATGCAGAGG
CAGTGGGCTGGCCTGGTGGAGAAGGTCCAGGCTGCTGTGGGCACCTCAGCAGCCCCTGTCCCCAGTGACAACC
ACTGA
ApoE3ch-31 (SEQ ID NO: 103)
ATGAAAGTGCTGTGGGCTGCCCTGCTGGTCACATTCCTGGCTGGCTGCCAGGCCAAGGTGGAGCAGGCTGTGG
AGACAGAGCCAGAGCCAGAACTGAGGCAGCAGACAGAGTGGCAGTCTGGCCAAAGATGGGAGCTAGCCCTGGG
AAGATTCTGGGACTACCTGAGATGGGTGCAGACCCTCTCAGAACAAGTCCAGGAGGAGCTGCTGTCCTCACAG
GTGACCCAGGAGCTGAGGGCCCTGATGGATGAAACAATGAAGGAGCTGAAGGCATATAAGTCAGAATTGGAGG
AGCAGCTGACTCCTGTGGCTGAGGAGACCAGGGCTAGACTCTCCAAGGAGCTCCAGGCTGCCCAGGCCAGATT
AGGAGCAGACATGGAGGATGTCTGTGGGAGGCTGGTGCAGTACAGTGGTGAGGTCCAGGCCATGCTGGGCCAA
AGCACAGAGGAGCTGAGGGTCAGGCTGGCCAGCCATCTGAGGAAGCTGAGGAAAAGGCTGCTCAGGGATGCTG
ATGACCTGCAGAAGAGACTGGCAGTGTACCAGGCTGGTGCCAGAGAGGGGGCAGAGAGGGGGCTCTCAGCCAT
CAGAGAGAGGCTGGGGCCTCTGGTGGAGCAGGGCAGAGTGAGGGCTGCCACTGTGGGCTCCTTGGCAGGACAG
CCCCTCCAAGAGAGAGCTCAGGCCTGGGGAGAGAGGCTCAGGGCCAGGATGGAGGAAATGGGCAGTAGGACCA
GGGACAGGCTTGATGAGGTGAAGGAGCAGGTGGCTGAGGTCAGAGCCAAGCTGGAGGAGCAGGCTCAGCAGAT
TAGGCTGCAGGCAGAAGCCTTCCAGGCCAGGCTGAAATCCTGGTTTGAGCCCCTGGTGGAAGACATGCAAAGG
CAGTGGGCAGGGCTGGTGGAGAAGGTGCAGGCTGCTGTGGGCACATCAGCTGCCCCTGTGCCCAGTGATAACC
ACTGA
ApoE3ch-33 (SEQ ID NO: 104)
ATGAAAGTGCTATGGGCAGCCCTCTTGGTGACCTTCCTGGCTGGCTGCCAGGCCAAGGTGGAGCAGGCTGTGG
AGACAGAGCCAGAACCAGAGCTGAGACAGCAAACTGAGTGGCAGTCAGGCCAGAGATGGGAGCTGGCCCTGGG
GAGATTTTGGGACTACTTAAGGTGGGTCCAGACCCTCAGTGAGCAGGTGCAGGAAGAGCTGCTGAGCTCCCAG
GTCACCCAGGAGCTGAGGGCCCTTATGGATGAAACCATGAAGGAGCTGAAGGCCTACAAGTCAGAACTGGAGG
AGCAGCTCACCCCTGTGGCTGAGGAGACCAGGGCAAGGCTCTCCAAGGAGCTACAGGCAGCCCAGGCCAGGCT
GGGTGCAGACATGGAGGATGTCTGTGGGAGACTGGTGCAGTACAGTGGTGAAGTACAGGCCATGCTTGGCCAA
TCCACAGAGGAGTTGAGGGTGAGGCTGGCCTCCCACCTGAGGAAGCTGAGAAAGAGACTCCTCAGGGATGCAG
ATGACCTCCAGAAGAGGCTGGCAGTTTACCAGGCAGGGGCCAGAGAGGGTGCAGAAAGAGGGCTGTCAGCCAT
CAGGGAAAGGCTGGGCCCTCTGGTGGAGCAGGGCAGAGTGAGAGCAGCAACTGTTGGCTCCTTGGCTGGGCAG
CCTCTGCAGGAGAGAGCTCAGGCCTGGGGGGAGAGACTCAGGGCCAGGATGGAGGAGATGGGCTCCAGGACAA
GGGACAGGTTGGATGAAGTGAAGGAGCAGGTGGCTGAGGTGAGAGCAAAGTTGGAGGAGCAGGCCCAGCAGAT
CAGGCTTCAGGCAGAGGCCTTCCAGGCCAGGCTGAAGTCCTGGTTTGAGCCACTGGTGGAGGATATGCAGAGA
CAGTGGGCAGGCCTGGTGGAGAAGGTCCAGGCAGCAGTGGGTACCTCTGCAGCCCCAGTTCCCAGTGACAATC
ACTGA
ApoE3chAGT-co3 (SEQ ID NO: 105)
ATGAAGGTGCTGTGGGCTGCCCTGCTGGTGACCTTCCTGGCTGGCTGTCAGGCTAAAGTGGAGCAGGCTGTGG
AGACTGAGCCTGAGCCAGAGCTGAGGCAGCAGACTGAGTGGCAGTCTGGCCAGAGATGGGAGCTGGCTCTGGG
CAGGTTCTGGGACTACCTGAGGTGGGTGCAGACCCTGTCTGAGCAGGTGCAGGAGGAGCTGCTGTCTTCTCAG
GTGACCCAGGAACTGAGGGCCCTGATGGATGAGACTATGAAGGAGCTGAAGGCCTACAAGTCTGAGCTGGAGG
AGCAGCTGACCCCTGTGGCTGAGGAGACCAGGGCCAGGCTGAGCAAGGAGCTGCAGGCTGCCCAGGCCAGACT
GGGGGCTGACATGGAGGATGTGTGTGGCAGACTGGTGCAGTACAGTGGGGAGGTGCAGGCCATGCTGGGGCAG
TCTACTGAGGAGCTGAGGGTGAGGCTGGCCAGCCACCTGAGGAAGCTGAGGAAGAGGCTGCTGAGGGATGCTG
ATGACCTGCAGAAGAGGCTGGCTGTGTATCAGGCTGGGGCTAGGGAGGGGGCTGAGAGGGGCCTGTCTGCCAT
CAGGGAGAGGCTGGGGCCTCTGGTGGAGCAGGGCAGGGTGAGGGCTGCCACTGTGGGGAGCCTGGCTGGCCAG
CCTCTGCAGGAGAGGGCTCAGGCCTGGGGGGAGAGGCTGAGGGCCAGGATGGAGGAGATGGGGAGCAGGACCA
GGGACAGGCTGGATGAGGTGAAGGAGCAGGTGGCTGAGGTGAGGGCCAAGCTGGAAGAGCAGGCCCAGCAGAT
CAGGCTGCAGGCTGAGGCCTTCCAGGCCAGGCTGAAGAGCTGGTTTGAGCCCCTGGTGGAGGACATGCAGAGG
CAGTGGGCTGGCCTGGTGGAGAAGGTGCAGGCTGCTGTGGGCACCTCTGCTGCCCCTGTGCCCTCTGACAATC
ACTGA
Intron Insertions
First intron inserted in signal peptide at codon 15 (split Gly G/GC) ;
second intron inserted in ApoE3ch sequence at codon 79 (split Arg AG/G)
ApoE3ch-41-RBPi-FIXi (SEQ ID NO: 106)
ATGAAGGTCCTCTGGGCAGCCCTGCTGGTCACATTCCTGGCAGGTCAGTGGCAGCCAGGGGCAGCTGCACCCT
TTGCAGCTCCAGGGTTCCCCAAGGGCCCTGCCTGCTGATGGCCAGTGGGCATTGTGAAGGGAAGGGAGCACCA
AATGGGTGGAGGGAGGGAGGAAGCCCTTTGCCCTGGCTTGGCTGAGGATCCCCTTGGCTTTTGCAGGCTGTCA
AGCCAAGGTGGAACAAGCTGTGGAGACTGAGCCAGAGCCAGAGCTCAGGCAGCAAACAGAGTGGCAGTCTGGC
CAGAGATGGGAATTGGCACTGGGAAGATTCTGGGACTACCTGAGGTGGGTTCAGACCCTGTCAGAGCAGGTGC
AGGAGGAGCTCCTGAGCTCACAAGTCACTCAAGAGCTGAGGTTTGTTTCCTTTTTTATAATACATTGAGTATG
CTTGCCTTTTAGATATAGAAATATCTGATTCTGTCTTCTTCACTAAATTTTGATTACATGATTTGACAGCAAT
ATTGAAGAGTCTAACAGCCAGCACCCAGGTTGGTAAGTACTGGTTCTTTGTTAGCTAGGTTTTCTTCTTCTTC
ACTTTTAAAACTAAATAGATGGACAATGCTTATGATGCAATAAGGTTTAATAAACACTGTTCAGTTCAGTATT
TGGTCATGTAATTCCTGTTAAAAAACAGTCATCTCCTTGGTTTAAAAAAATTAAAAGTGGGAAAACAAAGAAA
TAGCAGAATATAGTGAAAAAAAATAACCACAGTATTTTTGTTTGGACTTACCACTTTGAAATCAAATTAGAGG
AAGGCATACAGGGAAGAAATTATCCCATTTGGACAAACAGCATGTTCTCACAGTAAGCACTTATCACACTTAC
TTGTCAACTTTTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAAT
TAGGCTTCTGTTCTTCAGGAGACATTTGTTCAAAGTCATTTGGGCAACCATATTCTGAAAACAGCCCAGCCAG
GGTGATGGATCACTTTGCAAAGATCCTCAATGAGCTATTTTCAAGTGATGACAAAGTGTGAAGTTAAGGGCTC
ATTTGAGAACTTTCTTTTTCATCCAAAGTAAATTCAAATATGATTAGAAATCTGACCTTTTATTACTGGAATT
CTCTTGACTAAAAGTAAAATTGAATTTTAATTCCTAAATCTCCATGTGTATACAGTACTGTGGGAACATCACA
GATTTTGGCTCCATGCCCTAAAGAGAAATTGGCTTTCAGATTATTTGGATTAAAAACAAAGACTTTCTTAAGA
GATGTAAAATTTTCATGATGTTTTCTTTTTTGCTAAAACTAAAGAATTATTCTTTTACATTTCAGAGCACTTA
TGGATGAAACCATGAAGGAATTAAAGGCCTACAAATCAGAATTGGAAGAGCAGCTGACACCTGTGGCTGAAGA
GACCAGGGCCAGACTGAGTAAAGAACTGCAAGCAGCCCAGGCCAGACTTGGAGCTGACATGGAGGATGTTTGT
GGCAGGCTGGTGCAGTACAGTGGTGAAGTCCAAGCCATGCTGGGACAATCCACAGAGGAGCTGAGGGTCAGGC
TGGCCAGCCACCTAAGAAAACTGAGGAAGAGACTCCTGAGGGATGCTGATGATCTCCAGAAAAGGTTGGCTGT
GTACCAAGCTGGAGCCAGGGAAGGAGCTGAAAGGGGACTCTCTGCCATCAGAGAAAGACTGGGTCCCCTGGTG
GAGCAGGGCAGGGTGAGAGCAGCCACAGTGGGAAGCTTGGCAGGACAGCCACTCCAGGAAAGGGCACAGGCAT
GGGGAGAAAGGCTGAGGGCCAGGATGGAGGAGATGGGCAGCAGGACCAGGGACAGGCTGGATGAGGTGAAGGA
ACAAGTGGCAGAGGTAAGGGCAAAACTGGAGGAGCAGGCCCAGCAGATCAGGCTCCAGGCTGAAGCCTTCCAA
GCAAGGTTGAAAAGCTGGTTTGAGCCTCTTGTGGAAGACATGCAGAGACAGTGGGCTGGCCTTGTGGAAAAAG
TCCAAGCTGCAGTAGGCACCAGTGCAGCACCTGTCCCATCAGACAACCATTGA
ApoE3ch-42-RBPi-FIXi (SEQ ID NO: 107)
ATGAAAGTCCTCTGGGCTGCCTTGCTGGTAACTTTCTTGGCTGGTCAGTGGCAGCCAGGGGCAGCTGCACCCT
TTGCAGCTCCAGGGTTCCCCAAGGGCCCTGCCTGCTGATGGCCAGTGGGCATTGTGAAGGGAAGGGAGCACCA
AATGGGTGGAGGGAGGGAGGAAGCCCTTTGCCCTGGCTTGGCTGAGGATCCCCTTGGCTTTTGCAGGCTGCCA
GGCCAAGGTGGAGCAAGCTGTGGAAACAGAGCCAGAGCCAGAGCTGAGGCAACAGACAGAATGGCAGTCAGGC
CAGAGGTGGGAGCTGGCTCTGGGGAGATTCTGGGATTACCTGAGGTGGGTCCAGACACTCTCTGAACAAGTGC
AAGAGGAGCTGCTGTCCTCCCAAGTCACCCAGGAGCTGAGGTTTGTTTCCTTTTTTATAATACATTGAGTATG
CTTGCCTTTTAGATATAGAAATATCTGATTCTGTCTTCTTCACTAAATTTTGATTACATGATTTGACAGCAAT
ATTGAAGAGTCTAACAGCCAGCACCCAGGTTGGTAAGTACTGGTTCTTTGTTAGCTAGGTTTTCTTCTTCTTC
ACTTTTAAAACTAAATAGATGGACAATGCTTATGATGCAATAAGGTTTAATAAACACTGTTCAGTTCAGTATT
TGGTCATGTAATTCCTGTTAAAAAACAGTCATCTCCTTGGTTTAAAAAAATTAAAAGTGGGAAAACAAAGAAA
TAGCAGAATATAGTGAAAAAAAATAACCACAGTATTTTTGTTTGGACTTACCACTTTGAAATCAAATTAGAGG
AAGGCATACAGGGAAGAAATTATCCCATTTGGACAAACAGCATGTTCTCACAGTAAGCACTTATCACACTTAC
TTGTCAACTTTTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAAT
TAGGCTTCTGTTCTTCAGGAGACATTTGTTCAAAGTCATTTGGGCAACCATATTCTGAAAACAGCCCAGCCAG
GGTGATGGATCACTTTGCAAAGATCCTCAATGAGCTATTTTCAAGTGATGACAAAGTGTGAAGTTAAGGGCTC
ATTTGAGAACTTTCTTTTTCATCCAAAGTAAATTCAAATATGATTAGAAATCTGACCTTTTATTACTGGAATT
CTCTTGACTAAAAGTAAAATTGAATTTTAATTCCTAAATCTCCATGTGTATACAGTACTGTGGGAACATCACA
GATTTTGGCTCCATGCCCTAAAGAGAAATTGGCTTTCAGATTATTTGGATTAAAAACAAAGACTTTCTTAAGA
GATGTAAAATTTTCATGATGTTTTCTTTTTTGCTAAAACTAAAGAATTATTCTTTTACATTTCAGAGCCTTGA
TGGATGAAACCATGAAAGAGCTGAAAGCTTACAAGTCTGAGCTGGAGGAACAGTTGACCCCAGTGGCTGAGGA
GACCAGGGCCAGATTGTCCAAGGAACTGCAGGCTGCTCAGGCAAGGTTGGGGGCAGATATGGAGGATGTGTGT
GGGAGGTTGGTGCAGTACAGTGGTGAGGTGCAGGCCATGCTGGGACAGAGCACTGAAGAGCTAAGGGTCAGAT
TAGCAAGTCATTTGAGGAAGCTGAGAAAGAGGCTGCTGAGAGATGCTGATGACCTTCAGAAGAGACTGGCTGT
CTACCAAGCTGGTGCCAGAGAAGGGGCTGAAAGAGGTCTCTCAGCCATCAGGGAGAGGCTGGGGCCTCTGGTT
GAGCAGGGTAGGGTGAGAGCAGCCACTGTGGGATCCCTGGCTGGTCAACCACTCCAAGAAAGAGCACAGGCCT
GGGGGGAGAGACTGAGGGCCAGGATGGAGGAAATGGGCAGCAGGACAAGAGACAGGCTGGATGAGGTCAAGGA
GCAAGTAGCAGAAGTGAGGGCCAAACTGGAGGAACAGGCCCAGCAGATCAGACTCCAAGCTGAGGCCTTCCAG
GCCAGGCTGAAATCTTGGTTTGAGCCTTTGGTAGAAGACATGCAGAGGCAGTGGGCAGGGTTGGTAGAAAAGG
TGCAGGCTGCTGTGGGGACTTCAGCAGCCCCTGTGCCCTCAGACAATCACTGA
ApoE3ch-43-RBPi-FIXi (SEQ ID NO: 108)
ATGAAGGTCCTCTGGGCTGCCCTGCTGGTCACCTTTCTAGCTGGTCAGTGGCAGCCAGGGGCAGCTGCACCCT
TTGCAGCTCCAGGGTTCCCCAAGGGCCCTGCCTGCTGATGGCCAGTGGGCATTGTGAAGGGAAGGGAGCACCA
AATGGGTGGAGGGAGGGAGGAAGCCCTTTGCCCTGGCTTGGCTGAGGATCCCCTTGGCTTTTGCAGGGTGCCA
GGCCAAGGTGGAACAAGCAGTGGAAACTGAGCCTGAGCCTGAGCTGAGGCAACAGACAGAGTGGCAGTCTGGT
CAGAGGTGGGAGCTTGCACTGGGGAGGTTCTGGGATTACCTGAGGTGGGTCCAGACCCTGAGTGAGCAGGTGC
AGGAGGAGCTCCTTAGCTCACAGGTTACTCAGGAGTTGAGGTTTGTTTCCTTTTTTATAATACATTGAGTATG
CTTGCCTTTTAGATATAGAAATATCTGATTCTGTCTTCTTCACTAAATTTTGATTACATGATTTGACAGCAAT
ATTGAAGAGTCTAACAGCCAGCACCCAGGTTGGTAAGTACTGGTTCTTTGTTAGCTAGGTTTTCTTCTTCTTC
ACTTTTAAAACTAAATAGATGGACAATGCTTATGATGCAATAAGGTTTAATAAACACTGTTCAGTTCAGTATT
TGGTCATGTAATTCCTGTTAAAAAACAGTCATCTCCTTGGTTTAAAAAAATTAAAAGTGGGAAAACAAAGAAA
TAGCAGAATATAGTGAAAAAAAATAACCACAGTATTTTTGTTTGGACTTACCACTTTGAAATCAAATTAGAGG
AAGGCATACAGGGAAGAAATTATCCCATTTGGACAAACAGCATGTTCTCACAGTAAGCACTTATCACACTTAC
TTGTCAACTTTTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAAT
TAGGCTTCTGTTCTTCAGGAGACATTTGTTCAAAGTCATTTGGGCAACCATATTCTGAAAACAGCCCAGCCAG
GGTGATGGATCACTTTGCAAAGATCCTCAATGAGCTATTTTCAAGTGATGACAAAGTGTGAAGTTAAGGGCTC
ATTTGAGAACTTTCTTTTTCATCCAAAGTAAATTCAAATATGATTAGAAATCTGACCTTTTATTACTGGAATT
CTCTTGACTAAAAGTAAAATTGAATTTTAATTCCTAAATCTCCATGTGTATACAGTACTGTGGGAACATCACA
GATTTTGGCTCCATGCCCTAAAGAGAAATTGGCTTTCAGATTATTTGGATTAAAAACAAAGACTTTCTTAAGA
GATGTAAAATTTTCATGATGTTTTCTTTTTTGCTAAAACTAAAGAATTATTCTTTTACATTTCAGAGCACTCA
TGGATGAAACCATGAAGGAGCTGAAGGCCTACAAAAGTGAACTGGAAGAACAGCTGACCCCTGTTGCAGAAGA
AACCAGGGCAAGGCTCTCCAAAGAGCTGCAAGCTGCTCAGGCAAGACTGGGTGCTGACATGGAGGATGTCTGT
GGCAGGCTGGTGCAGTATAGTGGTGAGGTGCAGGCAATGCTGGGGCAGTCCACTGAAGAGCTGAGGGTGAGGC
TGGCCTCTCATCTTAGGAAGTTAAGGAAGAGGCTGCTCAGGGATGCTGATGACCTCCAGAAGAGGCTGGCTGT
GTATCAGGCTGGGGCAAGGGAGGGGGCAGAGAGGGGCCTCTCTGCCATAAGGGAGAGACTGGGTCCACTGGTG
GAACAGGGCAGGGTGAGAGCAGCCACAGTGGGCTCTCTGGCAGGGCAGCCCCTCCAAGAAAGAGCCCAGGCTT
GGGGAGAAAGACTGAGGGCCAGGATGGAGGAAATGGGATCCAGGACAAGGGACAGGCTGGATGAGGTGAAGGA
GCAGGTGGCTGAGGTGAGGGCAAAGCTGGAAGAGCAAGCCCAGCAGATCAGGCTCCAGGCTGAAGCTTTCCAA
GCAAGGCTGAAAAGCTGGTTTGAGCCCCTGGTGGAGGACATGCAGAGGCAGTGGGCAGGCCTGGTGGAGAAAG
TTCAGGCAGCTGTGGGTACTTCAGCTGCACCTGTCCCTTCTGACAATCACTGA
ApoE3ch-44-RBPi-FIXi (SEQ ID NO: 109)
ATGAAAGTCCTGTGGGCAGCCCTCCTGGTCACCTTCCTGGCTGGTCAGTGGCAGCCAGGGGCAGCTGCACCCT
TTGCAGCTCCAGGGTTCCCCAAGGGCCCTGCCTGCTGATGGCCAGTGGGCATTGTGAAGGGAAGGGAGCACCA
AATGGGTGGAGGGAGGGAGGAAGCCCTTTGCCCTGGCTTGGCTGAGGATCCCCTTGGCTTTTGCAGGCTGTCA
AGCTAAAGTGGAGCAGGCTGTGGAAACAGAGCCTGAGCCAGAACTTAGACAACAAACTGAGTGGCAGAGTGGG
CAGAGGTGGGAGCTGGCCTTGGGCAGATTCTGGGACTACCTGAGGTGGGTCCAGACTCTTTCTGAGCAGGTGC
AGGAGGAGCTCCTGTCCAGTCAGGTGACCCAGGAGCTGAGGTTTGTTTCCTTTTTTATAATACATTGAGTATG
CTTGCCTTTTAGATATAGAAATATCTGATTCTGTCTTCTTCACTAAATTTTGATTACATGATTTGACAGCAAT
ATTGAAGAGTCTAACAGCCAGCACCCAGGTTGGTAAGTACTGGTTCTTTGTTAGCTAGGTTTTCTTCTTCTTC
ACTTTTAAAACTAAATAGATGGACAATGCTTATGATGCAATAAGGTTTAATAAACACTGTTCAGTTCAGTATT
TGGTCATGTAATTCCTGTTAAAAAACAGTCATCTCCTTGGTTTAAAAAAATTAAAAGTGGGAAAACAAAGAAA
TAGCAGAATATAGTGAAAAAAAATAACCACAGTATTTTTGTTTGGACTTACCACTTTGAAATCAAATTAGAGG
AAGGCATACAGGGAAGAAATTATCCCATTTGGACAAACAGCATGTTCTCACAGTAAGCACTTATCACACTTAC
TTGTCAACTTTTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAAT
TAGGCTTCTGTTCTTCAGGAGACATTTGTTCAAAGTCATTTGGGCAACCATATTCTGAAAACAGCCCAGCCAG
GGTGATGGATCACTTTGCAAAGATCCTCAATGAGCTATTTTCAAGTGATGACAAAGTGTGAAGTTAAGGGCTC
ATTTGAGAACTTTCTTTTTCATCCAAAGTAAATTCAAATATGATTAGAAATCTGACCTTTTATTACTGGAATT
CTCTTGACTAAAAGTAAAATTGAATTTTAATTCCTAAATCTCCATGTGTATACAGTACTGTGGGAACATCACA
GATTTTGGCTCCATGCCCTAAAGAGAAATTGGCTTTCAGATTATTTGGATTAAAAACAAAGACTTTCTTAAGA
GATGTAAAATTTTCATGATGTTTTCTTTTTTGCTAAAACTAAAGAATTATTCTTTTACATTTCAGGGCTCTCA
TGGATGAGACAATGAAAGAGCTGAAGGCCTACAAAAGTGAGCTGGAAGAGCAGCTGACCCCAGTGGCTGAAGA
GACAAGGGCCAGGTTAAGCAAAGAGCTCCAGGCTGCCCAGGCCAGGCTGGGAGCAGACATGGAGGATGTCTGT
GGCAGGCTGGTGCAGTATAGTGGGGAGGTGCAAGCCATGTTAGGCCAGAGCACTGAAGAGCTGAGGGTGAGGT
TAGCTTCTCACCTGAGAAAATTAAGGAAGAGGCTGCTGAGGGATGCTGATGACCTGCAGAAGAGGTTGGCTGT
TTACCAGGCAGGGGCTAGAGAGGGAGCAGAGAGGGGCCTCTCTGCCATCAGAGAGAGGCTGGGCCCCCTTGTG
GAGCAGGGCAGGGTGAGAGCAGCCACTGTTGGCTCACTTGCAGGGCAGCCTCTGCAGGAAAGAGCCCAGGCAT
GGGGGGAGAGACTGAGGGCAAGAATGGAAGAGATGGGCTCAAGGACAAGAGACAGGCTGGATGAGGTGAAGGA
GCAGGTGGCAGAAGTGAGGGCCAAGCTAGAGGAACAGGCCCAGCAGATCAGGCTCCAGGCAGAAGCCTTCCAG
GCCAGGCTCAAGTCCTGGTTTGAGCCCTTAGTGGAGGACATGCAGAGGCAGTGGGCTGGCCTGGTGGAGAAGG
TACAGGCTGCTGTGGGGACTTCAGCAGCCCCAGTGCCCAGTGACAACCACTGA
ApoE3ch-45-RBPi-FIXi (SEQ ID NO: 110)
ATGAAGGTCCTCTGGGCAGCTCTCCTGGTTACCTTCTTAGCTGGTCAGTGGCAGCCAGGGGCAGCTGCACCCT
TTGCAGCTCCAGGGTTCCCCAAGGGCCCTGCCTGCTGATGGCCAGTGGGCATTGTGAAGGGAAGGGAGCACCA
AATGGGTGGAGGGAGGGAGGAAGCCCTTTGCCCTGGCTTGGCTGAGGATCCCCTTGGCTTTTGCAGGCTGTCA
AGCCAAAGTAGAACAAGCAGTGGAAACAGAGCCAGAGCCAGAGCTCAGACAGCAAACAGAATGGCAGTCTGGT
CAGAGATGGGAACTGGCACTGGGTAGGTTCTGGGACTACCTGAGGTGGGTCCAGACCCTGTCAGAGCAGGTCC
AGGAAGAGCTGCTGAGCAGCCAGGTGACTCAAGAGCTGAGGTTTGTTTCCTTTTTTATAATACATTGAGTATG
CTTGCCTTTTAGATATAGAAATATCTGATTCTGTCTTCTTCACTAAATTTTGATTACATGATTTGACAGCAAT
ATTGAAGAGTCTAACAGCCAGCACCCAGGTTGGTAAGTACTGGTTCTTTGTTAGCTAGGTTTTCTTCTTCTTC
ACTTTTAAAACTAAATAGATGGACAATGCTTATGATGCAATAAGGTTTAATAAACACTGTTCAGTTCAGTATT
TGGTCATGTAATTCCTGTTAAAAAACAGTCATCTCCTTGGTTTAAAAAAATTAAAAGTGGGAAAACAAAGAAA
TAGCAGAATATAGTGAAAAAAAATAACCACAGTATTTTTGTTTGGACTTACCACTTTGAAATCAAATTAGAGG
AAGGCATACAGGGAAGAAATTATCCCATTTGGACAAACAGCATGTTCTCACAGTAAGCACTTATCACACTTAC
TTGTCAACTTTTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAAT
TAGGCTTCTGTTCTTCAGGAGACATTTGTTCAAAGTCATTTGGGCAACCATATTCTGAAAACAGCCCAGCCAG
GGTGATGGATCACTTTGCAAAGATCCTCAATGAGCTATTTTCAAGTGATGACAAAGTGTGAAGTTAAGGGCTC
ATTTGAGAACTTTCTTTTTCATCCAAAGTAAATTCAAATATGATTAGAAATCTGACCTTTTATTACTGGAATT
CTCTTGACTAAAAGTAAAATTGAATTTTAATTCCTAAATCTCCATGTGTATACAGTACTGTGGGAACATCACA
GATTTTGGCTCCATGCCCTAAAGAGAAATTGGCTTTCAGATTATTTGGATTAAAAACAAAGACTTTCTTAAGA
GATGTAAAATTTTCATGATGTTTTCTTTTTTGCTAAAACTAAAGAATTATTCTTTTACATTTCAGGGCCCTCA
TGGATGAAACCATGAAGGAGCTGAAGGCCTACAAGAGTGAGTTAGAAGAGCAGCTCACACCTGTGGCTGAAGA
AACAAGGGCCAGGCTCAGCAAGGAGCTGCAAGCTGCACAGGCCAGGCTGGGTGCTGATATGGAGGATGTGTGT
GGCAGGCTGGTGCAGTATAGTGGTGAAGTCCAGGCCATGTTGGGCCAATCCACAGAGGAGCTCAGGGTCAGGC
TGGCCTCCCATCTCAGGAAACTAAGAAAGAGACTGCTCAGGGATGCTGATGACCTTCAGAAGAGGCTGGCTGT
GTACCAGGCAGGAGCCAGAGAGGGGGCTGAGAGGGGTCTCTCAGCCATCAGGGAAAGACTTGGGCCCTTGGTT
GAGCAGGGCAGGGTGAGAGCAGCCACAGTTGGGTCTCTGGCTGGCCAGCCCCTCCAGGAGAGAGCTCAGGCCT
GGGGTGAGAGACTGAGGGCCAGGATGGAGGAGATGGGGAGCAGGACCAGGGACAGGTTGGATGAGGTCAAGGA
GCAGGTGGCAGAAGTGAGAGCCAAGCTGGAAGAGCAGGCCCAGCAAATCAGGCTGCAAGCAGAGGCATTTCAG
GCCAGGCTGAAGTCATGGTTTGAGCCTCTGGTGGAGGACATGCAGAGGCAGTGGGCTGGGCTGGTGGAAAAGG
TACAGGCTGCTGTGGGCACCAGTGCAGCACCTGTCCCATCTGATAACCATTGA
ApoE3ch-46-RBPi-FIXi (SEQ ID NO: 111)
ATGAAAGTCCTGTGGGCAGCCCTCCTTGTGACCTTCCTGGCAGGTCAGTGGCAGCCAGGGGCAGCTGCACCCT
TTGCAGCTCCAGGGTTCCCCAAGGGCCCTGCCTGCTGATGGCCAGTGGGCATTGTGAAGGGAAGGGAGCACCA
AATGGGTGGAGGGAGGGAGGAAGCCCTTTGCCCTGGCTTGGCTGAGGATCCCCTTGGCTTTTGCAGGCTGTCA
AGCAAAGGTGGAGCAGGCTGTAGAGACTGAGCCAGAGCCTGAACTGAGGCAGCAGACAGAATGGCAGTCTGGC
CAGAGATGGGAGCTGGCCCTGGGAAGATTTTGGGACTACCTGAGGTGGGTCCAGACCCTGTCAGAACAGGTTC
AGGAAGAGCTGCTTTCCAGCCAAGTGACACAGGAGCTGAGGTTTGTTTCCTTTTTTATAATACATTGAGTATG
CTTGCCTTTTAGATATAGAAATATCTGATTCTGTCTTCTTCACTAAATTTTGATTACATGATTTGACAGCAAT
ATTGAAGAGTCTAACAGCCAGCACCCAGGTTGGTAAGTACTGGTTCTTTGTTAGCTAGGTTTTCTTCTTCTTC
ACTTTTAAAACTAAATAGATGGACAATGCTTATGATGCAATAAGGTTTAATAAACACTGTTCAGTTCAGTATT
TGGTCATGTAATTCCTGTTAAAAAACAGTCATCTCCTTGGTTTAAAAAAATTAAAAGTGGGAAAACAAAGAAA
TAGCAGAATATAGTGAAAAAAAATAACCACAGTATTTTTGTTTGGACTTACCACTTTGAAATCAAATTAGAGG
AAGGCATACAGGGAAGAAATTATCCCATTTGGACAAACAGCATGTTCTCACAGTAAGCACTTATCACACTTAC
TTGTCAACTTTTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAAT
TAGGCTTCTGTTCTTCAGGAGACATTTGTTCAAAGTCATTTGGGCAACCATATTCTGAAAACAGCCCAGCCAG
GGTGATGGATCACTTTGCAAAGATCCTCAATGAGCTATTTTCAAGTGATGACAAAGTGTGAAGTTAAGGGCTC
ATTTGAGAACTTTCTTTTTCATCCAAAGTAAATTCAAATATGATTAGAAATCTGACCTTTTATTACTGGAATT
CTCTTGACTAAAAGTAAAATTGAATTTTAATTCCTAAATCTCCATGTGTATACAGTACTGTGGGAACATCACA
GATTTTGGCTCCATGCCCTAAAGAGAAATTGGCTTTCAGATTATTTGGATTAAAAACAAAGACTTTCTTAAGA
GATGTAAAATTTTCATGATGTTTTCTTTTTTGCTAAAACTAAAGAATTATTCTTTTACATTTCAGAGCCCTTA
TGGATGAGACAATGAAAGAACTGAAGGCTTATAAAAGTGAGCTAGAGGAGCAGCTGACCCCAGTGGCTGAAGA
GACCAGGGCCAGGCTCAGCAAAGAGCTCCAGGCTGCACAGGCCAGATTGGGAGCTGACATGGAGGATGTGTGT
GGGAGACTAGTGCAGTACAGTGGTGAAGTGCAGGCCATGCTGGGACAAAGCACAGAAGAGTTGAGAGTCAGGC
TGGCATCCCACCTGAGGAAACTCAGGAAAAGACTGCTCAGGGATGCTGATGACCTCCAGAAGAGACTGGCAGT
CTATCAGGCTGGTGCCAGAGAGGGGGCAGAGAGGGGACTCTCAGCCATCAGGGAGAGGCTGGGGCCTCTGGTT
GAGCAGGGCAGAGTCAGAGCAGCAACAGTGGGGAGTCTGGCAGGCCAGCCCTTACAAGAGAGAGCTCAGGCAT
GGGGAGAGAGGCTGAGGGCCAGGATGGAAGAAATGGGATCAAGGACTAGAGACAGGTTGGATGAAGTAAAGGA
GCAGGTGGCAGAAGTGAGGGCCAAGCTTGAGGAGCAGGCCCAGCAGATCAGGCTACAGGCTGAGGCATTTCAG
GCCAGGCTGAAGAGCTGGTTTGAGCCCTTGGTGGAGGATATGCAGAGGCAGTGGGCTGGATTGGTGGAAAAGG
TACAGGCTGCTGTGGGCACCTCAGCAGCTCCTGTCCCCTCTGATAATCATTGA
ApoE3ch-47-RBPi-FIXi (SEQ ID NO: 112)
ATGAAGGTCCTCTGGGCAGCCCTGCTGGTCACCTTCCTGGCTGGTCAGTGGCAGCCAGGGGCAGCTGCACCCT
TTGCAGCTCCAGGGTTCCCCAAGGGCCCTGCCTGCTGATGGCCAGTGGGCATTGTGAAGGGAAGGGAGCACCA
AATGGGTGGAGGGAGGGAGGAAGCCCTTTGCCCTGGCTTGGCTGAGGATCCCCTTGGCTTTTGCAGGCTGCCA
GGCCAAGGTGGAGCAGGCTGTGGAGACTGAGCCAGAGCCAGAGCTGAGGCAGCAGACAGAGTGGCAGTCTGGC
CAGAGATGGGAGCTGGCCCTGGGAAGATTCTGGGACTACCTGAGGTGGGTCCAGACCCTGTCAGAGCAGGTGC
AGGAGGAGCTGCTGAGCTCCCAGGTGACCCAGGAGCTGAGGTTTGTTTCCTTTTTTATAATACATTGAGTATG
CTTGCCTTTTAGATATAGAAATATCTGATTCTGTCTTCTTCACTAAATTTTGATTACATGATTTGACAGCAAT
ATTGAAGAGTCTAACAGCCAGCACCCAGGTTGGTAAGTACTGGTTCTTTGTTAGCTAGGTTTTCTTCTTCTTC
ACTTTTAAAACTAAATAGATGGACAATGCTTATGATGCAATAAGGTTTAATAAACACTGTTCAGTTCAGTATT
TGGTCATGTAATTCCTGTTAAAAAACAGTCATCTCCTTGGTTTAAAAAAATTAAAAGTGGGAAAACAAAGAAA
TAGCAGAATATAGTGAAAAAAAATAACCACAGTATTTTTGTTTGGACTTACCACTTTGAAATCAAATTAGAGG
AAGGCATACAGGGAAGAAATTATCCCATTTGGACAAACAGCATGTTCTCACAGTAAGCACTTATCACACTTAC
TTGTCAACTTTTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAAT
TAGGCTTCTGTTCTTCAGGAGACATTTGTTCAAAGTCATTTGGGCAACCATATTCTGAAAACAGCCCAGCCAG
GGTGATGGATCACTTTGCAAAGATCCTCAATGAGCTATTTTCAAGTGATGACAAAGTGTGAAGTTAAGGGCTC
ATTTGAGAACTTTCTTTTTCATCCAAAGTAAATTCAAATATGATTAGAAATCTGACCTTTTATTACTGGAATT
CTCTTGACTAAAAGTAAAATTGAATTTTAATTCCTAAATCTCCATGTGTATACAGTACTGTGGGAACATCACA
GATTTTGGCTCCATGCCCTAAAGAGAAATTGGCTTTCAGATTATTTGGATTAAAAACAAAGACTTTCTTAAGA
GATGTAAAATTTTCATGATGTTTTCTTTTTTGCTAAAACTAAAGAATTATTCTTTTACATTTCAGGGCCCTGA
TGGATGAAACCATGAAGGAGCTGAAGGCCTACAAGAGTGAGCTGGAGGAGCAGCTGACCCCTGTGGCTGAAGA
GACCAGGGCCAGGCTCAGCAAGGAGCTGCAGGCTGCCCAGGCCAGGCTGGGAGCTGACATGGAGGATGTGTGT
GGCAGGCTGGTGCAGTACAGTGGTGAGGTGCAGGCCATGCTGGGACAGAGCACTGAAGAGCTGAGGGTCAGGC
TGGCCTCCCACCTGAGGAAGCTGAGGAAGAGACTGCTCAGGGATGCTGATGACCTCCAGAAGAGGCTGGCTGT
GTACCAGGCTGGGGCCAGAGAGGGGGCAGAGAGGGGACTCTCAGCCATCAGGGAGAGGCTGGGGCCTCTGGTG
GAGCAGGGCAGGGTGAGAGCAGCCACAGTGGGCTCTCTGGCAGGCCAGCCTCTCCAGGAAAGAGCTCAGGCCT
GGGGAGAGAGACTGAGGGCCAGGATGGAGGAGATGGGCAGCAGGACCAGGGACAGGCTGGATGAGGTGAAGGA
GCAGGTGGCTGAGGTGAGGGCCAAGCTGGAGGAGCAGGCCCAGCAGATCAGGCTCCAGGCAGAGGCCTTCCAG
GCCAGGCTGAAGAGCTGGTTTGAGCCCCTGGTGGAGGACATGCAGAGGCAGTGGGCTGGCCTGGTGGAGAAGG
TCCAGGCTGCTGTGGGCACCTCAGCAGCCCCTGTCCCCAGTGACAACCACTGA
ApoE3ch-31-RBPi-FIXi (SEQ ID NO: 113)
ATGAAAGTGCTGTGGGCTGCCCTGCTGGTCACATTCCTGGCTGGTCAGTGGCAGCCAGGGGCAGCTGCACCCT
TTGCAGCTCCAGGGTTCCCCAAGGGCCCTGCCTGCTGATGGCCAGTGGGCATTGTGAAGGGAAGGGAGCACCA
AATGGGTGGAGGGAGGGAGGAAGCCCTTTGCCCTGGCTTGGCTGAGGATCCCCTTGGCTTTTGCAGGCTGCCA
GGCCAAGGTGGAGCAGGCTGTGGAGACAGAGCCAGAGCCAGAACTGAGGCAGCAGACAGAGTGGCAGTCTGGC
CAAAGATGGGAGCTAGCCCTGGGAAGATTCTGGGACTACCTGAGATGGGTGCAGACCCTCTCAGAACAAGTCC
AGGAGGAGCTGCTGTCCTCACAGGTGACCCAGGAGCTGAGGTTTGTTTCCTTTTTTATAATACATTGAGTATG
CTTGCCTTTTAGATATAGAAATATCTGATTCTGTCTTCTTCACTAAATTTTGATTACATGATTTGACAGCAAT
ATTGAAGAGTCTAACAGCCAGCACCCAGGTTGGTAAGTACTGGTTCTTTGTTAGCTAGGTTTTCTTCTTCTTC
ACTTTTAAAACTAAATAGATGGACAATGCTTATGATGCAATAAGGTTTAATAAACACTGTTCAGTTCAGTATT
TGGTCATGTAATTCCTGTTAAAAAACAGTCATCTCCTTGGTTTAAAAAAATTAAAAGTGGGAAAACAAAGAAA
TAGCAGAATATAGTGAAAAAAAATAACCACAGTATTTTTGTTTGGACTTACCACTTTGAAATCAAATTAGAGG
AAGGCATACAGGGAAGAAATTATCCCATTTGGACAAACAGCATGTTCTCACAGTAAGCACTTATCACACTTAC
TTGTCAACTTTTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAAT
TAGGCTTCTGTTCTTCAGGAGACATTTGTTCAAAGTCATTTGGGCAACCATATTCTGAAAACAGCCCAGCCAG
GGTGATGGATCACTTTGCAAAGATCCTCAATGAGCTATTTTCAAGTGATGACAAAGTGTGAAGTTAAGGGCTC
ATTTGAGAACTTTCTTTTTCATCCAAAGTAAATTCAAATATGATTAGAAATCTGACCTTTTATTACTGGAATT
CTCTTGACTAAAAGTAAAATTGAATTTTAATTCCTAAATCTCCATGTGTATACAGTACTGTGGGAACATCACA
GATTTTGGCTCCATGCCCTAAAGAGAAATTGGCTTTCAGATTATTTGGATTAAAAACAAAGACTTTCTTAAGA
GATGTAAAATTTTCATGATGTTTTCTTTTTTGCTAAAACTAAAGAATTATTCTTTTACATTTCAGGGCCCTGA
TGGATGAAACAATGAAGGAGCTGAAGGCATATAAGTCAGAATTGGAGGAGCAGCTGACTCCTGTGGCTGAGGA
GACCAGGGCTAGACTCTCCAAGGAGCTCCAGGCTGCCCAGGCCAGATTAGGAGCAGACATGGAGGATGTCTGT
GGGAGGCTGGTGCAGTACAGTGGTGAGGTCCAGGCCATGCTGGGCCAAAGCACAGAGGAGCTGAGGGTCAGGC
TGGCCAGCCATCTGAGGAAGCTGAGGAAAAGGCTGCTCAGGGATGCTGATGACCTGCAGAAGAGACTGGCAGT
GTACCAGGCTGGTGCCAGAGAGGGGGCAGAGAGGGGGCTCTCAGCCATCAGAGAGAGGCTGGGGCCTCTGGTG
GAGCAGGGCAGAGTGAGGGCTGCCACTGTGGGCTCCTTGGCAGGACAGCCCCTCCAAGAGAGAGCTCAGGCCT
GGGGAGAGAGGCTCAGGGCCAGGATGGAGGAAATGGGCAGTAGGACCAGGGACAGGCTTGATGAGGTGAAGGA
GCAGGTGGCTGAGGTCAGAGCCAAGCTGGAGGAGCAGGCTCAGCAGATTAGGCTGCAGGCAGAAGCCTTCCAG
GCCAGGCTGAAATCCTGGTTTGAGCCCCTGGTGGAAGACATGCAAAGGCAGTGGGCAGGGCTGGTGGAGAAGG
TGCAGGCTGCTGTGGGCACATCAGCTGCCCCTGTGCCCAGTGATAACCACTGA
ApoE3ch-33-RBPi-FIXi (SEQ ID NO: 114)
ATGAAAGTGCTATGGGCAGCCCTCTTGGTGACCTTCCTGGCTGGTCAGTGGCAGCCAGGGGCAGCTGCACCCT
TTGCAGCTCCAGGGTTCCCCAAGGGCCCTGCCTGCTGATGGCCAGTGGGCATTGTGAAGGGAAGGGAGCACCA
AATGGGTGGAGGGAGGGAGGAAGCCCTTTGCCCTGGCTTGGCTGAGGATCCCCTTGGCTTTTGCAGGCTGCCA
GGCCAAGGTGGAGCAGGCTGTGGAGACAGAGCCAGAACCAGAGCTGAGACAGCAAACTGAGTGGCAGTCAGGC
CAGAGATGGGAGCTGGCCCTGGGGAGATTTTGGGACTACTTAAGGTGGGTCCAGACCCTCAGTGAGCAGGTGC
AGGAAGAGCTGCTGAGCTCCCAGGTCACCCAGGAGCTGAGGTTTGTTTCCTTTTTTATAATACATTGAGTATG
CTTGCCTTTTAGATATAGAAATATCTGATTCTGTCTTCTTCACTAAATTTTGATTACATGATTTGACAGCAAT
ATTGAAGAGTCTAACAGCCAGCACCCAGGTTGGTAAGTACTGGTTCTTTGTTAGCTAGGTTTTCTTCTTCTTC
ACTTTTAAAACTAAATAGATGGACAATGCTTATGATGCAATAAGGTTTAATAAACACTGTTCAGTTCAGTATT
TGGTCATGTAATTCCTGTTAAAAAACAGTCATCTCCTTGGTTTAAAAAAATTAAAAGTGGGAAAACAAAGAAA
TAGCAGAATATAGTGAAAAAAAATAACCACAGTATTTTTGTTTGGACTTACCACTTTGAAATCAAATTAGAGG
AAGGCATACAGGGAAGAAATTATCCCATTTGGACAAACAGCATGTTCTCACAGTAAGCACTTATCACACTTAC
TTGTCAACTTTTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAAT
TAGGCTTCTGTTCTTCAGGAGACATTTGTTCAAAGTCATTTGGGCAACCATATTCTGAAAACAGCCCAGCCAG
GGTGATGGATCACTTTGCAAAGATCCTCAATGAGCTATTTTCAAGTGATGACAAAGTGTGAAGTTAAGGGCTC
ATTTGAGAACTTTCTTTTTCATCCAAAGTAAATTCAAATATGATTAGAAATCTGACCTTTTATTACTGGAATT
CTCTTGACTAAAAGTAAAATTGAATTTTAATTCCTAAATCTCCATGTGTATACAGTACTGTGGGAACATCACA
GATTTTGGCTCCATGCCCTAAAGAGAAATTGGCTTTCAGATTATTTGGATTAAAAACAAAGACTTTCTTAAGA
GATGTAAAATTTTCATGATGTTTTCTTTTTTGCTAAAACTAAAGAATTATTCTTTTACATTTCAGGGCCCTTA
TGGATGAAACCATGAAGGAGCTGAAGGCCTACAAGTCAGAACTGGAGGAGCAGCTCACCCCTGTGGCTGAGGA
GACCAGGGCAAGGCTCTCCAAGGAGCTACAGGCAGCCCAGGCCAGGCTGGGTGCAGACATGGAGGATGTCTGT
GGGAGACTGGTGCAGTACAGTGGTGAAGTACAGGCCATGCTTGGCCAATCCACAGAGGAGTTGAGGGTGAGGC
TGGCCTCCCACCTGAGGAAGCTGAGAAAGAGACTCCTCAGGGATGCAGATGACCTCCAGAAGAGGCTGGCAGT
TTACCAGGCAGGGGCCAGAGAGGGTGCAGAAAGAGGGCTGTCAGCCATCAGGGAAAGGCTGGGCCCTCTGGTG
GAGCAGGGCAGAGTGAGAGCAGCAACTGTTGGCTCCTTGGCTGGGCAGCCTCTGCAGGAGAGAGCTCAGGCCT
GGGGGGAGAGACTCAGGGCCAGGATGGAGGAGATGGGCTCCAGGACAAGGGACAGGTTGGATGAAGTGAAGGA
GCAGGTGGCTGAGGTGAGAGCAAAGTTGGAGGAGCAGGCCCAGCAGATCAGGCTTCAGGCAGAGGCCTTCCAG
GCCAGGCTGAAGTCCTGGTTTGAGCCACTGGTGGAGGATATGCAGAGACAGTGGGCAGGCCTGGTGGAGAAGG
TCCAGGCAGCAGTGGGTACCTCTGCAGCCCCAGTTCCCAGTGACAATCACTGA
ApoE3chAGTco3-RBPi-FIXi (SEQ ID NO: 115)
ATGAAGGTGCTGTGGGCTGCCCTGCTGGTGACCTTCCTGGCTGGTCAGTGGCAGCCAGGGGCAGCTGCACCCT
TTGCAGCTCCAGGGTTCCCCAAGGGCCCTGCCTGCTGATGGCCAGTGGGCATTGTGAAGGGAAGGGAGCACCA
AATGGGTGGAGGGAGGGAGGAAGCCCTTTGCCCTGGCTTGGCTGAGGATCCCCTTGGCTTTTGCAGGCTGTCA
GGCTAAAGTGGAGCAGGCTGTGGAGACTGAGCCTGAGCCAGAGCTGAGGCAGCAGACTGAGTGGCAGTCTGGC
CAGAGATGGGAGCTGGCTCTGGGCAGGTTCTGGGACTACCTGAGGTGGGTGCAGACCCTGTCTGAGCAGGTGC
AGGAGGAGCTGCTGTCTTCTCAGGTGACCCAGGAACTGAGGTTTGTTTCCTTTTTTATAATACATTGAGTATG
CTTGCCTTTTAGATATAGAAATATCTGATTCTGTCTTCTTCACTAAATTTTGATTACATGATTTGACAGCAAT
ATTGAAGAGTCTAACAGCCAGCACCCAGGTTGGTAAGTACTGGTTCTTTGTTAGCTAGGTTTTCTTCTTCTTC
ACTTTTAAAACTAAATAGATGGACAATGCTTATGATGCAATAAGGTTTAATAAACACTGTTCAGTTCAGTATT
TGGTCATGTAATTCCTGTTAAAAAACAGTCATCTCCTTGGTTTAAAAAAATTAAAAGTGGGAAAACAAAGAAA
TAGCAGAATATAGTGAAAAAAAATAACCACAGTATTTTTGTTTGGACTTACCACTTTGAAATCAAATTAGAGG
AAGGCATACAGGGAAGAAATTATCCCATTTGGACAAACAGCATGTTCTCACAGTAAGCACTTATCACACTTAC
TTGTCAACTTTTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAAT
TAGGCTTCTGTTCTTCAGGAGACATTTGTTCAAAGTCATTTGGGCAACCATATTCTGAAAACAGCCCAGCCAG
GGTGATGGATCACTTTGCAAAGATCCTCAATGAGCTATTTTCAAGTGATGACAAAGTGTGAAGTTAAGGGCTC
ATTTGAGAACTTTCTTTTTCATCCAAAGTAAATTCAAATATGATTAGAAATCTGACCTTTTATTACTGGAATT
CTCTTGACTAAAAGTAAAATTGAATTTTAATTCCTAAATCTCCATGTGTATACAGTACTGTGGGAACATCACA
GATTTTGGCTCCATGCCCTAAAGAGAAATTGGCTTTCAGATTATTTGGATTAAAAACAAAGACTTTCTTAAGA
GATGTAAAATTTTCATGATGTTTTCTTTTTTGCTAAAACTAAAGAATTATTCTTTTACATTTCAGGGCCCTGA
TGGATGAGACTATGAAGGAGCTGAAGGCCTACAAGTCTGAGCTGGAGGAGCAGCTGACCCCTGTGGCTGAGGA
GACCAGGGCCAGGCTGAGCAAGGAGCTGCAGGCTGCCCAGGCCAGACTGGGGGCTGACATGGAGGATGTGTGT
GGCAGACTGGTGCAGTACAGTGGGGAGGTGCAGGCCATGCTGGGGCAGTCTACTGAGGAGCTGAGGGTGAGGC
TGGCCAGCCACCTGAGGAAGCTGAGGAAGAGGCTGCTGAGGGATGCTGATGACCTGCAGAAGAGGCTGGCTGT
GTATCAGGCTGGGGCTAGGGAGGGGGCTGAGAGGGGCCTGTCTGCCATCAGGGAGAGGCTGGGGCCTCTGGTG
GAGCAGGGCAGGGTGAGGGCTGCCACTGTGGGGAGCCTGGCTGGCCAGCCTCTGCAGGAGAGGGCTCAGGCCT
GGGGGGAGAGGCTGAGGGCCAGGATGGAGGAGATGGGGAGCAGGACCAGGGACAGGCTGGATGAGGTGAAGGA
GCAGGTGGCTGAGGTGAGGGCCAAGCTGGAAGAGCAGGCCCAGCAGATCAGGCTGCAGGCTGAGGCCTTCCAG
GCCAGGCTGAAGAGCTGGTTTGAGCCCCTGGTGGAGGACATGCAGAGGCAGTGGGCTGGCCTGGTGGAGAAGG
TGCAGGCTGCTGTGGGCACCTCTGCTGCCCCTGTGCCCTCTGACAATCACTGA
ApoE3ch exons
ApoE3ch exon1 (SEQ ID NO: 116)
ATGAAGGTGCTGTGGGCTGCCCTGCTGGTGACCTTCCTGGCTG
ApoE3ch exon2 (SEQ ID NO: 117)
GCTGTCAGGCTAAAGTGGAGCAGGCTGTGGAGACTGAGCCTGAGCCAGAGCTGAGGCAGCAGACTGAGTGGCA
GTCTGGCCAGAGATGGGAGCTGGCTCTGGGCAGGTTCTGGGACTACCTGAGGTGGGTGCAGACCCTGTCTGAG
CAGGTGCAGGAGGAGCTGCTGTCTTCTCAGGTGACCCAGGAACTGAG
ApoE3ch exon3 (SEQ ID NO: 118)
GGCCCTGATGGATGAGACTATGAAGGAGCTGAAGGCCTACAAGTCTGAGCTGGAGGAGCAGCTGACCCCTGTG
GCTGAGGAGACCAGGGCCAGGCTGAGCAAGGAGCTGCAGGCTGCCCAGGCCAGACTGGGGGCTGACATGGAGG
ATGTGTGTGGCAGACTGGTGCAGTACAGTGGGGAGGTGCAGGCCATGCTGGGGCAGTCTACTGAGGAGCTGAG
GGTGAGGCTGGCCAGCCACCTGAGGAAGCTGAGGAAGAGGCTGCTGAGGGATGCTGATGACCTGCAGAAGAGG
CTGGCTGTGTATCAGGCTGGGGCTAGGGAGGGGGCTGAGAGGGGCCTGTCTGCCATCAGGGAGAGGCTGGGGC
CTCTGGTGGAGCAGGGCAGGGTGAGGGCTGCCACTGTGGGGAGCCTGGCTGGCCAGCCTCTGCAGGAGAGGGC
TCAGGCCTGGGGGGAGAGGCTGAGGGCCAGGATGGAGGAGATGGGGAGCAGGACCAGGGACAGGCTGGATGAG
GTGAAGGAGCAGGTGGCTGAGGTGAGGGCCAAGCTGGAAGAGCAGGCCCAGCAGATCAGGCTGCAGGCTGAGG
CCTTCCAGGCCAGGCTGAAGAGCTGGTTTGAGCCCCTGGTGGAGGACATGCAGAGGCAGTGGGCTGGCCTGGT
GGAGAAGGTGCAGGCTGCTGTGGGCACCTCTGCTGCCCCTGTGCCCTCTGACAATCACTGA
Introns
FIXi (FIX-derived intron; CpG-free) (SEQ ID NO: 119)
GTTTGTTTCCTTTTTTATAATACATTGAGTATGCTTGCCTTTTAGATATAGAAATATCTGATTCTGTCTTCTT
CACTAAATTTTGATTACATGATTTGACAGCAATATTGAAGAGTCTAACAGCCAGCACCCAGGTTGGTAAGTAC
TGGTTCTTTGTTAGCTAGGTTTTCTTCTTCTTCACTTTTAAAACTAAATAGATGGACAATGCTTATGATGCAA
TAAGGTTTAATAAACACTGTTCAGTTCAGTATTTGGTCATGTAATTCCTGTTAAAAAACAGTCATCTCCTTGG
TTTAAAAAAATTAAAAGTGGGAAAACAAAGAAATAGCAGAATATAGTGAAAAAAAATAACCACAGTATTTTTG
TTTGGACTTACCACTTTGAAATCAAATTAGAGGAAGGCATACAGGGAAGAAATTATCCCATTTGGACAAACAG
CATGTTCTCACAGTAAGCACTTATCACACTTACTTGTCAACTTTTAGAATCAAATCTAGTAGCTGACAGTACC
AGGATCAGGGGTGCCAACCCTAAGCACCCCCAGAAAGCTGACTGGCCCTGTGGTTCCCACTCCAGACATGATG
TCAGCTGTGAAATCCACCTCCCTGGACCATAATTAGGCTTCTGTTCTTCAGGAGACATTTGTTCAAAGTCATT
TGGGCAACCATATTCTGAAAACAGCCCAGCCAGGGTGATGGATCACTTTGCAAAGATCCTCAATGAGCTATTT
TCAAGTGATGACAAAGTGTGAAGTTAAGGGCTCATTTGAGAACTTTCTTTTTCATCCAAAGTAAATTCAAATA
TGATTAGAAATCTGACCTTTTATTACTGGAATTCTCTTGACTAAAAGTAAAATTGAATTTTAATTCCTAAATC
TCCATGTGTATACAGTACTGTGGGAACATCACAGATTTTGGCTCCATGCCCTAAAGAGAAATTGGCTTTCAGA
TTATTTGGATTAAAAACAAAGACTTTCTTAAGAGATGTAAAATTTTCATGATGTTTTCTTTTTTGCTAAAACT
AAAGAATTATTCTTTTACATTTCAG
RBPi (RBP4 intron; CpG-free) (SEQ ID NO: 120)
GTCAGTGGCAGCCAGGGGCAGCTGCACCCTTTGCAGCTCCAGGGTTCCCCAAGGGCCCTGCCTGCTGATGGCC
AGTGGGCATTGTGAAGGGAAGGGAGCACCAAATGGGTGGAGGGAGGGAGGAAGCCCTTTGCCCTGGCTTGGCT
GAGGATCCCCTTGGCTTTTGCAG
VCLi (VCL intron; CpG-free) (SEQ ID NO: 121)
GTAGGCAATCTGAGACAAAAAGCCTACTCTTGAAGATAATAATGGAAGATTGATGATTGTTTAGAATGAAAAT
ATGTTGAATGCTGCACATCTGTGTTACCATGTTTGCTAGTTGTCCACCCATGTGATTGAATACTCTGAAATAT
TTTTTCAG
ApoE CpG reduced
ApoE3_h5 (SEQ ID NO: 124)
ATGAAGGTGTTGTGGGCAGCACTGCTGGTCACTTTCCTGGCAGGCTGCCAGGCGAAGGTGGAGCAGGCAGTAG
AGACTGAACCAGAACCTGAACTGAGGCAACAGACTGAATGGCAGAGTGGTCAAAGATGGGAACTGGCCCTTGG
AAGGTTTTGGGACTACCTGAGGTGGGTGCAGACCCTCAGTGAGCAGGTGCAGGAGGAGCTGCTGAGCTCCCAG
GTGACCCAAGAACTAAGGGCTCTGATGGATGAGACCATGAAGGAGCTGAAGGCCTACAAGTCTGAGCTGGAGG
AACAGCTCACCCCAGTGGCTGAAGAGACTAGAGCAAGGCTCAGCAAGGAGTTGCAGGCTGCCCAGGCCAGGCT
GGGGGCTGACATGGAAGATGTATGTGGAAGGCTTGTGCAGTACAGGGGCGAGGTGCAGGCCATGCTGGGACAG
AGCACTGAAGAGCTGAGGGTTCGGCTAGCCTCCCACTTAAGGAAGCTAAGAAAAAGACTGCTCAGAGATGCCG
ACGACCTCCAGAAGAGACTGGCTGTGTACCAGGCTGGAGCCAGAGAGGGAGCAGAGAGAGGACTGAGTGCCAT
TAGGGAGAGATTGGGTCCCCTGGTAGAGCAGGGCAGGGTCAGAGCTGCCACAGTGGGCTCTTTGGCAGGCCAG
CCTTTGCAGGAAAGAGCTCAGGCCTGGGGAGAGAGACTGAGAGCCAGGATGGAGGAGATGGGATCTAGGACCA
GGGACAGACTGGATGAAGTCAAAGAACAAGTTGCTGAGGTCCGGGCTAAGCTGGAGGAGCAAGCCCAGCAGAT
TAGGCTACAGGCAGAGGCCTTCCAGGCCAGGCTGAAGAGCTGGTTTGAGCCTTTGGTAGAGGACATGCAGAGA
CAGTGGGCTGGGTTGGTGGAGAAGGTCCAGGCTGCTGTTGGCACATCAGCAGCCCCTGTCCCCAGTGACAACC
ACTGA
ApoE3_h6 (SEQ ID NO: 125)
ATGAAGGTCCTCTGGGCAGCCCTGCTGGTCACATTCCTGGCAGGCTGTCAAGCCAAGGTGGAACAAGCTGTGG
AGACTGAGCCAGAGCCAGAGCTCAGGCAGCAAACAGAGTGGCAGTCGGGGCAGAGATGGGAATTGGCACTGGG
AAGATTCTGGGACTACCTGAGGTGGGTTCAGACCCTGTCAGAGCAGGTGCAGGAGGAGCTCCTGAGCTCACAA
GTCACTCAAGAGCTGAGAGCACTTATGGATGAAACCATGAAGGAATTAAAGGCCTACAAATCAGAATTGGAAG
AGCAGCTGACACCTGTGGCTGAAGAGACCAGGGCCAGACTGAGTAAAGAACTGCAAGCAGCCCAGGCCAGACT
TGGAGCTGACATGGAGGATGTTTGTGGCAGGCTGGTGCAGTACAGAGGTGAAGTCCAAGCCATGCTGGGACAA
TCCACAGAGGAGCTGCGTGTCCGGCTAGCCAGCCACCTAAGAAAACTGAGGAAGAGACTCCTGAGGGATGCTG
ATGATCTCCAGAAAAGGTTGGCTGTGTACCAAGCTGGAGCCAGGGAAGGAGCTGAAAGGGGACTCTCTGCCAT
CAGAGAAAGACTGGGTCCCCTGGTGGAGCAGGGCAGGGTGCGGGCAGCAACAGTGGGAAGCTTGGCAGGACAG
CCACTCCAGGAAAGGGCACAGGCATGGGGAGAAAGGCTGAGAGCACGGATGGAAGAGATGGGCAGCAGGACCA
GGGACAGGCTGGATGAGGTGAAGGAACAAGTGGCAGAGGTAAGGGCAAAACTGGAGGAGCAGGCCCAGCAGAT
TCGCCTCCAGGCTGAAGCCTTCCAAGCAAGGTTGAAAAGCTGGTTTGAGCCTCTTGTGGAAGACATGCAGAGA
CAGTGGGCTGGCCTTGTGGAAAAAGTCCAAGCTGCAGTAGGCACCAGTGCAGCACCTGTCCCATCAGACAACC
ATTGA
ApoE3_h7 ( (SEQ ID NO: 126)
ATGAAAGTCCTCTGGGCTGCCTTGCTGGTAACTTTCTTGGCTGGCTGCCAGGCCAAGGTGGAGCAAGCTGTGG
AAACAGAGCCTGAGCCGGAGCTGAGGCAACAGACAGAATGGCAGTCAGGCCAGAGGTGGGAGCTGGCTCTGGG
GAGATTCTGGGATTACCTTCGCTGGGTCCAGACACTCTCTGAACAAGTGCAAGAGGAGCTGCTGTCCTCCCAA
GTCACCCAGGAGCTGAGAGCCTTGATGGATGAAACCATGAAAGAGCTGAAAGCTTACAAGTCTGAGCTGGAGG
AACAGTTGACCCCAGTGGCTGAGGAGACCAGGGCCAGATTGTCCAAGGAACTGCAGGCTGCTCAGGCAAGGTT
GGGGGCAGATATGGAGGATGTGTGTGGGAGGTTGGTGCAGTACAGAGGTGAGGTGCAGGCCATGCTGGGACAG
AGCACTGAAGAGCTAAGGGTCAGATTAGCAAGTCATTTGAGGAAGCTGAGAAAGAGGCTGCTGAGAGACGCGG
ATGACCTTCAGAAGAGACTGGCTGTCTACCAAGCTGGTGCCAGAGAAGGGGCTGAAAGAGGTCTCTCAGCCAT
CAGGGAGAGGCTGGGGCCTCTGGTTGAGCAGGGTAGGGTGAGGGCGGCTACTGTGGGATCCCTGGCTGGTCAA
CCACTCCAAGAAAGAGCACAGGCCTGGGGGGAGAGACTCCGTGCCAGAATGGAGGAAATGGGCAGCAGGACAA
GAGACAGGCTGGATGAGGTCAAGGAGCAAGTAGCAGAAGTGAGGGCCAAACTGGAGGAACAGGCCCAGCAGAT
CAGACTCCAAGCTGAGGCCTTCCAGGCCAGGCTGAAATCTTGGTTTGAGCCTTTGGTAGAAGACATGCAGAGG
CAGTGGGCAGGGTTGGTAGAAAAGGTGCAGGCTGCTGTGGGGACTTCAGCAGCCCCTGTGCCCTCAGACAATC
ACTGA
ApoE3_h12 (SEQ ID NO: 127)
ATGAAGGTCCTCTGGGCTGCCCTGCTGGTCACCTTTCTAGCTGGGTGCCAGGCCAAGGTGGAACAAGCAGTGG
AAACTGAGCCTGAGCCTGAGCTGAGGCAACAGACAGAGTGGCAGTCTGGTCAGAGGTGGGAGCTTGCACTGGG
GAGGTTCTGGGATTACCTCCGCTGGGTGCAGACCCTGAGTGAGCAGGTGCAGGAGGAGCTCCTTAGCTCACAG
GTTACTCAGGAGTTGAGAGCACTCATGGATGAAACCATGAAGGAGCTGAAGGCCTACAAAAGTGAACTGGAAG
AACAGCTGACCCCTGTTGCAGAAGAAACCAGGGCAAGGCTCTCCAAAGAGCTGCAAGCTGCTCAGGCAAGACT
GGGTGCTGACATGGAGGATGTCTGTGGCAGGCTGGTGCAGTATCGTGGCGAAGTACAGGCAATGCTGGGGCAG
TCCACTGAAGAGCTGAGAGTGCGGCTGGCTTCTCATCTTAGGAAGTTAAGGAAGAGGCTGCTCCGAGATGCTG
ATGACCTCCAGAAGAGGCTGGCTGTGTATCAGGCTGGGGCAAGGGAGGGGGCAGAGAGGGGCCTCTCTGCCAT
AAGGGAGAGACTGGGTCCACTGGTGGAACAAGGCCGAGTGAGAGCAGCCACAGTCGGCTCTCTGGCAGGGCAG
CCCCTCCAAGAAAGAGCCCAGGCTTGGGGAGAAAGACTGAGGGCCAGGATGGAGGAAATGGGATCCAGGACAA
GAGATCGCCTGGATGAGGTGAAGGAGCAGGTGGCTGAGGTGAGGGCAAAGCTGGAAGAGCAAGCCCAGCAGAT
CAGGCTCCAGGCTGAAGCTTTCCAAGCAAGGCTGAAAAGCTGGTTTGAGCCCCTGGTGGAGGACATGCAGAGG
CAGTGGGCAGGCCTGGTGGAGAAAGTTCAGGCAGCTGTGGGTACTTCAGCTGCACCTGTCCCTTCTGACAATC
ACTGA
ApoE3_h15 (SEQ ID NO: 128)
ATGAAAGTCCTGTGGGCAGCGCTCCTGGTCACCTTCCTGGCTGGCTGTCAAGCTAAAGTGGAGCAGGCTGTGG
AAACAGAGCCTGAGCCAGAACTTAGACAACAAACTGAGTGGCAGAGTGGGCAGAGGTGGGAGCTGGCCTTGGG
CAGATTCTGGGACTACCTGCGCTGGGTCCAGACTCTTTCTGAGCAGGTGCAGGAGGAGCTCCTGTCCAGTCAG
GTGACCCAGGAGCTGAGGGCTCTCATGGATGAGACAATGAAAGAGCTGAAGGCCTACAAAAGTGAGCTGGAAG
AGCAGCTGACCCCAGTGGCTGAAGAGACAAGGGCCAGGTTAAGCAAAGAGCTCCAGGCTGCCCAGGCCAGGCT
GGGAGCAGACATGGAGGATGTCTGCGGCCGCCTGGTCCAGTATAGAGGGGAGGTGCAAGCCATGTTAGGCCAG
AGCACTGAAGAGCTGAGGGTGAGGTTAGCTTCTCACCTGAGAAAATTAAGGAAGAGGCTGCTGAGGGATGCTG
ATGACCTGCAGAAGAGGTTGGCTGTTTACCAGGCAGGGGCTAGAGAGGGAGCAGAGAGGGGCCTCTCTGCCAT
CAGAGAGAGGCTGGGCCCCCTTGTGGAGCAGGGCCGTGTCCGGGCAGCTACTGTTGGCTCACTTGCAGGGCAG
CCTCTGCAGGAAAGAGCCCAGGCATGGGGGGAGCGGTTAAGGGCAAGAATGGAAGAGATGGGCTCAAGGACAA
GAGACAGGCTGGACGAAGTGAAGGAGCAGGTGGCAGAAGTGAGGGCCAAGCTAGAGGAACAGGCCCAGCAGAT
TCGCCTCCAGGCAGAAGCCTTCCAGGCCAGGCTCAAGTCCTGGTTTGAGCCCTTAGTGGAGGACATGCAGAGG
CAGTGGGCTGGCCTGGTGGAGAAGGTACAGGCTGCTGTGGGGACTTCAGCAGCCCCAGTGCCCAGTGACAACC
ACTGA
ApoE3_h24 (SEQ ID NO: 129)
ATGAAGGTCCTCTGGGCAGCTCTCCTGGTTACCTTCTTAGCTGGCTGTCAAGCCAAAGTAGAACAAGCAGTGG
AAACAGAGCCGGAGCCAGAGCTCAGACAGCAAACAGAATGGCAGTCTGGTCAGAGATGGGAACTGGCACTGGG
TAGGTTCTGGGACTACCTGAGGTGGGTCCAGACCCTGTCAGAGCAGGTCCAGGAAGAGCTGCTGAGCAGCCAG
GTGACTCAAGAGCTGCGTGCCCTCATGGATGAAACCATGAAGGAGCTGAAGGCCTACAAGAGTGAGTTAGAAG
AGCAGCTCACACCTGTGGCTGAAGAAACAAGGGCCCGCCTCAGCAAGGAGCTGCAAGCTGCACAGGCCAGGCT
GGGTGCTGATATGGAGGATGTGTGCGGCAGGCTGGTGCAGTATCGTGGGGAAGTCCAGGCCATGTTGGGCCAA
TCCACAGAGGAGCTCAGAGTTCGCCTTGCCTCCCATCTCAGGAAACTAAGAAAGAGACTACTGCGGGATGCTG
ATGACCTTCAGAAGAGGCTGGCTGTGTACCAGGCAGGAGCCAGAGAGGGGGCTGAGAGGGGTCTCTCAGCCAT
CAGGGAAAGACTTGGGCCCTTGGTTGAGCAGGGCAGGGTGCGGGCAGCCACAGTTGGGTCTCTGGCTGGCCAG
CCCCTCCAGGAGAGAGCTCAGGCCTGGGGTGAGAGACTTCGAGCTAGGATGGAGGAGATGGGGAGCAGGACCA
GGGACAGGTTGGATGAGGTCAAGGAGCAGGTGGCAGAAGTGAGAGCCAAGCTGGAAGAGCAGGCCCAGCAAAT
CAGGCTGCAAGCAGAGGCATTTCAGGCCCGGCTCAAGTCATGGTTTGAGCCTCTGGTGGAGGACATGCAGAGG
CAGTGGGCTGGGCTGGTGGAAAAGGTACAGGCTGCTGTGGGCACCAGTGCAGCACCTGTCCCATCTGATAACC
ATTGA
ApoE3_h30 (SEQ ID NO: 130)
ATGAAAGTCCTGTGGGCAGCCCTCCTTGTGACCTTCCTGGCAGGCTGTCAAGCAAAGGTGGAGCAGGCTGTAG
AGACTGAGCCAGAGCCTGAACTGAGGCAGCAGACAGAATGGCAGTCTGGCCAGAGATGGGAGCTGGCCCTGGG
AAGATTTTGGGACTATCTCCGCTGGGTCCAGACCCTGTCAGAACAGGTTCAGGAAGAGCTGCTTTCCAGCCAA
GTGACACAGGAGCTGAGAGCCCTTATGGATGAGACAATGAAAGAACTGAAGGCTTATAAAAGTGAGCTAGAGG
AGCAGCTGACCCCAGTGGCTGAAGAGACCCGGGCCCGGCTGAGCAAAGAGCTCCAGGCTGCACAGGCCAGATT
GGGAGCTGACATGGAGGATGTGTGTGGGAGACTAGTGCAGTACAGGGGTGAAGTGCAGGCCATGCTGGGACAA
AGCACAGAAGAGTTGAGAGTCAGGCTGGCATCCCACCTGAGGAAACTCAGGAAAAGACTCCTCCGTGATGCAG
ATGACCTCCAGAAGAGACTGGCAGTCTATCAGGCTGGTGCCAGAGAGGGCGCCGAGAGAGGACTCTCAGCCAT
CAGGGAGCGGCTGGGGCCTCTGGTTGAGCAGGGCAGAGTCAGAGCAGCAACAGTGGGGAGTCTGGCCGGCCAG
CCCTTACAAGAGAGAGCTCAGGCATGGGGAGAGAGGCTTCGCGCTAGGATGGAAGAAATGGGATCAAGGACTA
GAGACAGGTTGGATGAAGTAAAGGAGCAGGTGGCAGAAGTGAGGGCCAAGCTTGAGGAGCAGGCCCAGCAGAT
CAGGCTACAGGCTGAGGCATTTCAGGCCAGGCTGAAGAGCTGGTTTGAGCCCTTGGTGGAGGATATGCAGAGG
CAGTGGGCTGGATTGGTGGAAAAGGTACAGGCTGCTGTTGGGACGAGTGCTGCTCCTGTCCCCTCTGATAATC
ATTGA

### EXAMPLES

Examples are provided below further illustrating different features of the present invention and methodology for practicing the invention. The provided examples do not limit the claimed invention.

### Example 1: Liver-Mediated ApoE3(Ch) and ApoE3 Transgene Expression

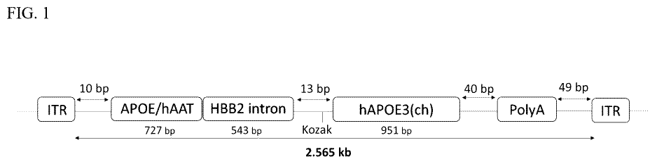

The impact of ApoE3(ch) and ApoE3 transgene expression on cholesterol was evaluated using wild-type C57BL/6 and ApoE knockout (KG) mice, and rAAV comprising a transgene expressing ApoE3(ch) or ApoE3. Recombinant AAV nucleic acid was made up rAAV polynucleotides comprising an ApoE/hAAT promoter/enhancer functionally coupled to either ApoE3 or ApoE(ch) encoding nucleic acid, along with other expression vector components and ITRs, as illustrated in FIG. 1.

Female wild-type C57BL/6 and ApoE knockout (KG) mice (B6.129P2-Apoetm1Unc/J, Jackson Labs, Strain #:002052)) were obtained from Jackson Laboratories (Bar Harbor, Maine, USA). ApoE knockout mice are hyperlipidemic and develop spontaneous atherosclerosis by six months of age. Baseline plasma was collected at 8 weeks of age. At 9 weeks of age, 200 mL of excipient diluent (PBS180/0.001% Pluronic F66, pH 7.3) as a control, or 200 mL of diluent containing a low dose or high dose of rAAV (transgene and capsid), were administered intravenously via tail vain. Recombinant AAV comprising ApoE3(ch) transgene was provided at a low dose of 3e12 vg/kg or a high dose of 1e13 vg/kg. Recombinant AAV comprising ApoE3 transgene was provided at a low dose of 3e12 vg/kg or a high dose of 6e12 vg/kg rAAV The overall design is provided in Table 2. Viral capsid comprised VP1 of SEQ ID NO: 83 (U.S. Pat. No. 9,169,299), VP2 of SEQ ID NO: 122 and VP3 of SEQ ID NO: 123.

TABLE 2

| | Number | | | | |
|---|---|---|---|---|---|
| | of | | | | |
| | Female | | | | Volume |
| Group | Mice | Genotype | Transgene | Dose | Injected |
| 1 | 8 | C57BL/6 | Excipient control | | 200 μL |
| 2 | 8 | ApoE KO | Excipient control | | 200 μL |
| 3 | 8 | ApoE KO | ApoE/hAAT.hApoE3ch | Low | 200 μL |
| 4 | 8 | ApoE KO | ApoE/hAAT.hApoE3ch | High | 200 μL |
| 5 | 8 | ApoE KO | ApoE/hAAT.hApoE3 | Low | 200 μL |
| 6 | 8 | ApoE KO | ApoE/hAAT.hApoE3 | High | 200 μL |

Plasma was collected weekly for the first 8 weeks after AAV injection, and monthly thereafter for the measurement of total cholesterol, high density lipoprotein (HDL) and low density lipoprotein/very low density lipoprotein (LDL/VLDL) cholesterol using the EnzyChrom™ AF HDL and LDL/VLDL Assay Kit (Bioassay Systems, Hayward, CA, USA) and ApoE3 protein by ELISA (Mabtech Ab, Sweden), according to manufacturer instructions.

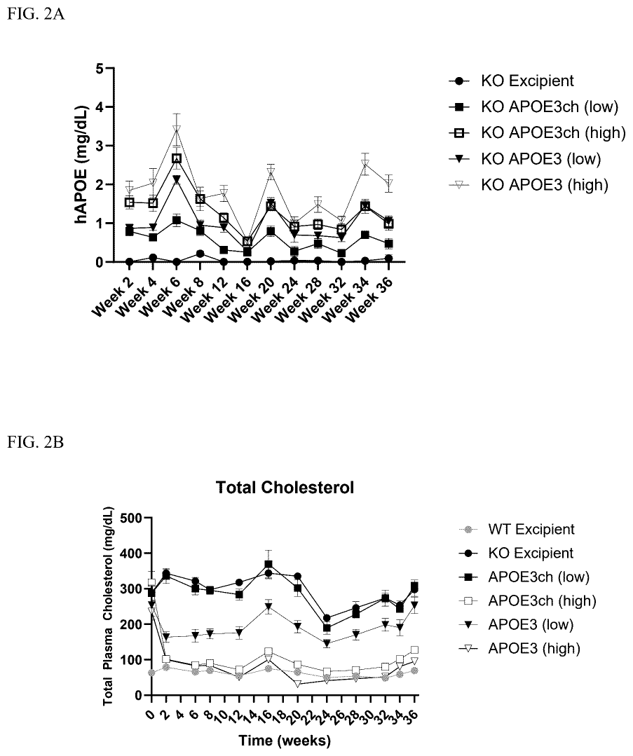

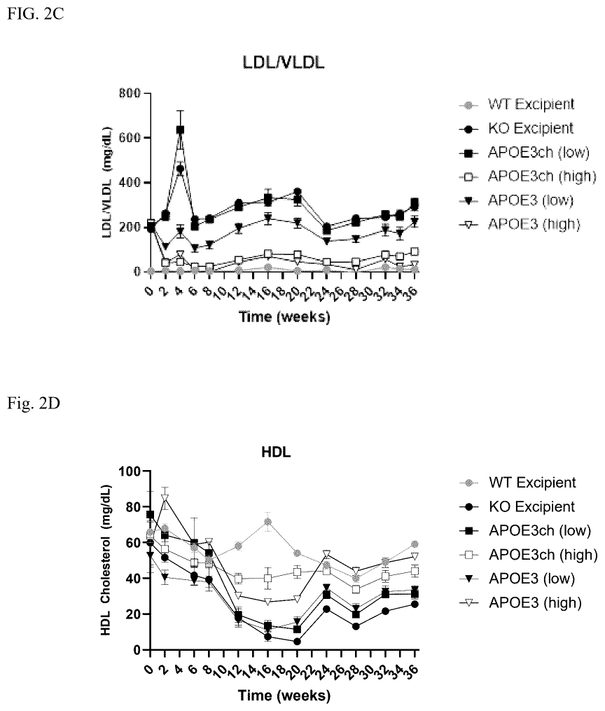

FIGS. 2A-2E illustrate ApoE3 and ApoE3(ch) transgene expression, and the impact of transgene expression on cholesterol, in ApoE knockout mice over the course of 36 weeks. Recombinant AAV comprising an ApoE(ch) transgene were provided at a low dose of 3e12 vg/kg or a high dose of 1e13 vg/kg. Recombinant AAV comprising an ApoE3 transgene were provided at a low dose of 3e12 vg/kg or a high dose of 6e12 vg/kg rAAV. FIG. 2A illustrates hAPOE production, FIG. 2B illustrates total cholesterol, FIG. 2C illustrates HDL, FIG. 2D illustrates the LDL/VLDL cholesterol ratio and FIG. 2E illustrates the total/HDL cholesterol ratio. Plasma hAPOE levels were significantly negatively correlated with Total Cholesterol (p<0.0001; Spearman r=−0.6214), LDL/VLDL Cholesterol (p<0.0001; Spearman r=−0.6854), Total Cholesterol/HDL ratio (p<0.0001; Spearman r=−0.6792), but significantly positively correlated with HDL cholesterol (p<0.0001, Spearman r=0.4584).

### Example 2: CpG-Depleted ApoE3(ch) and ApoE3 Transgene Expression

The impact of different CpG-depleted ApoE(ch) and ApoE constructs on cholesterol was evaluated using wild-type C57BL/6 and ApoE knockout (KO) mice, and rAAV comprising different CpG-depleted ApoE3(ch) and ApoE3 transgenes. Recombinant AAV nucleic acid contained an ApoE/hAAT promoter/enhancer functionally coupled to either ApoE3 or ApoE(ch) encoding nucleic acid, along with other expression vector components and ITRs, as illustrated in FIG. 1.

Female wild-type C57BL/6 and ApoE knockout (KO) mice (B6.129P2-Apoetm1Unc/J, Jackson Labs, Strain #:002052) were obtained from Jackson Laboratories (Bar Harbor, Maine, USA). Baseline plasma was collected at 8 weeks of age. At 9 weeks of age, 200 mL of excipient diluent (PBS180/0.001% Pluronic F66, pH 7.3) as a control, or 200 mL of diluent containing 1e13 vg/kg (2e11 total vg) of rAAV (transgene and capsid) were administered intravenously via tail vain. Viral capsid comprised VP1 of SEQ ID NO: 83, VP2 of SEQ ID NO: 122 and VP3 of SEQ ID NO: 123.

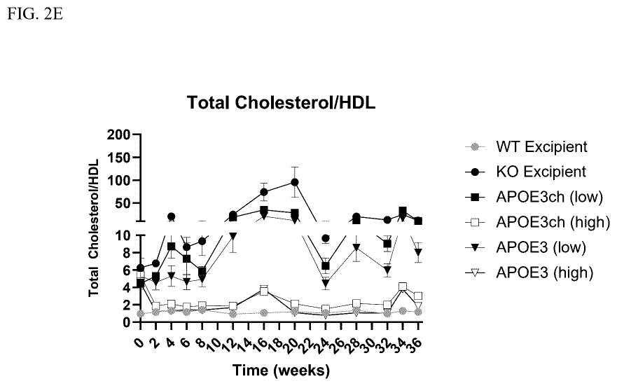

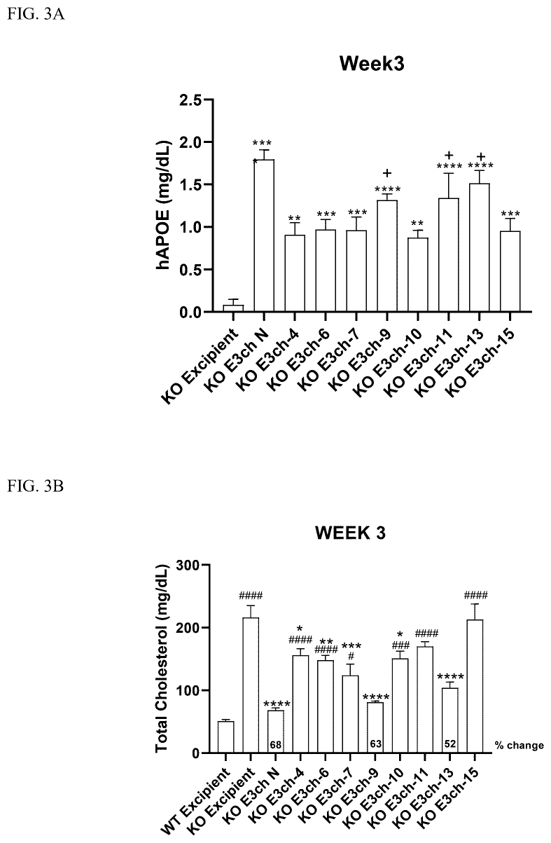

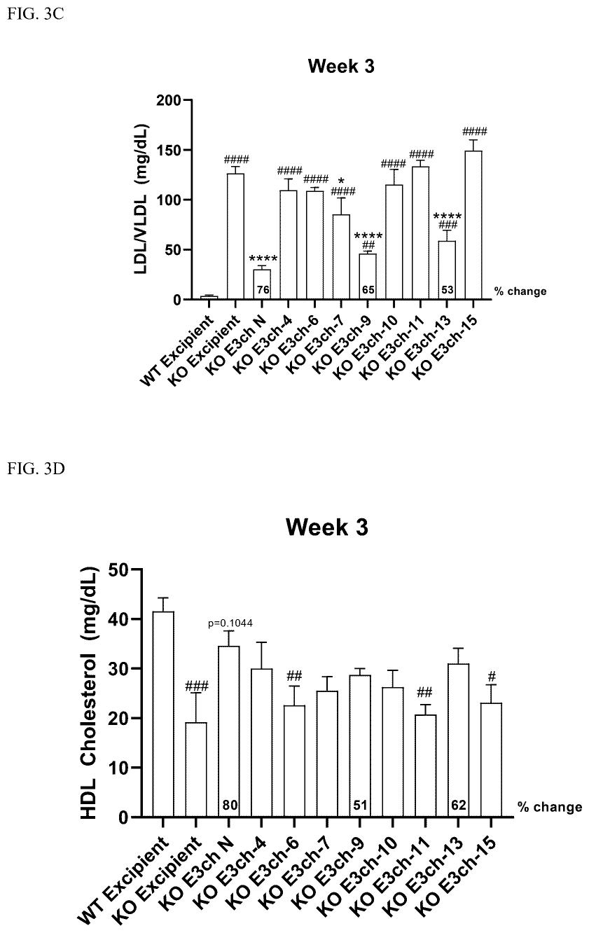

Plasma was collected at week 3 and week 6 following rAAV injection for the measurement of total cholesterol, high density lipoprotein (HDL) and low density lipoprotein/very low density lipoprotein (LDL/VLDL) cholesterol using the EnzyChrom™ AF HDL and LDL/VLDL Assay Kit (Bioassay Systems, Hayward, CA, USA) and ApoE3 protein by ELISA (Mabtech Ab, Sweden), according to manufacturer instructions. The results are shown in FIGS. 3A-3E, 4A-4E, 5A-5E, and 6A-6E. The SEQ ID NOs: for the different constructs are provided in Table 3.

TABLE 3

| ApoE3(ch) | SEQ ID NO: | ApoE3 | SEQ ID NO: |
|---|---|---|---|
| APOE3ch-N | 1 | APOE3-N | 2 |
| APOE3ch-4 | 6 | APOE3-3 | 20 |
| APOE3ch-6 | 8 | APOE3-8 | 25 |
| APOE3ch-7 | 9 | APOE3-9 | 26 |
| APOE3ch-9 | 11 | APOE3-11 | 28 |
| APOE3ch-10 | 12 | APOE3-12 | 29 |
| APOE3ch-11 | 13 | APOE3-15 | 31 |
| APOE3ch-13 | 15 | | |
| APOE3ch-15 | 17 | | |

FIGS. 3A-3E illustrate ApoE3(ch) transgene expression from different constructs, and the impact of transgene expression on cholesterol, at 3 weeks. FIG. 3A illustrates hAPOE levels, FIG. 3B illustrates total cholesterol, FIG. 3C illustrates the LDL/VLDL cholesterol ratio, FIG. 3D illustrates HDL cholesterol, and FIG. 3E illustrates the total cholesterol/HDL ratio.

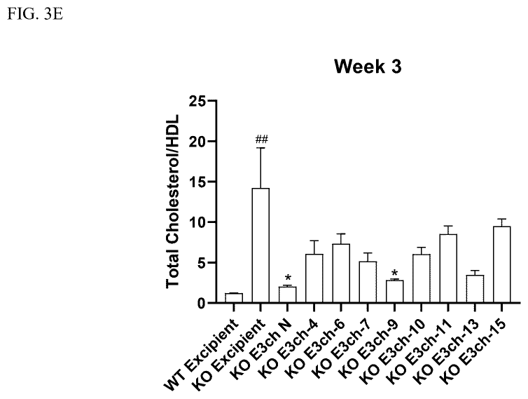

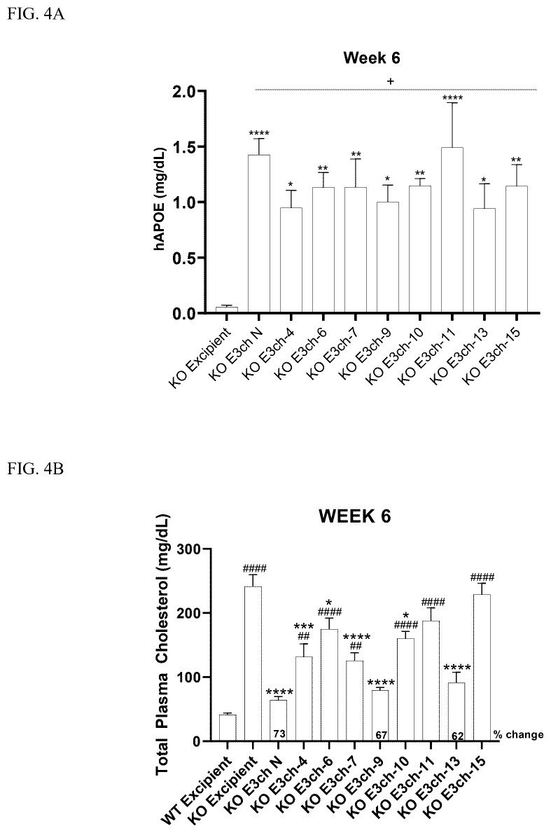

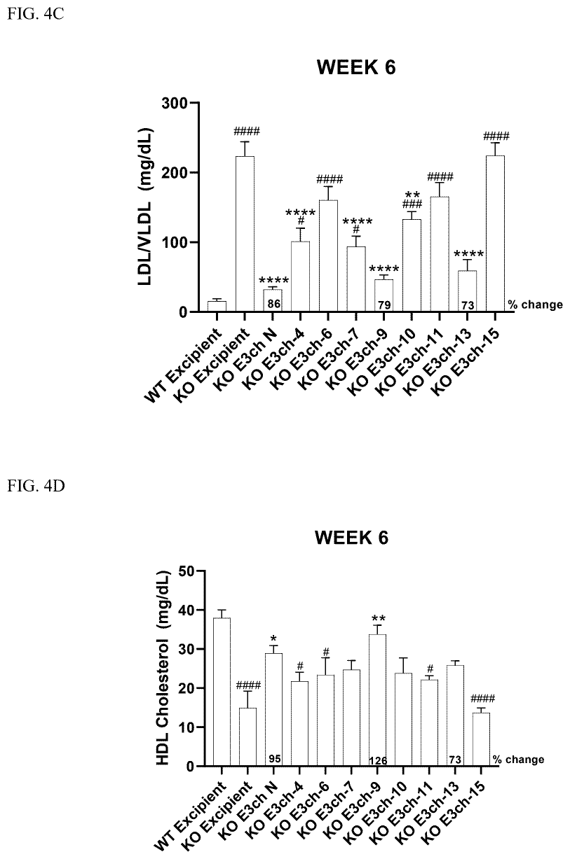

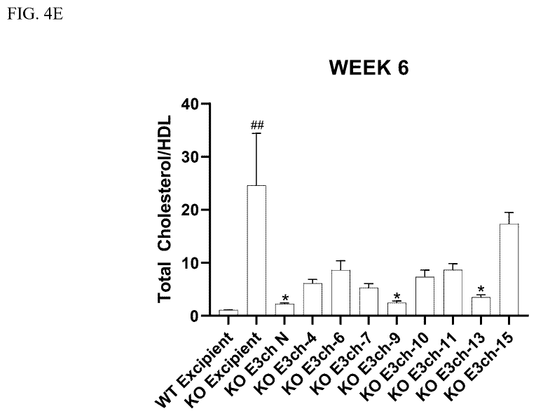

FIGS. 4A-4E illustrate ApoE3(ch) transgene expression from different constructs, and the impact of transgene expression on cholesterol, at 6 weeks. FIG. 4A illustrates hAPOE levels, FIG. 4B illustrates total cholesterol, FIG. 4C illustrates the LDL/VLDL cholesterol ratio, FIG. 4D illustrates HDL cholesterol, and FIG. 4E illustrates the total cholesterol/HDL ratio.

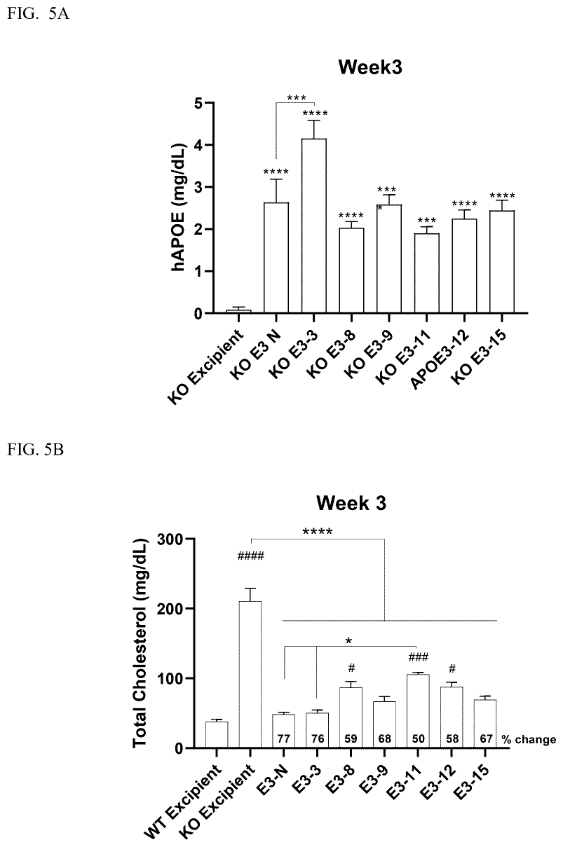

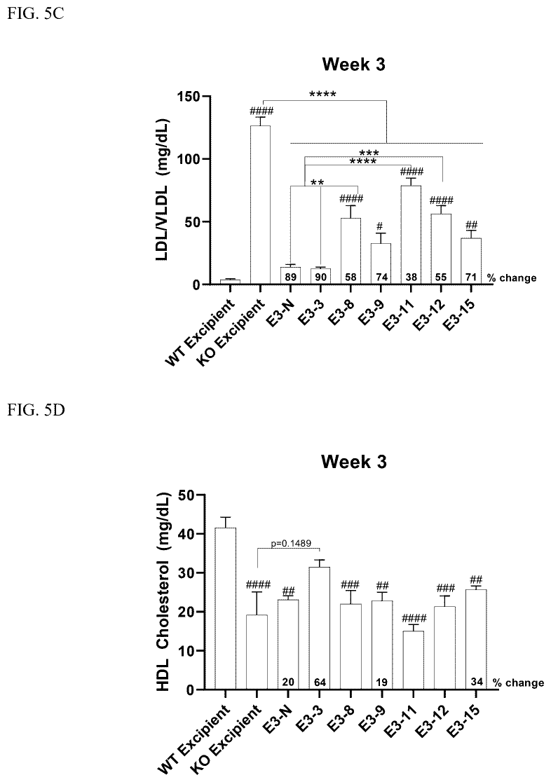

FIGS. 5A-5E illustrate ApoE3 transgene expression from different constructs, and the impact of transgene expression on cholesterol, at 3 weeks. FIG. 5A illustrates hAPOE levels, FIG. 5B illustrates total cholesterol, FIG. 5C illustrates the LDL/VLDL cholesterol ratio, FIG. 5D illustrates HDL cholesterol, and FIG. 5E illustrates the total cholesterol/HDL ratio.

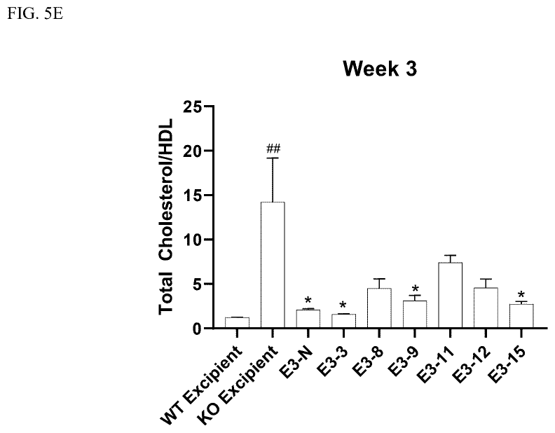

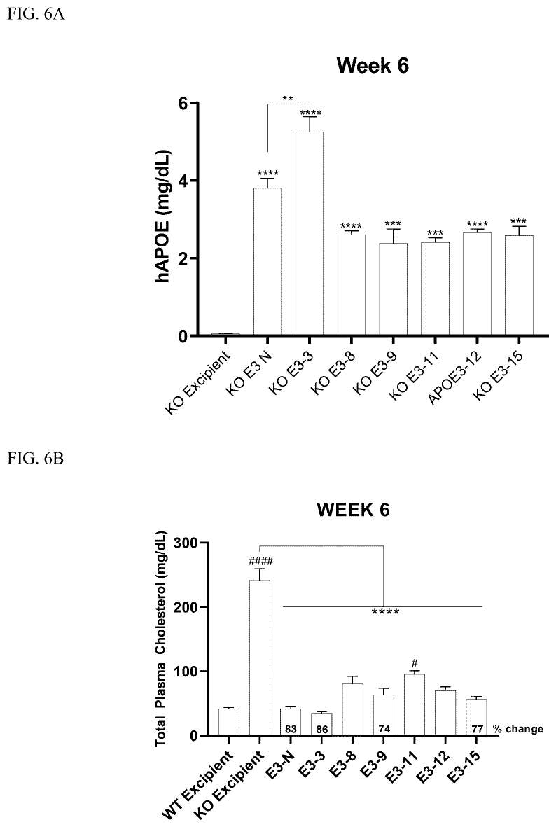

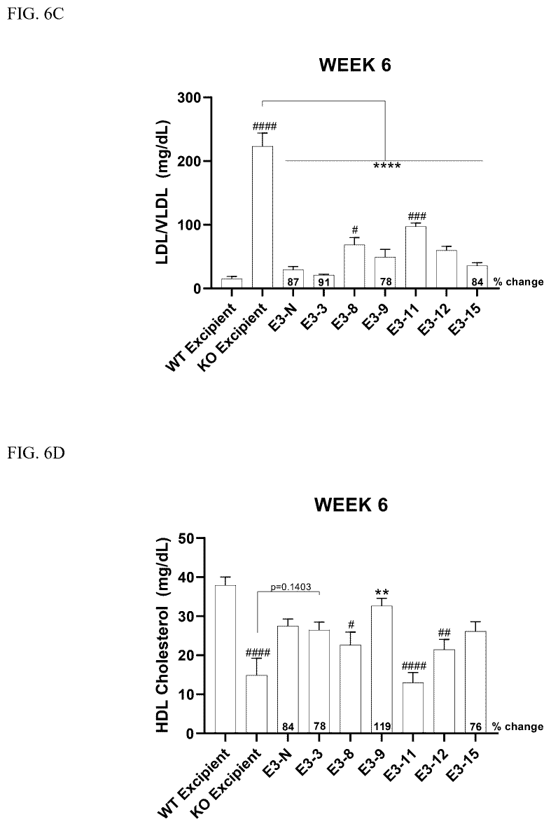

FIGS. 6A-6E illustrate ApoE3 transgene expression from different constructs, and the impact of transgene expression on cholesterol, at 6 weeks. FIG. 6A illustrates hAPOE levels, FIG. 6B illustrates total cholesterol, FIG. 6C illustrates the LDL/VLDL cholesterol ratio, FIG. 6D illustrates HDL cholesterol, and FIG. 6E illustrates the total cholesterol/HDL ratio.

### Example 3: Liver Expression

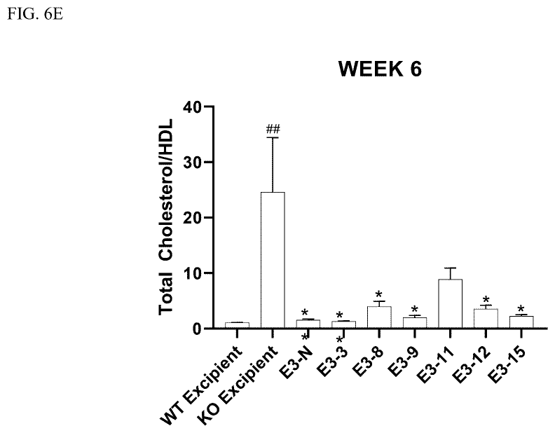

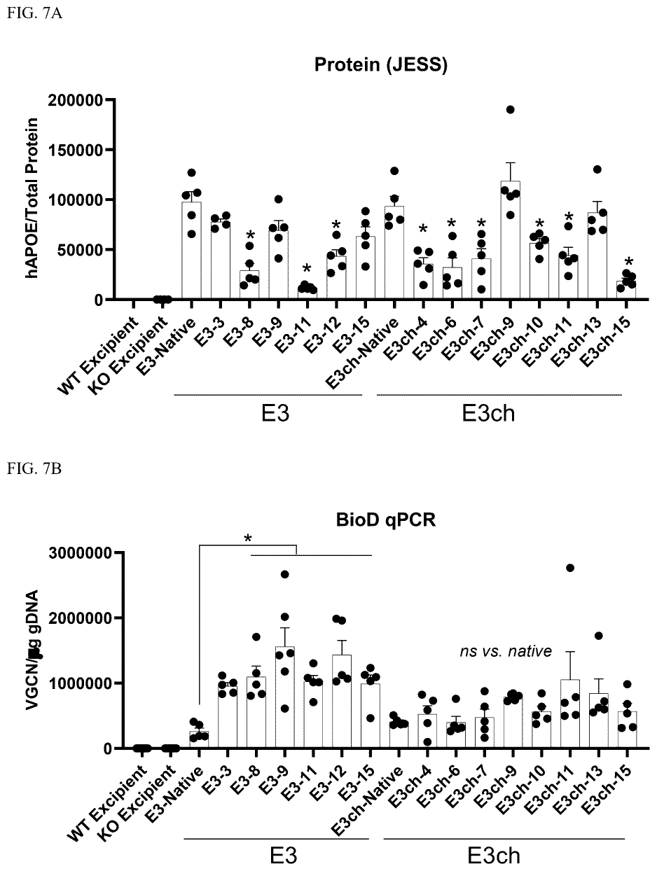

Liver tissue samples were obtained from mice treated in Example 2 at six weeks, and hAPOE/total protein (FIG. 7A) and vector genome copy number (VCGN)/ug gDNA (FIG. 7B) were determined. hAPOE levels was determined by JESS. VCGN was assessed qPCR per microgram of gDNA. No immune infiltration of liver tissue was observed by H&E pathology assessment (data not shown).

### Example 4: Atheroma Lesions

The impact of ApoE3(ch) and ApoE3 transgene expression on atheroma lesions was evaluated using wild-type and ApoE knockout mice, and rAAV comprising a transgene expressing ApoE3(ch) or ApoE3. Aortas were evaluated for atheroma lesions by visualizing and quantifying atheroma fatty inclusions 40-weeks post-AAV injection from mice used in Example 1.

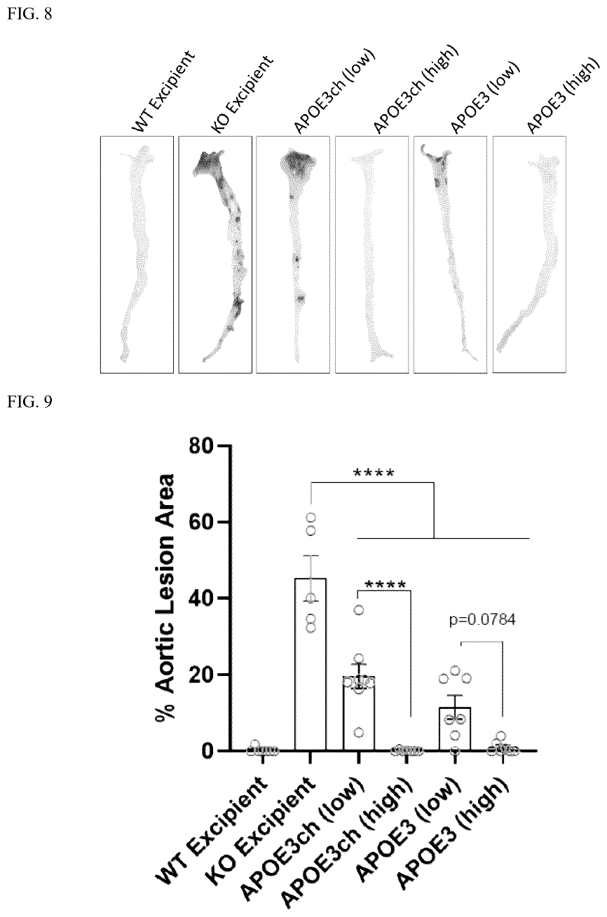

Aortas were collected 40-weeks post-AAV injection by immersion fixation in paraformaldehyde (4%) for 24 hours before transferring to a sucrose gradient. Aortas were washed in dPBS, equilibrated in 60% isopropanol, and atheroma fatty inclusions stained with Oil Red O. Aortas were whole-mount dissected in an enface preparation for imaging, representative back and while images from the different groups is provided in FIG. 8. FIG. 9 illustrates quantification of percent lesion area, where each dot on the graph is a data from a mouse.

### Example 5: Anti-Inflammatory Effects of ApoE3 and ApoE3ch Transgene Expression

The mice treated in Example 1 were further evaluated to determine the anti-inflammatory effects of rAAV comprising an APOE3ch or an APOE transgene. Plasma was collected monthly from APOE KO mice treated with rAAV. Plasma from weeks 20, 24, and 28 (n=5/group) were combined to provide sufficient volume to run a Mouse Cytokine panel manufactured by MesoScale Diagnostics to measure inflammatory markers. The MesoScale Diagnostics Mouse Cytokine panel measures 19 inflammatory markers (V-PLEX Mouse Cytokine 19-Plex Kit). Six inflammatory plasma proteins were elevated in excipient (vehicle control) treated APOE KO mice vs. WT mice and normalized with APOE3(ch) gene therapy.

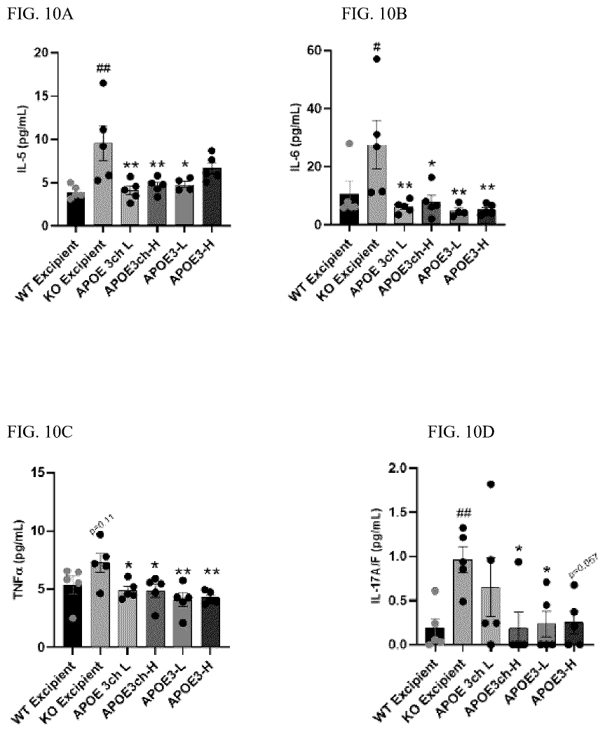

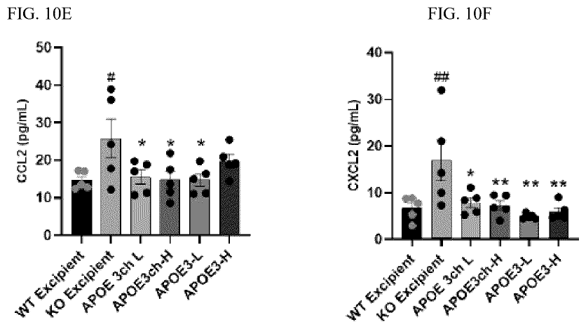

The results are shown in FIGS. 10A-10F. APOE3ch L and APOE3-L refer to KO mice provided a low dose of 3e12 vg/kg rAAV. APOE3ch-H and APOE3-H refers to KO mice provided a high dose of 1e13 vg/kg rAAV. FIG. 10A illustrates IL-5 levels, FIG. 10B illustrates IL-6 levels, FIG. 10C illustrates TNF-α levels, FIG. 10D illustrates IL-17A/F levels, FIG. 10E illustrates CCL2 levels, and FIG. 10F illustrates CXCL2. One-way ANOVA, Dunnett's post-hoc test. #p<0.05, ##p<0.01 vs. WT Excipient, *p<0.05, **p<0.01 vs. KO Excipient.

### Example 6: Effects of ApoE3 and ApoE3ch Transgene Expression on GFAP

The mice treated in Example 1 were further evaluated to determine the effect of effects of rAAV comprising an APOEch or an APOE transgene on glial fibrillar acidic protein (GFAP) levels. GFAP levels in different brain regions were measured by protein quantification by capillary electrophoresis (JESS, Protein Simple) or immunofluorescence quantification. Brain samples were collected 40-weeks following APOE3(ch) gene therapy treatment of APOE KO mice.

The results are shown in FIGS. 11A-11D. APOE3ch-H and APOE3-H refers to KO mice provided a high dose of 1e13 vg/kg rAAV. APOE3ch L and APOE3-L refer to KO mice provided a low dose of 3e12 vg/kg rAAV. FIG. 11A illustrates GFAP/total protein in the cortex determined by JESS. FIG. 11B illustrates GFAP/total protein in the hippocampus determined by JESS. FIG. 11C illustrates % area of GFAP in whole brain determined by immunofluorescence quantification. FIG. 11D illustrates GFAP % area in the hippocampus determined by immunofluorescence quantification.

Immunofluorescence staining for brain inflammatory astrocyte marker GFAP was performed with Anti-GFAP Antibody (AB5541, Millipore; 1:500) and percent GFAP area quantified using HALO® image analysis software (IndicaLabs), demonstrated an increase in GFAP immunoreactivity throughout the brain, and in the hippocampus of APOE KO mice. Elevated GFAP immunoreactivity was decreased in a dose-dependent manner in mice administered APOE3(ch) and APOE3 gene therapy. Similar results were observed by GFAP protein and protein quantification in the cortex and hippocampus.

### Example 7: Effects of ApoE3 and ApoE3ch Transgene Expression on Pre-Synaptic and Postsynaptic Proteins

The mice treated in Example 1 were further evaluated to determine the effect of rAAV comprising an APOE3ch or an APOE3 transgene on pre-Synaptic and postsynaptic proteins. Brain lysates were prepared from mice 40-weeks following APOE3(ch) gene therapy, or vehicle treatment. To determine whether APOE3(ch) and APOE transgene expression has an impact on the number of neuronal connections within the brain, pre-synaptic and postsynaptic proteins synaptophysin and PSD-95, respectively, were quantified by capillary electrophoresis (JESS, Protein Simple) in the cortex, and hippocampus.

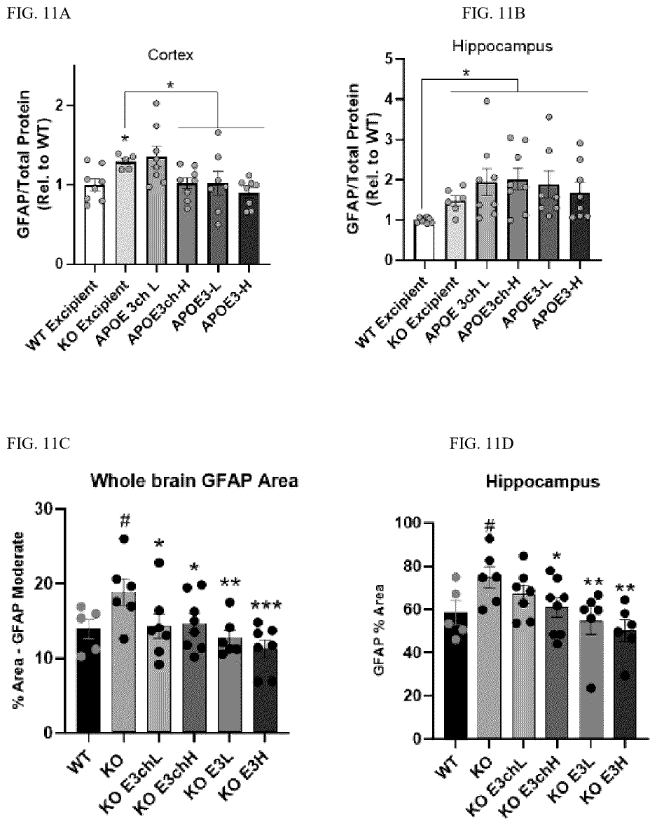

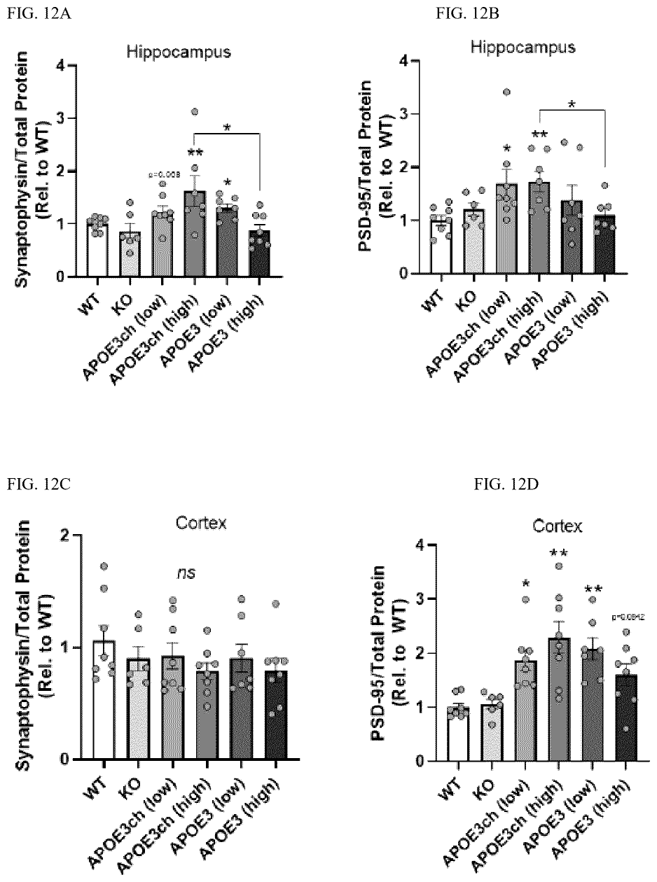

The results are shown in FIGS. 12A-12D. APOE3ch (low) and APOE3 (low) refer to KO mice provided a low dose of 3e12 vg/kg rAAV. APOE3ch (high) and APOE3 (high) refer to KO mice provided a high dose of 1e13 vg/kg rAAV. FIG. 12A illustrates synaptophysin/total protein in the hippocampus. FIG. 12B illustrates PSD-95/total protein in the hippocampus. FIG. 12C illustrates synaptophysin/total protein in the cortex. FIG. 12D illustrates PSD-95/total protein in the cortex.

### Example 7: Atheroma Lesions (9 Weeks)

The effect of rAAV comprising an APOE3ch or an APOE3 transgene on atherosclerosis was evaluated by quantified atheroma lesions in ApoE knockout mice (B6.129P2-Apoetm1Unc/J, Jackson Labs, Strain #:002052) treated with the rAAV. Aortas were collected from the 1-year old APOE KO mice, having severe pre-existing atherosclerosis. Recombinant AAV nucleic acid contained an ApoE/hAAT promoter/enhancer functionally coupled to either ApoE3 or ApoE3ch encoding nucleic acid, along with other expression vector components and ITRs, as illustrated in FIG. 1. Table 4 summarizes the ApoE encoding sequences.

TABLE 4

| | SEQ ID NO: |
|---|---|
| APOE3ch-N | 1 |
| APOE3-N | 2 |
| APOE3-3 | 20 |
| APOE3ch-9 | 11 |

Aortas from the baseline group served as a measurement of atherosclerosis prior to treatment. Remaining mice were treated with rAAV comprising native ApoE3 (ApoE3-N) and native ApoE3ch (ApoE3ch-N) transgene sequences as well as CpG-0, codon optimized variants APOE3-3 and APOE3ch-9. The rAAV were administered at two doses (2e11 vg/mouse or 2e12 total vg/mouse) for 9 weeks followed by collection of Aortas, staining atherosclerosis pathology with Oil Red O, and quantifying the % Lesion volume/Aorta.

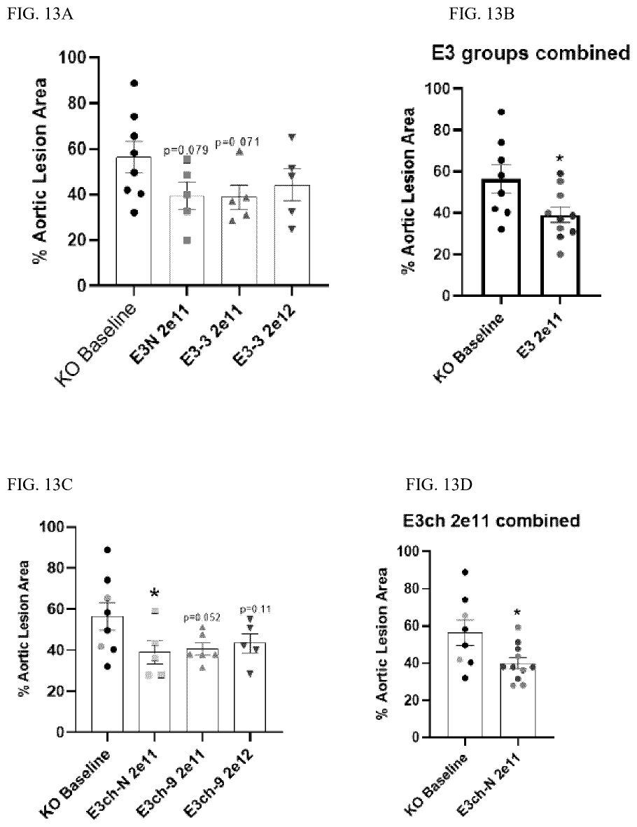

FIGS. 13A-13D illustrates quantification of percent lesion area, where each dot on the graph represents one mouse. FIG. 13A illustrates % aortic lesion area in KO mice and KO mice administered different transgene sequences encoding for ApoE as follows: ε3N (native ApoE3) 2e¹¹ vg/kg, ε3-3 (ApoE3-3) 2e¹¹ vg/kg, and ε3-3 (ApoE3-3) 2e¹² vg/kg. FIG. 13B illustrates the KO baseline from FIG. 13A and a combination of the ε3N (native ApoE3) 2e¹¹ vg/kg and ε3-3 (ApoE3-3) 2e¹¹ vg/kg groups from FIG. 13A. FIG. 13C illustrates % aortic legion area in KO mice and KO mice administered different transgene sequences encoding for the following: ε3chN (native ApoE3ch) 2e¹¹ vg/kg, ε3ch-9 (ApoE3ch-9) 2e¹¹ vg/kg, and ε3ch-9 (ApoE3ch-9) 2e¹² vg/kg. FIG. 13D illustrates the KO baseline from FIG. 13C and a combination of ε3chN (native ApoE3ch) 2e¹¹ vg/kg and ε3ch-9 (ApoE3ch-9) 2e¹¹ vg/kg.

APOE3(ch) and APOE(ch) gene therapy of all constructs demonstrated greater than 17% reduction compared to baseline groups in pre-existing atherosclerosis pathology supporting the reversal of atherosclerosis is possible with APOE3(ch) gene therapy.

### Example 8: Cognitive Studies

APOE KO mice have been reported to have learning and memory cognitive deficits at ≥1 year of age. One year old APOE KO mice (B6.129P2-Apoetm1Unc/J, Jackson Labs, Strain #:002052) were tested on a Novel Object Recognition (NOR) memory test (Antunes and Biala G, Cogn Process. 2012 May; 13(2):93-110), and found to have significantly decreased recognition memory prior to APOE3 and APOE3ch gene therapy treatment. The test measures the memory of the familiar object as measured by the preference for a novel object. Preference for a novel object (>50%) of time is normal cognitive behavior indicating intact long-term recognition memory in mice.

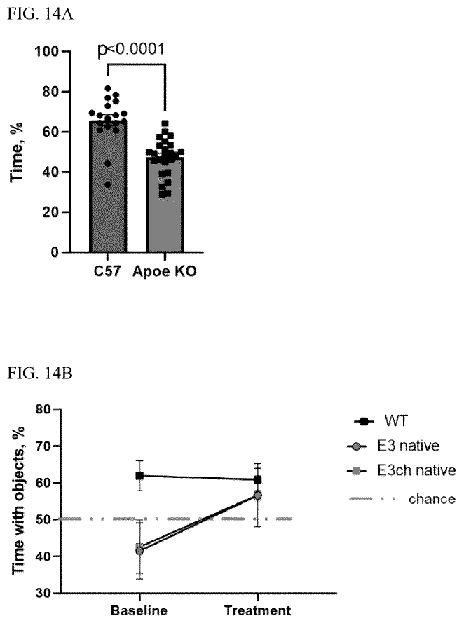

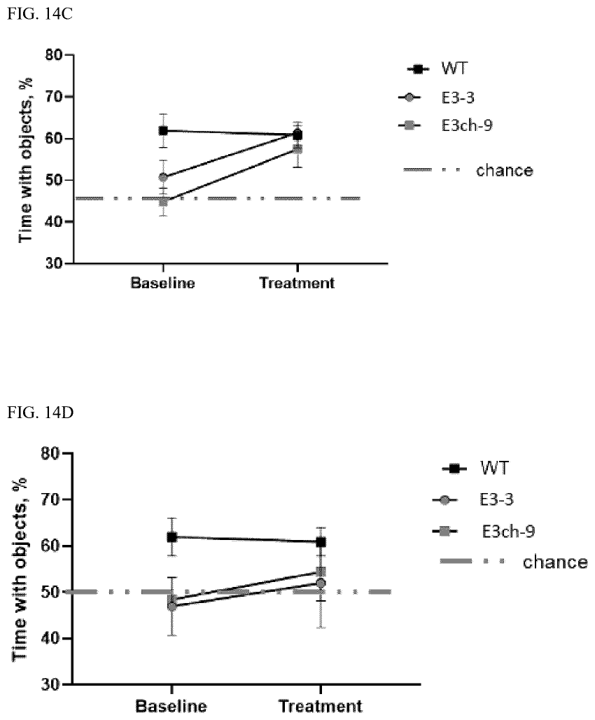

Native ApoE3 (E3 native), native ApoE3ch (E3ch native), ApoE3-3 (E3-3) and ApoE3ch-9 (E3ch-9) were administered intravenously via tail vein at a low dose of 2e11 vg/mouse or high dose of 2e12 total vg/mouse. The constructs are described in Table 4. FIG. 14A illustrates NOR results for C57BL/6 and ApoE knockout mice (*p<0.05, unpaired 2-tailed t-test). FIG. 14B illustrates the NOR results administering native ApoE and ApoE(ch) sequences. FIG. 14C illustrate NOR results with a low dose of rAAV encoding CpG-0 ApoE3 (E3-3) and ApoE(Ech-9) rAAV FIG. 14D illustrate NOR results with a high dose of rAAV encoding CpG-0 ApoE (E3-3) and ApoE3ch (Ech-9) rAAV NOR memory performance was completely restored to age-matched wild-type (WT) mouse performance 5-weeks following a single intravenous via tail vein administration of native APOE3 and APOE3ch sequences, as well as CpG-0, codon optimized variants E3-3 and E3ch-9 at two doses (2e11 vg/mouse or 2e12 total vg/mouse).

### Example 9: Additional ApoE3 Constructs

The ability of different codon optimized CpG reduced transgenes encoding ApoE were evaluated. The transgenes were inserted into a plasmid, which were transfected in triplicate into AMLL-12 cells. ApoE3 and antigen levels were measured in cell culture supernatants at 72 hours post-transfection. ApoE3 levels were assayed by ELISA and plotted as the mean+/−the standard deviation.

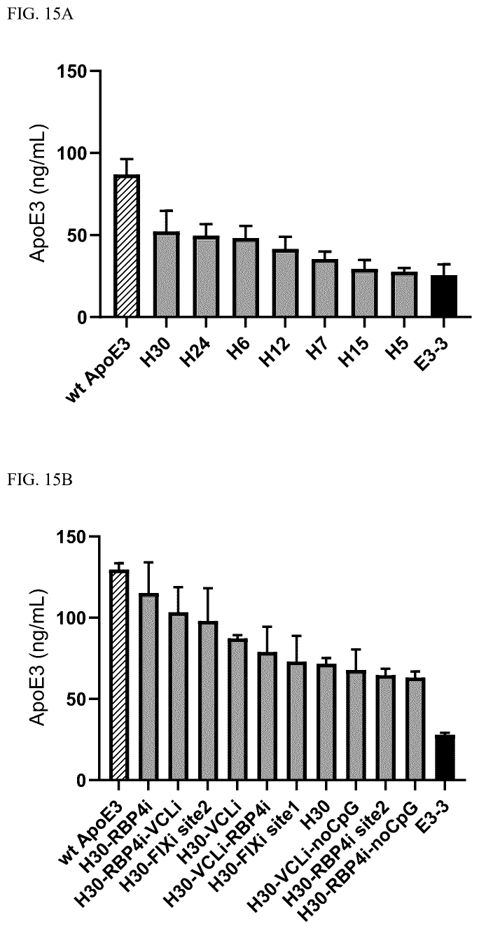

FIG. 15A provides a bar graph showing the performance of codon-optimized, CpG-reduced cDNAs encoding ApoE3 and the CpG free construct ApoE3-3. The different constructs and sequence identity to some fully CpGs constructs also described in the application is provided in Table 5.

TABLE 5

| | SEQ | |
|---|---|---|
| | ID NO: | Precent Identity |
| wtApoE3 | 1 | |
| H30 | 130 | 97.0% identical to ApoE3ch-46 (SEQ ID NO: 101) |
| H24 | 129 | 97.5% identical to ApoE3ch-45 (SEQ ID NO: 100) |
| H6 | 125 | 98.2% identical to ApoE3ch-41 (SEQ ID NO: 96) |
| H12 | 127 | 97.8% identical to ApoE3ch-43 (SEQ ID NO: 98) |
| H7 | 126 | 98.4% identical to ApoE3ch-42 (SEQ ID NO: 97) |
| H15 | 128 | 97.6% identical to ApoE3ch-44 (SEQ ID NO: 99) |
| H5 | 124 | 98.8% identical to ApoE3ch-40 (SEQ ID NO: 95) |
| E3-3 | 20 | |

FIG. 15B provides a bar graph showing the performance of codon-optimized ApoE3 cDNA functionalized by the addition of intron sequences. Introns were inserted into and the signal peptide at codon 15 G/GC (site 1), and/or at codon 79 AG/G (site 2). In those cases where one intron is indicated and no site provided, the intron was inserted into site 1. Reference to intron RBP4i, VCLi, and FIXi, without the indication of no CpG indicates the wild-type sequence. VCLi-noCpG refers to the intron of SEQ ID NO: 121. RBP4i-noCpG refers to the intron of SEQ ID NO: 120. For comparison purposes, the non-intron containing codon-optimized ApoE3-3 and H30 variants were included as benchmarks. ApoE3 levels were assayed by ELISA and plotted as the mean+/−the standard deviation.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

I/We claim:

1. A polynucleotide comprising an ApoE encoding nucleotide sequence having at least 85% sequence identity to the sequence of any of SEQ ID NOs: 63-67 or nucleotides 55-951 of SEQ ID NOs: 95-105 and 124-130, wherein the polynucleotide encodes an ApoE3 related protein comprising an amino acid sequence at least 90% identical to SEQ ID NO: 32, wherein the protein comprises a cysteine in a location corresponding to amino acid 112 of SEQ ID NO: 32 and an arginine at a location corresponding to amino acid 158 of SEQ ID NO: 32, wherein the ApoE encoding nucleotide sequence optionally comprises one or more introns.

2. The polynucleotide of claim 1, wherein the ApoE encoding nucleotide sequence comprises at least one intron.

3. The polynucleotide of claim 1, wherein the ApoE encoding nucleotide sequence does not comprise any intron.

4. The polynucleotide of any one of claims 1-3, wherein the ApoE encoding nucleotide sequence has at least 95% sequence identity to nucleotides 55-951 of any of SEQ ID NOs: 3-10, 12-14, 16-19, 21-25, 27-30, 95-105 and 124-130.

5. The polynucleotide of any one of claims 1-3, wherein the ApoE encoding nucleotide sequence has at least 95% sequence identity to any of SEQ ID NOs: 63-67.

6. The polynucleotide of any one of claims 1-5, wherein the ApoE3 related protein further comprises a serine at a position corresponding to amino acid 136 of SEQ ID NO: 32.

7. The polynucleotide of any one of claims 1-5, wherein the ApoE3 related protein comprises the amino acid sequence of SEQ ID NO: 32.

8. The polynucleotide of any one of claims 1-5, wherein the ApoE3 related protein comprises the amino acid sequence of SEQ ID NO: 33.

9. The polynucleotide of any one of claims 1-3, wherein the ApoE encoding nucleotide sequence has a sequence identity of at least 95% SEQ ID NO: 63 and the ApoE3 related protein comprises the amino acid sequence of SEQ ID NO: 33.

10. The polynucleotide of any one of claims 1-9, wherein the ApoE3 related protein further comprises a 5′ signal peptide and the ApoE3 encoding nucleic acid sequence further comprises a nucleotide sequence encoding the signal peptide.

11. The polynucleotide of claim 10, wherein the signal peptide has at least 90% identity to the amino acid sequence of any of SEQ ID NOs: 36, 42, 44, 46, 48, 50, 52, 54, 56, and 68-71.

12. The polynucleotide of claim 11, wherein the nucleotide sequence encoding the signal peptide comprises at least 90% identity to the sequence of any of SEQ ID NOs: 37-41, 43, 45, 47, 49, 51, 53, 55, 57 and 72-75.

13. The polynucleotide of any one of claims 1-12, wherein the ApoE encoding nucleotide sequence comprises a sequence having a sequence identity of at least 95% to the sequence of any of SEQ ID NOs: 20, 26, 31, 11, 15, and 95-105.

14. The polynucleotide of any one of claims 1-13, wherein the ApoE3 related protein comprises the sequence of SEQ ID NO: 34 or SEQ ID NO: 35.

15. The polynucleotide of claim 1, wherein the ApoE encoding nucleotide sequence comprises a sequence of (i) nucleotides 55 to 951 of any of SEQ ID NOs: 3-10, 12-14, 16-19, 21-25, 27-30, 95-105 and 124-130; (ii) nucleotides 55 to 954 of any of SEQ ID NOs: 3-10, 12-14, 16-19, 21-25, 27-30, 95-105 and 124-130; (iii) nucleotides 1-897 of any of SEQ ID NOs: 63-67; (iv) any of SEQ ID NOs: 63-67; (v) nucleotides 1 to 951 of any of SEQ ID NOs: 3-31, 95-105 and 124-140; or (vi) any of SEQ ID NOs: 3-31, 95-105 and 124-130.

16. The polynucleotide of claim 1, wherein the ApoE encoding nucleotide sequence comprises a sequence at least 95% identical to (i) nucleotides 55 to 951 of any of SEQ ID NOs: 3-10, 12-14, 16-19, 21-25, 27-30, 95-105 and 124-130; (ii) nucleotides 55 to 954 of any of SEQ ID NOs: 3-10, 12-14, 16-19, 21-25, 27-30, 95-105 and 124-130; (iii) nucleotides 1-897 of any of SEQ ID NOs: 63-67; (iv) any of SEQ ID NOs: 63-67; (v) nucleotides 1 to 951 of any of SEQ ID NOs: 3-31, 95-105 and 124-130; or (vi) any of SEQ ID NOs: 3-31, 95-105, and 124-130; and the ApoE3 related protein comprises an amino acid sequence of SEQ ID NOs: 34 or 35.

17. The polynucleotide of claim 2, wherein the ApoE encoding nucleotide sequence comprises 5′ to 3′:
(a) a first exon corresponding to nucleotides 1-43 of SEQ ID NO: 1, wherein the first exon has a sequence identity of at least 85% to nucleotides 1-43 of any of SEQ ID NOs: 3-31, 95-105 and 124-130, provided that the terminal 3′ nucleotide of the first exon is G;
(b) a first intron at a position corresponding to between nucleotides 43 and 44 of SEQ ID NO: 1;
(c) a second exon corresponding to nucleotides 44-236 of SEQ ID NO: 1; wherein the second exon has a sequence identity of at least 85% to nucleotides 44-236 of any of SEQ ID NOs: 3-31, 95-105, and 124-130, provided that the terminal 5′ nucleotides of the second exon is G and the terminal 3′ nucleotides of the second exon is AG;
(d) a second intron at a position corresponding to between nucleotides 236 and 237 of SEQ ID NO: 1; and
(e) a third exon corresponding to nucleotides 237-951 of SEQ ID NO: 1, wherein the third exon has a sequence identity of at least 85% to nucleotides 237-951 of any of SEQ ID NOs: 3-31, 95-105, and 124-130, provided that the terminal 5′ nucleotide of the third exon is G;
wherein the first, second and third exons together encode the ApoE3 related protein, wherein the ApoE related protein comprises an amino acid sequence at least 95% identical to SEQ ID NO: 35.

18. The polynucleotide of claim 17, wherein the first intron comprises a sequence with a sequence identity of at least 50% to any of SEQ ID NOs: 119-122 and the second intron independently comprises a sequence with a sequence identity of at least 50% to any of SEQ ID NOs: 119-121.

19. The polynucleotide of claim 18, wherein said first intron consists of a sequence of any of SEQ ID NOs: 119-121 or a sequence differing from any of SEQ ID NOs: 119-121 by 1 to 10 nucleotides and the second intron independently consists of a sequence of any of SEQ ID NOs: 119-121 or a sequence differing from any of SEQ ID NOs: 119-121 by 1 to 10 nucleotides.

20. The polynucleotide of any one of claims 17-19, wherein,
the first exon has a sequence identity of at least 95% to nucleotides 1-43 of any of SEQ ID NOs: 11, 20, or 95-105;
the second exon has a sequence identity of at least 95% to nucleotides 44-236 of any of SEQ ID NOs: 11, 20, or 95-105; and
the third exon has a sequence identity of at least 95% to nucleotides 237-951 of any of SEQ ID NOs: 11, 20, or 95-105;
wherein the ApoE related protein comprises has a sequence identity of at least 98% to SEQ ID NO: 35.

21. The polynucleotide of claim 20, wherein the ApoE encoding nucleotide sequence comprises a sequence with a sequence identity of at least 95% to any of SEQ ID NOs: 106-115.

22. The polynucleotide of claim 21, wherein the ApoE related protein comprises the sequence of SEQ ID NOs: 34 or 35.

23. The polynucleotide of claim 22, wherein the ApoE encoding nucleotide sequence comprises the sequence of any of SEQ ID NOs: 106-115.

24. The polynucleotide of any one of claims 17-21, wherein the ApoE3 related protein comprises the sequence of SEQ ID NOs: 34 or 35.

25. The polynucleotide of any one of claims 1-24, wherein the ApoE3 encoding nucleotide sequence contains 0 to 10 CpGs.

26. The polynucleotide of any one of claims 1-25, wherein the polynucleotide is an expression cassette comprising one or more expression control elements operably linked to the ApoE encoding nucleotide sequence.

27. The polynucleotide of claim 26, wherein the expression cassette comprises a 5′ promoter operably linked to the ApoE3 encoding nucleotide sequence and a 3′ polyadenylation site operably linked to the ApoE3 encoding nucleotide sequence.

28. The polynucleotide of claim 27, wherein the expression cassette comprises 5′ to 3′, operably linked to the ApoE3 related encoding nucleotide sequence, a promoter or promoter/enhancer, an intron, a Kozak sequence, the ApoE3 encoding nucleic acid sequence, and a polyadenylation signal.

29. The polynucleotide of claims 27 or 28, wherein the promoter is a liver specific promoter.

30. The polynucleotide of any of claims 27-29, comprising an promoter/enhancer, wherein the enhancer has a sequence identity of at least 95% with SEQ ID NO: 58.

31. The polynucleotide of any of claims 27-30, wherein the promoter is a hAAT promoter.

32. The polynucleotide of claims 27 or 28, wherein the promoter provides for expression in CNS cells.

33. The polynucleotide of claim 32, wherein the expression cassette further comprises one or more miRNA target sequences to inhibit dorsal root ganglion, liver, or immune cell expression.

34. The polynucleotide of any one of claims 28-33, wherein the intron comprises the sequence of SEQ ID NO: 60, and the polyadenylation signal comprises the sequence of SEQ ID NO: 61 or SEQ ID NO: 62.

35. The polynucleotide of any one of claims 26-34, wherein the expression cassette further comprises one or more inhibitory nucleic acids selectively targeting ApoE2, ApoE3, or ApoE4 encoding nucleic acid.

36. The polynucleotide of claim 35, wherein the inhibitory nucleic acid selectively targets ApoE4 encoding nucleic acid.

37. The polynucleotide of any one of claims 26-36, wherein the expression cassette contains up to about 10% CpGs.

38. The polynucleotide of any one of claims 26-37, wherein the expression cassette comprises a nucleotide sequence having a sequence identity of at least 95% to any of SEQ ID NOs: 76-80.

39. The polynucleotide of any one of claims 1-38, wherein the polynucleotide is DNA.

40. The polynucleotide of any one of claims 26-39, wherein the expression cassette comprises a nucleotide sequence having a sequence identity of at least 95% to a modified SEQ ID NO: 76, wherein SEQ ID NO: 76 is modified by replacing nucleotides 1295-2248 with any of SEQ ID NOs: 3-10, 12-14, 16-19, 21-25, 27-30, 95-115 and 124-130.

41. A recombinant viral vector nucleic acid comprising the polynucleotide of any one of claims 26-40 and a 5′ and/or 3′ viral elements providing for viral packaging and/or replication.

42. The recombinant viral vector nucleic acid of claim 41, wherein the recombinant viral vector nucleic acid is DNA and comprises an adeno-associated virus (AAV) inverted repeat (ITR) flanking the 5′ terminus of the polynucleotide and/or an AAV ITR flanking the 3′ terminus of the polynucleotide.

43. The recombinant viral vector nucleic acid of claim 42, wherein the 5′ and/or 3′ viral elements are each selected from the group consisting of AAV1, AAV2, AAV3, AAV4, AAV5, AAV6, AAV7, AAV8, AAV9, AAV10, AAV11, AAV12, AAVrh.10, AAVrh.74 and AAV3B.

44. The recombinant viral vector nucleic acid of claim 43, wherein the 5′ ITR comprises a sequence with a sequence identity of at least 95% to SEQ ID NO: 81, and the 3′ ITR comprises a sequence with a sequence identity of at least 95% to SEQ ID NO: 82.

45. The recombinant viral vector nucleic acid of any one of claims 41-44, wherein the recombinant viral vector nucleic acid contains up to about 10% CpGs.

46. A gene delivery vehicle, wherein the gene delivery vehicle is a viral or a non-viral vector comprising the polynucleotide of any one of claims 1-40 or the recombinant viral vector nucleic acid of any one of claims 41-45.

47. The gene delivery vehicle of claim 46, wherein the gene delivery vehicle is the viral vector.

48. The gene delivery vehicle of claim 47, wherein the viral vector is a recombinant AAV, a recombinant lentivirus vector, or a recombinant adenovirus vector.

49. The gene delivery vehicle of claim 48, wherein the viral vector is the recombinant AAV, and the recombinant AAV vector comprises a capsid comprising a VP1, VP2 or VP3 having a sequence identity of at least 90% to a VP1, VP2 or VP3 of any of AAV1, AAV2, AAV3, AAV4, AAV5, AAV6, AAV7, AAV8, AAV9, AAV10, AAV11, AAV12, AAVrh.74, AAV3B, AAV-2i8, AAVrh.10, AAVrh.8, AAVHSC, AAV-B1, AAV-AS, AAV1/rh.10, SEQ ID NO: 83 and SEQ ID NO: 84.

50. The gene delivery vehicle of claim 49, wherein the capsid is an AAV1, AAV2, AAV3, AAV4, AAV5, AAV6, AAV7, AAV8, AAV9, AAV10, AAV11, AAV12, AAVrh.74, AAV3B, AAV-2i8, AAVrh.10, AAVrh.8, AAVHSC, AAV-B1, AAV-AS, AAV1/rh.10 capsid; or the capsid comprises VP1 of SEQ ID NO: 83 or SEQ ID NO: 84.

51. The gene delivery vehicle of claim 46, wherein the gene delivery vehicle is the non-viral vector.

52. The gene delivery vehicle of claim 51, wherein the non-viral vector is a nanoparticle selected from the group consisting of a lipid nanoparticle (LNP), a polymeric nanoparticle, a lipid polymer nanoparticle (LPNP), a protein or peptide-based nanoparticle, a DNA dendrimer or DNA-based nanocarrier, a carbon nanotube, a microparticle, a microcapsule, an inorganic nanoparticle, a peptide cage nanoparticle, and an exosome.

53. The gene delivery of vehicle of claim 52, wherein the non-viral vector is an LNP or LPNP.

54. A pharmaceutical composition comprising the polynucleotide of any one of claims 1-40, the recombinant viral vector nucleic acid of any one of claims 41-45, or the gene delivery vehicle of any one of claims 46-53 and a pharmaceutically acceptable carrier.

55. The pharmaceutical composition of claim 54, wherein the composition comprises recombinant AAV and empty AAV capsids, wherein the ratio of the empty AAV capsids to the recombinant AAV is 100:1 to 1:100.

56. A method of treating a subject to reduce cholesterol, reduce LDL/VLDL, increase HDL or reduce total cholesterol/HDL ratio; or treat or reduce the likelihood of hypercholesteremia, Type III Familial hyperlipoproteinemia, Familial Hypercholesterolemia, dementia, post-stent restenosis, atherosclerosis, coronary heart disease or Alzheimer's disease; comprising administering to the subject an effective amount of the polynucleotide of any one of claims 1-40, the recombinant viral vector nucleic acid of any one of claims 41-45, the gene delivery vehicle of any one of claims 46-53, or the pharmaceutical composition of claims 54 or 55.

57. The method of claim 56, wherein the method reduces cholesterol, reduces the LDL/VLDL, increases HDL or reduces the total cholesterol/HDL ratio in a subject in need thereof.

58. The method of claim 57, where the subject has hypercholesteremia.

59. The method of claim 56, wherein said method treats or reduces the likelihood of hypercholesteremia, Type III Familial, hyperlipoproteinemia, vascular dementia, frontotemporal dementia, cerebral amyloid angiopathy, post-stent restenosis, atherosclerosis, coronary heart disease or Alzheimer's disease in a subject.

60. The method of any one of claims 56-59, wherein the subject is a statin hyporesponder or a statin intolerant.

61. The method of claim 56, wherein the method treats or reduces the likelihood of Alzheimer's disease in a subject.

62. The method of claim 61, wherein the administering comprises intraparenchymal, intracisternal or intraventricular administration.

63. The method of claim 61, wherein the polynucleotide, the recombinant viral vector nucleic acid, the gene delivery vehicle, or the pharmaceutical composition is administered intravenously.

64. The method of any of claims 56-63, wherein the subject has at least one ApoE4 allele, is an EpoE4 homozygote, or is a PSEN1 mutation carrier.

65. The method of claim 64, further comprises administering an effective amount of an inhibitory nucleic acid to decrease native ApoE4 expression in the subject.

66. The method of any one of claims 56-65, wherein the subject is a human.

67. An AAV vector genome plasmid comprising recombinant viral nucleic acid of any one of claims 41-45.

68. The AAV genome plasmid of claim 67, wherein said plasmid lacks rep and cap genes.

69. A method of producing a rAAV vector comprising the step of culturing an rAAV production cell line comprising rAAV helper virus activity, wherein the genome of said production cell comprises the recombinant viral vector nucleic acid of any one of claims 41-45, a rep gene and a cap gene, wherein said rAAV vector is produced.

70. A method of producing rAAV vector comprising the step of culturing an rAAV permissive cell comprising the AAV genome plasmid of claims 67 or 68, wherein said rAAV permissive cell further comprises (a) rep and cap genes provided either as part of the cell genome and/or by one or more separate plasmids, and (b) helper virus activity provided by the cell genome and/or provided by one or more separate plasmids.

71. The method of claim 70, wherein said rAAV permissive cell is a packaging cell, wherein the genome of said packaging comprises a cap gene and a rep gene.

72. The method of claim 70, wherein either (a) said rep gene, said cap gene and said helper activity is provided in a single plasmid or (b) said rep gene and said cap gene is provided by a rep cap plasmid and said helper activity is provided by a helper plasmid.

73. A method of obtaining an rAAV vector comprising the steps of (a) producing the rAAV using the method of anyone of claims 70-72 and (b) purifying the rAAV.

74. A polynucleotide comprising a sequence with at least 50% sequence identity to the sequence of any of SEQ ID NOs: 119-121, wherein the polynucleotide has 0-5 CpGs.

75. The polynucleotide of claim 74, where the polynucleotide comprises the sequence of any of SEQ ID NOs: 119-121, or a sequence differing from any of SEQ ID NOs: 119-121 by 1 to 10 nucleotides, wherein the polynucleotide has no CpGs.

76. The polynucleotide of claim 75 consisting of the sequence of any of SEQ ID NOs: 119-121.
